(19) United States
(12) Patent Application Publication
(10) Pub. No.: US 2023/0183201 A1
(43) Pub. Date: Jun. 15, 2023

(54) COMPOUND FOR ORGANIC ELECTRIC ELEMENT, ORGANIC ELECTRIC ELEMENT USING SAME, AND ELECTRONIC DEVICE HAVING SAME

(71) Applicant: DUK SAN NEOLUX CO., LTD., Cheonan-si, Chungcheongnam-do (KR)

(72) Inventors: Se Hoon LEE, Cheonan-si, Chungcheongnam-do (KR); Hyung Dong LEE, Cheonan-si, Chungcheongnam-do (KR); Dae Hwan OH, Cheonan-si, Chungcheongnam-do (KR); Won Sam KIM, Cheonan-si, Chungcheongnam-do (KR); Sun Hee LEE, Cheonan-si, Chungcheongnam-do (KR); Soung Yun MUN, Cheonan-si, Chungcheongnam-do (KR); Jeong Seok KIM, Cheonan-si, Chungcheongnam-do (KR)

(73) Assignee: DUK SAN NEOLUX CO., LTD., Cheonan-si, Chungcheongnam-do (KR)

(21) Appl. No.: 17/759,010

(22) PCT Filed: Jan. 19, 2021
(86) PCT No.: PCT/KR2021/000737

§ 371 Date: Jul. 18, 2022

(30) Foreign Application Priority Data
Jan. 20, 2020 (KR) 10-2020-0007340

(51) Int. Cl. C07D 311/78; H01L 51/00; C07D 405/12; C07D 335/10; C07D 409/12; C07D 407/12; H01L 51/50

(52) U.S. Cl. CPC C07D 311/78; C07D 335/10; C07D 405/12; C07D 407/12; C07D 409/12; H01L 51/0061; H01L 51/0064; H01L 51/0067; H01L 51/0072; H01L 51/0073; H01L 51/0074; H01L 51/504; H01L 51/5056; C07B 2200/05

(57) ABSTRACT

Provided are a compound capable of improving the luminous efficiency, stability and lifespan of a device employing the same, an organic electronic element using the same, and an electronic device thereof.

### BACKGROUND

### Technical Field

The present invention relates to a compound for an organic electronic element, an organic electronic element using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electronic element may be classified into a light emitting material and a charge transport material, such as a hole injection material, a hole transport material, an electron transport material, an electron injection material and the like depending on its function.

Lifespan and efficiency are the most problematic in organic electroluminescent device, and as displays become larger, these problems of efficiency and lifespan must be solved. Efficiency, lifespan, and driving voltage are related to each other, and when the efficiency is increased, the driving voltage is relatively decreased, and as the driving voltage is decreased, crystallization of the organic material due to Joule heating generated during driving decreases, and as a result, the lifespan tends to increase.

However, the efficiency cannot be maximized simply by improving the organic material layer. This is because, when the energy level and T1 value between each organic material layer, and the intrinsic properties of the material (mobility, interfacial properties, etc.) are optimally combined, a long lifespan and high efficiency can be achieved at the same time.

Also, in order to solve the problem of light emission in the hole transport layer in recent organic electroluminescent devices, an emitting-auxiliary layer must exist between the hole transport layer and the emitting layer, and it is time to develop different emitting-auxiliary layers according to each emitting layer (R, G, B).

In general, electrons are transferred from the electron transport layer to the emitting layer, and holes are transferred from the hole transport layer to the emitting layer, and excitons are generated by recombination.

However, since the material used for the hole transport layer should have a low HOMO value, most have a low T1 value. As a result, excitons generated in the emitting layer are transferred to the hole transport layer, resulting in charge unbalance in the emitting layer to emit light at the hole transport layer interface.

When light is emitted at the hole transport layer interface, the color purity and efficiency of the organic electronic element are lowered, and the lifespan is shortened. Therefore, it is urgently required to develop an emitting-auxiliary layer having a high T1 value and having a HOMO level between the HOMO energy level of the hole transport layer and the HOMO energy level of the emitting layer.

Furthermore, it is necessary to develop a hole injection layer material that delays the penetration and diffusion of metal oxides from the anode electrode (ITO) into the organic layer, which is one of the causes of shortening the lifespan of organic electronic element, and that has stable characteristics, that is, a high glass transition temperature, even against Joule heating generated during device driving. The low glass transition temperature of the hole transport layer material has a characteristic of lowering the uniformity of the thin film surface during device driving, which is reported to have a significant effect on device lifespan. Moreover, OLED devices are mainly formed by a deposition method, and it is necessary to develop a material that can withstand a long time during deposition, that is, a material with strong heat resistance.

In other words, in order to fully exhibit the excellent characteristics of an organic electronic element, it should be preceded that the material constituting the organic material layer in the device, such as a hole injection material, a hole transport material, a light emitting material, an electron transport material, an electron injection material, emitting auxiliary layer material, etc., is supported by a stable and efficient material. but the development of a stable and efficient organic material layer material for an organic electronic device has not yet been sufficiently made. Therefore, the development of new materials is continuously required.

As a reference prior art document, KR1020130076842 A was used.

### DETAILED DESCRIPTION OF THE INVENTION

### Summary

In order to solve the problems of the above-mentioned background art, the present invention has revealed a compound having a novel structure, and when this compound is applied to an organic electronic element, it has been found that the luminous efficiency, stability and lifespan of the device can be significantly improved.

Accordingly, an object of the present invention is to provide a novel compound, an organic electronic element using the same, and an electronic device thereof.

### Technical Solution

The present invention provides a compound represented by Formula 1.

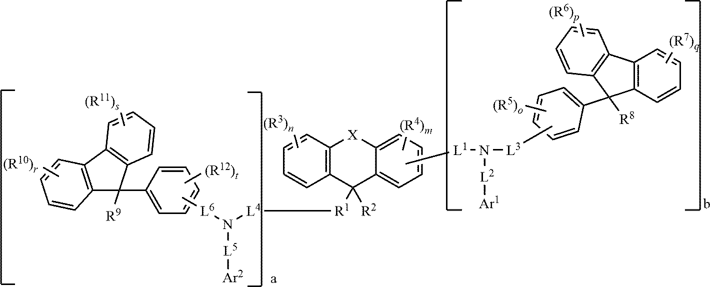

In another aspect, the present invention provides an organic electronic element comprising the compound represented by Formula 1 and an electronic device thereof.

### Effects of the Invention

By using the compound according to the present invention, high luminous efficiency, low driving voltage and high heat resistance of the device can be achieved, and color purity and lifespan of the device can be greatly improved.

### BRIEF DESCRIPTION OF THE DRAWINGS

FIGS. 1 to 3 are exemplary views of an organic electroluminescent device according to the present invention.

FIG. 4 shows a Formula according to an aspect of the present invention.

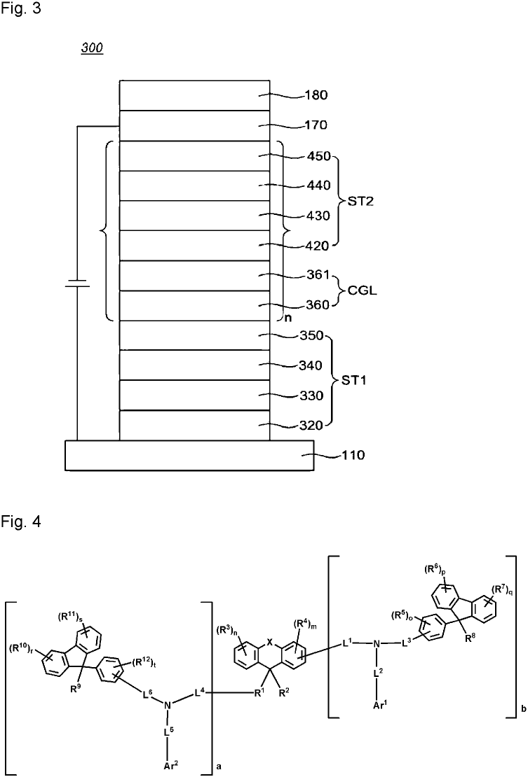

100, 200, 300: organic electronic element
110 : the first electrode
120 : hole injection layer
130 : hole transport layer
140 : emitting layer
150 : electron transport layer
170 : second electrode
180 : light efficiency enhancing Layer
210 : buffer layer
220 : emitting-auxiliary layer
330 : first hole transport layer
340 : first emitting layer
350 : first electron transport layer
360 : first charge generation layer
361 : second charge generation layer
420 : second hole injection layer
430 : second hole transport layer
440 : second emitting layer
450 : second electron transport layer
CGL: charge generation layer
ST1 : first stack
ST2 : second stack

### DETAILED DESCRIPTION

Hereinafter, some embodiments of the present invention will be described in detail.

Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected”, “coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

The terms “aryl group” and “arylene group” used in the present invention have 6 to 60 carbon atoms, respectively, unless otherwise specified, but are not limited thereto. In the present invention, an aryl group or an arylene group means a single ring or multiple ring aromatic, and includes an aromatic ring formed by an adjacent substituent joining or participating in a reaction.

For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of a single ring or multiple ring, and may include heteroaliphadic ring and heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes the following compound.

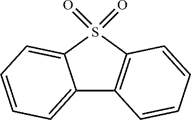 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all hydrogen in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

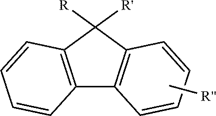

The term “spiro compound”, as used herein, has a ‘spiro union’, and a spiro union means a connection in which two rings share only one atom. At this time, atoms shared in the two rings are called ‘spiro atoms’, and these compounds are called ‘monospiro-’, ‘di-spiro-’ and ‘tri-spiro-’, respectively, depending on the number of spiro atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

Also, unless there is an explicit explanation, the formula used in the present invention is the same as the definition of the substituent by the exponent definition of the following formula.

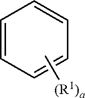

Here, when a is an integer of zero, the substituent R¹ is absent, when a is an integer of 1, the sole substituent R¹ is linked to any one of the carbon constituting the benzene ring, when a is an integer of 2 or 3, each is bonded as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

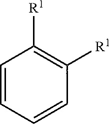

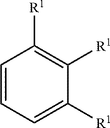

Hereinafter, a compound according to an aspect of the present invention and an organic electronic element including the same will be described.

The present invention provides a compound represented by Formula 1.

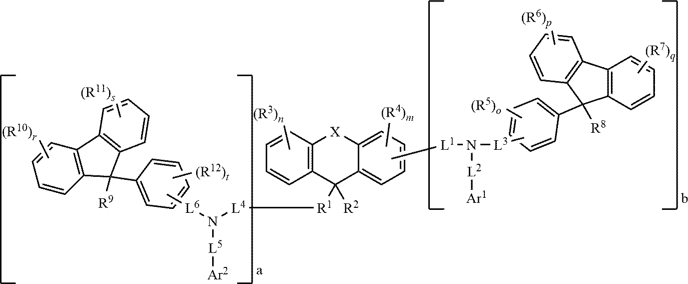

wherein, each symbol may be defined as follows.
1) X is O or S,
2) a and b are each independently 0 or 1, provided that a+b is 1 or more,
3) Ar¹ and Ar² are each independently selected from the group consisting of a C₆-C₆₀ aryl group; a fluorenyl group; a C₂-C₆₀ heterocyclic group including at least one heteroatom of O, N, S, Si or P; and a fused ring group of a C₃-C₆₀ aliphatic ring and a C₆-C₆₀ aromatic ring.
   Wherein in case Ar¹ and Ar² are an aryl group, it may be preferably a C₆-C₃₀ aryl group, and more preferably a C₆-C₂₅ aryl group, for example, it may be phenylene, biphenyl, naphthalene, terphenyl, etc.
   In case Ar¹ and Ar² are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.
   In case Ar¹ and Ar² are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.
4) L¹, L², L³, L⁴, L⁵ and L⁶ are each independently selected from the group consisting of a single bond; an C₆-C₆₀ arylene group; a fluorenylene group; a C₂-C₆₀ heterocyclic group including at least one heteroatom of O, N, S, Si or P; and a fused ring group of a C₃-C₆₀ aliphatic ring and a C₆-C₆₀ aromatic ring.
   Wherein in case L¹, L², L³, L⁴, L⁵ and L⁶ are an arylene group, it may be preferably a C₆-C₃₀ arylene group, more preferably a C₆-C₂₄ arylene group, for example, phenylene, biphenyl, naphthalene, terphenyl, etc.
   In case L¹, L², L³, L⁴, L⁵ and L⁶ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.
   In case L¹, L², L³, L⁴, L⁵ and L⁶ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.
5) i) In case a is 0, R¹ is independently selected from the group consisting of hydrogen; deuterium; tritium; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; or R¹ and R² are bonded to each other to form a ring.
   Wherein in case R¹ is an alkyl group, it may be preferably a C₁-C₃₀ alkyl group, and more preferably a C₁-C₂₄ alkyl group.
   In case R¹ is an alkenyl group, they may be preferably an C₂~C₃₀ alkenyl group, more preferably an C₂~C₂₄ alkenyl group.
   In case R¹ is an alkynyl group, they may be preferably an C₂~C₃₀ alkynyl group, more preferably an C₂~C₂₄ alkynyl group.
   In case R¹ is an alkoxyl group, it may be preferably a C₁-C₃₀ alkoxyl group, more preferably an C₁~C₂₄ alkoxyl group.
   In case R¹ is an aryloxy group, it may be preferably a C₆-C₃₀ aryloxy group, more preferably an C₆~C₂₄ aryloxy group.
   In case R¹ is an aryl group, it may be preferably a C₆-C₃₀ aryl group, more preferably an C₆~C₂₅ aryl group.
   In case R¹ is a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.
   In case R¹ is a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.
   ii) when a is 1, R¹ is Z; wherein Z is selected from the group consisting of a C₆-C₆₀ arylene group; a fluorenylene group; a C₂-C₆₀ heterocyclic group including at least one heteroatom of O, N, S, Si or P; a fused ring group of a C₃-C₆₀ aliphatic ring and a C₆-C₆₀ aromatic ring; or Z and R² may be bonded to each other to form a ring.
   Wherein in case Z is an arylene group, it may be preferably a C₆-C₃₀ arylene group, and more preferably a C₆-C₂₄ arylene group, for example, it may be phenylene, biphenyl, naphthalene, terphenyl, etc.
   In case Z is a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.
   In case Z is a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.
6) R² is independently selected from the group consisting of hydrogen; deuterium; tritium; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring.
   Wherein in case R² is an alkyl group, it may be preferably a C₁-C₃₀ alkyl group, and more preferably a C₁-C₂₄ alkyl group.
   In case R² is an alkenyl group, they may be preferably an C₂~C₃₀ alkenyl group, more preferably an C₂~C₂₄ alkenyl group.
   In case R² is an alkynyl group, they may be preferably an C₂~C₃₀ alkynyl group, more preferably an C₂~C₂₄ alkynyl group.
   In case R² is an alkoxyl group, it may be preferably a C₁-C₃₀ alkoxyl group, more preferably an C₁~C₂₄ alkoxyl group.
   In case R² is an aryloxy group, it may be preferably a C₆-C₃₀ aryloxy group, more preferably an C6-C24 aryloxy group.
   In case R² is an aryl group, it may be preferably a C₆-C₃₀ aryl group, more preferably an C₆~C₂₅ aryl group, for example, it may be phenylene, biphenyl, naphthalene, terphenyl, etc.
   In case R² is a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.
   In case R² is a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.
7) R³, R⁴, R⁵, R⁶, R⁷, R⁸, R⁹, R¹⁰, R¹¹ and R¹² are independently selected from the group consisting of hydrogen; deuterium; tritium; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring, or wherein in case m, n, o, p, q, r, s and t are 2 or more, R³, R⁴, R⁵, R⁶, R⁷, R⁸, R⁹, R¹⁰, R¹¹ and R¹² are each in plural being the same or different, or a plurality of R³ or a plurality of R⁴ or a plurality of R⁵ or a plurality of R⁶ or a plurality of R⁷ or a plurality of R¹⁰ or a plurality of R¹¹ or a plurality of R¹² may be bonded to each other to form a ring.
   Wherein R³, R⁴, R⁵, R⁶, R⁷, R⁸, R⁹, R¹⁰, R¹¹ and R¹² are an alkyl group, it may be preferably a C₁-C₃₀ alkyl group, and more preferably a C₁-C₂₄ alkyl group.
   In case R³, R⁴, R⁵, R⁶, R⁷, R⁸, R⁹, R¹⁰, R¹¹ and R¹² are an alkenyl group, it may be preferably an C₂~C₃₀ alkenyl group, more preferably an C₂~C₂₄ alkenyl group.
   In case R³, R⁴, R⁵, R⁶, R⁷, R⁸, R⁹, R¹⁰, R¹¹ and R¹² are an alkynyl group, it may be preferably an C₂~C₃₀ alkynyl group, more preferably an C₂~C₂₄ alkynyl group.
   In case R³, R⁴, R⁵, R⁶, R⁷, R⁸, R⁹, R¹⁰, R¹¹ and R¹² are an alkoxyl group, it may be preferably a C₁-C₃₀ alkoxyl group, more preferably an C₁~C₂₄ alkoxyl group.
   In case R³, R⁴, R⁵, R⁶, R⁷, R⁸, R⁹, R¹⁰, R¹¹ and R¹² are an aryloxy group, it may be preferably a C₆-C₃₀ aryloxy group, more preferably an C₆~C₂₄ aryloxy group.
   In case R³, R⁴, R⁵, R⁶, R⁷, R⁸, R⁹, R¹⁰, R¹¹ and R¹² are an aryl group, it may be preferably a C₆-C₃₀ aryl group, more preferably an C₆~C₂₅ aryl group, for example, it may be phenylene, biphenyl, naphthalene, terphenyl, etc.
   In case R³, R⁴, R⁵, R⁶, R⁷, R⁸, R⁹, R¹⁰, R¹¹ and R¹² are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.
   In case R³, R⁴, R⁵, R⁶, R⁷, R⁸, R⁹, R¹⁰, R¹¹ and R¹² are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.
8) R⁵, R⁶, R⁷, R⁸, R⁹, R¹⁰, R¹¹ and R¹² are may be bonded to each other to form a ring, however, unless R⁵ and R⁸ or R⁹ and R¹² are bonded to each other to form a ring,
9) m, n, o, p, q, r, s and t are each independently an integer from 0 to 4,
10) wherein the aryl group, arylene group, heterocyclic group, fluorenyl group, fluorenylene group, fused ring group, alkyl group, alkenyl group, alkynyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂~C₂₀ heterocyclic group; C₃-C₂₀ cycloalkyl group; C₇-C₂₀ arylalkyl group; and C₈-C₂₀ arylalkenyl group; also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, Z is represented by any one of Formulas 2-1 to 2-3

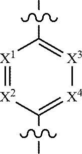

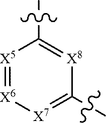

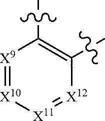

In Formulas 2-1 to 2-3, each symbol may be defined as follows.
1) X¹, X², X³, X⁴, X⁵, X⁶, X⁷, X⁸, X⁹, X¹⁰, X¹¹ and X¹², are each independently CR^{a} or N,
2) R^{a} is independently selected from the group consisting of hydrogen; deuterium; tritium; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; or a plurality of R^{a} may be bonded to each other to form a ring.
   Wherein in case R^{a} is an alkyl group, it may be preferably a C₁-C₃₀ alkyl group, and more preferably a C₁-C₂₄ alkyl group.
   In case R^{a} is an alkenyl group, they may be preferably an C₂~C₃₀ alkenyl group, more preferably an C₂~C₂₄ alkenyl group.
   In case R^{a} is an alkynyl group, they may be preferably an C₂~C₃₀ alkynyl group, more preferably an C₂~C₂₄ alkynyl group.
   In case R^{a} is an alkoxyl group, it may be preferably a C₁-C₃₀ alkoxyl group, more preferably an C₁~C₂₄ alkoxyl group.
   In case R^{a} is an aryloxy group, it may be preferably a C₆-C₃₀ aryloxy group, more preferably an C₆~C₂₄ aryloxy group.
   In case R^{a} is an aryl group, it may be preferably a C₆-C₃₀ aryl group, more preferably an C₆~C₂₅ aryl group, for example, it may be phenylene, biphenyl, naphthalene, terphenyl, etc.
   In case R^{a} is a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.
   In case R^{a} is a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.
3)

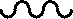

represents a bonding position.

Also, Formula 1 is represented by Formula 2 or Formula

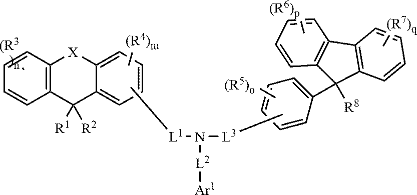

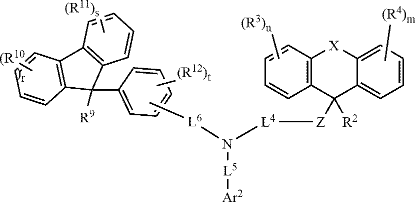

{wherein, X, R¹, R², R³, R⁴, R⁵, R⁶, R⁷, R⁸, R⁹, R¹⁰, R¹¹, R¹², L¹, L², L³, L⁴, L⁵, L⁶, Ar¹, Ar², Z, m, n, o, p, q, r, s and t are the same as defined above.}

Also, Formula 1 is represented by any one of Formulas 4 to 9. Preferably, Formula 1 is a compound represented by Formula 5, Formula 6, Formula 8, or Formula 9.

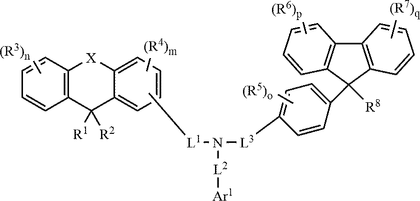

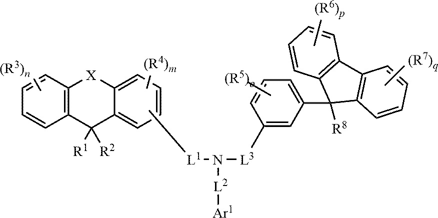

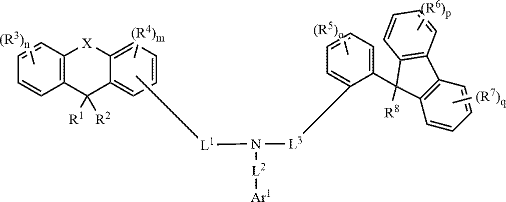

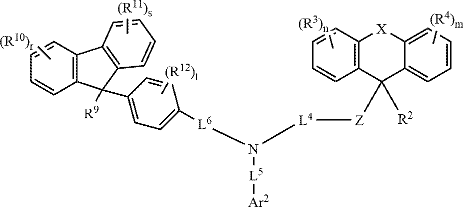

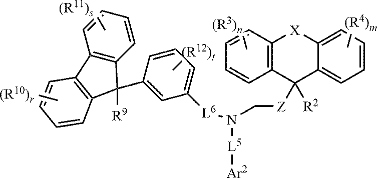

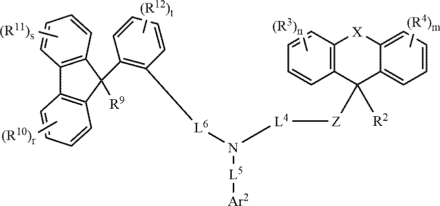

{wherein, X, R¹, R², R³, R⁴, R⁵, R⁶, R⁷, R⁸, R⁹, R¹⁰, R¹¹, R¹², L¹, L², L³, L⁴, L⁵, L⁶, Ar¹, Ar², Z, m, n, o, p, q, r, s and t are the same as defined above.}

Also, Formula 1 is represented by any one of Formulas 10 to 13

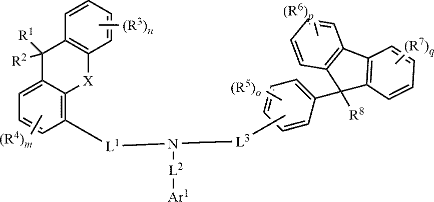

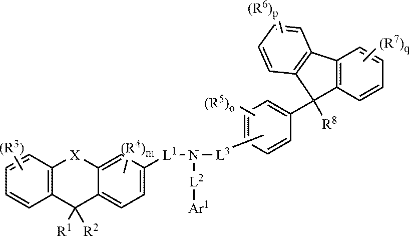

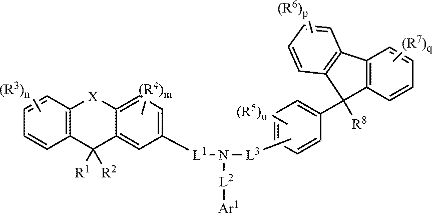

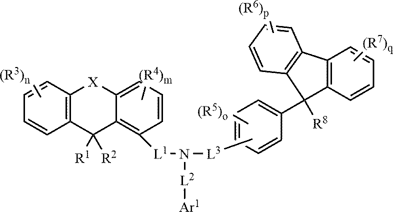

{wherein, X, R¹, R², R³, R⁴, R⁵, R⁶, R⁷, R⁸, L¹, L², L³, Ar¹, m, n, o, p, and q are the same as defined above.}

Also, Ar¹ or Ar² is represented by Formula 1-1

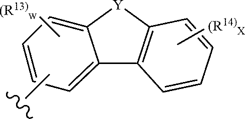

In Formula 1-1, each symbol may be defined as follows.
1) Y is O, S, NR^{b} or CR’R″,
2) R¹³, R¹⁴, R^{b}, R′ and R″ are independently selected from the group consisting of hydrogen; deuterium; tritium; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; or, a plurality of adjacent R¹³ or a plurality of R¹⁴ or R′ and R″ may be bonded to each other to form a ring.
   Wherein in case R¹³, R¹⁴, R^{b}, R′ and R″ are an alkyl group, it may be preferably a C₁-C₃₀ alkyl group, and more preferably a C₁-C₂₄ alkyl group.
   In case R¹³, R¹⁴, R^{b}, R′ and R″ are an alkenyl group, they may be preferably an C₂~C₃₀ alkenyl group, more preferably an C₂~C₂₄ alkenyl group.
   In case R¹³, R¹⁴, R^{b}, R′ and R″ are an alkynyl group, they may be preferably an C₂~C₃₀ alkynyl group, more preferably an C₂~C₂₄ alkynyl group.
   In case R¹³, R¹⁴, R^{b}, R′ and R″ are an alkoxyl group, it may be preferably a C₁-C₃₀ alkoxyl group, more preferably an C₁~C₂₄ alkoxyl group.
   In case R¹³, R¹⁴, R^{b}, R′ and R″ are an aryloxy group, it may be preferably a C₆-C₃₀ aryloxy group, more preferably an C₆~C₂₄ aryloxy group.
   In case R¹³, R¹⁴, R^{b}, R′ and R″ are an aryl group, it may be preferably a C₆-C₃₀ aryl group, more preferably an C₆~C₂₅ aryl group, for example, it may be phenylene, biphenyl, naphthalene, terphenyl, etc.
   In case R¹³, R¹⁴, R^{b}, R′ and R″ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.
   In case R¹³, R¹⁴, R^{b}, R′ and R″ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.
3) w is an integer of 0 to 3, x is an integer of 0 to 4,
4)

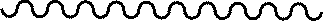

indicates the position to be bonded

Also, Formula 1 is represented by Formula 14 or Formula 15.

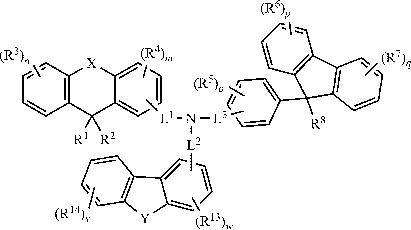

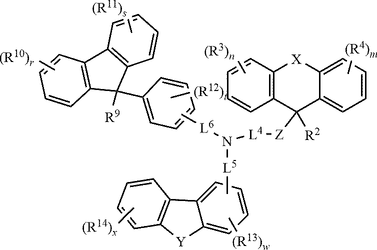

{wherein,
1) X, R¹, R², R³, R⁴, R⁵, R⁶, R⁷, R⁸, R⁹, R¹⁰, R¹¹, R¹², L¹, L², L³, L⁴, L⁵, L⁶, Z, m, n, o, p, q, r, s, t and
2) Y, R¹³, R¹⁴, w and x are the same as defined above.}

Also, Formula 1 is represented by any one of Formulas 16 to 21, and more preferably represented by Formula 17, Formula 18, Formula 20, or Formula 21.

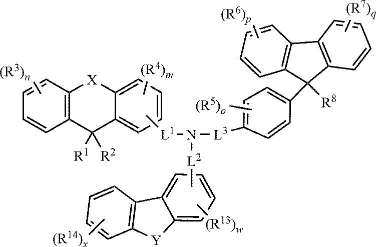

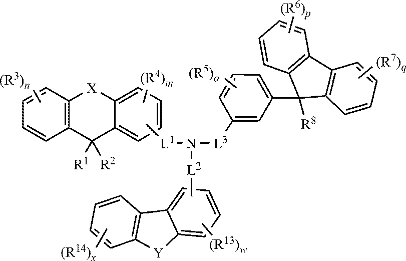

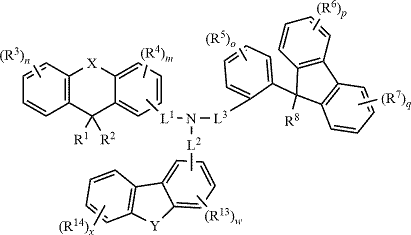

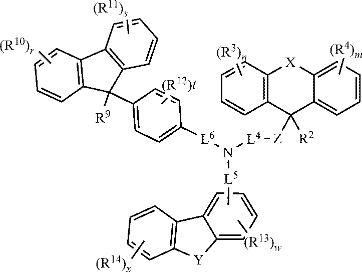

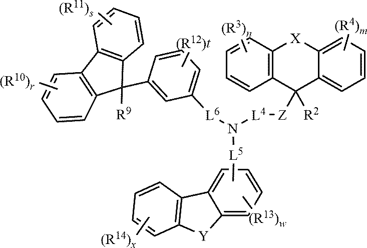

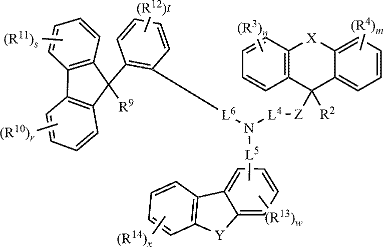

{wherein,
1) X, R¹, R², R³, R⁴, R⁵, R⁶, R⁷, R⁸, R⁹, R¹⁰, R¹¹, R¹², L¹, L², L³, L⁴, L⁵, L⁶, Z, m, n, o, p, q, r, s, t and
2) Y, R¹³, R¹⁴, w and x are the same as defined above.}

Also, Formula 1 is represented by any one of Formulas 22 to 25

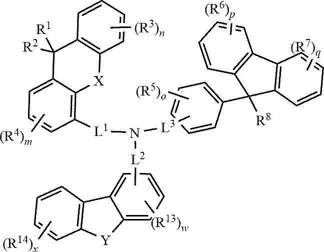

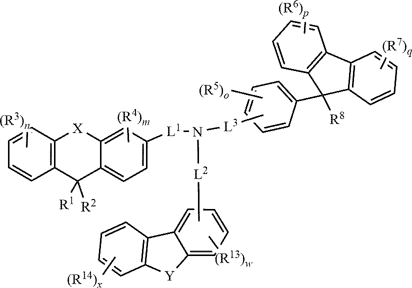

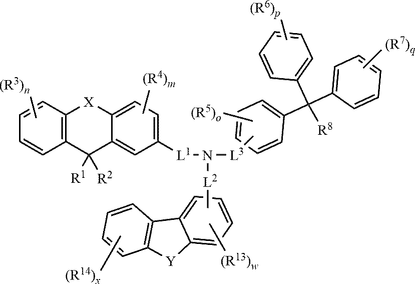

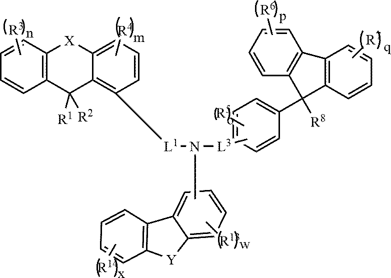

{wherein,
1) X, R¹, R², R³, R⁴, R⁵, R⁶, R⁷, R⁸, L¹, L², L³, m, n, o, p, q and
2) Y, R¹³, R¹⁴, w and x are the same as defined above.}

Also, Formula 1-1 is represented by any one of Formulas 1-2 to 1-5.

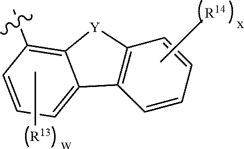

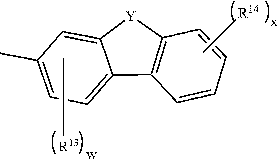

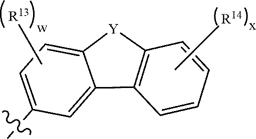

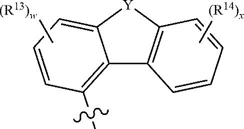

{wherein, Y, R¹³, R¹⁴, w and x are the same as the definition of Formula 1-1,

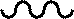

indicates the position to be bonded.}

Specifically, the compound represented by Formula 1 may be any one of the compounds P1-1 to P7-4, but is not limited thereto.

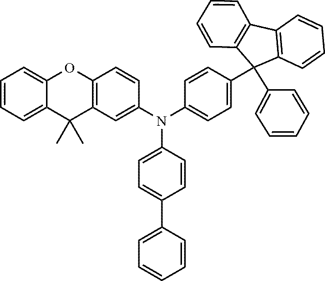 [SMILES: CC1(C)c2ccccc2Oc2ccc(N(c3ccc(-c4ccccc4)cc3)c3ccc(C4(c5ccccc5)c5ccccc5-c5ccccc54)cc3)cc21]

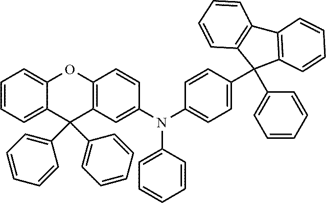 [SMILES: c1ccc(N(c2ccc(C3(c4ccccc4)c4ccccc4-c4ccccc43)cc2)c2ccc3c(c2)C(c2ccccc2)(c2ccccc2)c2ccccc2O3)cc1]

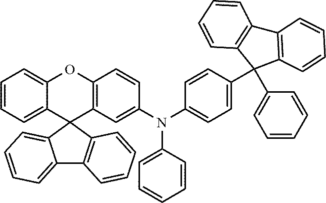 [SMILES: c1ccc(N(c2ccc(C3(c4ccccc4)c4ccccc4-c4ccccc43)cc2)c2ccc3c(c2)C2(c4ccccc4O3)c3ccccc3-c3ccccc32)cc1]

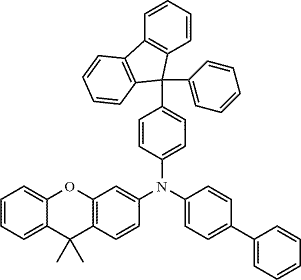 [SMILES: CC1(C)c2ccccc2Oc2cc(N(c3ccc(-c4ccccc4)cc3)c3ccc(C4(c5ccccc5)c5ccccc5-c5ccccc54)cc3)ccc21]

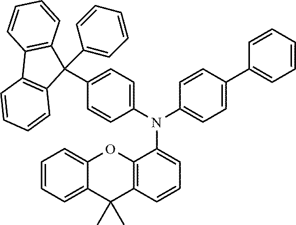 [SMILES: CC1(C)c2ccccc2Oc2c(N(c3ccc(-c4ccccc4)cc3)c3ccc(C4(c5ccccc5)c5ccccc5-c5ccccc54)cc3)cccc21]

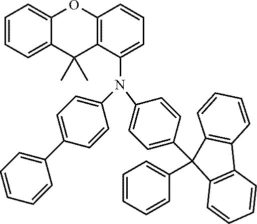 [SMILES: CC1(C)c2ccccc2Oc2cccc(N(c3ccc(-c4ccccc4)cc3)c3ccc(C4(c5ccccc5)c5ccccc5-c5ccccc54)cc3)c21]

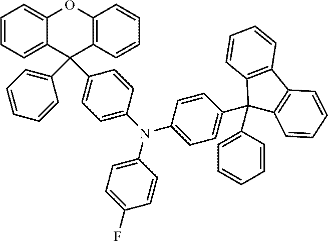 [SMILES: Fc1ccc(N(c2ccc(C3(c4ccccc4)c4ccccc4Oc4ccccc43)cc2)c2ccc(C3(c4ccccc4)c4ccccc4-c4ccccc43)cc2)cc1]

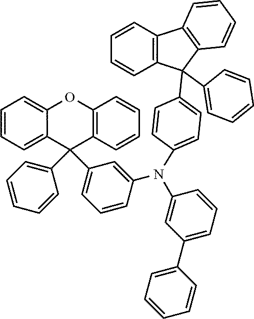 [SMILES: c1ccc(-c2cccc(N(c3ccc(C4(c5ccccc5)c5ccccc5-c5ccccc54)cc3)c3cccc(C4(c5ccccc5)c5ccccc5Oc5ccccc54)c3)c2)cc1]

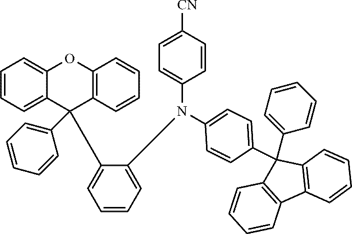 [SMILES: N#Cc1ccc(N(c2ccc(C3(c4ccccc4)c4ccccc4-c4ccccc43)cc2)c2ccccc2C2(c3ccccc3)c3ccccc3Oc3ccccc32)cc1]

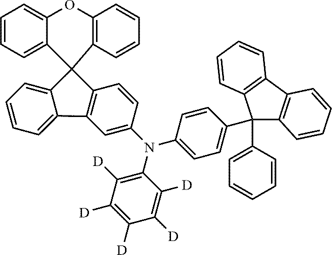 [SMILES: [2H]c1c([2H])c([2H])c(N(c2ccc(C3(c4ccccc4)c4ccccc4-c4ccccc43)cc2)c2ccc3c(c2)-c2ccccc2C32c3ccccc3Oc3ccccc32)c([2H])c1[2H]]

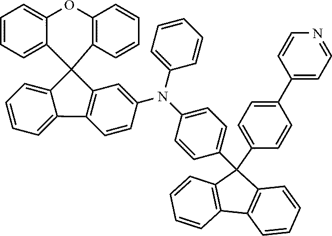 [SMILES: c1ccc(N(c2ccc(C3(c4ccc(-c5ccncc5)cc4)c4ccccc4-c4ccccc43)cc2)c2ccc3c(c2)C2(c4ccccc4Oc4ccccc42)c2ccccc2-3)cc1]

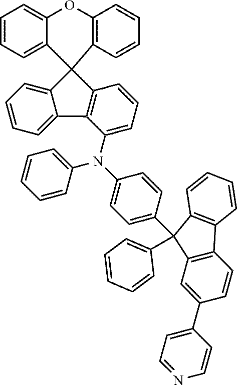 [SMILES: c1ccc(N(c2ccc(C3(c4ccccc4)c4ccccc4-c4ccc(-c5ccncc5)cc43)cc2)c2cccc3c2-c2ccccc2C32c3ccccc3Oc3ccccc32)cc1]

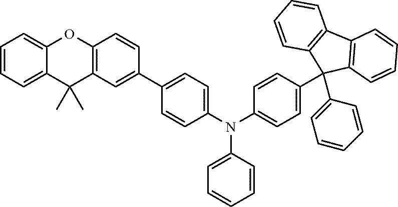 [SMILES: CC1(C)c2ccccc2Oc2ccc(-c3ccc(N(c4ccccc4)c4ccc(C5(c6ccccc6)c6ccccc6-c6ccccc65)cc4)cc3)cc21]

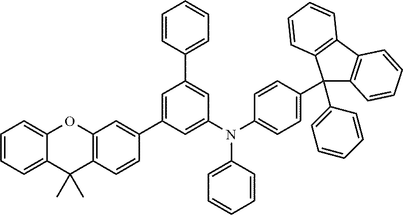 [SMILES: CC1(C)c2ccccc2Oc2cc(-c3cc(-c4ccccc4)cc(N(c4ccccc4)c4ccc(C5(c6ccccc6)c6ccccc6-c6ccccc65)cc4)c3)ccc21]

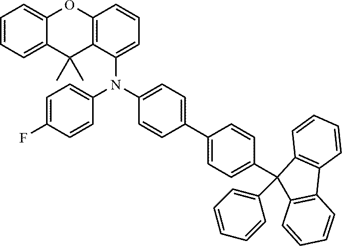 [SMILES: CC1(C)c2ccccc2Oc2cccc(N(c3ccc(F)cc3)c3ccc(-c4ccc(C5(c6ccccc6)c6ccccc6-c6ccccc65)cc4)cc3)c21]

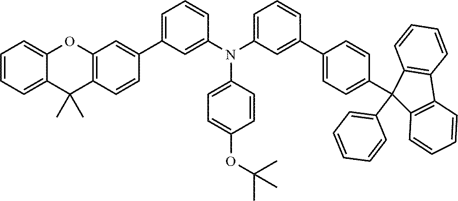 [SMILES: CC(C)(C)Oc1ccc(N(c2cccc(-c3ccc(C4(c5ccccc5)c5ccccc5-c5ccccc54)cc3)c2)c2cccc(-c3ccc4c(c3)Oc3ccccc3C4(C)C)c2)cc1]

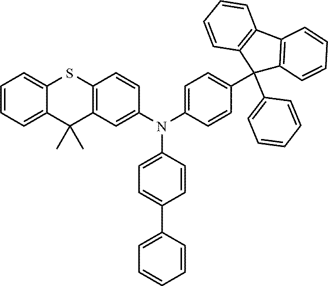 [SMILES: CC1(C)c2ccccc2Sc2ccc(N(c3ccc(-c4ccccc4)cc3)c3ccc(C4(c5ccccc5)c5ccccc5-c5ccccc54)cc3)cc21]

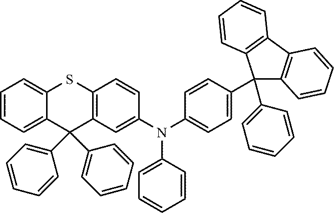 [SMILES: c1ccc(N(c2ccc(C3(c4ccccc4)c4ccccc4-c4ccccc43)cc2)c2ccc3c(c2)C(c2ccccc2)(c2ccccc2)c2ccccc2S3)cc1]

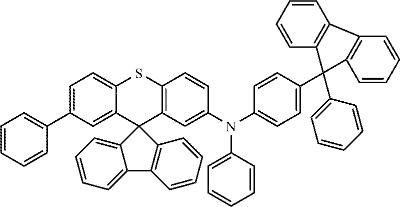 [SMILES: c1ccc(-c2ccc3c(c2)C2(c4cc(N(c5ccccc5)c5ccc(C6(c7ccccc7)c7ccccc7-c7ccccc76)cc5)ccc4S3)c3ccccc3-c3ccccc32)cc1]

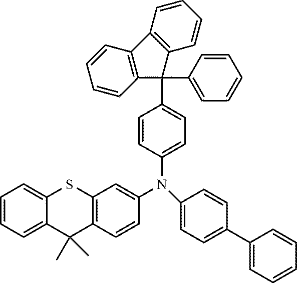 [SMILES: CC1(C)c2ccccc2Sc2cc(N(c3ccc(-c4ccccc4)cc3)c3ccc(C4(c5ccccc5)c5ccccc5-c5ccccc54)cc3)ccc21]

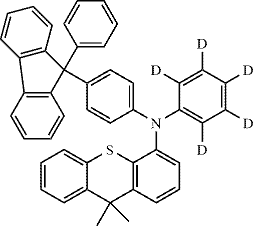 [SMILES: [2H]c1c([2H])c([2H])c(N(c2ccc(C3(c4ccccc4)c4ccccc4-c4ccccc43)cc2)c2cccc3c2Sc2ccccc2C3(C)C)c([2H])c1[2H]]

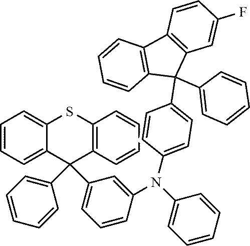 [SMILES: Fc1ccc2c(c1)C(c1ccccc1)(c1ccc(N(c3ccccc3)c3cccc(C4(c5ccccc5)c5ccccc5Sc5ccccc54)c3)cc1)c1ccccc1-2]

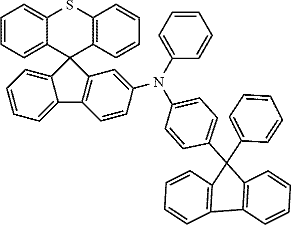 [SMILES: c1ccc(N(c2ccc(C3(c4ccccc4)c4ccccc4-c4ccccc43)cc2)c2ccc3c(c2)C2(c4ccccc4Sc4ccccc42)c2ccccc2-3)cc1]

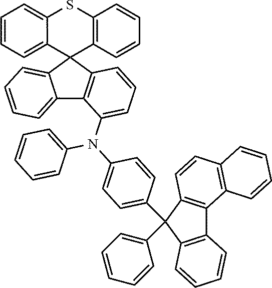 [SMILES: c1ccc(N(c2ccc(C3(c4ccccc4)c4ccccc4-c4c3ccc3ccccc43)cc2)c2cccc3c2-c2ccccc2C32c3ccccc3Sc3ccccc32)cc1]

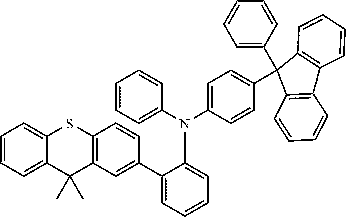 [SMILES: CC1(C)c2ccccc2Sc2ccc(-c3ccccc3N(c3ccccc3)c3ccc(C4(c5ccccc5)c5ccccc5-c5ccccc54)cc3)cc21]

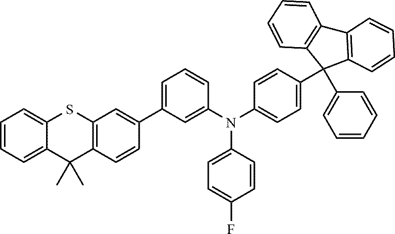 [SMILES: CC1(C)c2ccccc2Sc2cc(-c3cccc(N(c4ccc(F)cc4)c4ccc(C5(c6ccccc6)c6ccccc6-c6ccccc65)cc4)c3)ccc21]

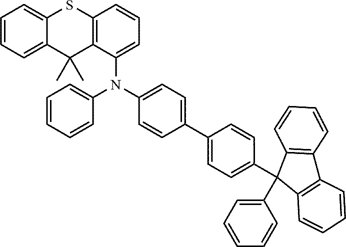 [SMILES: CC1(C)c2ccccc2Sc2cccc(N(c3ccccc3)c3ccc(-c4ccc(C5(c6ccccc6)c6ccccc6-c6ccccc65)cc4)cc3)c21]

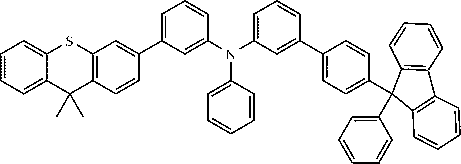 [SMILES: CC1(C)c2ccccc2Sc2cc(-c3cccc(N(c4ccccc4)c4cccc(-c5ccc(C6(c7ccccc7)c7ccccc7-c7ccccc76)cc5)c4)c3)ccc21]

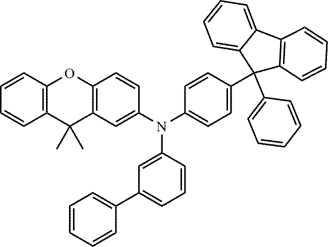 [SMILES: CC1(C)c2ccccc2Oc2ccc(N(c3ccc(C4(c5ccccc5)c5ccccc5-c5ccccc54)cc3)c3cccc(-c4ccccc4)c3)cc21]

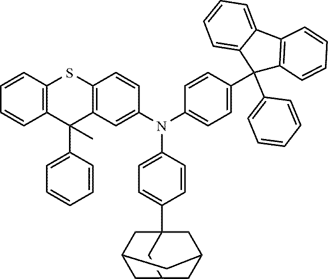 [SMILES: CC1(c2ccccc2)c2ccccc2Sc2ccc(N(c3ccc(C45CC6CC(CC(C6)C4)C5)cc3)c3ccc(C4(c5ccccc5)c5ccccc5-c5ccccc54)cc3)cc21]

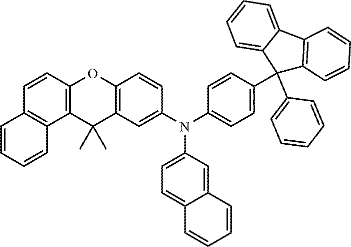 [SMILES: CC1(C)c2cc(N(c3ccc(C4(c5ccccc5)c5ccccc5-c5ccccc54)cc3)c3ccc4ccccc4c3)ccc2Oc2ccc3ccccc3c21]

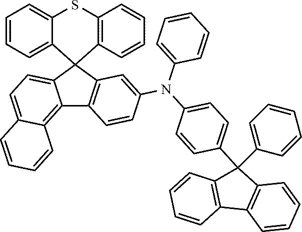 [SMILES: c1ccc(N(c2ccc(C3(c4ccccc4)c4ccccc4-c4ccccc43)cc2)c2ccc3c(c2)C2(c4ccccc4Sc4ccccc42)c2ccc4ccccc4c2-3)cc1]

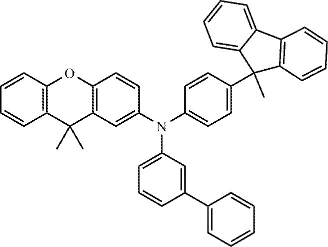 [SMILES: CC1(C)c2ccccc2Oc2ccc(N(c3ccc(C4(C)c5ccccc5-c5ccccc54)cc3)c3cccc(-c4ccccc4)c3)cc21]

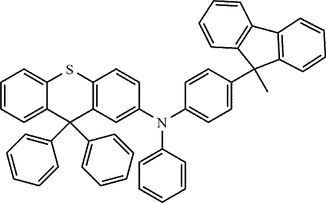 [SMILES: CC1(c2ccc(N(c3ccccc3)c3ccc4c(c3)C(c3ccccc3)(c3ccccc3)c3ccccc3S4)cc2)c2ccccc2-c2ccccc21]

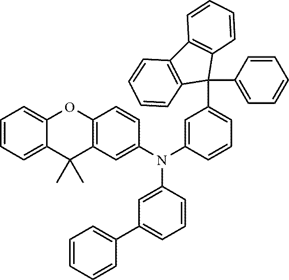 [SMILES: CC1(C)c2ccccc2Oc2ccc(N(c3cccc(-c4ccccc4)c3)c3cccc(C4(c5ccccc5)c5ccccc5-c5ccccc54)c3)cc21]

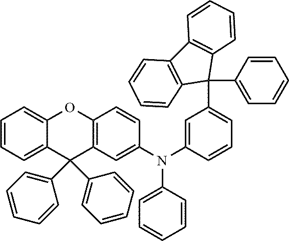 [SMILES: c1ccc(N(c2cccc(C3(c4ccccc4)c4ccccc4-c4ccccc43)c2)c2ccc3c(c2)C(c2ccccc2)(c2ccccc2)c2ccccc2O3)cc1]

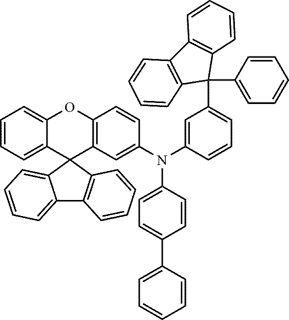 [SMILES: c1ccc(-c2ccc(N(c3cccc(C4(c5ccccc5)c5ccccc5-c5ccccc54)c3)c3ccc4c(c3)C3(c5ccccc5O4)c4ccccc4-c4ccccc43)cc2)cc1]

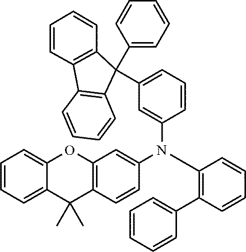 [SMILES: CC1(C)c2ccccc2Oc2cc(N(c3cccc(C4(c5ccccc5)c5ccccc5-c5ccccc54)c3)c3ccccc3-c3ccccc3)ccc21]

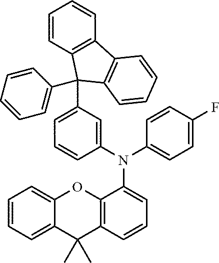 [SMILES: CC1(C)c2ccccc2Oc2c(N(c3ccc(F)cc3)c3cccc(C4(c5ccccc5)c5ccccc5-c5ccccc54)c3)cccc21]

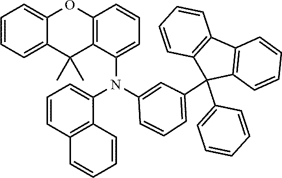 [SMILES: CC1(C)c2ccccc2Oc2cccc(N(c3cccc(C4(c5ccccc5)c5ccccc5-c5ccccc54)c3)c3cccc4ccccc34)c21]

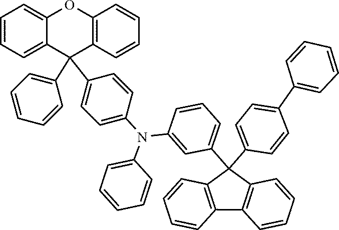 [SMILES: c1ccc(-c2ccc(C3(c4cccc(N(c5ccccc5)c5ccc(C6(c7ccccc7)c7ccccc7Oc7ccccc76)cc5)c4)c4ccccc4-c4ccccc43)cc2)cc1]

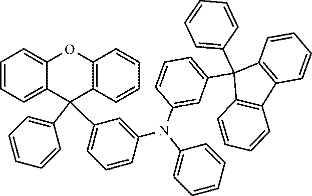 [SMILES: c1ccc(N(c2cccc(C3(c4ccccc4)c4ccccc4Oc4ccccc43)c2)c2cccc(C3(c4ccccc4)c4ccccc4-c4ccccc43)c2)cc1]

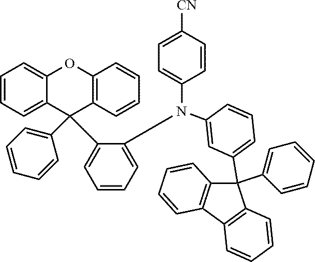 [SMILES: N#Cc1ccc(N(c2cccc(C3(c4ccccc4)c4ccccc4-c4ccccc43)c2)c2ccccc2C2(c3ccccc3)c3ccccc3Oc3ccccc32)cc1]

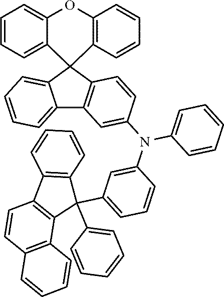 [SMILES: c1ccc(N(c2cccc(C3(c4ccccc4)c4ccccc4-c4ccc5ccccc5c43)c2)c2ccc3c(c2)-c2ccccc2C32c3ccccc3Oc3ccccc32)cc1]

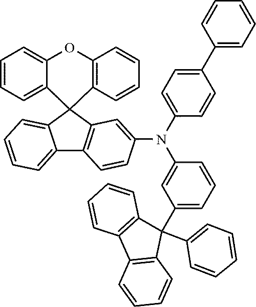 [SMILES: c1ccc(-c2ccc(N(c3cccc(C4(c5ccccc5)c5ccccc5-c5ccccc54)c3)c3ccc4c(c3)C3(c5ccccc5Oc5ccccc53)c3ccccc3-4)cc2)cc1]

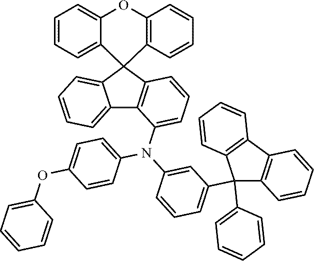 [SMILES: c1ccc(Oc2ccc(N(c3cccc(C4(c5ccccc5)c5ccccc5-c5ccccc54)c3)c3cccc4c3-c3ccccc3C43c4ccccc4Oc4ccccc43)cc2)cc1]

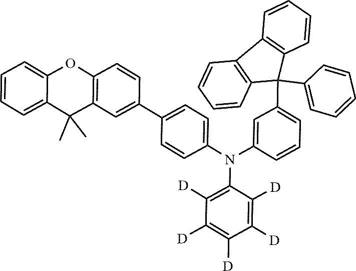 [SMILES: [2H]c1c([2H])c([2H])c(N(c2ccc(-c3ccc4c(c3)C(C)(C)c3ccccc3O4)cc2)c2cccc(C3(c4ccccc4)c4ccccc4-c4ccccc43)c2)c([2H])c1[2H]]

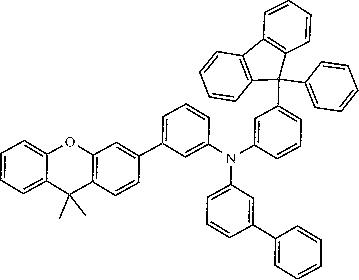 [SMILES: CC1(C)c2ccccc2Oc2cc(-c3cccc(N(c4cccc(-c5ccccc5)c4)c4cccc(C5(c6ccccc6)c6ccccc6-c6ccccc65)c4)c3)ccc21]

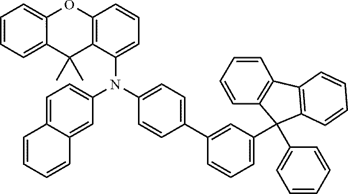 [SMILES: CC1(C)c2ccccc2Oc2cccc(N(c3ccc(-c4cccc(C5(c6ccccc6)c6ccccc6-c6ccccc65)c4)cc3)c3ccc4ccccc4c3)c21]

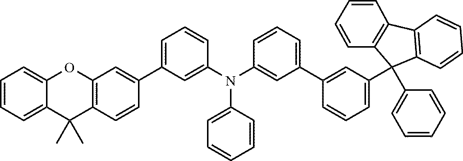 [SMILES: CC1(C)c2ccccc2Oc2cc(-c3cccc(N(c4ccccc4)c4cccc(-c5cccc(C6(c7ccccc7)c7ccccc7-c7ccccc76)c5)c4)c3)ccc21]

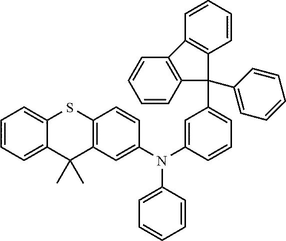 [SMILES: CC1(C)c2ccccc2Sc2ccc(N(c3ccccc3)c3cccc(C4(c5ccccc5)c5ccccc5-c5ccccc54)c3)cc21]

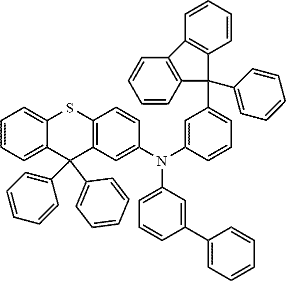 [SMILES: c1ccc(-c2cccc(N(c3cccc(C4(c5ccccc5)c5ccccc5-c5ccccc54)c3)c3ccc4c(c3)C(c3ccccc3)(c3ccccc3)c3ccccc3S4)c2)cc1]

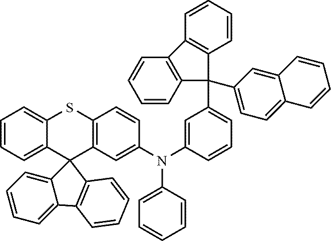 [SMILES: c1ccc(N(c2cccc(C3(c4ccc5ccccc5c4)c4ccccc4-c4ccccc43)c2)c2ccc3c(c2)C2(c4ccccc4S3)c3ccccc3-c3ccccc32)cc1]

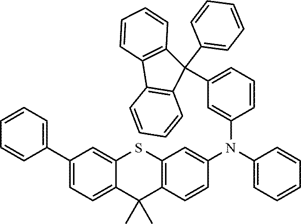 [SMILES: CC1(C)c2ccc(-c3ccccc3)cc2Sc2cc(N(c3ccccc3)c3cccc(C4(c5ccccc5)c5ccccc5-c5ccccc54)c3)ccc21]

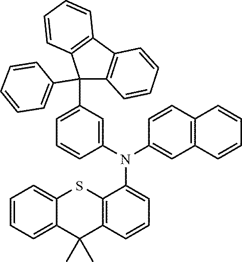 [SMILES: CC1(C)c2ccccc2Sc2c(N(c3cccc(C4(c5ccccc5)c5ccccc5-c5ccccc54)c3)c3ccc4ccccc4c3)cccc21]

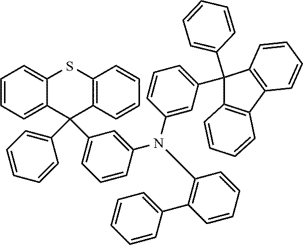 [SMILES: c1ccc(-c2ccccc2N(c2cccc(C3(c4ccccc4)c4ccccc4Sc4ccccc43)c2)c2cccc(C3(c4ccccc4)c4ccccc4-c4ccccc43)c2)cc1]

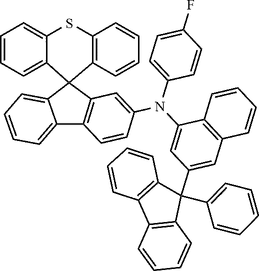 [SMILES: Fc1ccc(N(c2ccc3c(c2)C2(c4ccccc4Sc4ccccc42)c2ccccc2-3)c2cc(C3(c4ccccc4)c4ccccc4-c4ccccc43)cc3ccccc23)cc1]

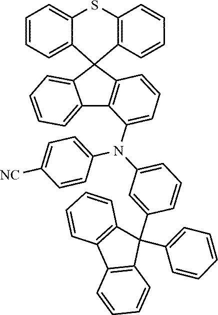

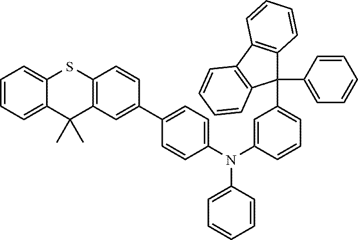 [SMILES: CC1(C)c2ccccc2Sc2ccc(-c3ccc(N(c4ccccc4)c4cccc(C5(c6ccccc6)c6ccccc6-c6ccccc65)c4)cc3)cc21]

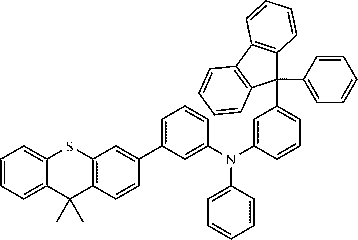 [SMILES: CC1(C)c2ccccc2Sc2cc(-c3cccc(N(c4ccccc4)c4cccc(C5(c6ccccc6)c6ccccc6-c6ccccc65)c4)c3)ccc21]

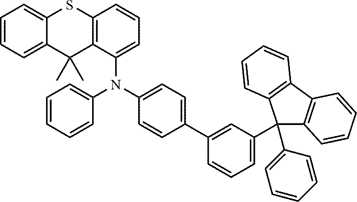 [SMILES: CC1(C)c2ccccc2Sc2cccc(N(c3ccccc3)c3ccc(-c4cccc(C5(c6ccccc6)c6ccccc6-c6ccccc65)c4)cc3)c21]

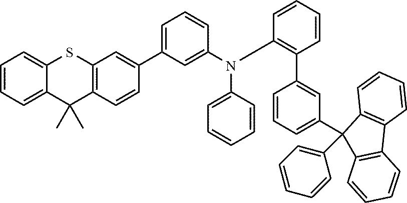 [SMILES: CC1(C)c2ccccc2Sc2cc(-c3cccc(N(c4ccccc4)c4ccccc4-c4cccc(C5(c6ccccc6)c6ccccc6-c6ccccc65)c4)c3)ccc21]

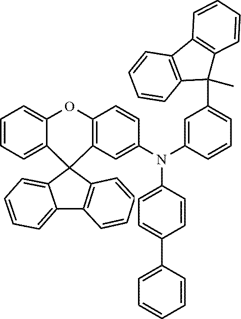 [SMILES: CC1(c2cccc(N(c3ccc(-c4ccccc4)cc3)c3ccc4c(c3)C3(c5ccccc5O4)c4ccccc4-c4ccccc43)c2)c2ccccc2-c2ccccc21]

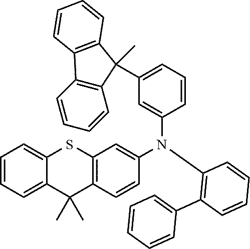 [SMILES: CC1(C)c2ccccc2Sc2cc(N(c3cccc(C4(C)c5ccccc5-c5ccccc54)c3)c3ccccc3-c3ccccc3)ccc21]

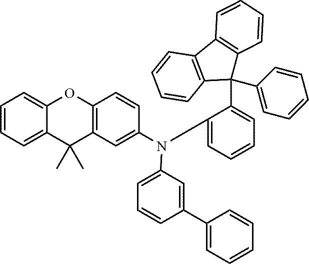 [SMILES: CC1(C)c2ccccc2Oc2ccc(N(c3cccc(-c4ccccc4)c3)c3ccccc3C3(c4ccccc4)c4ccccc4-c4ccccc43)cc21]

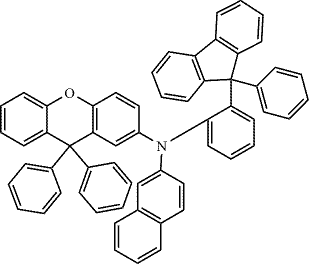 [SMILES: c1ccc(C2(c3ccccc3)c3ccccc3Oc3ccc(N(c4ccc5ccccc5c4)c4ccccc4C4(c5ccccc5)c5ccccc5-c5ccccc54)cc32)cc1]

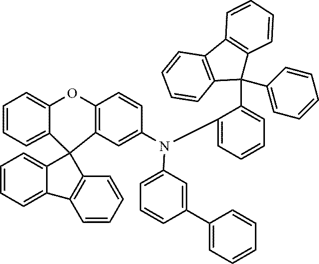 [SMILES: c1ccc(-c2cccc(N(c3ccc4c(c3)C3(c5ccccc5O4)c4ccccc4-c4ccccc43)c3ccccc3C3(c4ccccc4)c4ccccc4-c4ccccc43)c2)cc1]

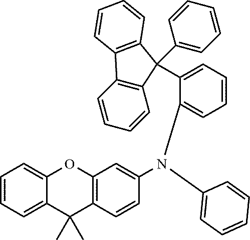 [SMILES: CC1(C)c2ccccc2Oc2cc(N(c3ccccc3)c3ccccc3C3(c4ccccc4)c4ccccc4-c4ccccc43)ccc21]

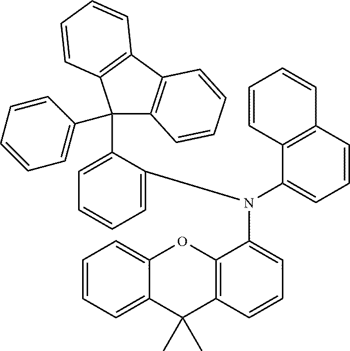 [SMILES: CC1(C)c2ccccc2Oc2c(N(c3ccccc3C3(c4ccccc4)c4ccccc4-c4ccccc43)c3cccc4ccccc34)cccc21]

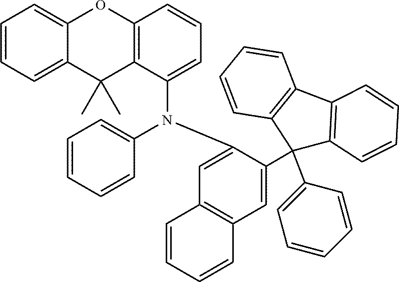 [SMILES: CC1(C)c2ccccc2Oc2cccc(N(c3ccccc3)c3cc4ccccc4cc3C3(c4ccccc4)c4ccccc4-c4ccccc43)c21]

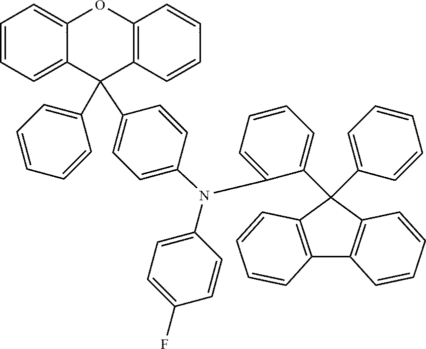 [SMILES: Fc1ccc(N(c2ccc(C3(c4ccccc4)c4ccccc4Oc4ccccc43)cc2)c2ccccc2C2(c3ccccc3)c3ccccc3-c3ccccc32)cc1]

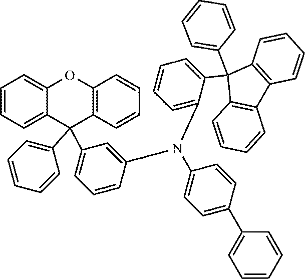 [SMILES: c1ccc(-c2ccc(N(c3cccc(C4(c5ccccc5)c5ccccc5Oc5ccccc54)c3)c3ccccc3C3(c4ccccc4)c4ccccc4-c4ccccc43)cc2)cc1]

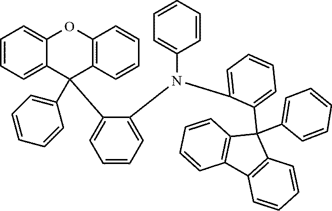 [SMILES: c1ccc(N(c2ccccc2C2(c3ccccc3)c3ccccc3Oc3ccccc32)c2ccccc2C2(c3ccccc3)c3ccccc3-c3ccccc32)cc1]

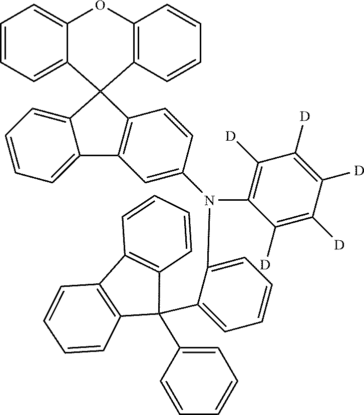 [SMILES: [2H]c1c([2H])c([2H])c(N(c2ccc3c(c2)-c2ccccc2C32c3ccccc3Oc3ccccc32)c2ccccc2C2(c3ccccc3)c3ccccc3-c3ccccc32)c([2H])c1[2H]]

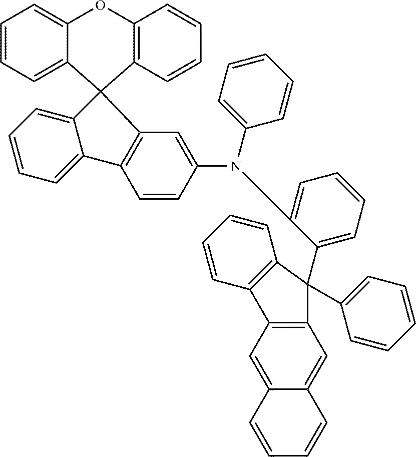 [SMILES: c1ccc(N(c2ccc3c(c2)C2(c4ccccc4Oc4ccccc42)c2ccccc2-3)c2ccccc2C2(c3ccccc3)c3ccccc3-c3cc4ccccc4cc32)cc1]

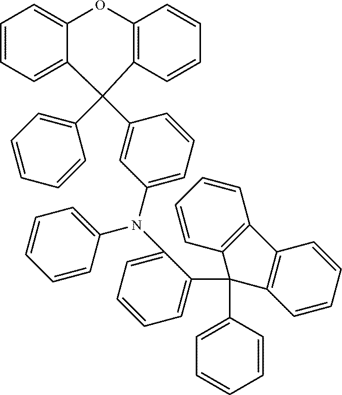 [SMILES: c1ccc(N(c2cccc(C3(c4ccccc4)c4ccccc4Oc4ccccc43)c2)c2ccccc2C2(c3ccccc3)c3ccccc3-c3ccccc32)cc1]

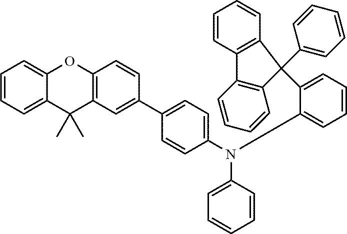 [SMILES: CC1(C)c2ccccc2Oc2ccc(-c3ccc(N(c4ccccc4)c4ccccc4C4(c5ccccc5)c5ccccc5-c5ccccc54)cc3)cc21]

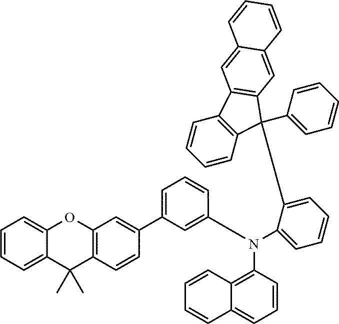 [SMILES: CC1(C)c2ccccc2Oc2cc(-c3cccc(N(c4ccccc4C4(c5ccccc5)c5ccccc5-c5cc6ccccc6cc54)c4cccc5ccccc45)c3)ccc21]

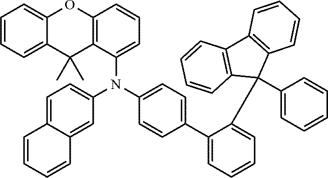 [SMILES: CC1(C)c2ccccc2Oc2cccc(N(c3ccc(-c4ccccc4CC4(c5ccccc5)c5ccccc5-c5ccccc54)cc3)c3ccc4ccccc4c3)c21]

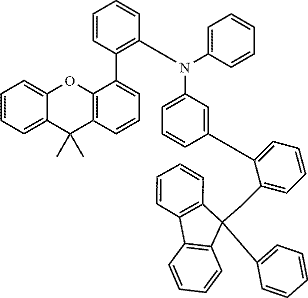 [SMILES: CC1(C)c2ccccc2Oc2c(-c3ccccc3CN(c3ccccc3)c3cccc(Cc4ccccc4CC4(Cc5ccccc5)c5ccccc5-c5ccccc54)c3)cccc21]

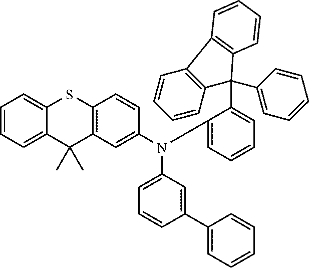 [SMILES: CC1(C)c2ccccc2Sc2ccc(N(Cc3ccccc3C3(c4ccccc4)c4ccccc4-c4ccccc43)c3cccc(-c4ccccc4)c3)cc21]

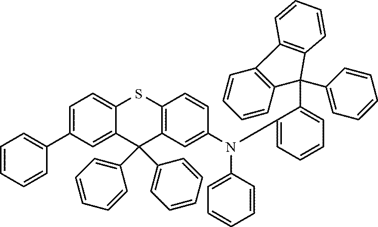 [SMILES: c1ccc(-c2ccc3c(c2)C(c2ccccc2)(c2ccccc2)c2cc(N(Cc4ccccc4C4(c5ccccc5)c5ccccc5-c5ccccc54)c4ccccc4)ccc2S3)cc1]

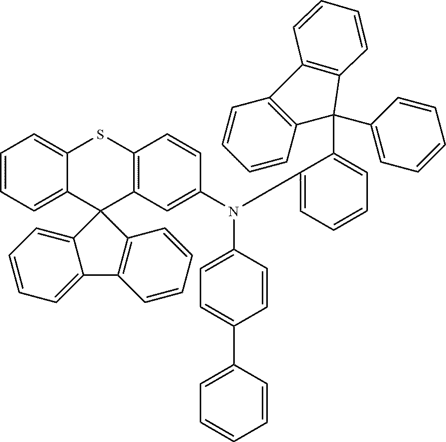 [SMILES: c1ccc(-c2ccc(N(Cc3ccccc3C3(c4ccccc4)c4ccccc4-c4ccccc43)c3ccc4c(c3)C3(c5ccccc5S4)c4ccccc4-c4ccccc43)cc2)cc1]

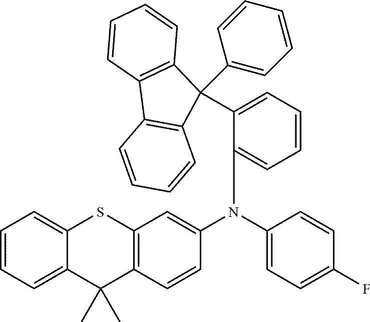 [SMILES: CC1(C)c2ccccc2Sc2cc(N(c3ccc(F)cc3)c3ccccc3C3(c4ccccc4)c4ccccc4-c4ccccc43)ccc21]

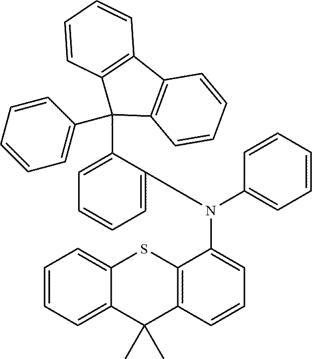 [SMILES: CC1(C)c2ccccc2Sc2c(N(c3ccccc3)c3ccccc3C3(c4ccccc4)c4ccccc4-c4ccccc43)cccc21]

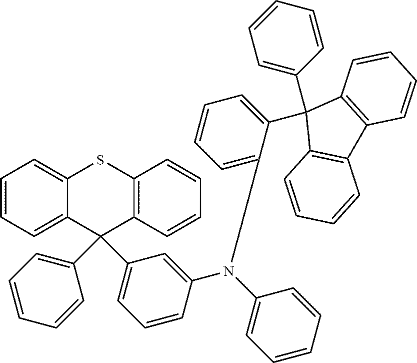 [SMILES: c1ccc(N(c2cccc(C3(c4ccccc4)c4ccccc4Sc4ccccc43)c2)c2ccccc2C2(c3ccccc3)c3ccccc3-c3ccccc32)cc1]

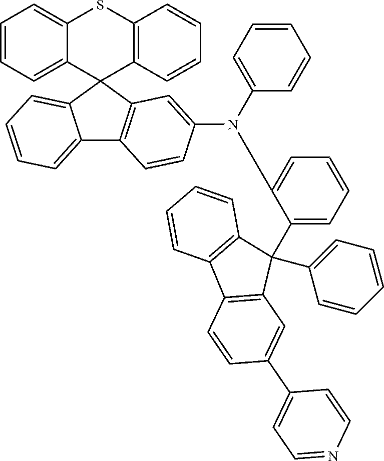 [SMILES: c1ccc(N(c2ccc3c(c2)C2(c4ccccc4Sc4ccccc42)c2ccccc2-3)c2ccccc2C2(c3ccccc3)c3ccccc3-c3ccc(-c4ccncc4)cc32)cc1]

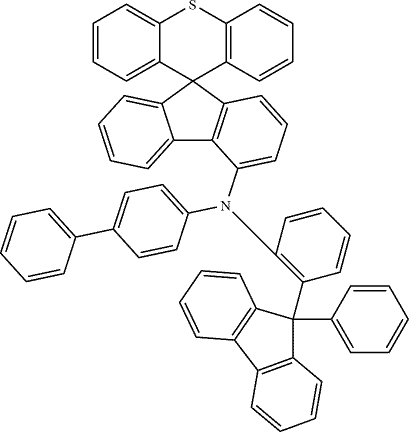 [SMILES: c1ccc(-c2ccc(N(c3ccccc3C3(c4ccccc4)c4ccccc4-c4ccccc43)c3cccc4c3-c3ccccc3C43c4ccccc4Sc4ccccc43)cc2)cc1]

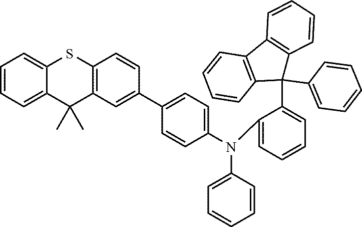 [SMILES: CC1(C)c2ccccc2Sc2ccc(-c3ccc(N(c4ccccc4)c4ccccc4C4(c5ccccc5)c5ccccc5-c5ccccc54)cc3)cc21]

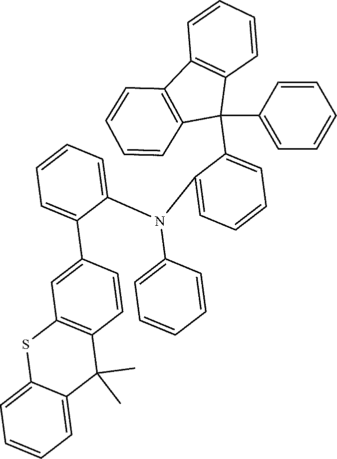 [SMILES: CC1(C)c2ccccc2Sc2cc(-c3ccccc3N(c3ccccc3)c3ccccc3C3(c4ccccc4)c4ccccc4-c4ccccc43)ccc21]

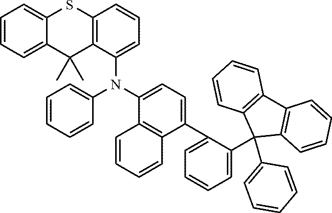 [SMILES: CC1(C)c2ccccc2Sc2cccc(N(c3ccccc3)c3ccc(-c4ccccc4C4(c5ccccc5)c5ccccc5-c5ccccc54)c4ccccc34)c21]

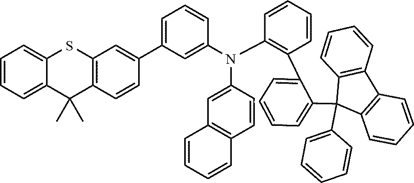 [SMILES: CC1(C)c2ccccc2Sc2cc(-c3cccc(N(c4ccc5ccccc5c4)c4ccccc4-c4ccccc4C4(c5ccccc5)c5ccccc5-c5ccccc54)c3)ccc21]

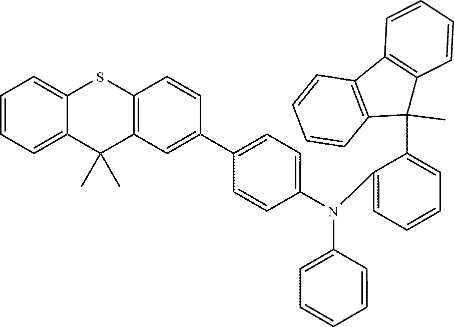 [SMILES: CC1(C)c2ccccc2Sc2ccc(-c3ccc(N(c4ccccc4)c4ccccc4C4(C)c5ccccc5-c5ccccc54)cc3)cc21]

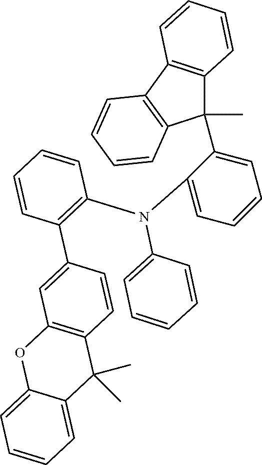 [SMILES: CC1(C)c2ccccc2Oc2cc(-c3ccccc3N(c3ccccc3)c3ccccc3C3(C)c4ccccc4-c4ccccc43)ccc21]

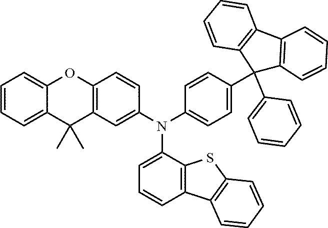 [SMILES: CC1(C)c2ccccc2Oc2ccc(N(c3ccc(C4(c5ccccc5)c5ccccc5-c5ccccc54)cc3)c3cccc4c3sc3ccccc34)cc21]

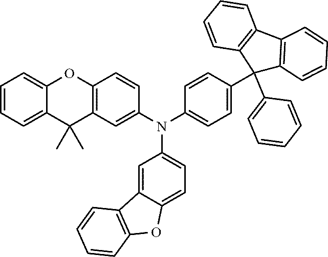 [SMILES: CC1(C)c2ccccc2Oc2ccc(N(c3ccc(C4(c5ccccc5)c5ccccc5-c5ccccc54)cc3)c3ccc4oc5ccccc5c4c3)cc21]

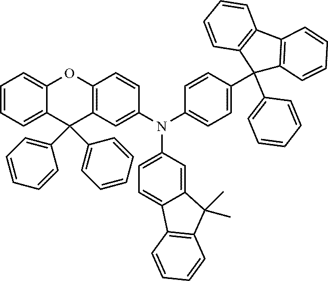 [SMILES: CC1(C)c2ccccc2-c2ccc(N(c3ccc(C4(c5ccccc5)c5ccccc5-c5ccccc54)cc3)c3ccc4c(c3)C(c3ccccc3)(c3ccccc3)c3ccccc3O4)cc21]

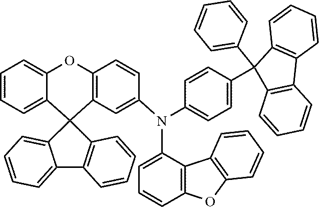 [SMILES: c1ccc(C2(c3ccc(N(c4ccc5c(c4)C4(c6ccccc6O5)c5ccccc5-c5ccccc54)c4cccc5oc6ccccc6c45)cc3)c3ccccc3-c3ccccc32)cc1]

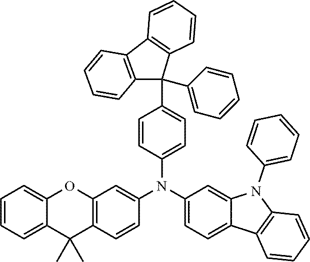 [SMILES: CC1(C)c2ccccc2Oc2cc(N(c3ccc(C4(c5ccccc5)c5ccccc5-c5ccccc54)cc3)c3ccc4c5ccccc5n(-c5ccccc5)c4c3)ccc21]

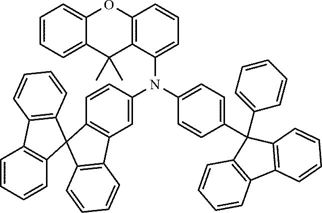 [SMILES: CC1(C)c2ccccc2Oc2cccc(N(c3ccc(C4(c5ccccc5)c5ccccc5-c5ccccc54)cc3)c3ccc4c(c3)-c3ccccc3C43c4ccccc4-c4ccccc43)c21]

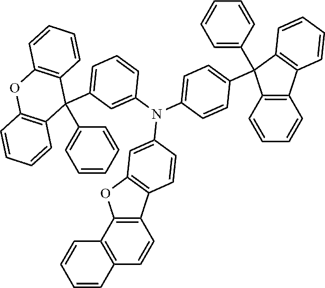 [SMILES: c1ccc(C2(c3cccc(N(c4ccc(C5(c6ccccc6)c6ccccc6-c6ccccc65)cc4)c4ccc5c(c4)oc4c6ccccc6ccc54)c3)c3ccccc3Oc3ccccc32)cc1]

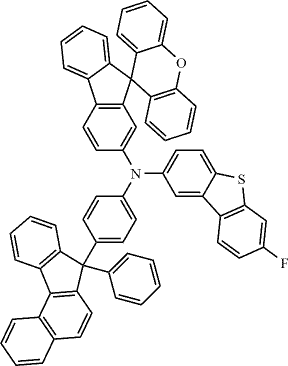 [SMILES: Fc1ccc2c(c1)sc1ccc(N(c3ccc(C4(c5ccccc5)c5ccccc5-c5c4ccc4ccccc54)cc3)c3ccc4c(c3)C3(c5ccccc5Oc5ccccc53)c3ccccc3-4)cc12]

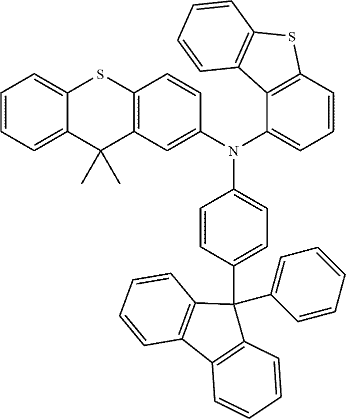 [SMILES: CC1(C)c2ccccc2Sc2ccc(N(c3ccc(C4(c5ccccc5)c5ccccc5-c5ccccc54)cc3)c3cccc4sc5ccccc5c34)cc21]

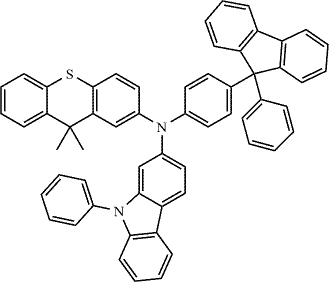 [SMILES: CC1(C)c2ccccc2Sc2ccc(N(c3ccc(C4(c5ccccc5)c5ccccc5-c5ccccc54)cc3)c3ccc4c5ccccc5n(-c5ccccc5)c4c3)cc21]

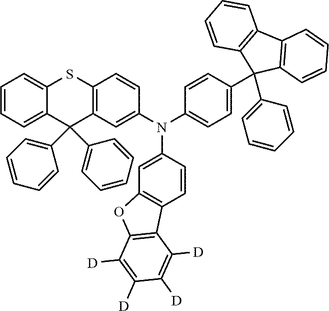 [SMILES: [2H]c1c([2H])c([2H])c2c(oc3cc(N(c4ccc(C5(c6ccccc6)c6ccccc6-c6ccccc65)cc4)c4ccc5c(c4)C(c4ccccc4)(c4ccccc4)c4ccccc4S5)ccc32)c1[2H]]

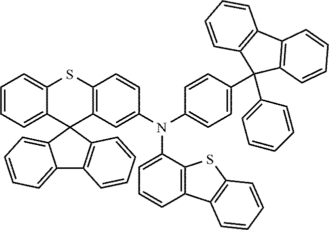 [SMILES: c1ccc(C2(c3ccc(N(c4ccc5c(c4)C4(c6ccccc6S5)c5ccccc5-c5ccccc54)c4cccc5c4sc4ccccc45)cc3)c3ccccc3-c3ccccc32)cc1]

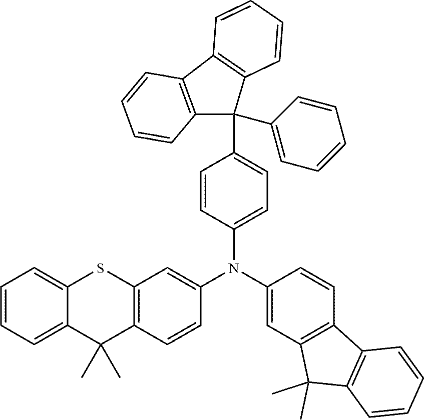 [SMILES: CC1(C)c2ccccc2Sc2cc(N(c3ccc(C4(c5ccccc5)c5ccccc5-c5ccccc54)cc3)c3ccc4c(c3)C(C)(C)c3ccccc3-4)ccc21]

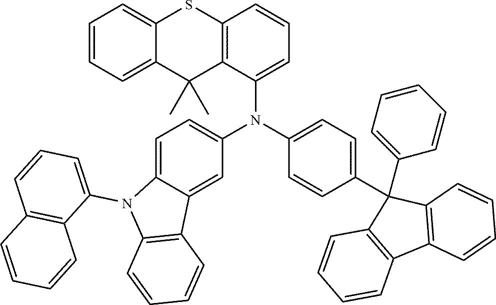 [SMILES: CC1(C)c2ccccc2Sc2cccc(N(c3ccc(C4(c5ccccc5)c5ccccc5-c5ccccc54)cc3)c3ccc4c(c3)c3ccccc3n4-c3cccc4ccccc34)c21]

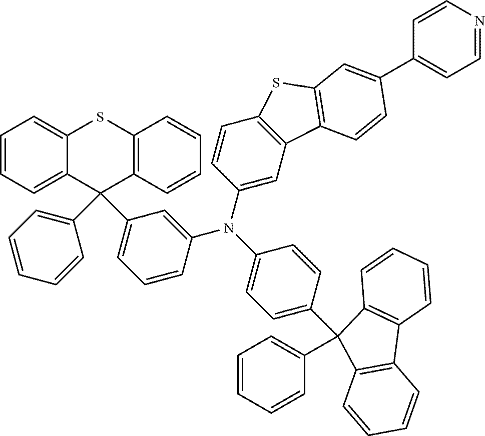 [SMILES: c1ccc(C2(c3cccc(N(c4ccc(C5(c6ccccc6)c6ccccc6-c6ccccc65)cc4)c4ccc5sc6cc(-c7ccncc7)ccc6c5c4)c3)c3ccccc3Sc3ccccc32)cc1]

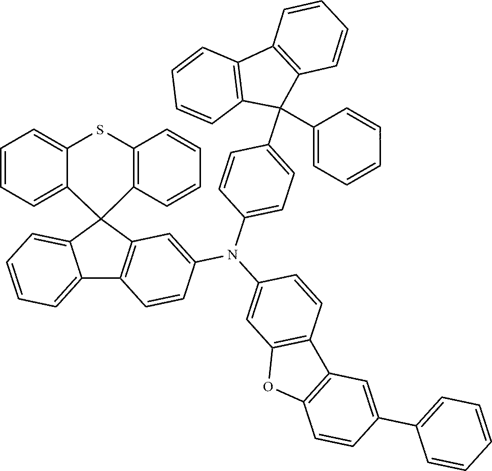 [SMILES: c1ccc(-c2ccc3oc4cc(N(c5ccc(C6(c7ccccc7)c7ccccc7-c7ccccc76)cc5)c5ccc6c(c5)C5(c7ccccc7Sc7ccccc75)c5ccccc5-6)ccc4c3c2)cc1]

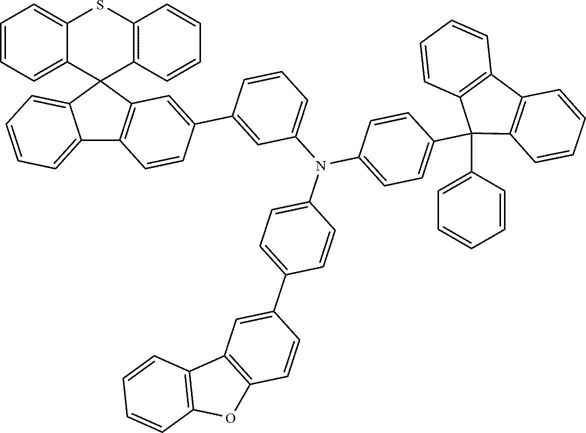 [SMILES: c1ccc(C2(c3ccc(N(c4ccc(-c5ccc6oc7ccccc7c6c5)cc4)c4cccc(-c5ccc6c(c5)C5(c7ccccc7Sc7ccccc75)c5ccccc5-6)c4)cc3)c3ccccc3-c3ccccc32)cc1]

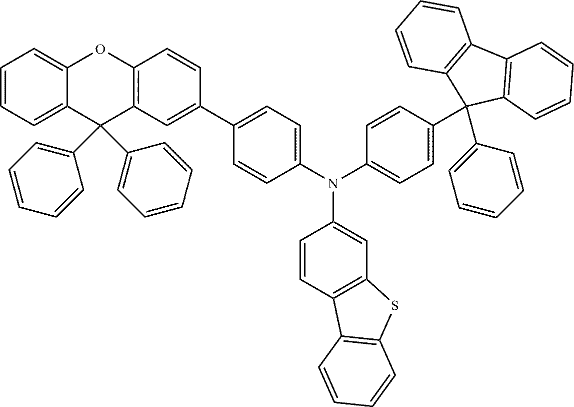 [SMILES: c1ccc(C2(c3ccccc3)c3ccccc3Oc3ccc(-c4ccc(N(c5ccc(C6(c7ccccc7)c7ccccc7-c7ccccc76)cc5)c5ccc6c(c5)sc5ccccc56)cc4)cc32)cc1]

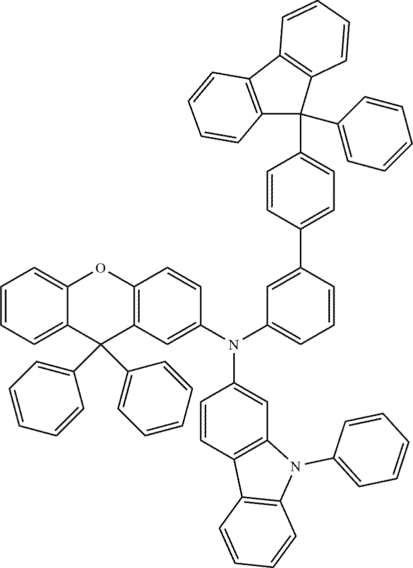 [SMILES: c1ccc(-n2c3ccccc3c3ccc(N(c4cccc(-c5ccc(C6(c7ccccc7)c7ccccc7-c7ccccc76)cc5)c4)c4ccc5c(c4)C(c4ccccc4)(c4ccccc4)c4ccccc4O5)cc32)cc1]

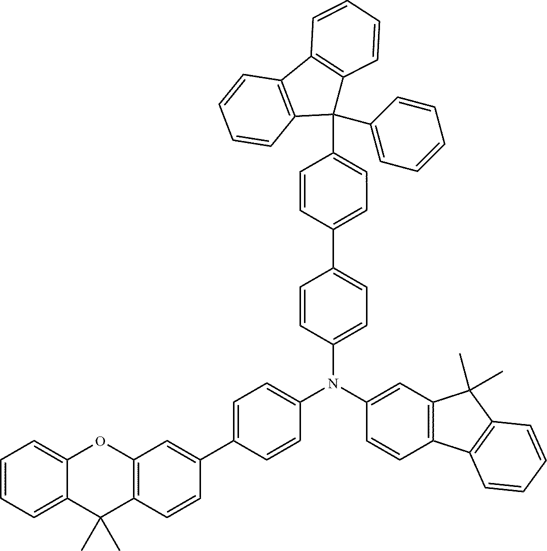 [SMILES: CC1(C)c2ccccc2Oc2cc(-c3ccc(N(c4ccc(-c5ccc(C6(c7ccccc7)c7ccccc7-c7ccccc76)cc5)cc4)c4ccc5c(c4)C(C)(C)c4ccccc4-5)cc3)ccc21]

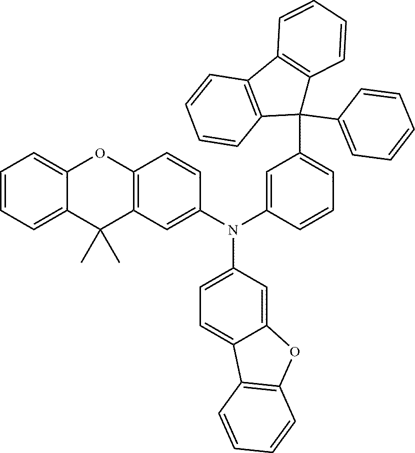 [SMILES: CC1(C)c2ccccc2Oc2ccc(N(c3cccc(C4(c5ccccc5)c5ccccc5-c5ccccc54)c3)c3ccc4c(c3)oc3ccccc34)cc21]

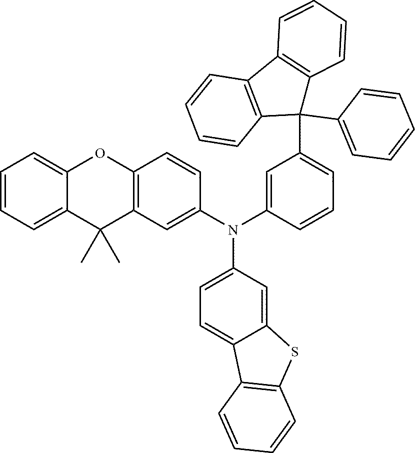 [SMILES: CC1(C)c2ccccc2Oc2ccc(N(c3cccc(C4(c5ccccc5)c5ccccc5-c5ccccc54)c3)c3ccc4c(c3)sc3ccccc34)cc21]

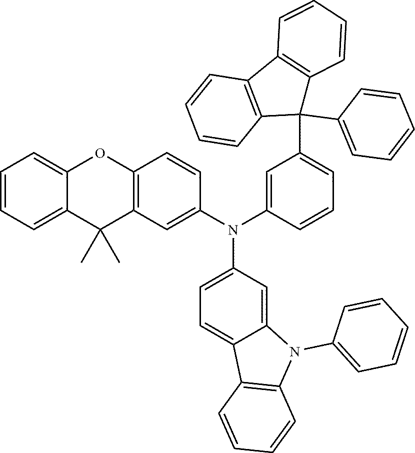 [SMILES: CC1(C)c2ccccc2Oc2ccc(N(c3cccc(C4(c5ccccc5)c5ccccc5-c5ccccc54)c3)c3ccc4c5ccccc5n(-c5ccccc5)c4c3)cc21]

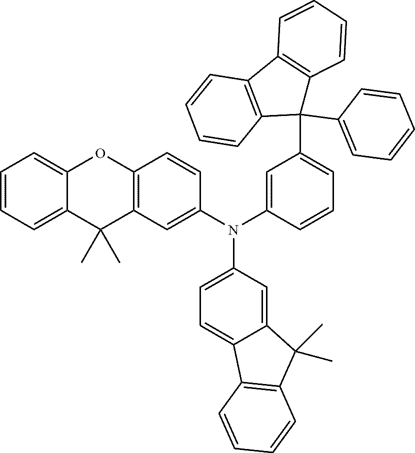 [SMILES: CC1(C)c2ccccc2Oc2ccc(N(c3cccc(C4(c5ccccc5)c5ccccc5-c5ccccc54)c3)c3ccc4c(c3)C(C)(C)c3ccccc3-4)cc21]

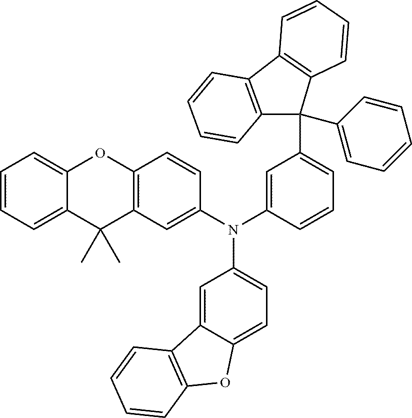 [SMILES: CC1(C)c2ccccc2Oc2ccc(N(c3cccc(C4(c5ccccc5)c5ccccc5-c5ccccc54)c3)c3ccc4oc5ccccc5c4c3)cc21]

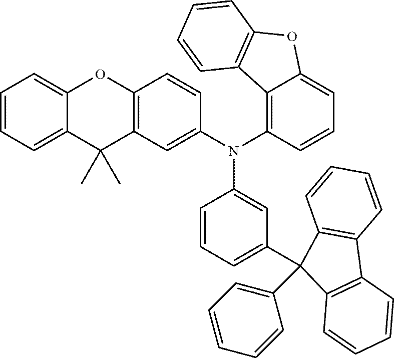 [SMILES: CC1(C)c2ccccc2Oc2ccc(N(c3cccc(C4(c5ccccc5)c5ccccc5-c5ccccc54)c3)c3cccc4oc5ccccc5c34)cc21]

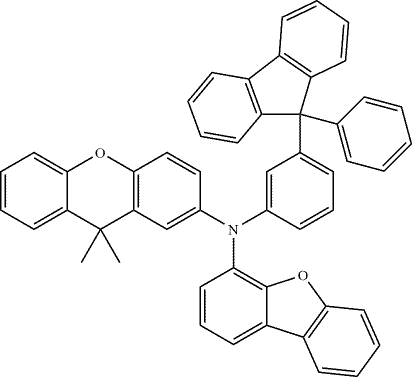 [SMILES: CC1(C)c2ccccc2Oc2ccc(N(c3cccc(C4(c5ccccc5)c5ccccc5-c5ccccc54)c3)c3cccc4c3oc3ccccc34)cc21]

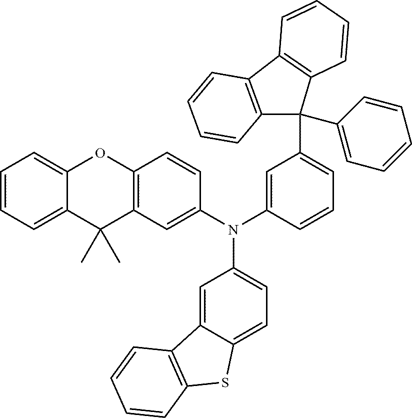 [SMILES: CC1(C)c2ccccc2Oc2ccc(N(c3cccc(C4(c5ccccc5)c5ccccc5-c5ccccc54)c3)c3ccc4sc5ccccc5c4c3)cc21]

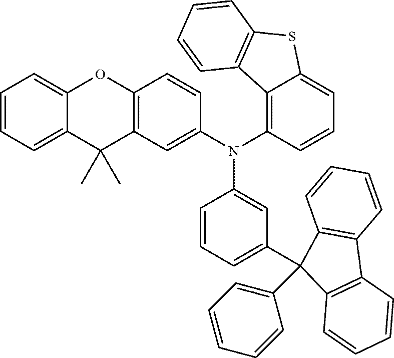 [SMILES: CC1(C)c2ccccc2Oc2ccc(N(c3cccc(C4(c5ccccc5)c5ccccc5-c5ccccc54)c3)c3cccc4sc5ccccc5c34)cc21]

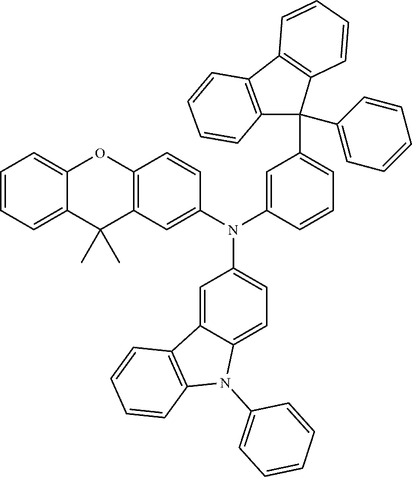 [SMILES: CC1(C)c2ccccc2Oc2ccc(N(c3cccc(C4(c5ccccc5)c5ccccc5-c5ccccc54)c3)c3ccc4c(c3)c3ccccc3n4-c3ccccc3)cc21]

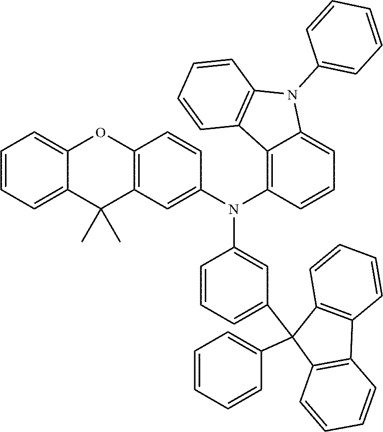 [SMILES: CC1(C)c2ccccc2Oc2ccc(N(c3cccc(C4(c5ccccc5)c5ccccc5-c5ccccc54)c3)c3cccc4c3c3ccccc3n4-c3ccccc3)cc21]

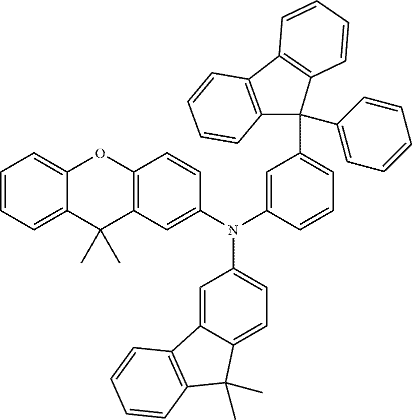 [SMILES: CC1(C)c2ccccc2Oc2ccc(N(c3cccc(C4(c5ccccc5)c5ccccc5-c5ccccc54)c3)c3ccc4c(c3)-c3ccccc3C4(C)C)cc21]

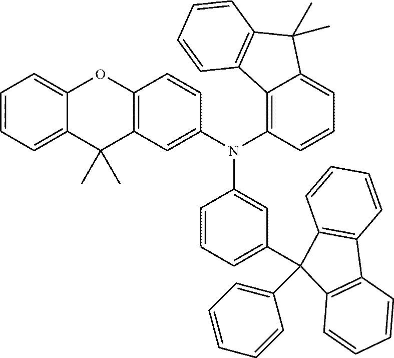 [SMILES: CC1(C)c2ccccc2Oc2ccc(N(c3cccc(C4(c5ccccc5)c5ccccc5-c5ccccc54)c3)c3cccc4c3-c3ccccc3C4(C)C)cc21]

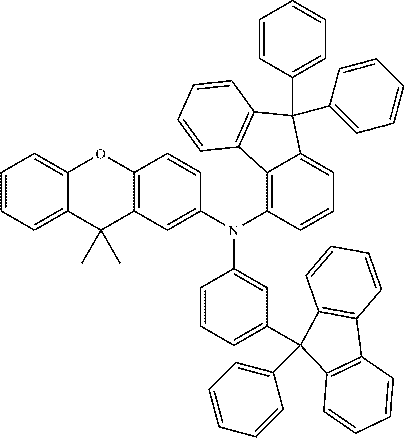 [SMILES: CC1(C)c2ccccc2Oc2ccc(N(c3cccc(C4(c5ccccc5)c5ccccc5-c5ccccc54)c3)c3cccc4c3-c3ccccc3C4(c3ccccc3)c3ccccc3)cc21]

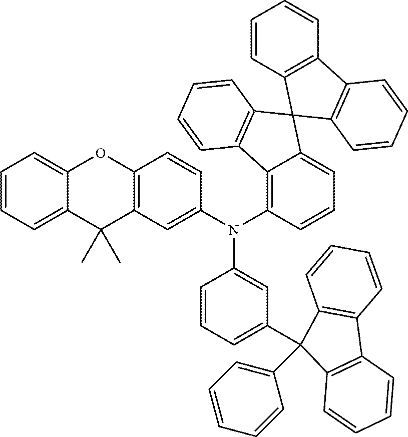 [SMILES: CC1(C)c2ccccc2Oc2ccc(N(c3cccc(C4(c5ccccc5)c5ccccc5-c5ccccc54)c3)c3cccc4c3-c3ccccc3C43c4ccccc4-c4ccccc43)cc21]

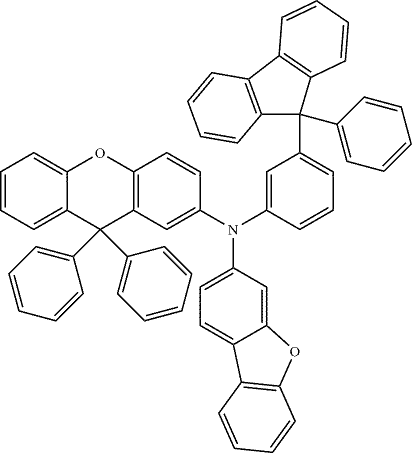 [SMILES: c1ccc(C2(c3ccccc3)c3ccccc3Oc3ccc(N(c4cccc(C5(c6ccccc6)c6ccccc6-c6ccccc65)c4)c4ccc5c(c4)oc4ccccc45)cc32)cc1]

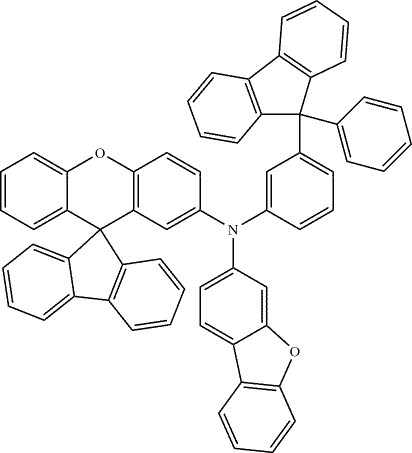 [SMILES: c1ccc(C2(c3cccc(N(c4ccc5c(c4)C4(c6ccccc6O5)c5ccccc5-c5ccccc54)c4ccc5c(c4)oc4ccccc45)c3)c3ccccc3-c3ccccc32)cc1]

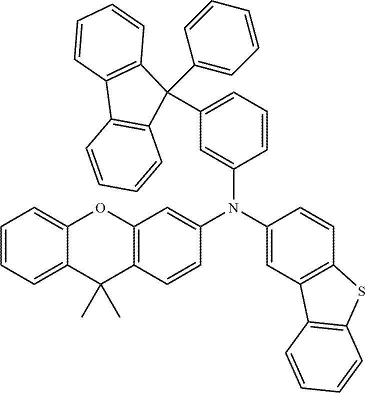 [SMILES: CC1(C)c2ccccc2Oc2cc(N(c3cccc(C4(c5ccccc5)c5ccccc5-c5ccccc54)c3)c3ccc4sc5ccccc5c4c3)ccc21]

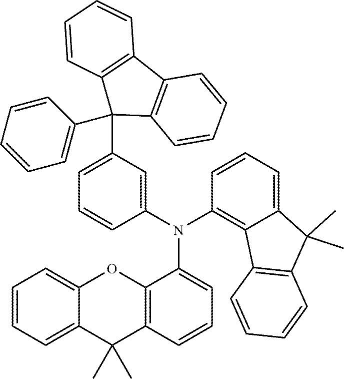 [SMILES: CC1(C)c2ccccc2Oc2c(N(c3cccc(C4(c5ccccc5)c5ccccc5-c5ccccc54)c3)c3cccc4c3-c3ccccc3C4(C)C)cccc21]

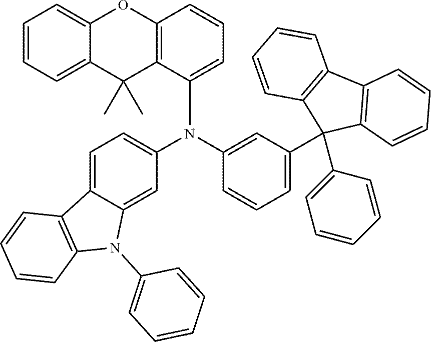 [SMILES: CC1(C)c2ccccc2Oc2cccc(N(c3cccc(C4(c5ccccc5)c5ccccc5-c5ccccc54)c3)c3ccc4c5ccccc5n(-c5ccccc5)c4c3)c21]

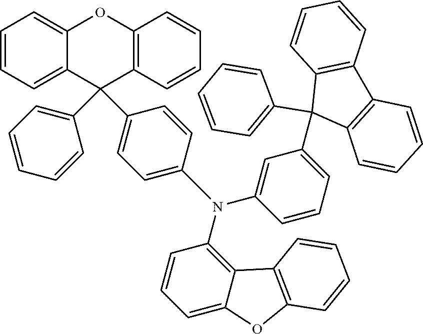 [SMILES: c1ccc(C2(c3ccc(N(c4cccc(C5(c6ccccc6)c6ccccc6-c6ccccc65)c4)c4cccc5oc6ccccc6c45)cc3)c3ccccc3Oc3ccccc32)cc1]

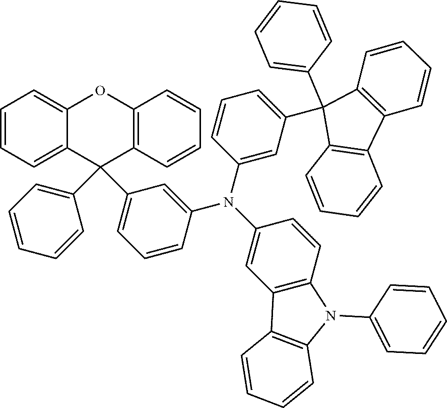 [SMILES: c1ccc(-n2c3ccccc3c3cc(N(c4cccc(C5(c6ccccc6)c6ccccc6Oc6ccccc65)c4)c4cccc(C5(c6ccccc6)c6ccccc6-c6ccccc65)c4)ccc32)cc1]

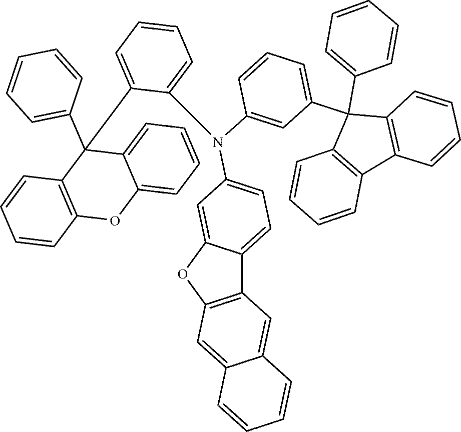

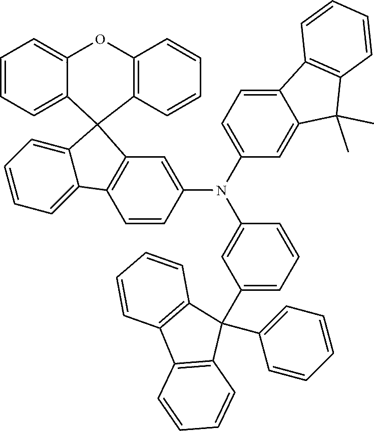 [SMILES: CC1(C)c2ccccc2-c2ccc(N(c3cccc(C4(c5ccccc5)c5ccccc5-c5ccccc54)c3)c3ccc4c(c3)C3(c5ccccc5Oc5ccccc53)c3ccccc3-4)cc21]

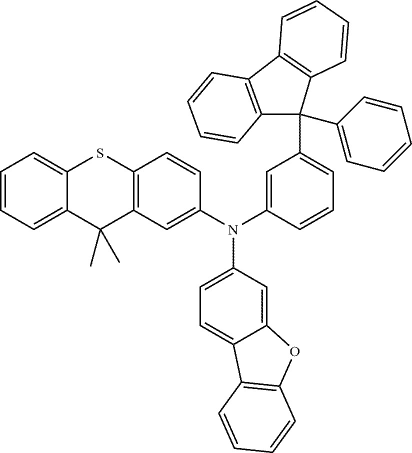 [SMILES: CC1(C)c2ccccc2Sc2ccc(N(c3cccc(C4(c5ccccc5)c5ccccc5-c5ccccc54)c3)c3ccc4c(c3)oc3ccccc34)cc21]

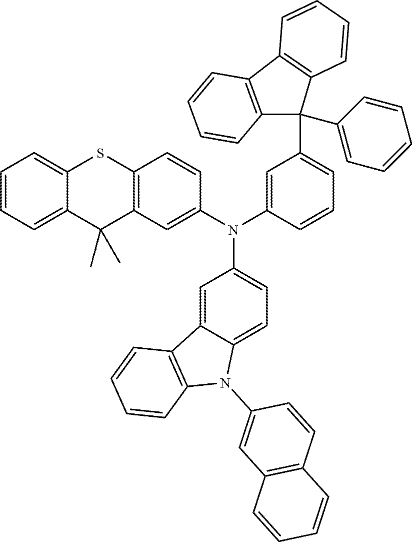 [SMILES: CC1(C)c2ccccc2Sc2ccc(N(c3cccc(C4(c5ccccc5)c5ccccc5-c5ccccc54)c3)c3ccc4c(c3)c3ccccc3n4-c3ccc4ccccc4c3)cc21]

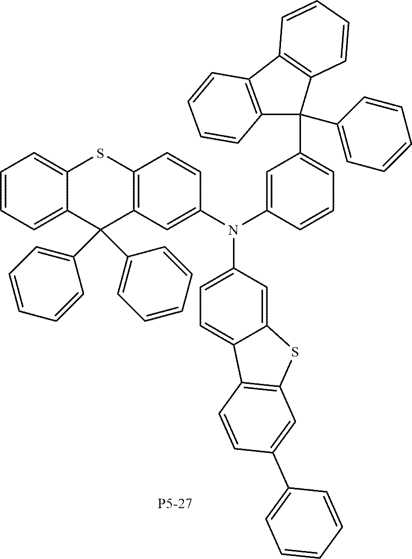

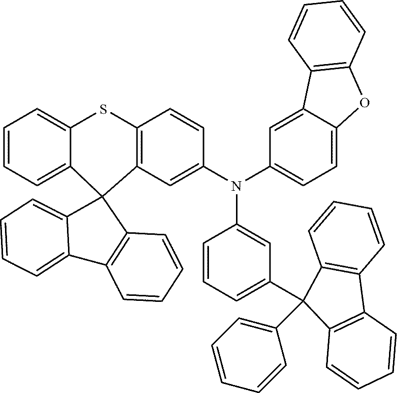 [SMILES: c1ccc(C2(c3cccc(N(c4ccc5c(c4)C4(c6ccccc6S5)c5ccccc5-c5ccccc54)c4ccc5oc6ccccc6c5c4)c3)c3ccccc3-c3ccccc32)cc1]

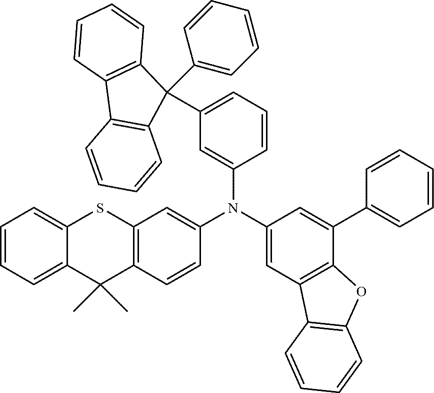 [SMILES: CC1(C)c2ccccc2Sc2cc(N(c3cccc(C4(c5ccccc5)c5ccccc5-c5ccccc54)c3)c3cc(-c4ccccc4)c4oc5ccccc5c4c3)ccc21]

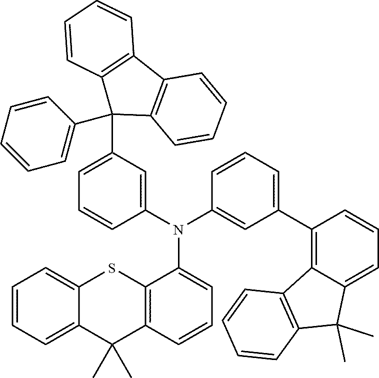 [SMILES: CC1(C)c2ccccc2Sc2c(N(c3cccc(-c4cccc5c4-c4ccccc4C5(C)C)c3)c3cccc(C4(c5ccccc5)c5ccccc5-c5ccccc54)c3)cccc21]

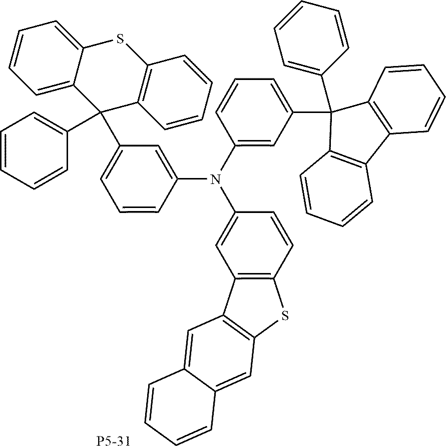

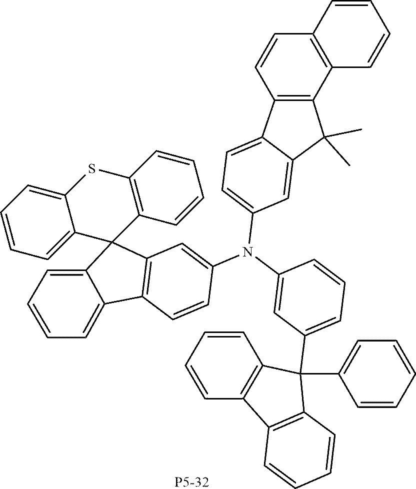

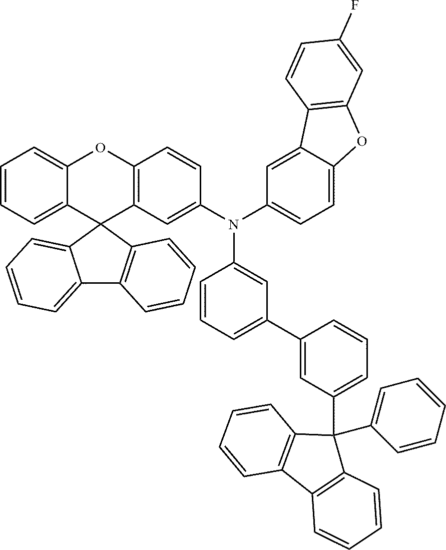 [SMILES: Fc1ccc2c(c1)oc1ccc(N(c3cccc(-c4cccc(C5(c6ccccc6)c6ccccc6-c6ccccc65)c4)c3)c3ccc4c(c3)C3(c5ccccc5O4)c4ccccc4-c4ccccc43)cc12]

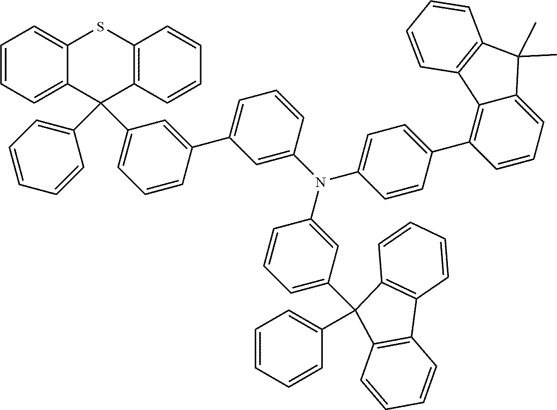 [SMILES: CC1(C)c2ccccc2-c2c(-c3ccc(N(c4cccc(-c5cccc(C6(c7ccccc7)c7ccccc7Sc7ccccc76)c5)c4)c4cccc(C5(c6ccccc6)c6ccccc6-c6ccccc65)c4)cc3)cccc21]

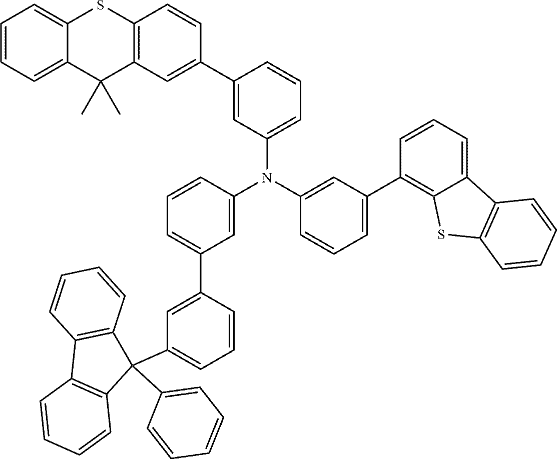 [SMILES: CC1(C)c2ccccc2Sc2ccc(-c3cccc(N(c4cccc(-c5cccc(C6(c7ccccc7)c7ccccc7-c7ccccc76)c5)c4)c4cccc(-c5cccc6c5sc5ccccc56)c4)c3)cc21]

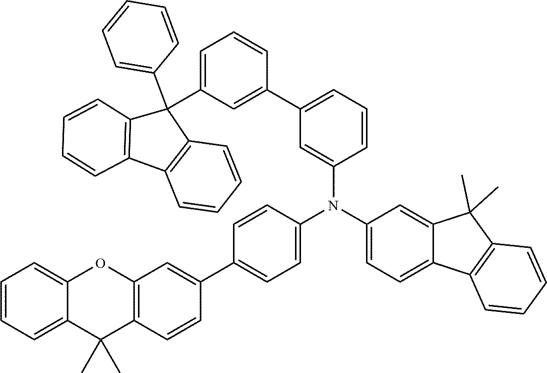 [SMILES: CC1(C)c2ccccc2Oc2cc(-c3ccc(N(c4cccc(-c5cccc(C6(c7ccccc7)c7ccccc7-c7ccccc76)c5)c4)c4ccc5c(c4)C(C)(C)c4ccccc4-5)cc3)ccc21]

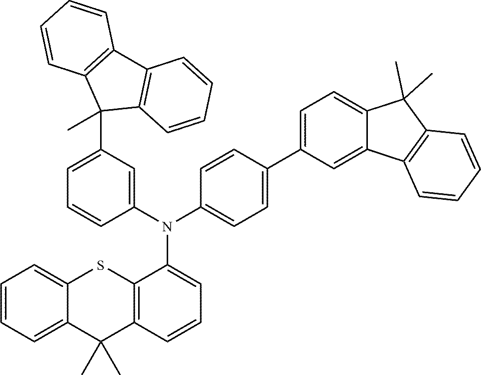 [SMILES: CC1(C)c2ccccc2-c2cc(-c3ccc(N(c4cccc(C5(C)c6ccccc6-c6ccccc65)c4)c4cccc5c4Sc4ccccc4C5(C)C)cc3)ccc21]

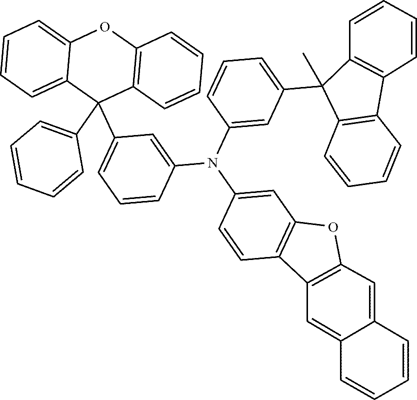 [SMILES: CC1(c2cccc(N(c3cccc(C4(c5ccccc5)c5ccccc5Oc5ccccc54)c3)c3ccc4c(c3)oc3cc5ccccc5cc34)c2)c2ccccc2-c2ccccc21]

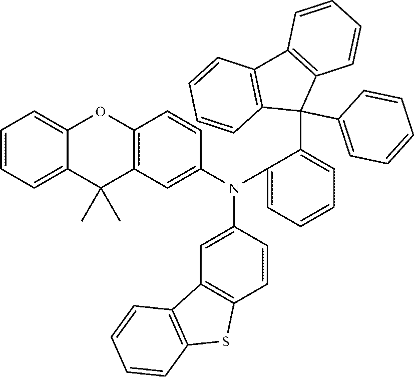 [SMILES: CC1(C)c2ccccc2Oc2ccc(N(c3ccc4sc5ccccc5c4c3)c3ccccc3C3(c4ccccc4)c4ccccc4-c4ccccc43)cc21]

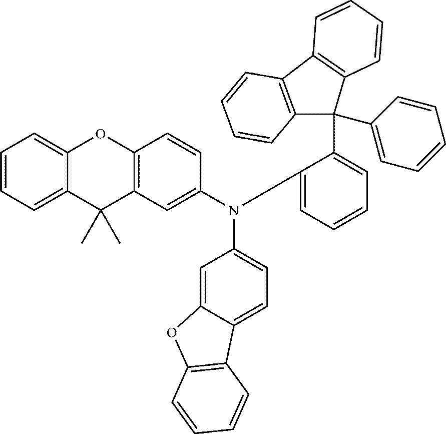 [SMILES: CC1(C)c2ccccc2Oc2ccc(N(c3ccc4c(c3)oc3ccccc34)c3ccccc3C3(c4ccccc4)c4ccccc4-c4ccccc43)cc21]

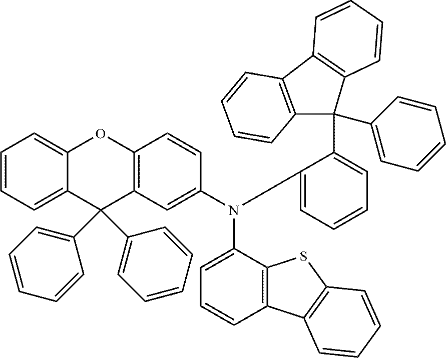 [SMILES: c1ccc(C2(c3ccccc3)c3ccccc3Oc3ccc(N(c4ccccc4C4(c5ccccc5)c5ccccc5-c5ccccc54)c4cccc5c4sc4ccccc45)cc32)cc1]

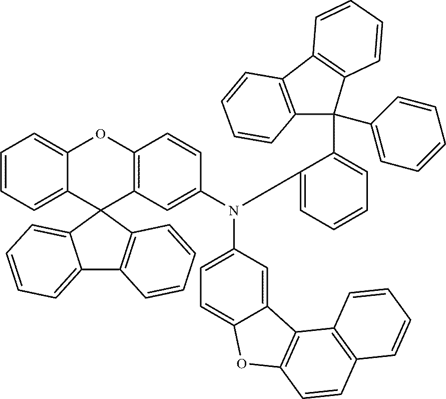 [SMILES: c1ccc(C2(c3ccccc3N(c3ccc4c(c3)C3(c5ccccc5O4)c4ccccc4-c4ccccc43)c3ccc4oc5ccc6ccccc6c5c4c3)c3ccccc3-c3ccccc32)cc1]

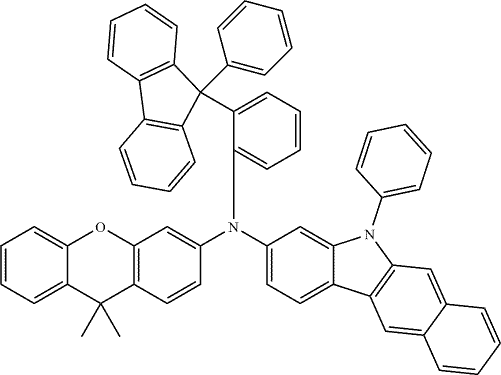 [SMILES: CC1(C)c2ccccc2Oc2cc(N(c3ccc4c5cc6ccccc6cc5n(-c5ccccc5)c4c3)c3ccccc3C3(c4ccccc4)c4ccccc4-c4ccccc43)ccc21]

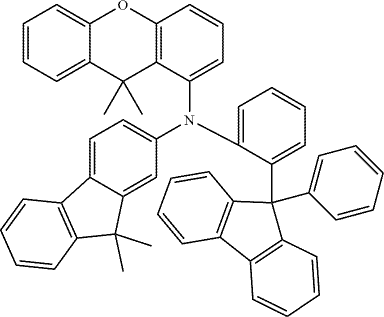 [SMILES: CC1(C)c2ccccc2-c2ccc(N(c3ccccc3C3(c4ccccc4)c4ccccc4-c4ccccc43)c3cccc4c3C(C)(C)c3ccccc3O4)cc21]

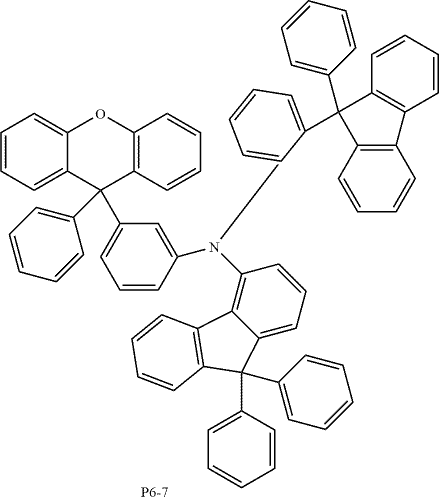

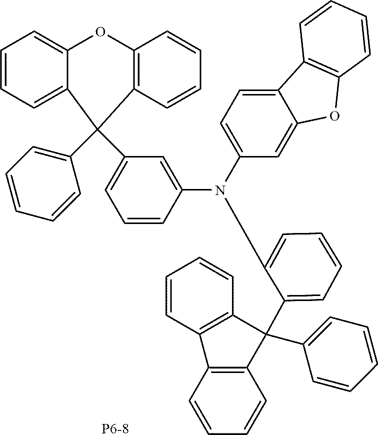

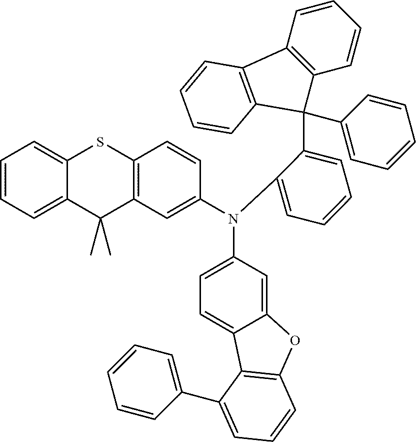 [SMILES: CC1(C)c2ccccc2Sc2ccc(N(c3ccc4c(c3)oc3cccc(-c5ccccc5)c34)c3ccccc3C3(c4ccccc4)c4ccccc4-c4ccccc43)cc21]

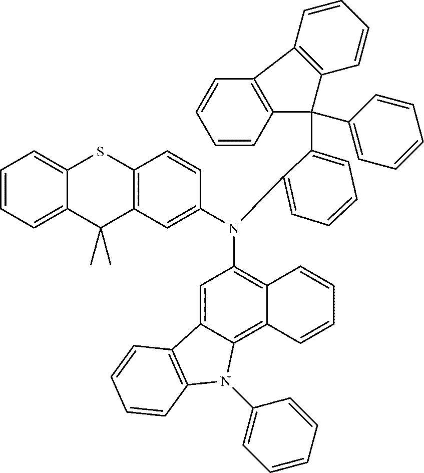 [SMILES: CC1(C)c2ccccc2Sc2ccc(N(c3ccccc3C3(c4ccccc4)c4ccccc4-c4ccccc43)c3cc4c5ccccc5n(-c5ccccc5)c4c4ccccc34)cc21]

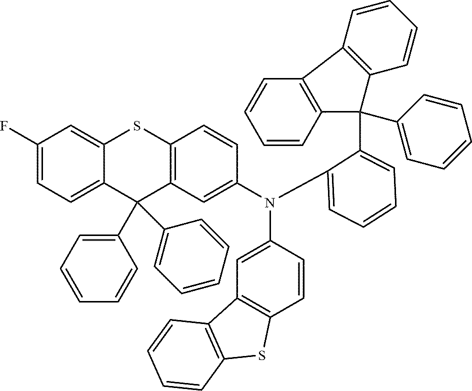 [SMILES: Fc1ccc2c(c1)Sc1ccc(N(c3ccc4sc5ccccc5c4c3)c3ccccc3C3(c4ccccc4)c4ccccc4-c4ccccc43)cc1C2(c1ccccc1)c1ccccc1]

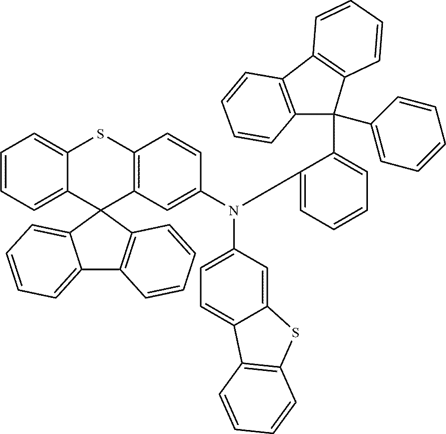 [SMILES: c1ccc(C2(c3ccccc3N(c3ccc4c(c3)C3(c5ccccc5S4)c4ccccc4-c4ccccc43)c3ccc4c(c3)sc3ccccc34)c3ccccc3-c3ccccc32)cc1]

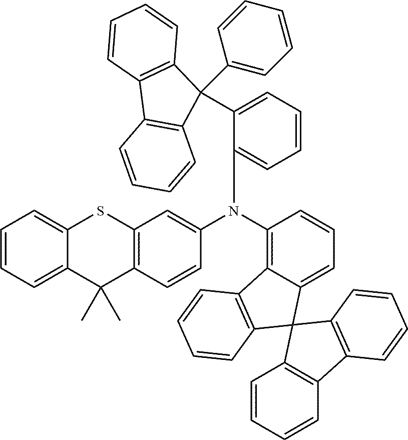 [SMILES: CC1(C)c2ccccc2Sc2cc(N(c3ccccc3C3(c4ccccc4)c4ccccc4-c4ccccc43)c3cccc4c3-c3ccccc3C43c4ccccc4-c4ccccc43)ccc21]

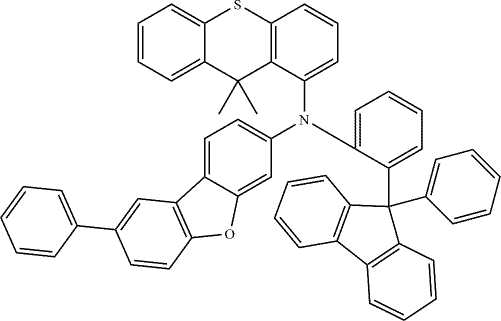 [SMILES: CC1(C)c2ccccc2Sc2cccc(N(c3ccc4c(c3)oc3ccc(-c5ccccc5)cc34)c3ccccc3C3(c4ccccc4)c4ccccc4-c4ccccc43)c21]

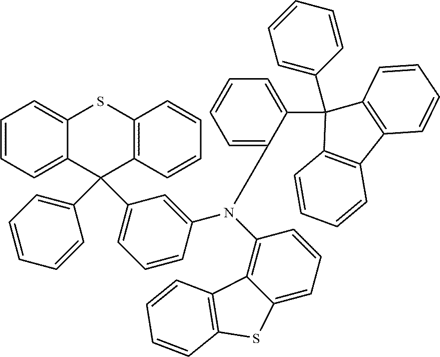 [SMILES: c1ccc(C2(c3cccc(N(c4ccccc4C4(c5ccccc5)c5ccccc5-c5ccccc54)c4cccc5sc6ccccc6c45)c3)c3ccccc3Sc3ccccc32)cc1]

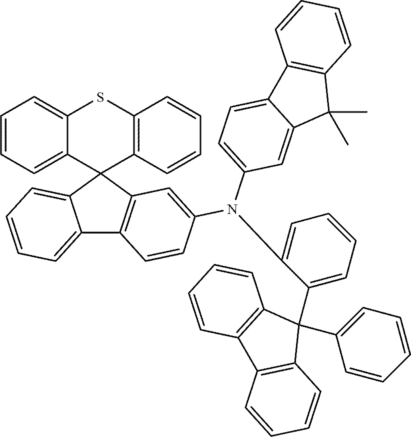 [SMILES: CC1(C)c2ccccc2-c2ccc(N(c3ccc4c(c3)C3(c5ccccc5Sc5ccccc53)c3ccccc3-4)c3ccccc3C3(c4ccccc4)c4ccccc4-c4ccccc43)cc21]

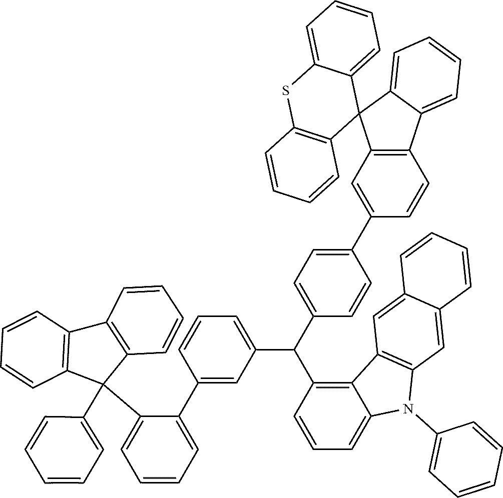 [SMILES: c1ccc(-n2c3cc4ccccc4cc3c3c(C(c4ccc(-c5ccc6c(c5)C5(c7ccccc7Sc7ccccc75)c5ccccc5-6)cc4)c4cccc(-c5ccccc5C5(c6ccccc6)c6ccccc6-c6ccccc65)c4)cccc32)cc1]

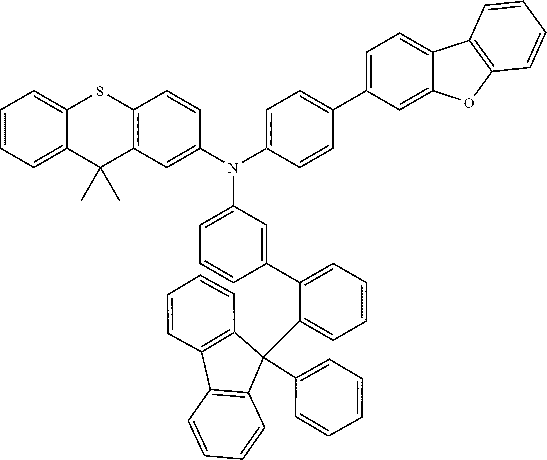 [SMILES: CC1(C)c2ccccc2Sc2ccc(N(c3ccc(-c4ccc5c(c4)oc4ccccc45)cc3)c3cccc(-c4ccccc4C4(c5ccccc5)c5ccccc5-c5ccccc54)c3)cc21]

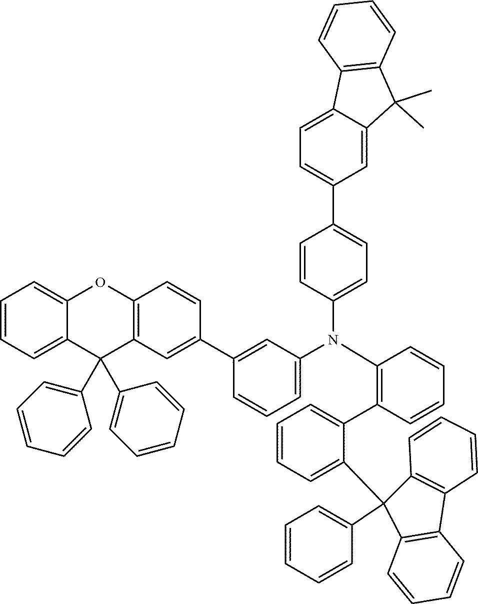 [SMILES: CC1(C)c2ccccc2-c2ccc(-c3ccc(N(c4cccc(-c5ccc6c(c5)C(c5ccccc5)(c5ccccc5)c5ccccc5O6)c4)c4ccccc4-c4ccccc4C4(c5ccccc5)c5ccccc5-c5ccccc54)cc3)cc21]

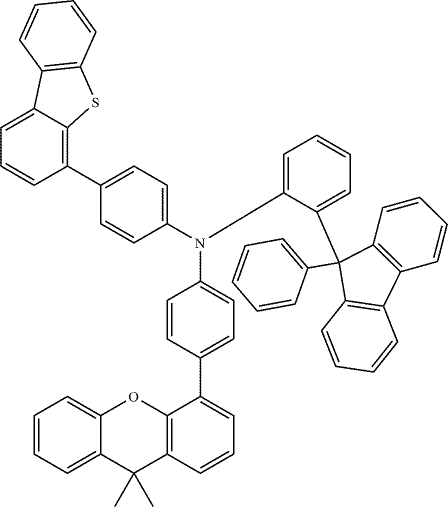 [SMILES: CC1(C)c2ccccc2Oc2c(-c3ccc(N(c4ccc(-c5cccc6c5sc5ccccc56)cc4)c4ccccc4C4(c5ccccc5)c5ccccc5-c5ccccc54)cc3)cccc21]

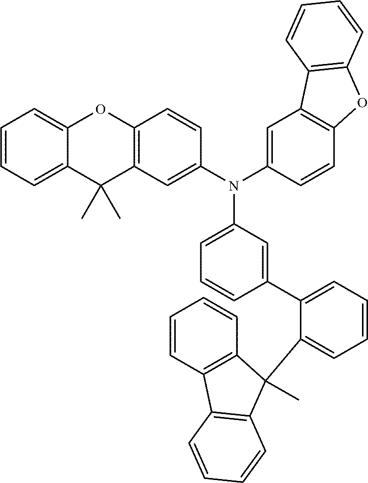 [SMILES: CC1(C)c2ccccc2Oc2ccc(N(c3cccc(-c4ccccc4C4(C)c5ccccc5-c5ccccc54)c3)c3ccc4oc5ccccc5c4c3)cc21]

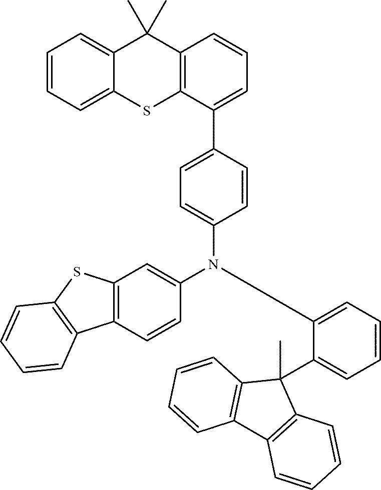 [SMILES: CC1(C)c2ccccc2Sc2c(-c3ccc(N(c4ccc5c(c4)sc4ccccc45)c4ccccc4C4(C)c5ccccc5-c5ccccc54)cc3)cccc21]

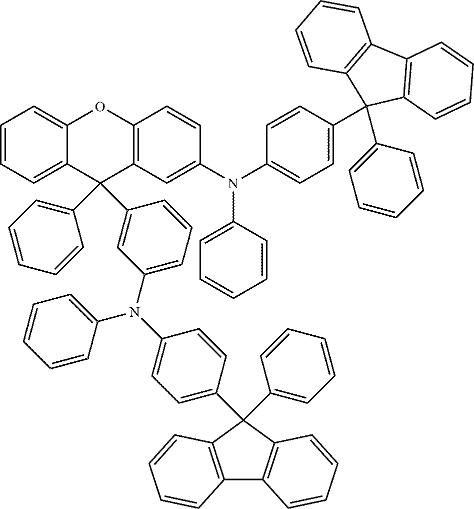 [SMILES: c1ccc(N(c2ccc(C3(c4ccccc4)c4ccccc4-c4ccccc43)cc2)c2cccc(C3(c4ccccc4)c4ccccc4Oc4ccc(N(c5ccccc5)c5ccc(C6(c7ccccc7)c7ccccc7-c7ccccc76)cc5)cc43)c2)cc1]

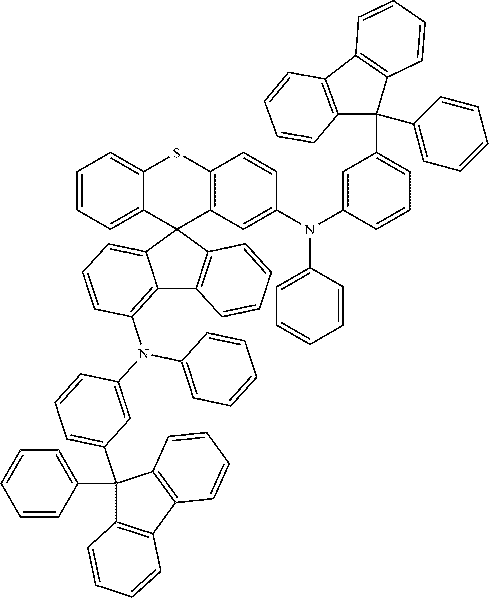 [SMILES: c1ccc(N(c2cccc(C3(c4ccccc4)c4ccccc4-c4ccccc43)c2)c2ccc3c(c2)C2(c4ccccc4S3)c3ccccc3-c3c(N(c4ccccc4)c4cccc(C5(c6ccccc6)c6ccccc6-c6ccccc65)c4)cccc32)cc1]

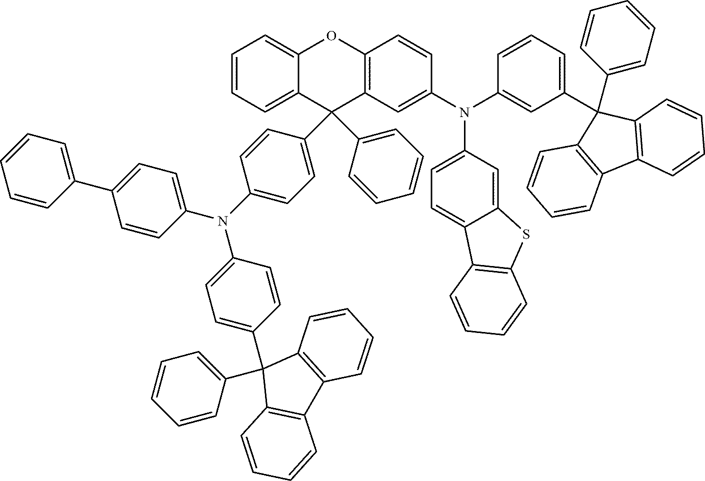 [SMILES: c1ccc(-c2ccc(N(c3ccc(C4(c5ccccc5)c5ccccc5Oc5ccc(N(c6cccc(C7(c8ccccc8)c8ccccc8-c8ccccc87)c6)c6ccc7c(c6)sc6ccccc67)cc54)cc3)c3ccc(C4(c5ccccc5)c5ccccc5-c5ccccc54)cc3)cc2)cc1]

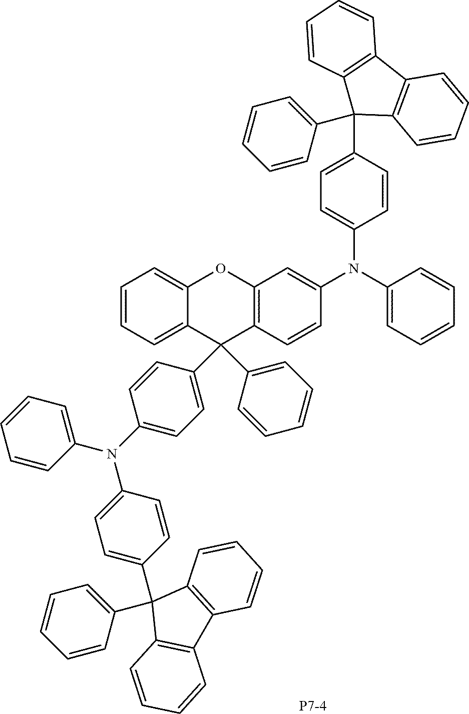

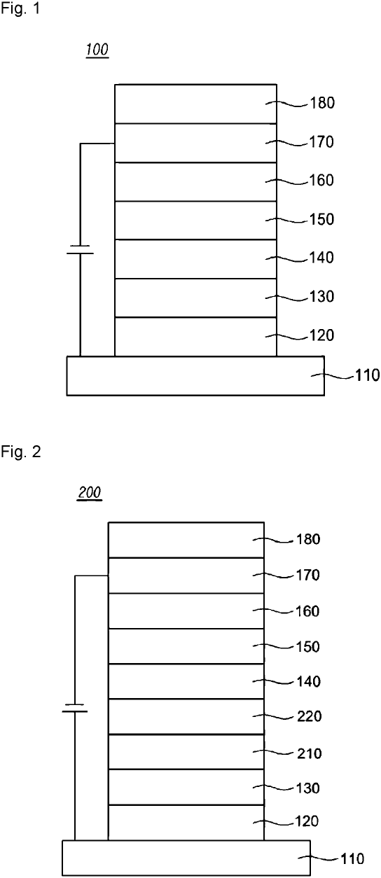

Referring to FIG. 1, the organic electronic element (100) according to the present invention includes a first electrode (110), a second electrode (170), and an organic material layer including a single compound or 2 or more compounds represented by Formula 1 between the first electrode (110) and the second electrode (170). In this case, the first electrode (110) may be an anode, and the second electrode (170) may be a cathode. In the case of an inverted type, the first electrode may be a cathode and the second electrode may be an anode.

The organic material layer may sequentially include a hole injection layer (120), a hole transport layer (130), an emitting layer (140), an electron transport layer (150), and an electron injection layer (160) on the first electrode (110). In this case, the remaining layers except for the emitting layer (140) may not be formed. It may further include a hole blocking layer, an electron blocking layer, an emitting-auxiliary layer (220), a buffer layer (210), etc. and the electron transport layer (150) and the like may serve as a hole blocking layer. (See FIG. 2)

Also, the organic electronic element according to an embodiment of the present invention may further include a protective layer or a light efficiency enhancing layer (180). The light efficiency enhancing layer may be formed on one of both surfaces of the first electrode not in contact with the organic material layer or on one of both surfaces of the second electrode not in contact with the organic material layer. The compound according to an embodiment of the present invention applied to the organic material layer may be used as a host or dopant of the hole injection layer (120), the hole transport layer (130), the emitting-auxiliary layer (220), electron transport auxiliary layer, the electron transport layer (150), and an electron injection layer (160), the emitting layer (140) or as a material for the light efficiency enhancing layer. Preferably, for example, the compound according to Formula 1 of the present invention may be used as a material for a hole transport layer, a host of an emitting layer, and/or an emitting auxiliary layer.

The organic material layer may include 2 or more stacks including a hole transport layer, an emitting layer and an electron transport layer sequentially formed on the anode, further include a charge generation layer formed between the 2 or more stacks (see FIG. 3).

Otherwise, even with the same core, the band gap, electrical characteristics, interface characteristics, etc. may vary depending on which position the substituent is bonded to, therefore the choice of core and the combination of sub-substituents bound thereto are also very important, and in particular, when the optimal combination of energy levels and T1 values and unique properties of materials(mobility, interfacial characteristics, etc.) of each organic material layer is achieved, a long lifespan and high efficiency can be achieved at the same time.

The organic electroluminescent device according to an embodiment of the present invention may be manufactured using a PVD (physical vapor deposition) method. For example, depositing a metal or a metal oxide having conductivity or an alloy thereof on a substrate to form an anode, and after forming an organic material layer including the hole injection layer(120), the hole transport layer(130), the emitting layer(140), the electron transport layer(150) and the electron injection layer(160) thereon, it can be prepared by depositing a material that can be used as a cathode thereon.

Also, in the present invention, the organic material layer is formed by any one of a spin coating process, a nozzle printing process, an inkjet printing process, a slot coating process, a dip coating process, and a roll-to-roll process, and the organic material layer provides an organic electronic element comprising the compound as an electron transport material.

As another specific example, the same or different compounds of the compound represented by Formula 1 are mixed and used in the organic material layer.

Also, the present invention provides a composition for a hole transport layer, an emitting auxiliary layer or an emitting layer comprising the compound represented by Formula 1, and provides an organic electronic element comprising the hole transport layer, the emitting auxiliary layer or the emitting layer.

Also, the present invention provides an electronic device comprising a display device including the organic electronic element; and a control unit for driving the display device;

In another aspect, the organic electronic element is at least one of an organic electroluminescent device, an organic solar cell, an organic photo conductor, an organic transistor, and a device for monochromatic or white lighting. At this time, the electronic device may be a current or future wired/wireless communication terminal, and covers all kinds of electronic devices including mobile communication terminals such as mobile phones, a personal digital assistant(PDA), an electronic dictionary, a point-to-multipoint(PMP), a remote controller, a navigation unit, a game player, various kinds of TVs, and various kinds of computers.

Hereinafter, a synthesis example of the compound represented by Formula 1 of the present invention and a manufacturing example of an organic electronic element of the present invention will be described in detail with reference to Examples, but the present invention is not limited to the following Examples.

### Synthesis Example 1

The compound (final product) represented by Formula 1 according to the present invention is synthesized as shown in Scheme 1 below, but is not limited thereto. Hal¹ and Hal² are I, Br or Cl.

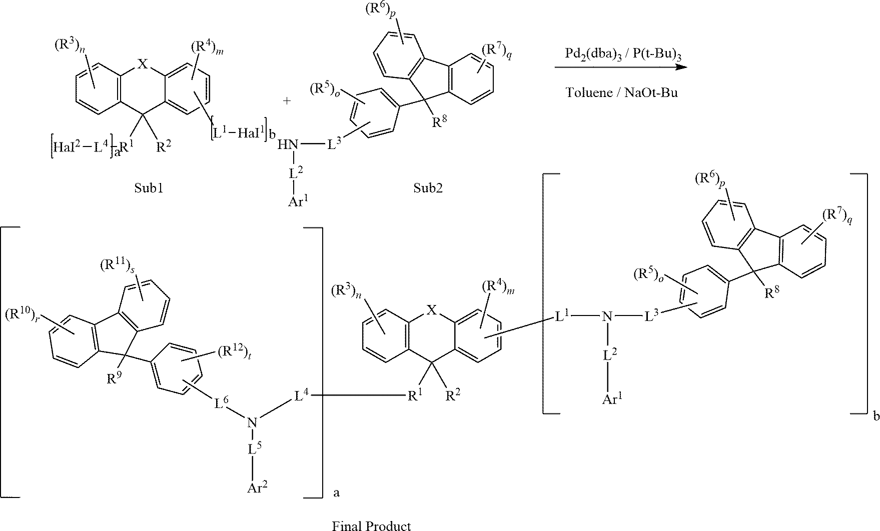

### I. Synthesis of Sub 1

Sub1 of Reaction Scheme 1 is synthesized by the reaction route of Reaction Scheme 2, but is not limited thereto. Hal¹ and Hal² are I, Br or Cl. Hal³ is I or Br.

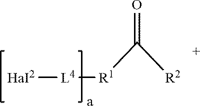

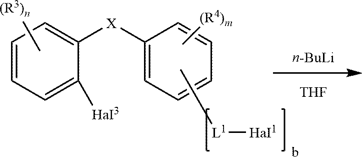

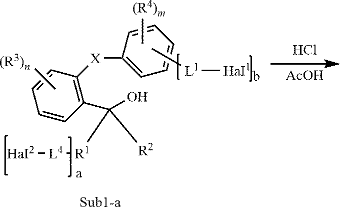

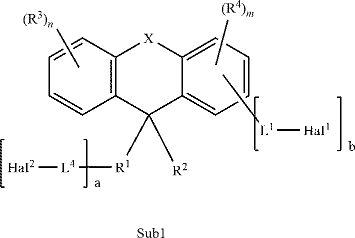

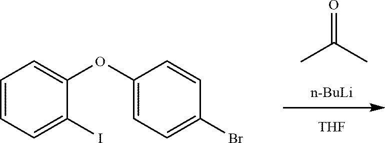

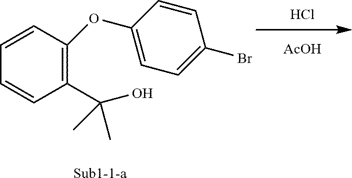

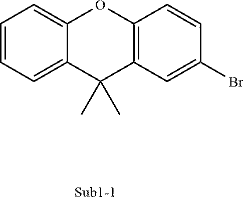

### Synthesis Example of Sub1-1-a

1-bromophenoxy)-2-iodobenzene (50.0 g, 133 mmol) was dissolved in THF (300 mL) in a round-bottom flask under a nitrogen atmosphere, and then cooled to -78° C. Then, n-BuLi (53 mL) is slowly titrated and the mixture is stirred for 30 minutes. Then, propan-2-one (7.7 g, 133 mmol) is dissolved in THF (145 mL) and slowly titrated to the round-bottom flask being reacted. After stirring for an additional 1 hour at -78° C., it is gradually raised to room temperature. When the reaction was completed, the organic layer was extracted with ethyl acetate and water, dried over MgSO₄, concentrated, and the resulting compound was recrystallized by silicagel column to obtain 33.6 g (yield 82%) of the product.

### Synthesis Example of Sub1-1

The obtained Sub1-1-a (33.6 g, 109 mmol), acetic acid (273 mL) and concentrated hydrochloric acid (44 mL) were placed in a round-bottom flask and stirred at 60-80° C. under nitrogen atmosphere for 3 hours. When the reaction was completed, the mixture was extracted with CH₂Cl₂ and water, the organic layer was dried over MgSO₄, concentrated, and the resulting compound was recrystallized using silicagel column to obtain 27.2 g (yield 86%) of the product.

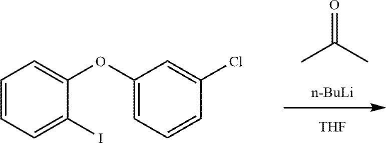

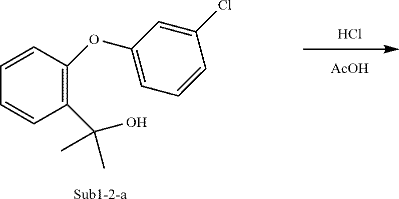

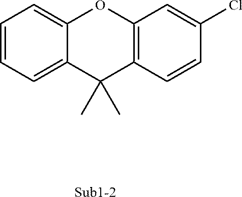

or

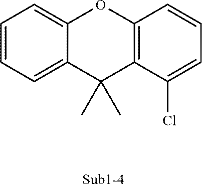

### Synthesis Example of Sub1-2-a

1-chlorophenoxy)-2-iodobenzene (50.0 g, 151 mmol), n-BuLi (61 mL), and propan-2-one (8.8 g, 151 mmol) were used to obtain 31.8 g of the product (yield 80%) using the Synthesis method of Sub1-1-a.

### Synthesis Example of Sub1-2, Sub1-4

The obtained Sub1-2-a (31.8 g, 121 mmol), acetic acid (300 mL) and concentrated hydrochloric acid (48 mL) were used to obtain 17.5 g (yield: 59%) of Sub1-2 and 7.4 g (yield: 25%) of Sub1-4 by using the Synthesis method of Sub1-1.

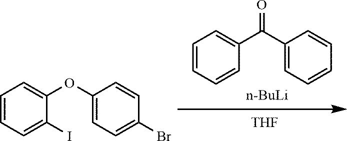

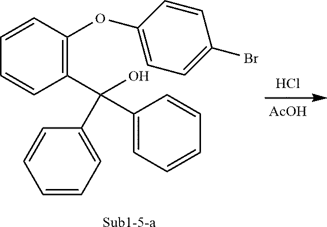

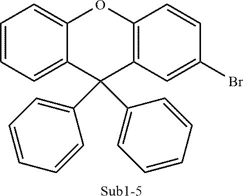

### Synthesis Example of Sub1-5-a

1-bromophenoxy)-2-iodobenzene (25.0 g, 66.7 mmol), n-BuLi (27 mL), benzophenone (12.1 g, 66.7 mmol) were used to obtain 22.4 g of the product (yield 78%) using the Synthesis method of Sub1-1-a.

### Synthesis Example of Sub1-5

The obtained Sub1-5-a (22.4 g, 52.0 mmol), acetic acid (130 mL) and concentrated hydrochloric acid (21 mL) were used to obtain 17.6 g of the product (yield 82%) using the synthesis of Sub1-1.

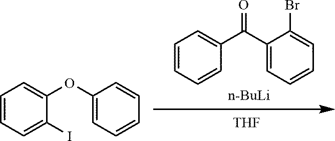

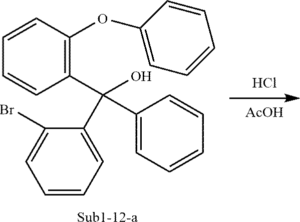

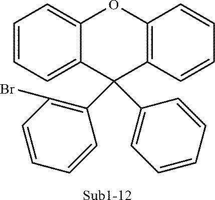

### Synthesis Example of Sub1-12-a

1-iodophenoxybenzene (25.0 g, 84.4 mmol), n-BuLi (34 mL), (2-bromophenyl)(phenyl)methanone (22.0 g, 84.4 mmol) were used to obtain 29.5 g of the product (yield 81%) using the Synthesis method of Sub1-1-a.

### Synthesis Example of Sub1-12

The obtained Sub1-12-a (29.5 g, 68.4 mmol), acetic acid (170 mL) and concentrated hydrochloric acid (27 mL) were used to obtain 22.6 g of the product (yield 80%) using the synthesis of Sub1-1.

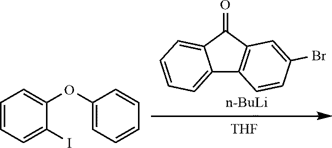

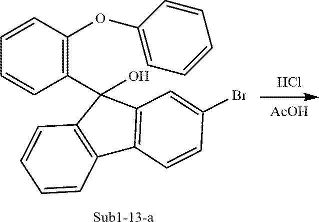

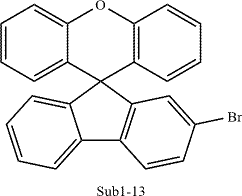

### Synthesis Example of Sub1-13-a

1-iodophenoxybenzene (50.0 g, 169 mmol), n-BuLi (68 mL), 2-bromo-9H-fluoren-9-one (43.8 g, 169 mmol) were used to obtain 59.4 g of the product (yield 82%) using the Synthesis method of Sub1-1-a.

### Synthesis Example of Sub1-13

The obtained Sub1-13-a (59.4 g, 138 mmol), acetic acid (345 mL) and concentrated hydrochloric acid (55 mL) were used to obtain 47.3 g of the product (yield 83%) using the synthesis of Sub1-1.

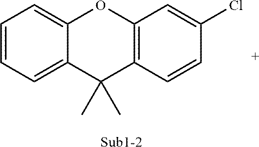

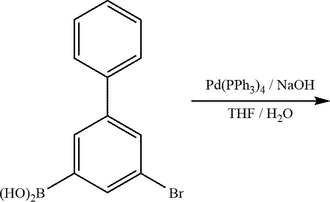

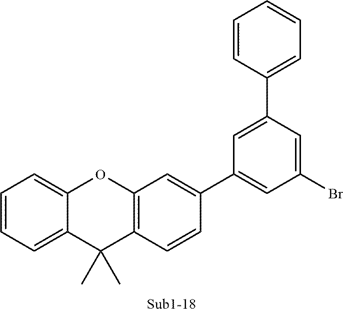

After dissolving Sub1-2 (5.0 g, 20.4 mmol) in THF (102 mL), (5-bromo-[1,1′-biphenyl]-3-yl)boronic acid (5.7 g, 20.4 mmol), NaOH (2.5 g, 61.3 mmol), Pd(PPh₃)₄ (1.42 g, 1.23 mmol) and Water (51 mL) were added and stirred at 80° C. When the reaction was completed, the mixture was extracted with CH₂CI₂ and water, and the organic layer was dried over MgSO₄ and concentrated.

Thereafter, the resulting compound was recrystallized after applying a silica gel column to obtain 6.6 g (yield 71%) of the product.

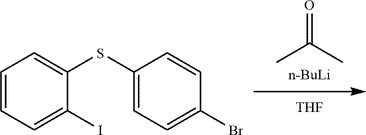

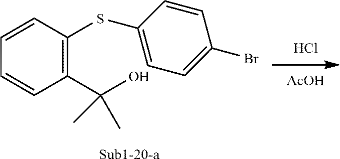

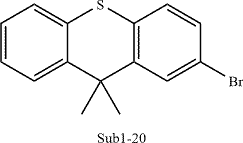

### Synthesis Example of Sub1-20-a

bromophenyl)(2-iodophenyl)sulfane (50.0 g, 128 mmol), n-BuLi (51 mL), propan-2-one (7.4 g, 128 mmol) were used to obtain 34.3 g of the product (yield 83%) using the Synthesis method of Sub1-1-a.

### Synthesis Example of Sub1-20

The obtained Sub1-20-a (34.3 g, 106 mmol), acetic acid (265 mL) and concentrated hydrochloric acid (42 mL) were used to obtain 26.2 g of the product (yield 81%) using the synthesis of Sub1-1

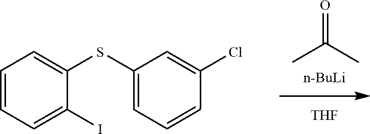

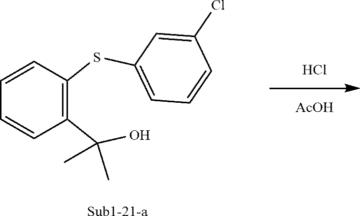

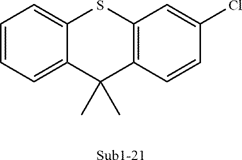

or

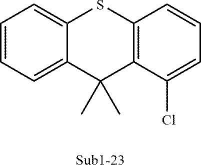

### Synthesis Example of Sub1-21-a

chlorophenyl)(2-iodophenyl)sulfane (50.0 g, 144 mmol), n-BuLi (58 mL), propan-2-one (8.4 g, 144 mmol) were used to obtain 32.3 g of the product (yield 80%) using the Synthesis method of Sub1-1-a.

### Synthesis Example of Sub1-21

The obtained Sub1-21-a (32.2 g, 115 mmol), acetic acid (290 mL) and concentrated hydrochloric acid (46 mL) was used to obtain 17.2 g of Sub1-21 (yield 57%) and 7.5 g (yield of 25%) of Sub1-23 by using the method for Sub1-1.

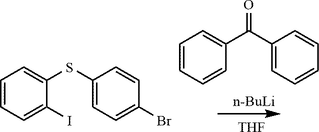

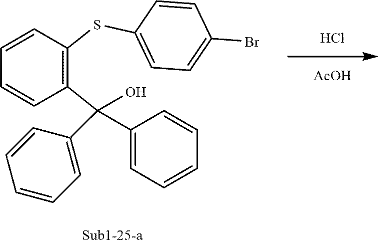

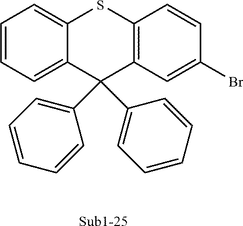

### Synthesis Example of Sub1-25-a

bromophenyl)(2-iodophenyl)sulfane (30.0 g, 76.7 mmol), n-BuLi (31 mL), benzophenone (14.0 g, 76.7 mmol) were used to obtain 28.1 g of the product (yield 82%) using the Synthesis method of Sub1-1-a.

### Synthesis Example of Sub1-25

The obtained Sub1-25-a (28.1 g, 62.9 mmol), acetic acid (157 mL) and concentrated hydrochloric acid (25 mL) were used to obtain 23.0 g of the product (yield 85%) using the synthesis of Sub1-1

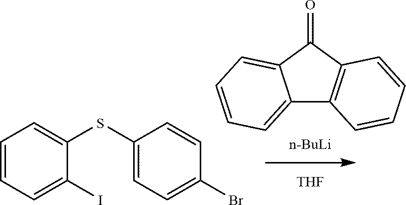

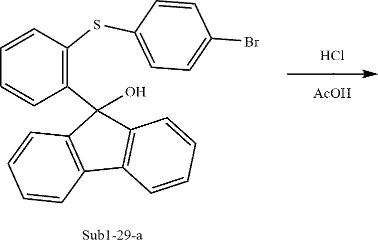

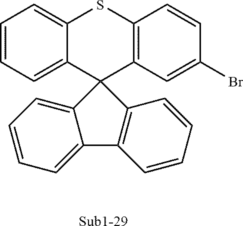

### Synthesis Example of Sub1-29-a

bromophenyl)(2-iodophenyl)sulfane (50.0 g, 128 mmol), n-BuLi (51 mL), 9H-fluoren-9-one (23.0 g, 128 mmol) were used to obtain 46.7 g of the product (yield 82%) using the Synthesis method of Sub1-1-a.

### Synthesis Example of Sub1-29

The obtained Sub1-29-a (46.7 g, 105 mmol), acetic acid (262 mL) and concentrated hydrochloric acid (42 mL) were used to obtain 36.3 g of the product (yield 81%) using the synthesis of Sub1-1

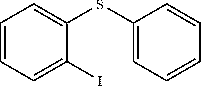 [SMILES: Ic1ccccc1Sc1ccccc1]

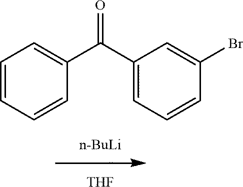 [SMILES: O=C(c1ccccc1)c1cccc(Br)c1 ; "n-BuLi" ; "THF"]

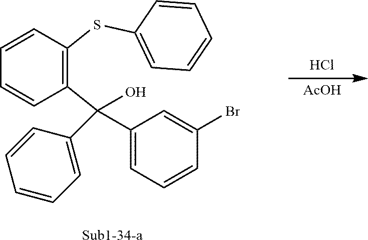

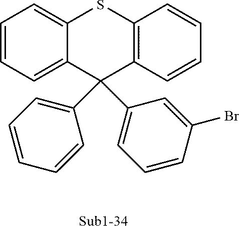

### Synthesis Example of Sub1-34-a

iodophenyl)(phenyl)sulfane (25.0 g, 80.1 mmol), n-BuLi (32 mL), (3-bromophenyl)(phenyl)methanone (20.9 g, 80.1 mmol) were used to obtain 27.9 g of the product (yield 78%) using the Synthesis method of Sub1-1-a.

### Synthesis Example of Sub1-34

The obtained Sub1-34-a (27.9 g, 62.5 mmol), acetic acid (156 mL) and concentrated hydrochloric acid (25 mL) were used to obtain 22.0 g of the product (yield 82%) using the synthesis of Sub1-1

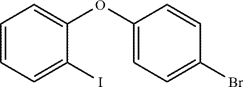 [SMILES: Brc1ccc(Oc2ccccc2I)cc1]

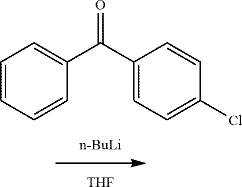 [SMILES: O=C(c1ccccc1)c1ccc(Cl)cc1 ; "n-BuLi" ; "THF"]

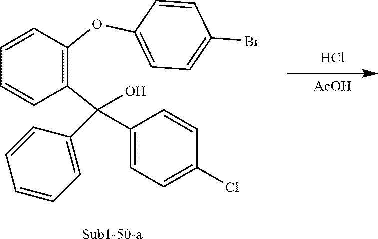

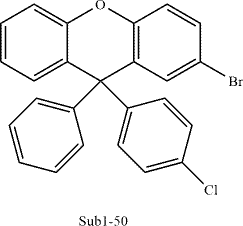

### Synthesis Example of Sub1-50-a

1-bromophenoxy)-2-iodobenzene (5.0 g, 13.3 mmol), n-BuLi (5 mL), (4-chlorophenyl)(phenyl)methanone (2.9 g, 13.3 mmol) were used to obtain 4.9 g of the product (yield 79%) using the Synthesis method of Sub1-1-a.

### Synthesis Example of Sub1-50

The obtained Sub1-50-a (4.9 g, 10.5 mmol), acetic acid (26 mL) and concentrated hydrochloric acid (4 mL) were used to obtain 3.7 g of the product (yield 78%) using the synthesis of Sub1-1

The compound belonging to Sub1 may be a compound as follows, but is not limited thereto, Table 1 below shows FD-MS (Field Desorption-Mass Spectrometry) values of some compounds belonging to Sub1.

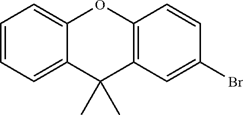 [SMILES: CC1(C)c2ccccc2Oc2ccc(Br)cc21]

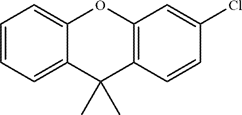 [SMILES: CC1(C)c2ccccc2Oc2cc(Cl)ccc21]

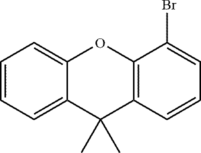 [SMILES: CC1(C)c2ccccc2Oc2c(Br)cccc21]

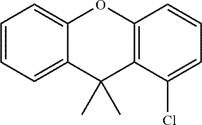 [SMILES: CC1(C)c2ccccc2Oc2cccc(Cl)c21]

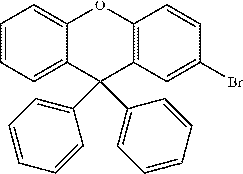 [SMILES: Brc1ccc2c(c1)C(c1ccccc1)(c1ccccc1)c1ccccc1O2]

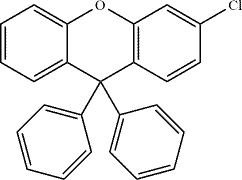 [SMILES: Clc1ccc2c(c1)Oc1ccccc1C2(c1ccccc1)c1ccccc1]

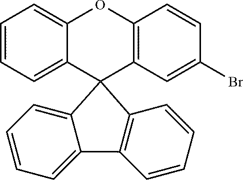 [SMILES: Brc1ccc2c(c1)C1(c3ccccc3O2)c2ccccc2-c2ccccc21]

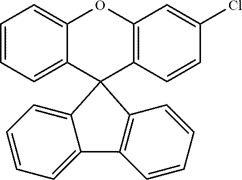 [SMILES: Clc1ccc2c(c1)Oc1ccccc1C21c2ccccc2-c2ccccc21]

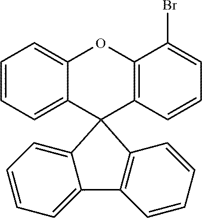 [SMILES: Brc1cccc2c1Oc1ccccc1C21c2ccccc2-c2ccccc21]

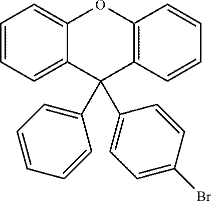 [SMILES: Brc1ccc(C2(c3ccccc3)c3ccccc3Oc3ccccc32)cc1]

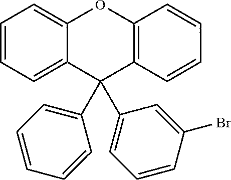 [SMILES: Brc1cccc(C2(c3ccccc3)c3ccccc3Oc3ccccc32)c1]

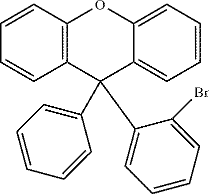 [SMILES: Brc1ccccc1C1(c2ccccc2)c2ccccc2Oc2ccccc21]

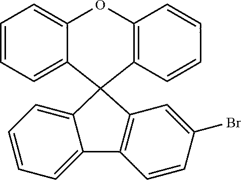 [SMILES: Brc1ccc2c(c1)C1(c3ccccc3Oc3ccccc31)c1ccccc1-2]

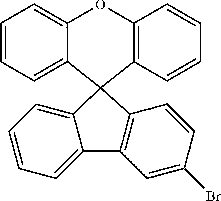 [SMILES: Brc1ccc2c(c1)-c1ccccc1C21c2ccccc2Oc2ccccc21]

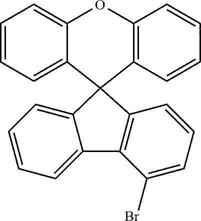 [SMILES: Brc1cccc2c1-c1ccccc1C21c2ccccc2Oc2ccccc21]

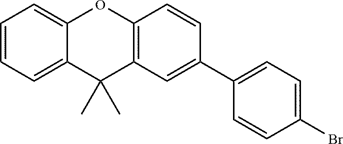 [SMILES: CC1(C)c2ccccc2Oc2ccc(-c3ccc(Br)cc3)cc21]

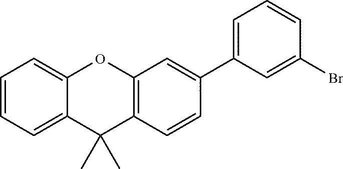 [SMILES: CC1(C)c2ccccc2Oc2cc(-c3cccc(Br)c3)ccc21]

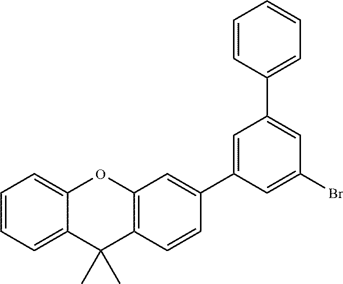 [SMILES: CC1(C)c2ccccc2Oc2cc(-c3cc(Br)cc(-c4ccccc4)c3)ccc21]

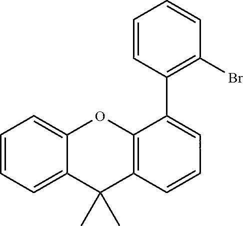 [SMILES: CC1(C)c2ccccc2Oc2c(-c3ccccc3Br)cccc21]

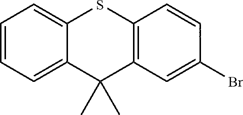 [SMILES: CC1(C)c2ccccc2Sc2ccc(Br)cc21]

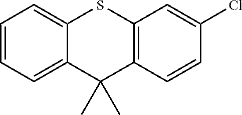 [SMILES: CC1(C)c2ccccc2Sc2cc(Cl)ccc21]

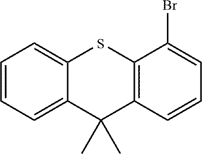 [SMILES: CC1(C)c2ccccc2Sc2c(Br)cccc21]

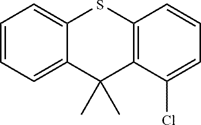 [SMILES: CC1(C)c2ccccc2Sc2cccc(Cl)c21]

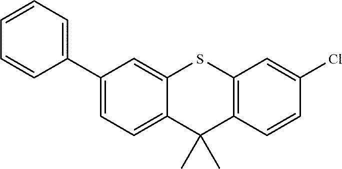 [SMILES: CC1(C)c2ccc(Cl)cc2Sc2cc(-c3ccccc3)ccc21]

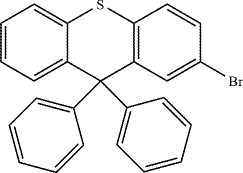 [SMILES: Brc1ccc2c(c1)C(c1ccccc1)(c1ccccc1)c1ccccc1S2]

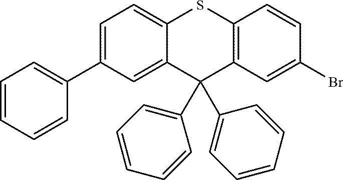 [SMILES: Brc1ccc2c(c1)C(c1ccccc1)(c1ccccc1)c1cc(-c3ccccc3)ccc1S2]

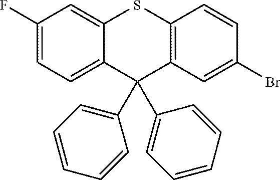 [SMILES: Fc1ccc2c(c1)Sc1ccc(Br)cc1C2(c1ccccc1)c1ccccc1]

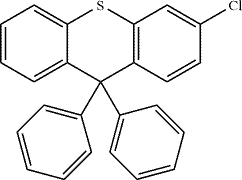 [SMILES: Clc1ccc2c(c1)Sc1ccccc1C2(c1ccccc1)c1ccccc1]

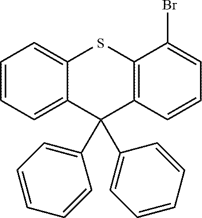 [SMILES: Brc1cccc2c1Sc1ccccc1C2(c1ccccc1)c1ccccc1]

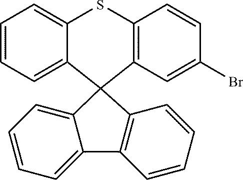 [SMILES: Brc1ccc2c(c1)C1(c3ccccc3S2)c2ccccc2-c2ccccc21]

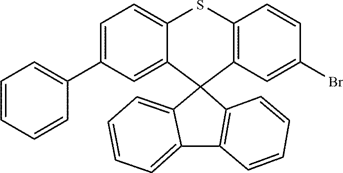 [SMILES: Brc1ccc2c(c1)C1(c3cc(-c4ccccc4)ccc3S2)c2ccccc2-c2ccccc21]

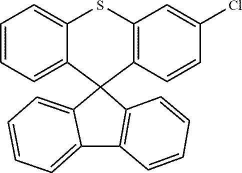 [SMILES: Clc1ccc2c(c1)Sc1ccccc1C21c2ccccc2-c2ccccc21]

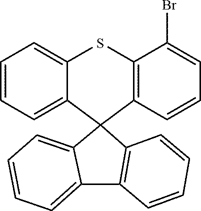 [SMILES: Brc1cccc2c1Sc1ccccc1C21c2ccccc2-c2ccccc21]

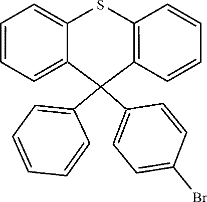 [SMILES: Brc1ccc(C2(c3ccccc3)c3ccccc3Sc3ccccc32)cc1]

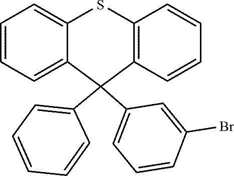 [SMILES: Brc1cccc(C2(c3ccccc3)c3ccccc3Sc3ccccc32)c1]

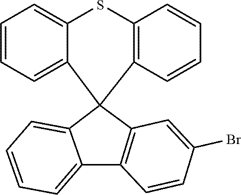 [SMILES: Brc1ccc2c(c1)C1(c3ccccc3Sc3ccccc31)c1ccccc1-2]

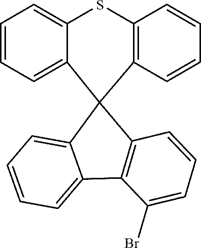 [SMILES: Brc1cccc2c1-c1ccccc1C21c2ccccc2Sc2ccccc21]

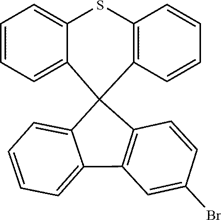 [SMILES: Brc1ccc2c(c1)-c1ccccc1C21c2ccccc2Sc2ccccc21]

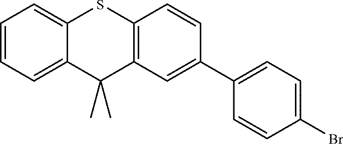 [SMILES: CC1(C)c2ccccc2Sc2ccc(-c3ccc(Br)cc3)cc21]

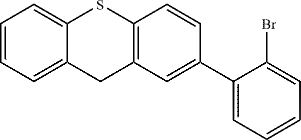 [SMILES: Brc1ccccc1-c1ccc2c(c1)Cc1ccccc1S2]

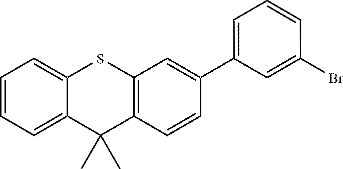 [SMILES: CC1(C)c2ccccc2Sc2cc(-c3cccc(Br)c3)ccc21]

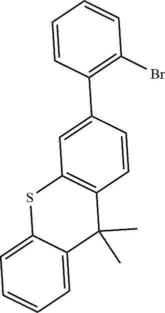 [SMILES: CC1(C)c2ccccc2Sc2cc(-c3ccccc3Br)ccc21]

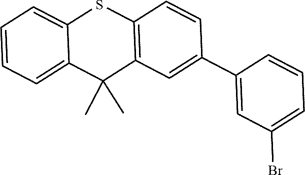 [SMILES: CC1(C)c2ccccc2Sc2ccc(-c3cccc(Br)c3)cc21]

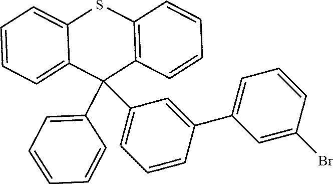 [SMILES: Brc1cccc(-c2cccc(C3(c4ccccc4)c4ccccc4Sc4ccccc43)c2)c1]

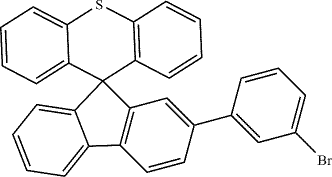 [SMILES: Brc1cccc(-c2ccc3c(c2)C2(c4ccccc4Sc4ccccc42)c2ccccc2-3)c1]

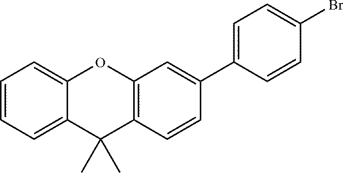 [SMILES: CC1(C)c2ccccc2Oc2cc(-c3ccc(Br)cc3)ccc21]

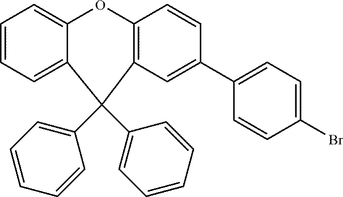 [SMILES: Brc1ccc(-c2ccc3c(c2)C(c2ccccc2)(c2ccccc2)c2ccccc2O3)cc1]

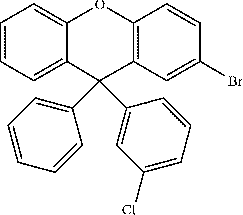 [SMILES: Clc1cccc(C2(c3ccccc3)c3ccccc3Oc3ccc(Br)cc32)c1]

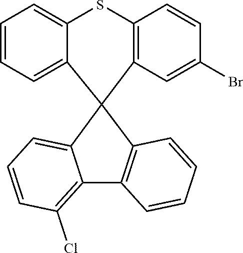 [SMILES: Clc1cccc2c1-c1ccccc1C21c2ccccc2Sc2ccc(Br)cc21]

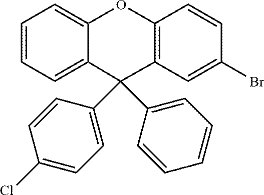 [SMILES: Clc1ccc(C2(c3ccccc3)c3ccccc3Oc3ccc(Br)cc32)cc1]

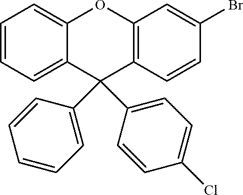 [SMILES: Clc1ccc(C2(c3ccccc3)c3ccccc3Oc3cc(Br)ccc32)cc1]

TABLE 1

| | | | |
|---|---|---|---|
| compound | FD-MS | compound | FD-MS |
| Sub1-1 | m/z=288.01 (C₁₅H₁₃BrO=289.17) | Sub1-2 | m/z=244.07(C₁₅H₁₃CIO=244.72) |
| Sub1-3 | m/z=288.01 (C₁₅H₁₃BrO=289.17) | Sub1-4 | m/z=244.07(C₁₅H₁₃CIO=244.72) |
| Sub1-5 | m/z=412.05(C₂₅H₁₇BrO=413.31) | Sub1-6 | m/z=368.10(C₂₅H₁₇CIO=368.86) |
| Sub1-7 | m/z=410.03(C₂₅H₁₅BrO=411.30) | Sub1-8 | m/z=366.08(C₂₅H₁₅CIO=366.84) |
| Sub1-9 | m/z=410.03(C₂₅H₁₅BrO=411.30) | Sub1-10 | m/z=412.05(C₂₅H₁₇BrO=413.31) |
| Sub1-11 | m/z=412.05(C₂₅H₁₇BrO=413.31) | Sub1-12 | m/z=412.05(C₂₅H₁₇BrO=413.31) |
| Sub1-13 | m/z=410.03(C₂₅H₁₅BrO=411.30) | Sub1-14 | m/z=410.03(C₂₅H₁₅BrO=411.30) |
| Sub1-15 | m/z=410.03(C₂₅H₁₅BrO=411.30) | Sub1-16 | m/z=364.05(C₂₁H₁₇BrO=365.27) |
| Sub1-17 | m/z=364.05(C₂₁H₁₇BrO=365.27) | Sub1-18 | m/z=440.08(C₂₇H₂₁BrO=441.37) |
| Sub1-19 | m/z=364.05(C₂₁H₁₇BrO=365.27) | Sub1-20 | m/z=303.99(C₁₅H₁₃BrS=305.23) |
| Sub1-21 | m/z=260.04(C₁₅H₁₃CIS=260.78) | Sub1-22 | m/z=303.99(C₁₅H₁₃BrS=305.23) |
| Sub1-23 | m/z=260.04(C₁₅H₁₃CIS=260.78) | Sub1-24 | m/z=336.07(C₂₁H₁₇ClS=336.88) |
| Sub1-25 | m/z=428.02(C₂₅H₁₇BrS=429.38) | Sub1-26 | m/z=504.05(C₃₁H₂₁ BrS=505.47) |
| Sub1-27 | m/z=446.01(C₂₅H₁₆BrFS=447.37) | Sub1-28 | m/z=384.07(C₂₅H₁₇CIS=384.92) |
| Sub1-29 | m/z=428.02(C₂₅H₁₇BrS=429.38) | Sub1-30 | m/z=426.01 (C₂₅H₁₅BrS=427.36) |
| Sub1-31 | m/z=502.04(C₃₁H₁₉BrS=503.46) | Sub1-32 | m/z=382.06(C₂₅H₁₅ClS=382.91) |
| Sub1-33 | m/z=426.01 (C₂₅H₁₅BrS=427.36) | Sub1-34 | m/z=428.02(C₂₅H₁₇BrS=429.38) |
| Sub1-35 | m/z=428.02(C₂₅H₁₇BrS=429.38) | Sub1-36 | m/z=426.01 (C₂₅H₁₅BrS=427.36) |
| Sub1-37 | m/z=426.01 (C₂₅H₁₅BrS=427.36) | Sub1-38 | m/z=426.01 (C₂₅H₁₅BrS=427.36) |
| Sub1-39 | m/z=380.02(C₂₁H₁₇BrS=381.33) | Sub1-40 | m/z=380.02(C₂₁H₁₇BrS=381.33) |
| Sub1-41 | m/z=380.02(C₂₁H₁₇BrS=381.33) | Sub1-42 | m/z=380.02(C₂₁H₁₇BrS=381.33) |
| Sub1-43 | m/z=380.02(C₂₁H₁₇BrS=381.33) | Sub1-44 | m/z=504.05(C₃₁H₂₁BrS=505.47) |
| Sub1-45 | m/z=502.04(C₃₁H₁₉BrS=503.46) | Sub1-46 | m/z=364.05(C₂₁H₁₇BrO=365.27) |
| Sub1-47 | m/z=488.08(C₃₁H₂₁BrO=489.41) | Sub1-48 | m/z=446.01 (C₂₅H₁₆BrClO= 447.76) |
| Sub1-49 | m/z=459.97(C₂₅H₁₄BrClS= 461.80) | Sub1-50 | m/z=446.01 (C₂₅H₁₆BrClO= 447.76) |
| Sub1-51 | m/z=446.01 (C₂₅H₁₆BrClO= 447.76) | | |

### II. Synthesis of Sub2

Sub2 of Reaction Scheme 1 is synthesized by the reaction route of Reaction Scheme 3, but is not limited thereto. Hal⁴ is I, Br or Cl.

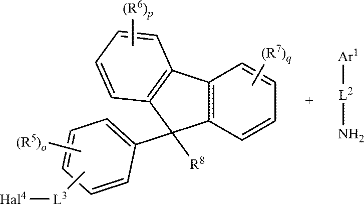

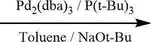

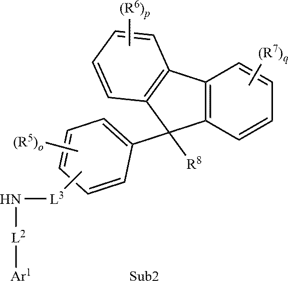

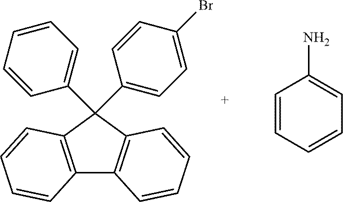

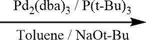

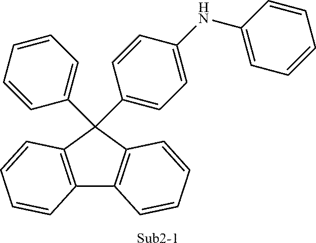

9-(4-bromophenyl)-9-phenyl-9H-fluorene (30.0 g, 75.5 mmol) was dissolved in toluene (380 mL) in a round-bottom flask, aniline (7.0 g, 75.5 mmol), Pd₂(dba)₃ (2.07 g, 2.27 mmol), P(t-Bu)₃ (0.92 g, 4.53 mmol), NaOt—Bu (14.5 g, 151 mmol) were added thereto, and the mixture was stirred at 60° C. When the reaction was completed, the mixture was extracted with CH₂Cl₂ and water, the organic layer was dried over MgSO₄, concentrated, and the resulting compound was recrystallized using silicagel column to obtain 23.2 g (yield 75%) of the product.

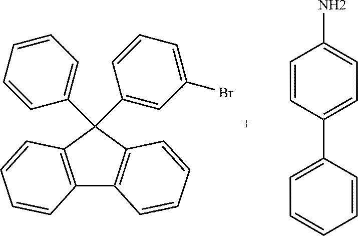

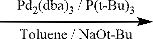

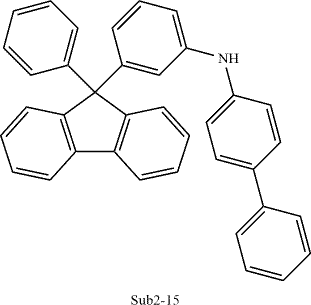

9-(3-bromophenyl)-9-phenyl-9H-fluorene (30.0 g, 75.5 mmol), [1,1′-biphenyl]-4-amine (12.8 g, 75.5 mmol), Pd₂(dba)₃ (2.07 g, 2.27 mmol), P(t-Bu)₃ (0.92 g, 4.53 mmol) and NaOt—Bu (14.5 g, 151 mmol) were used to obtain 26.8 g (yield 73%) of the product using the synthesis method of Sub2-1

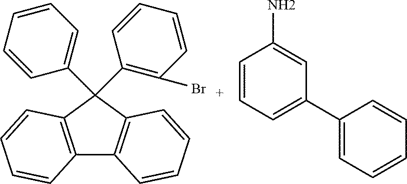

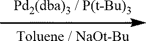

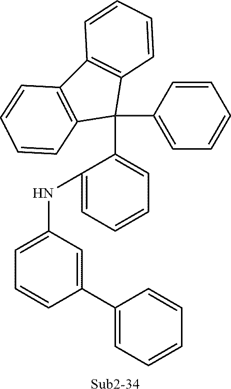

9-(2-bromophenyl)-9-phenyl-9H-fluorene (5.0 g, 12.6 mmol), [1,1′-biphenyl]-3-amine (2.1 g, 12.6 mmol), Pd₂(dba)₃ (0.35 g, 0.38 mmol), P(t-Bu)₃ (0.15 g, 0.76 mmol), NaOt—Bu (2.4 g, 25.2 mmol) were used to obtain 4.2 g (yield 68%) of the product using the synthesis method of Sub2-1

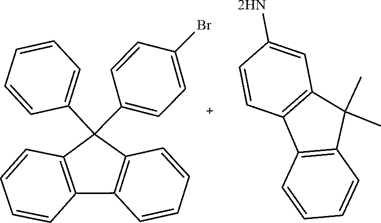

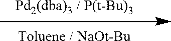

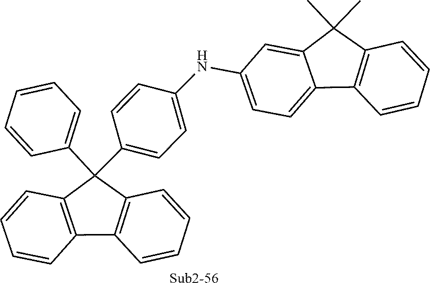

9-(4-bromophenyl)-9-phenyl-9H-fluorene (5.0 g, 12.6 mmol), 9,9-dimethyl-9H-fluoren-2-amine (2.6 g, 12.6 mmol), Pd₂(dba)₃ (0.35 g, 0.38 mmol), P(t-Bu)₃ (0.15 g, 0.76 mmol), NaOt—Bu (2.4 g, 25.2 mmol) were used to obtain 4.9 g (yield 74%) of the product using the synthesis method of Sub2-1

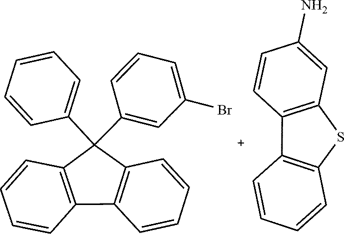

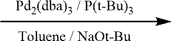

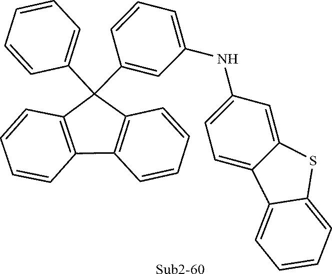

9-(3-bromophenyl)-9-phenyl-9H-fluorene (5.0 g, 12.6 mmol), dibenzo[b,d]thiophen-3-amine (2.5 g, 12.6 mmol), Pd₂(dba)₃ (0.35 g, 0.38 mmol), P(t-Bu)₃ (0.15 g, 0.76 mmol), NaOt—Bu (2.4 g, 25.2 mmol) were used to obtain 4.7 g (yield 73%) of the product using the synthesis method of Sub2-1

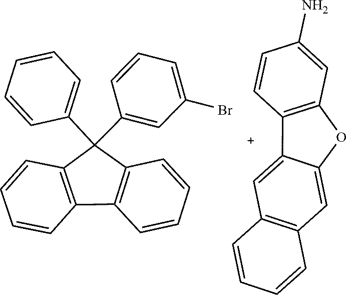

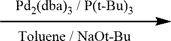

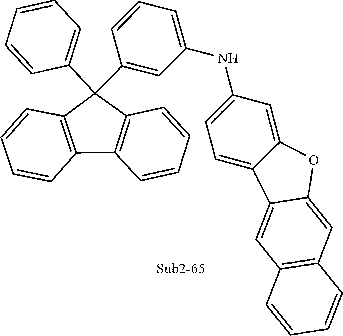

9-(3-bromophenyl)-9-phenyl-9H-fluorene (5.0 g, 12.6 mmol), naphtho[2,3-b]benzofuran-3-amine (2.9 g, 12.6 mmol), Pd₂(dba)₃ (0.35 g, 0.38 mmol), P(t-Bu)₃ (0.15 g, 0.76 mmol), NaOt—Bu (2.4 g, 25.2 mmol) were used to obtain 4.9 g (yield 71%) of the product using the synthesis method of Sub2-1

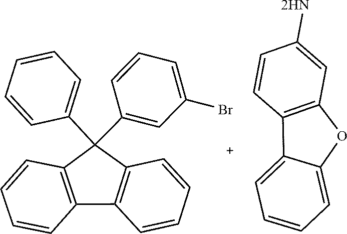

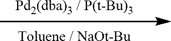

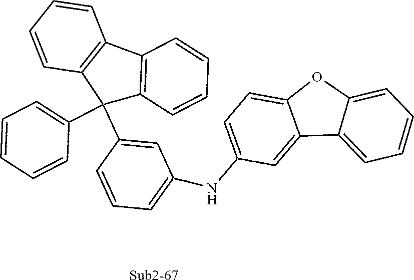

9-(3-bromophenyl)-9-phenyl-9H-fluorene (5.0 g, 12.6 mmol), dibenzo[b,d]furan-2-amine (2.3 g, 12.6 mmol), Pd₂(dba)₃ (0.35 g, 0.38 mmol), P(t—Bu)₃ (0.15 g, 0.76 mmol), NaOt—Bu (2.4 g, 25.2 mmol) were used to obtain 4.6 g (yield 73%) of the product using the synthesis method of Sub2-1

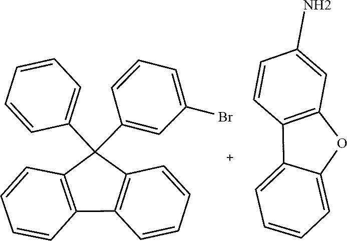

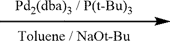

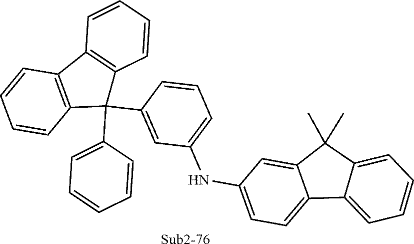

9-(3-bromophenyl)-9-phenyl-9H-fluorene (5.0 g, 12.6 mmol), 9,9-dimethyl-9H-fluoren-2-amine (2.6 g, 12.6 mmol), Pd₂(dba)₃ (0.35 g, 0.38 mmol), P(t—Bu)₃ (0.15 g, 0.76 mmol), NaOt—Bu (2.4 g, 25.2 mmol) were used to obtain 4.6 g (yield 70%) of the product using the synthesis method of Sub2-1

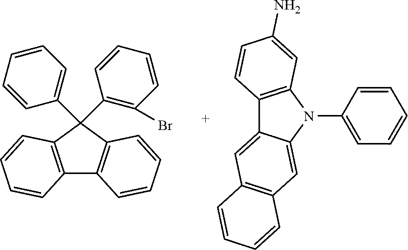

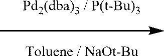

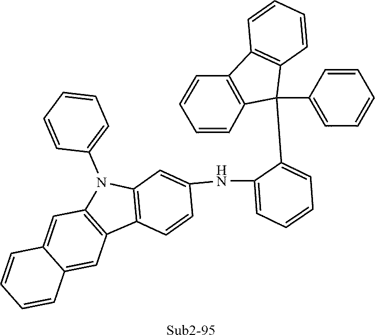

9-(2-bromophenyl)-9-phenyl-9H-fluorene (5.0 g, 12.6 mmol), 5-phenyl-5H-benzo[b]carbazol-3-amine (3.9 g, 12.6 mmol), Pd₂(dba)₃ (0.35 g, 0.38 mmol), P(t-Bu)₃ (0.15 g, 0.76 mmol), NaOt—Bu (2.4 g, 25.2 mmol) were used to obtain 5.1 g (yield 65%) of the product using the synthesis method of Sub2-1

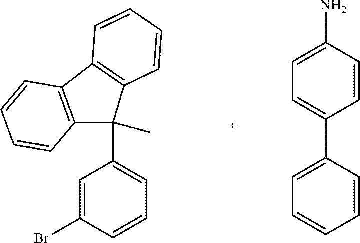

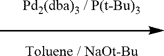

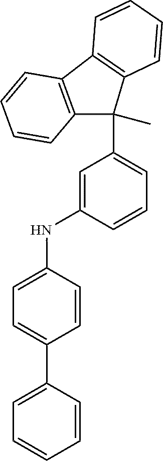 [SMILES: CC1(c2cccc(Nc3ccc(-c4ccccc4)cc3)c2)c2ccccc2-c2ccccc21]

9-(3-bromophenyl)-9-methyl-9H-fluorene (2.0 g, 6.0 mmol), [1,1′-biphenyl]-4-amine (1.0 g, 6.0 mmol), Pd₂(dba)₃ (0.16 g, 0.18 mmol), P(t-Bu)₃ (0.07 g, 0.36 mmol), NaOt—Bu (1.1 g, 11.9 mmol) were used to obtain 1.8 g (yield 71%) of the product using the synthesis method of Sub2-1

The compound belonging to Sub 2 may be a compound as follows, but is not limited thereto, Table 2 shows FD-MS (Field Desorption-Mass Spectrometry) values of some compounds belonging to Sub 2.

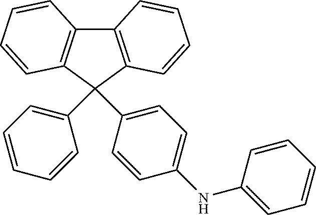 [SMILES: c1ccc(Nc2ccc(C3(c4ccccc4)c4ccccc4-c4ccccc43)cc2)cc1]

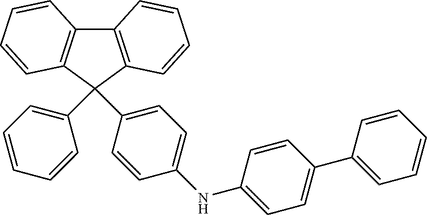 [SMILES: c1ccc(-c2ccc(Nc3ccc(C4(c5ccccc5)c5ccccc5-c5ccccc54)cc3)cc2)cc1]

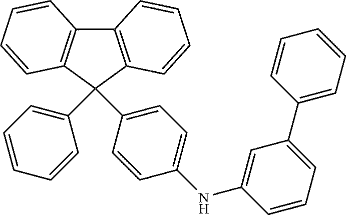 [SMILES: c1ccc(-c2cccc(Nc3ccc(C4(c5ccccc5)c5ccccc5-c5ccccc54)cc3)c2)cc1]

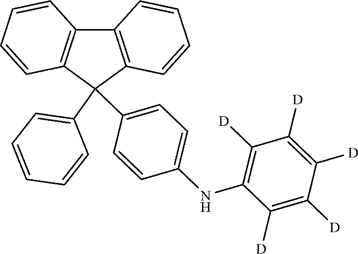 [SMILES: [2H]c1c([2H])c([2H])c(Nc2ccc(C3(c4ccccc4)c4ccccc4-c4ccccc43)cc2)c([2H])c1[2H]]

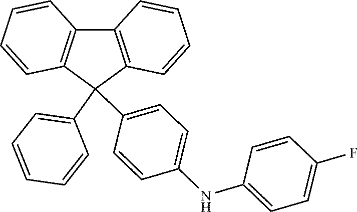 [SMILES: Fc1ccc(Nc2ccc(C3(c4ccccc4)c4ccccc4-c4ccccc43)cc2)cc1]

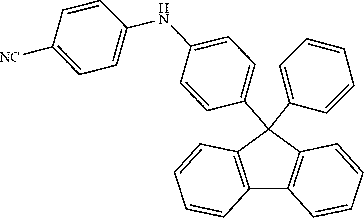

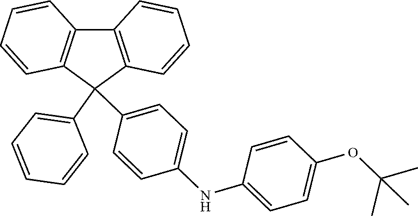 [SMILES: CC(C)(C)Oc1ccc(Nc2ccc(C3(c4ccccc4)c4ccccc4-c4ccccc43)cc2)cc1]

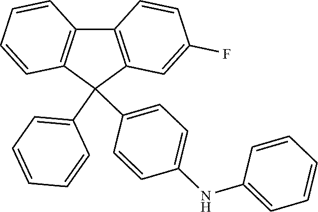 [SMILES: Fc1ccc2c(c1)C(c1ccccc1)(c1ccc(Nc3ccccc3)cc1)c1ccccc1-2]

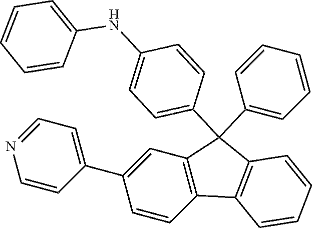 [SMILES: c1ccc(Nc2ccc(C3(c4ccccc4)c4ccccc4-c4ccc(-c5ccncc5)cc43)cc2)cc1]

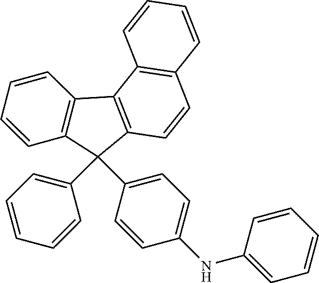 [SMILES: c1ccc(Nc2ccc(C3(c4ccccc4)c4ccccc4-c4c3ccc3ccccc43)cc2)cc1]

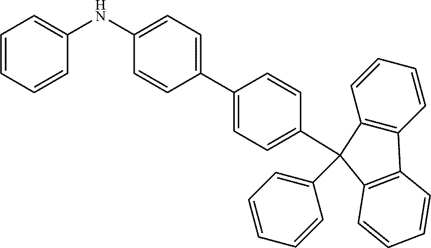 [SMILES: c1ccc(Nc2ccc(-c3ccc(C4(c5ccccc5)c5ccccc5-c5ccccc54)cc3)cc2)cc1]

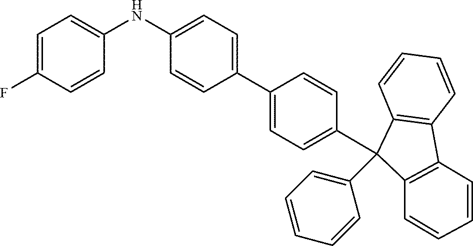 [SMILES: Fc1ccc(Nc2ccc(-c3ccc(C4(c5ccccc5)c5ccccc5-c5ccccc54)cc3)cc2)cc1]

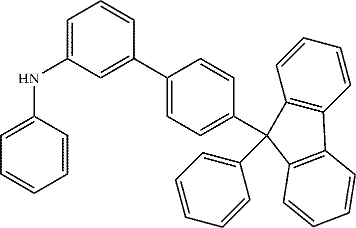 [SMILES: c1ccc(Nc2cccc(-c3ccc(C4(c5ccccc5)c5ccccc5-c5ccccc54)cc3)c2)cc1]

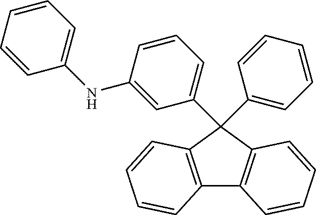 [SMILES: c1ccc(Nc2cccc(C3(c4ccccc4)c4ccccc4-c4ccccc43)c2)cc1]

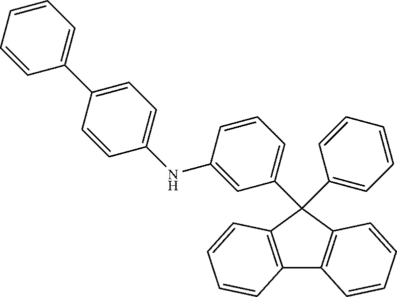 [SMILES: c1ccc(-c2ccc(Nc3cccc(C4(c5ccccc5)c5ccccc5-c5ccccc54)c3)cc2)cc1]

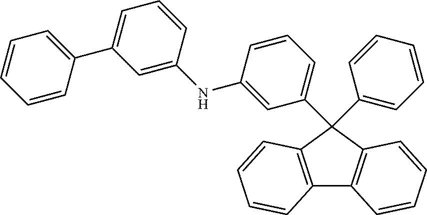 [SMILES: c1ccc(-c2cccc(Nc3cccc(C4(c5ccccc5)c5ccccc5-c5ccccc54)c3)c2)cc1]

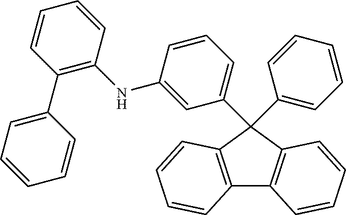 [SMILES: c1ccc(-c2ccccc2Nc2cccc(C3(c4ccccc4)c4ccccc4-c4ccccc43)c2)cc1]

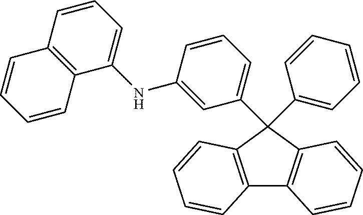 [SMILES: c1ccc(C2(c3cccc(Nc4cccc5ccccc45)c3)c3ccccc3-c3ccccc32)cc1]

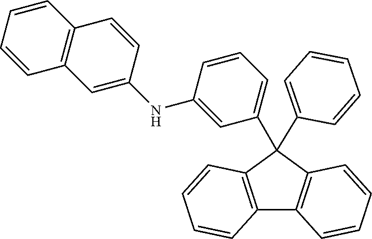 [SMILES: c1ccc(C2(c3cccc(Nc4ccc5ccccc5c4)c3)c3ccccc3-c3ccccc32)cc1]

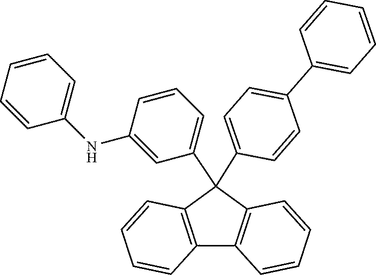 [SMILES: c1ccc(Nc2cccc(C3(c4ccc(-c5ccccc5)cc4)c4ccccc4-c4ccccc43)c2)cc1]

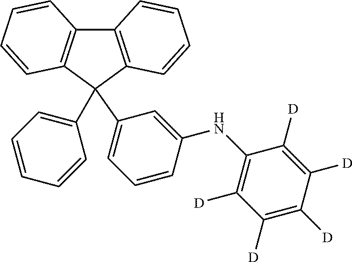 [SMILES: [2H]c1c([2H])c([2H])c(Nc2cccc(C3(c4ccccc4)c4ccccc4-c4ccccc43)c2)c([2H])c1[2H]]

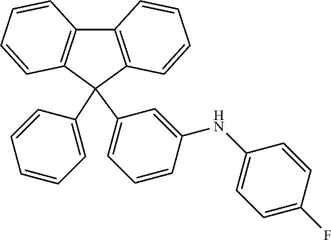 [SMILES: Fc1ccc(Nc2cccc(C3(c4ccccc4)c4ccccc4-c4ccccc43)c2)cc1]

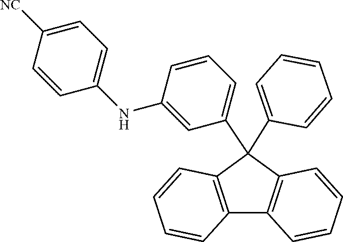

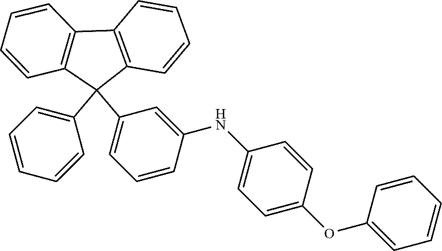 [SMILES: c1ccc(Oc2ccc(Nc3cccc(C4(c5ccccc5)c5ccccc5-c5ccccc54)c3)cc2)cc1]

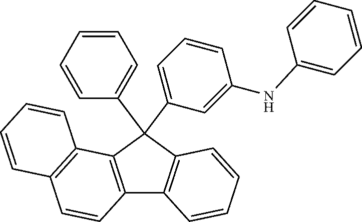 [SMILES: c1ccc(Nc2cccc(C3(c4ccccc4)c4ccccc4-c4ccc5ccccc5c43)c2)cc1]

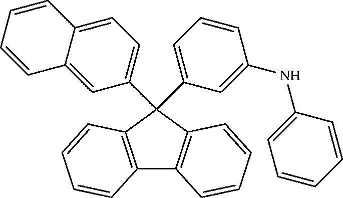 [SMILES: c1ccc(Nc2cccc(C3(c4ccc5ccccc5c4)c4ccccc4-c4ccccc43)c2)cc1]

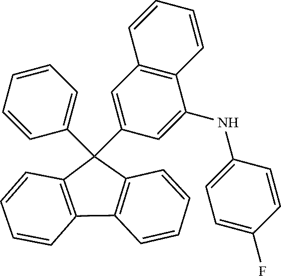 [SMILES: Fc1ccc(Nc2cc(C3(c4ccccc4)c4ccccc4-c4ccccc43)cc3ccccc23)cc1]

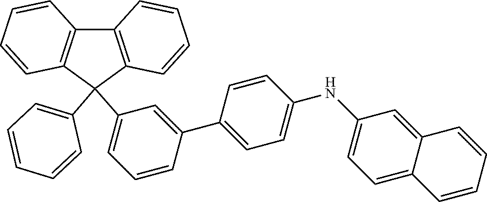 [SMILES: c1ccc(C2(c3cccc(-c4ccc(Nc5ccc6ccccc6c5)cc4)c3)c3ccccc3-c3ccccc32)cc1]

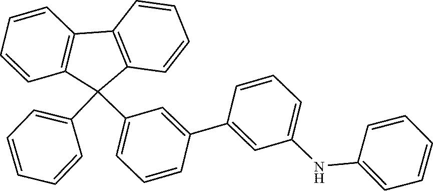 [SMILES: c1ccc(Nc2cccc(-c3cccc(C4(c5ccccc5)c5ccccc5-c5ccccc54)c3)c2)cc1]

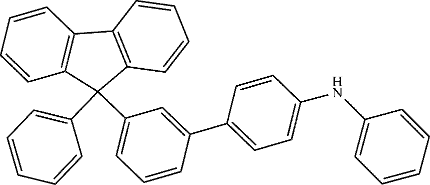 [SMILES: c1ccc(Nc2ccc(-c3cccc(C4(c5ccccc5)c5ccccc5-c5ccccc54)c3)cc2)cc1]

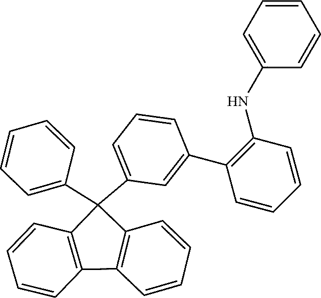 [SMILES: c1ccc(Nc2ccccc2-c2cccc(C3(c4ccccc4)c4ccccc4-c4ccccc43)c2)cc1]

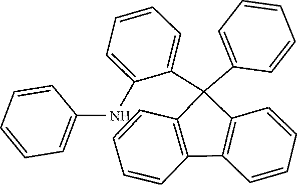 [SMILES: c1ccc(Nc2ccccc2C2(c3ccccc3)c3ccccc3-c3ccccc32)cc1]

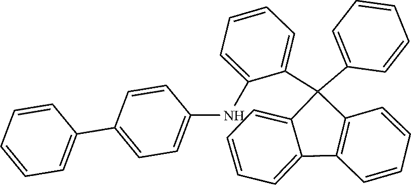 [SMILES: c1ccc(-c2ccc(Nc3ccccc3C3(c4ccccc4)c4ccccc4-c4ccccc43)cc2)cc1]

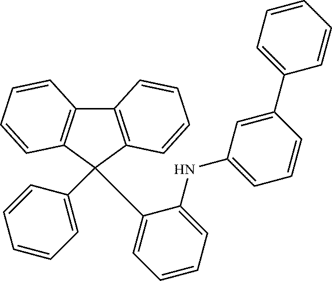 [SMILES: c1ccc(-c2cccc(Nc3ccccc3C3(c4ccccc4)c4ccccc4-c4ccccc43)c2)cc1]

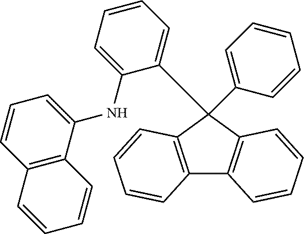 [SMILES: c1ccc(C2(c3ccccc3Nc3cccc4ccccc34)c3ccccc3-c3ccccc32)cc1]

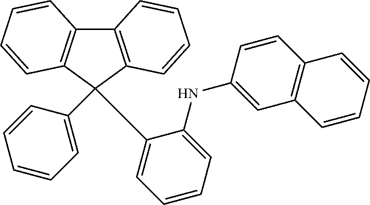 [SMILES: c1ccc(C2(c3ccccc3Nc3ccc4ccccc4c3)c3ccccc3-c3ccccc32)cc1]

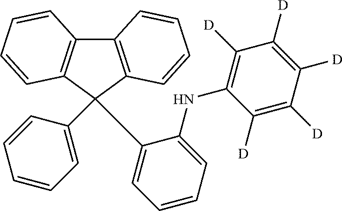 [SMILES: [2H]c1c([2H])c([2H])c(Nc2ccccc2C2(c3ccccc3)c3ccccc3-c3ccccc32)c([2H])c1[2H]]

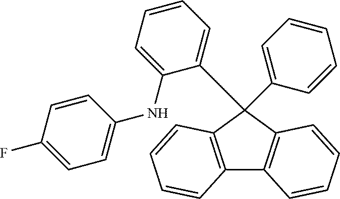 [SMILES: Fc1ccc(Nc2ccccc2C2(c3ccccc3)c3ccccc3-c3ccccc32)cc1]

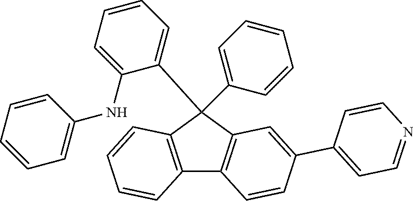 [SMILES: c1ccc(Nc2ccccc2C2(c3ccccc3)c3ccccc3-c3ccc(-c4ccncc4)cc32)cc1]

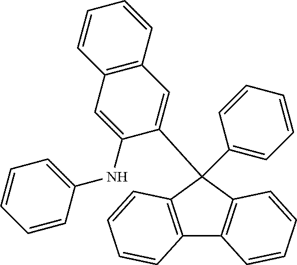 [SMILES: c1ccc(Nc2cc3ccccc3cc2C2(c3ccccc3)c3ccccc3-c3ccccc32)cc1]

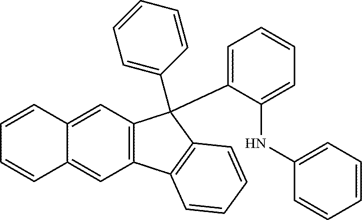 [SMILES: c1ccc(Nc2ccccc2C2(c3ccccc3)c3ccccc3-c3cc4ccccc4cc32)cc1]

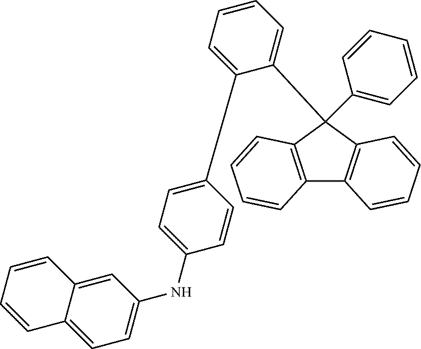 [SMILES: c1ccc(C2(c3ccccc3-c3ccc(Nc4ccc5ccccc5c4)cc3)c3ccccc3-c3ccccc32)cc1]

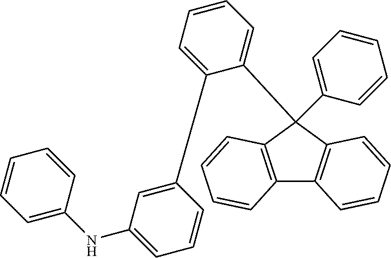 [SMILES: c1ccc(Nc2cccc(-c3ccccc3C3(c4ccccc4)c4ccccc4-c4ccccc43)c2)cc1]

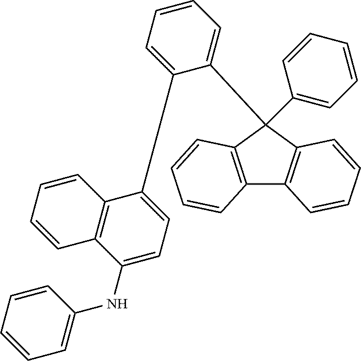 [SMILES: c1ccc(Nc2ccc(-c3ccccc3C3(c4ccccc4)c4ccccc4-c4ccccc43)c3ccccc23)cc1]

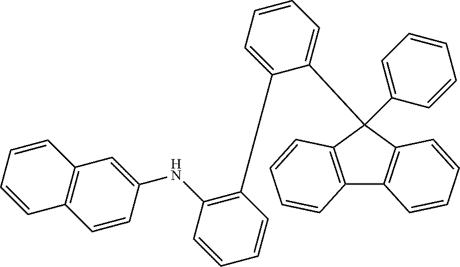 [SMILES: c1ccc(C2(c3ccccc3-c3ccccc3Nc3ccc4ccccc4c3)c3ccccc3-c3ccccc32)cc1]

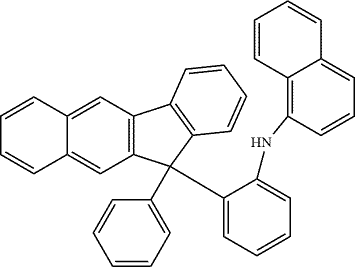 [SMILES: c1ccc(C2(c3ccccc3Nc3cccc4ccccc34)c3ccccc3-c3cc4ccccc4cc32)cc1]

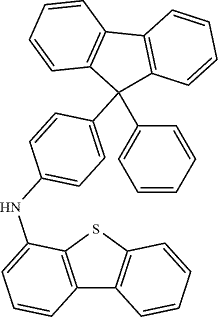 [SMILES: c1ccc(C2(c3ccc(Nc4cccc5c4sc4ccccc45)cc3)c3ccccc3-c3ccccc32)cc1]

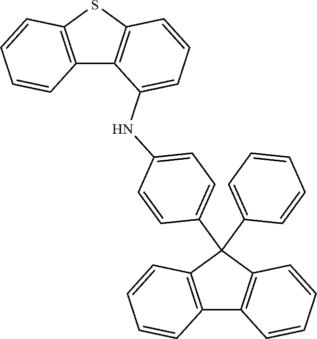 [SMILES: c1ccc(C2(c3ccc(Nc4cccc5sc6ccccc6c45)cc3)c3ccccc3-c3ccccc32)cc1]

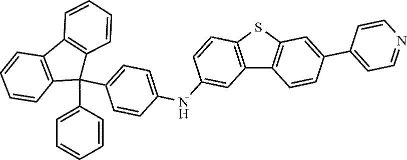 [SMILES: c1ccc(C2(c3ccc(Nc4ccc5sc6cc(-c7ccncc7)ccc6c5c4)cc3)c3ccccc3-c3ccccc32)cc1]

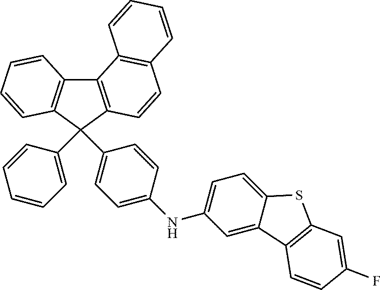 [SMILES: Fc1ccc2c(c1)sc1ccc(Nc3ccc(C4(c5ccccc5)c5ccccc5-c5c4ccc4ccccc54)cc3)cc12]

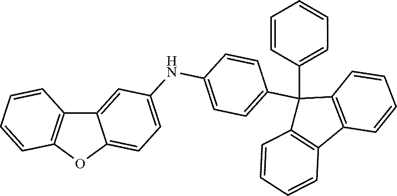 [SMILES: c1ccc(C2(c3ccc(Nc4ccc5oc6ccccc6c5c4)cc3)c3ccccc3-c3ccccc32)cc1]

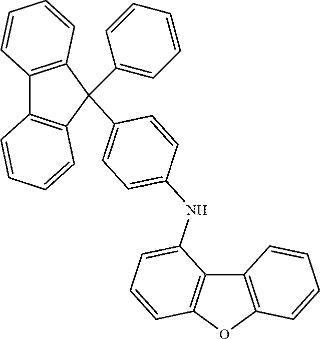 [SMILES: c1ccc(C2(c3ccc(Nc4cccc5oc6ccccc6c45)cc3)c3ccccc3-c3ccccc32)cc1]

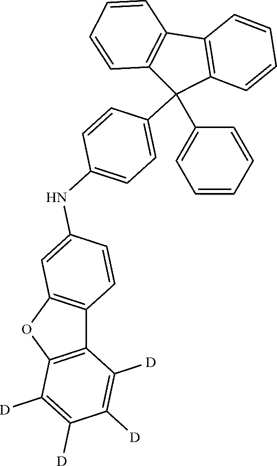 [SMILES: [2H]c1c([2H])c([2H])c2c(oc3cc(Nc4ccc(C5(c6ccccc6)c6ccccc6-c6ccccc65)cc4)ccc32)c1[2H]]

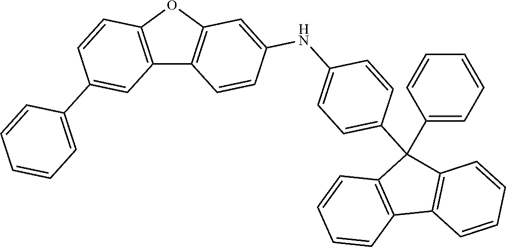 [SMILES: c1ccc(-c2ccc3oc4cc(Nc5ccc(C6(c7ccccc7)c7ccccc7-c7ccccc76)cc5)ccc4c3c2)cc1]

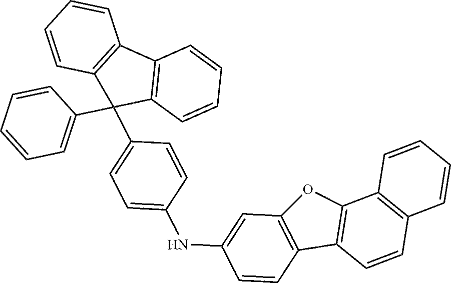 [SMILES: c1ccc(C2(c3ccc(Nc4ccc5c(c4)oc4c6ccccc6ccc54)cc3)c3ccccc3-c3ccccc32)cc1]

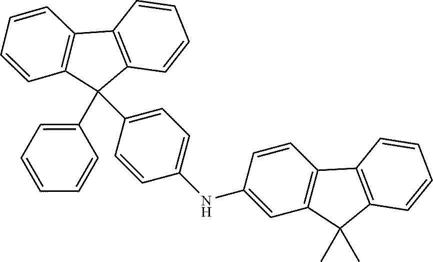 [SMILES: CC1(C)c2ccccc2-c2ccc(Nc3ccc(C4(c5ccccc5)c5ccccc5-c5ccccc54)cc3)cc21]

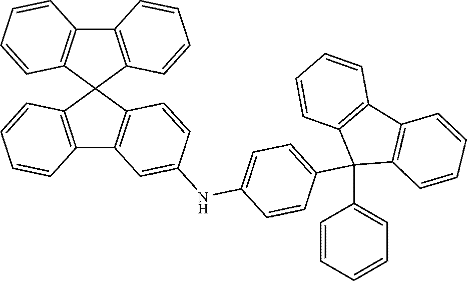 [SMILES: c1ccc(C2(c3ccc(Nc4ccc5c(c4)-c4ccccc4C54c5ccccc5-c5ccccc54)cc3)c3ccccc3-c3ccccc32)cc1]

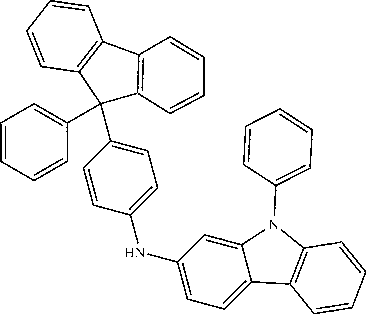 [SMILES: c1ccc(-n2c3ccccc3c3ccc(Nc4ccc(C5(c6ccccc6)c6ccccc6-c6ccccc65)cc4)cc32)cc1]

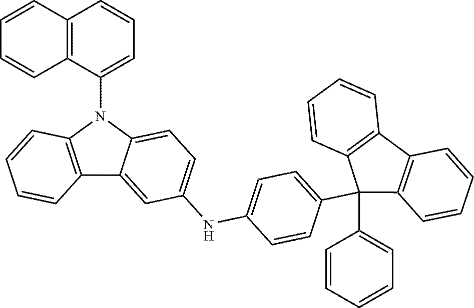 [SMILES: c1ccc(C2(c3ccc(Nc4ccc5c(c4)c4ccccc4n5-c4cccc5ccccc45)cc3)c3ccccc3-c3ccccc32)cc1]

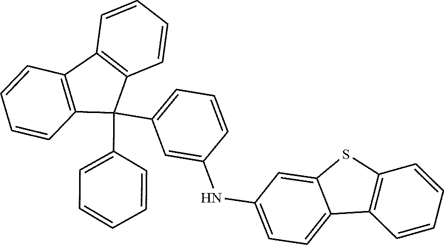 [SMILES: c1ccc(C2(c3cccc(Nc4ccc5c(c4)sc4ccccc45)c3)c3ccccc3-c3ccccc32)cc1]

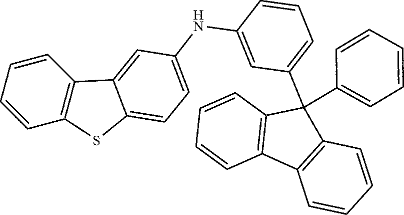 [SMILES: c1ccc(C2(c3cccc(Nc4ccc5sc6ccccc6c5c4)c3)c3ccccc3-c3ccccc32)cc1]

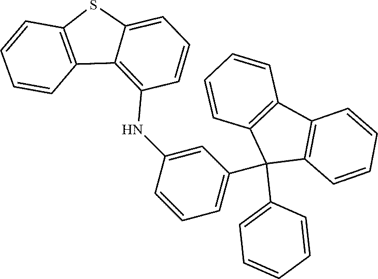 [SMILES: c1ccc(C2(c3cccc(Nc4cccc5sc6ccccc6c45)c3)c3ccccc3-c3ccccc32)cc1]

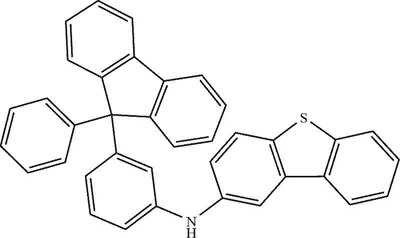 [SMILES: c1ccc(C2(c3cccc(Nc4ccc5sc6ccccc6c5c4)c3)c3ccccc3-c3ccccc32)cc1]

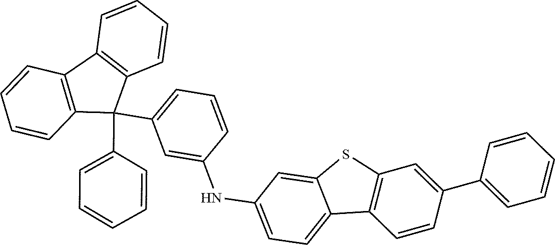 [SMILES: c1ccc(-c2ccc3c(c2)sc2cc(Nc4cccc(C5(c6ccccc6)c6ccccc6-c6ccccc65)c4)ccc23)cc1]

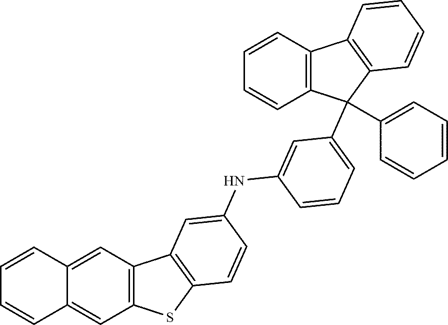 [SMILES: c1ccc(C2(c3cccc(Nc4ccc5sc6cc7ccccc7cc6c5c4)c3)c3ccccc3-c3ccccc32)cc1]

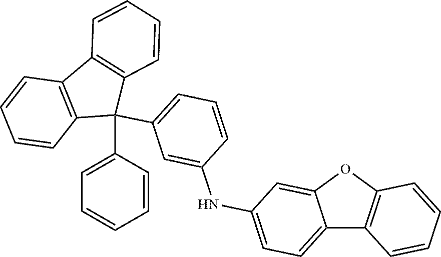 [SMILES: c1ccc(C2(c3cccc(Nc4ccc5c(c4)oc4ccccc45)c3)c3ccccc3-c3ccccc32)cc1]

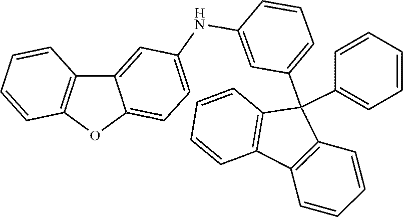 [SMILES: c1ccc(C2(c3cccc(Nc4ccc5oc6ccccc6c5c4)c3)c3ccccc3-c3ccccc32)cc1]

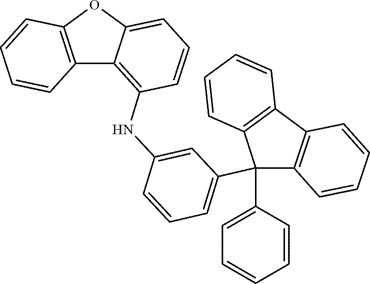 [SMILES: c1ccc(C2(c3cccc(Nc4cccc5oc6ccccc6c45)c3)c3ccccc3-c3ccccc32)cc1]

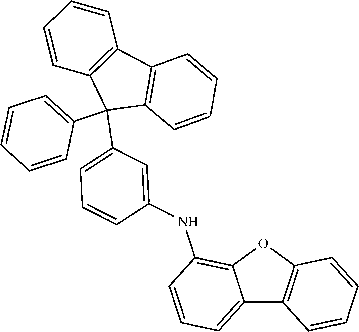 [SMILES: c1ccc(C2(c3cccc(Nc4cccc5c4oc4ccccc45)c3)c3ccccc3-c3ccccc32)cc1]

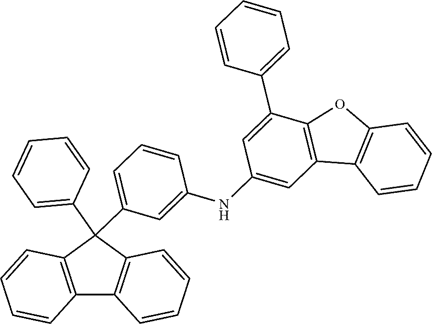 [SMILES: c1ccc(-c2cc(Nc3cccc(C4(c5ccccc5)c5ccccc5-c5ccccc54)c3)cc3c2oc2ccccc23)cc1]

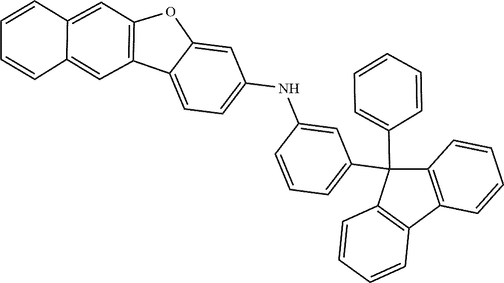 [SMILES: c1ccc(C2(c3cccc(Nc4ccc5c(c4)oc4cc6ccccc6cc45)c3)c3ccccc3-c3ccccc32)cc1]

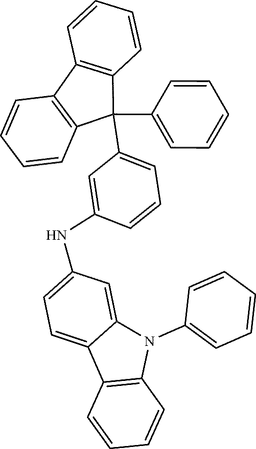 [SMILES: c1ccc(-n2c3ccccc3c3ccc(Nc4cccc(C5(c6ccccc6)c6ccccc6-c6ccccc65)c4)cc32)cc1]

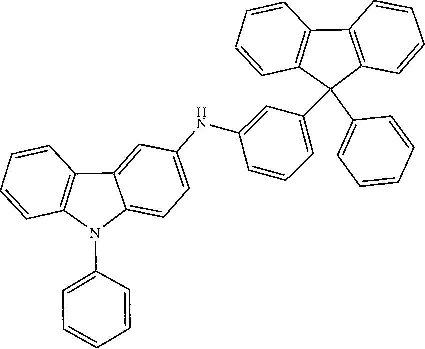 [SMILES: c1ccc(-n2c3ccccc3c3cc(Nc4cccc(C5(c6ccccc6)c6ccccc6-c6ccccc65)c4)ccc32)cc1]

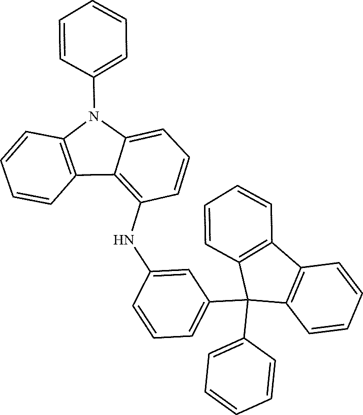 [SMILES: c1ccc(-n2c3ccccc3c3c(Nc4cccc(C5(c6ccccc6)c6ccccc6-c6ccccc65)c4)cccc32)cc1]

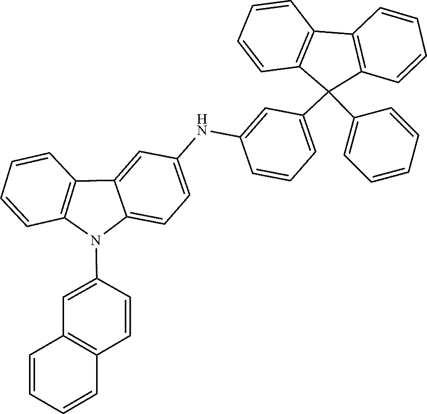 [SMILES: c1ccc(C2(c3cccc(Nc4ccc5c(c4)c4ccccc4n5-c4ccc5ccccc5c4)c3)c3ccccc3-c3ccccc32)cc1]

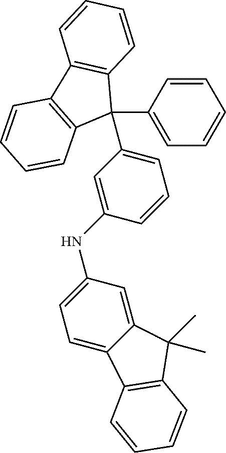 [SMILES: CC1(C)c2ccccc2-c2ccc(Nc3cccc(C4(c5ccccc5)c5ccccc5-c5ccccc54)c3)cc21]

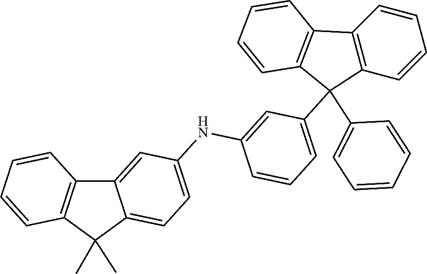 [SMILES: CC1(C)c2ccccc2-c2cc(Nc3cccc(C4(c5ccccc5)c5ccccc5-c5ccccc54)c3)ccc21]

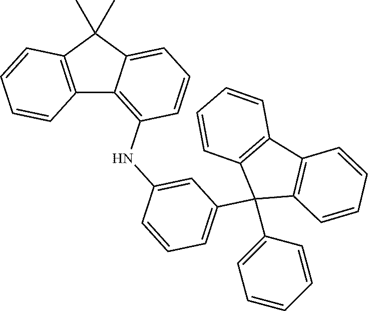 [SMILES: CC1(C)c2ccccc2-c2c(Nc3cccc(C4(c5ccccc5)c5ccccc5-c5ccccc54)c3)cccc21]

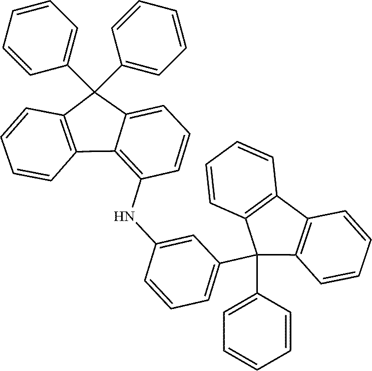 [SMILES: c1ccc(C2(c3cccc(Nc4cccc5c4-c4ccccc4C5(c4ccccc4)c4ccccc4)c3)c3ccccc3-c3ccccc32)cc1]

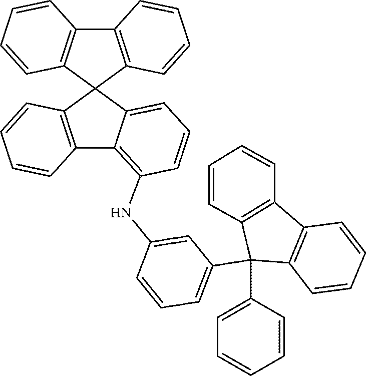 [SMILES: c1ccc(C2(c3cccc(Nc4cccc5c4-c4ccccc4C54c5ccccc5-c5ccccc54)c3)c3ccccc3-c3ccccc32)cc1]

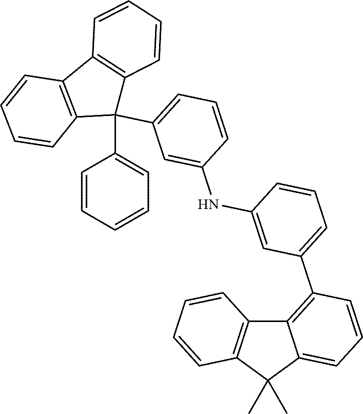 [SMILES: CC1(C)c2ccccc2-c2c(-c3cccc(Nc4cccc(C5(c6ccccc6)c6ccccc6-c6ccccc65)c4)c3)cccc21]

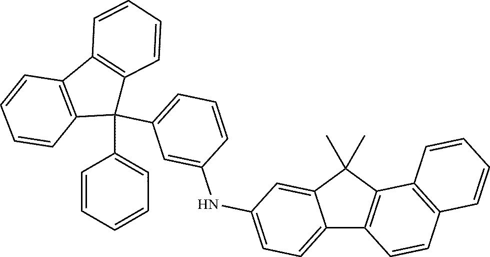 [SMILES: CC1(C)c2cc(Nc3cccc(C4(c5ccccc5)c5ccccc5-c5ccccc54)c3)ccc2-c2ccc3ccccc3c21]

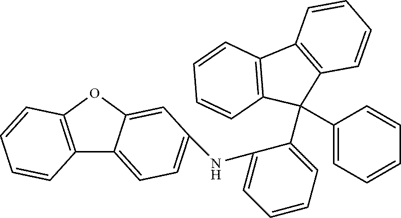 [SMILES: c1ccc(C2(c3ccccc3Nc3ccc4c(c3)oc3ccccc34)c3ccccc3-c3ccccc32)cc1]

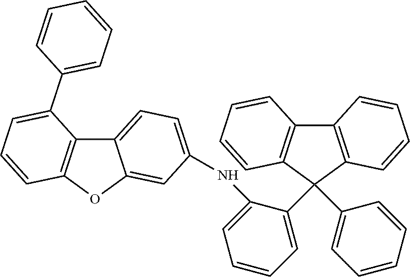 [SMILES: c1ccc(-c2cccc3oc4cc(Nc5ccccc5C5(c6ccccc6)c6ccccc6-c6ccccc65)ccc4c23)cc1]

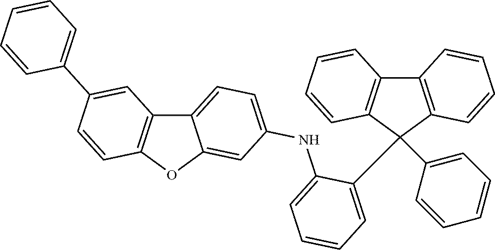 [SMILES: c1ccc(-c2ccc3oc4cc(Nc5ccccc5C5(c6ccccc6)c6ccccc6-c6ccccc65)ccc4c3c2)cc1]

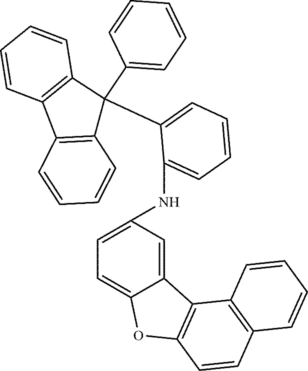 [SMILES: c1ccc(C2(c3ccccc3Nc3ccc4oc5ccc6ccccc6c5c4c3)c3ccccc3-c3ccccc32)cc1]

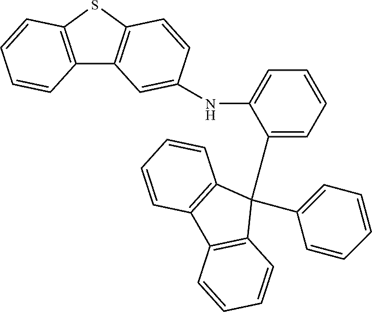 [SMILES: c1ccc(C2(c3ccccc3Nc3ccc4sc5ccccc5c4c3)c3ccccc3-c3ccccc32)cc1]

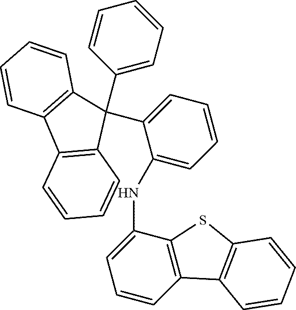 [SMILES: c1ccc(C2(c3ccccc3Nc3cccc4c3sc3ccccc34)c3ccccc3-c3ccccc32)cc1]

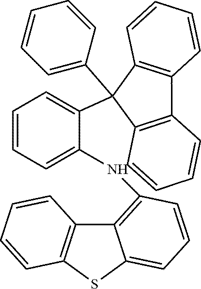 [SMILES: c1ccc(C2(c3ccccc3Nc3cccc4sc5ccccc5c34)c3ccccc3-c3ccccc32)cc1]

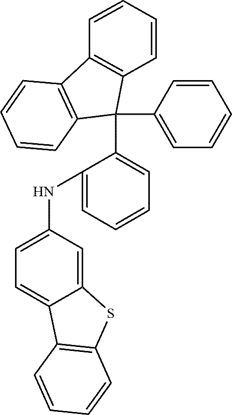 [SMILES: c1ccc(C2(c3ccccc3Nc3ccc4c(c3)sc3ccccc34)c3ccccc3-c3ccccc32)cc1]

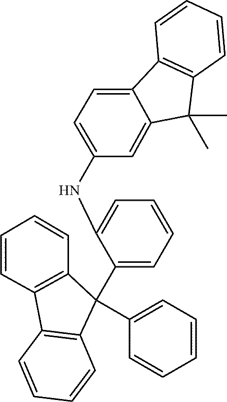 [SMILES: CC1(C)c2ccccc2-c2ccc(Nc3ccccc3C3(c4ccccc4)c4ccccc4-c4ccccc43)cc21]

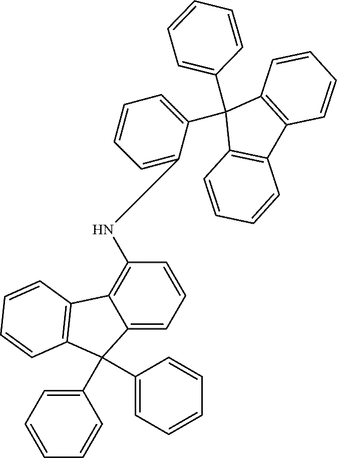 [SMILES: c1ccc(C2(c3ccccc3)c3ccccc3-c3c(Nc4ccccc4C4(c5ccccc5)c5ccccc5-c5ccccc54)cccc32)cc1]

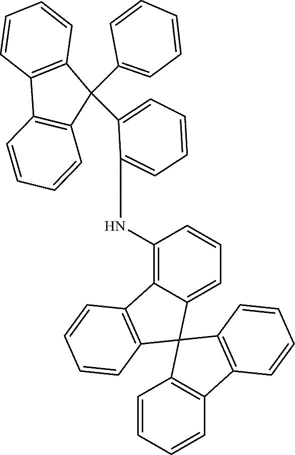 [SMILES: c1ccc(C2(c3ccccc3Nc3cccc4c3-c3ccccc3C43c4ccccc4-c4ccccc43)c3ccccc3-c3ccccc32)cc1]

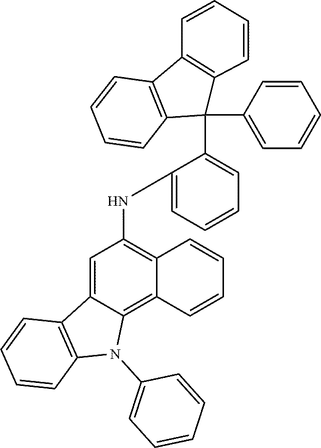 [SMILES: c1ccc(-n2c3ccccc3c3cc(Nc4ccccc4C4(c5ccccc5)c5ccccc5-c5ccccc54)c4ccccc4c32)cc1]

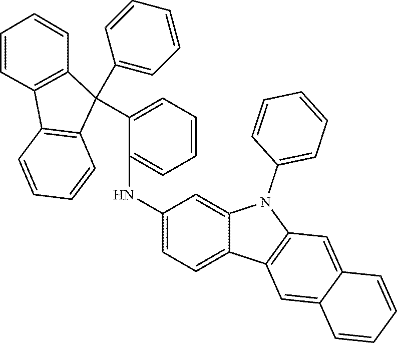 [SMILES: c1ccc(-n2c3cc(Nc4ccccc4C4(c5ccccc5)c5ccccc5-c5ccccc54)ccc3c3cc4ccccc4cc32)cc1]

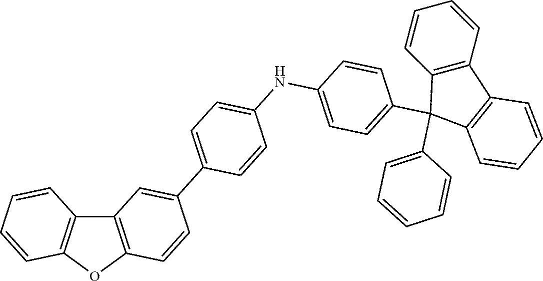 [SMILES: c1ccc(C2(c3ccc(Nc4ccc(-c5ccc6oc7ccccc7c6c5)cc4)cc3)c3ccccc3-c3ccccc32)cc1]

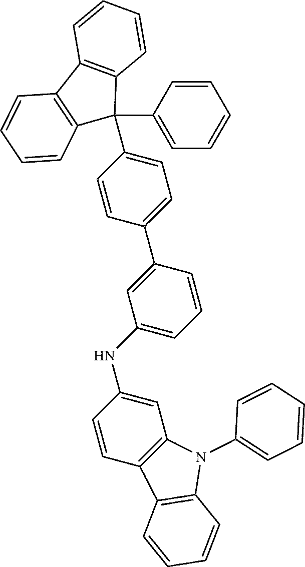 [SMILES: c1ccc(-n2c3ccccc3c3ccc(Nc4cccc(-c5ccc(C6(c7ccccc7)c7ccccc7-c7ccccc76)cc5)c4)cc32)cc1]

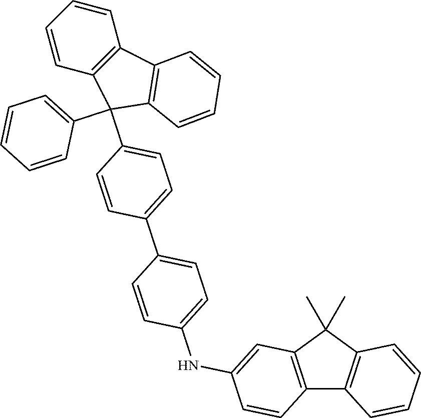 [SMILES: CC1(C)c2ccccc2-c2ccc(Nc3ccc(-c4ccc(C5(c6ccccc6)c6ccccc6-c6ccccc65)cc4)cc3)cc21]

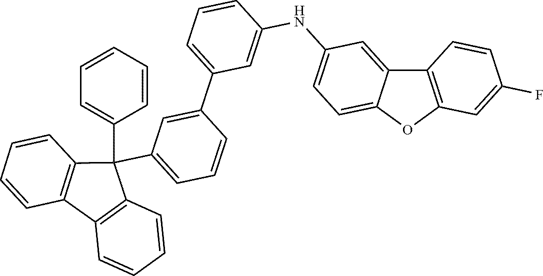 [SMILES: Fc1ccc2c(c1)oc1ccc(Nc3cccc(-c4cccc(C5(c6ccccc6)c6ccccc6-c6ccccc65)c4)c3)cc12]

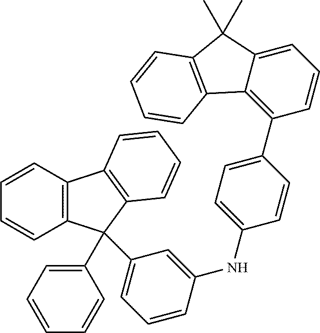 [SMILES: CC1(C)c2ccccc2-c2c(-c3ccc(Nc4cccc(C5(c6ccccc6)c6ccccc6-c6ccccc65)c4)cc3)cccc21]

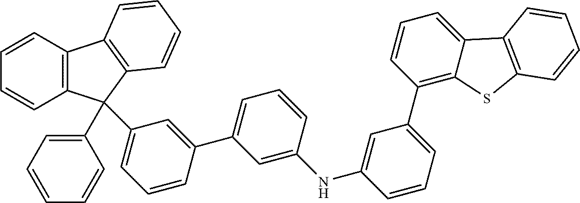 [SMILES: c1ccc(C2(c3cccc(-c4cccc(Nc5cccc(-c6cccc7c6sc6ccccc67)c5)c4)c3)c3ccccc3-c3ccccc32)cc1]

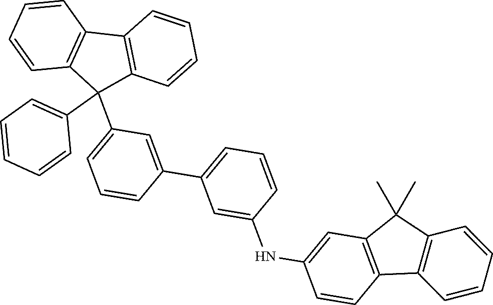 [SMILES: CC1(C)c2ccccc2-c2ccc(Nc3cccc(-c4cccc(C5(c6ccccc6)c6ccccc6-c6ccccc65)c4)c3)cc21]

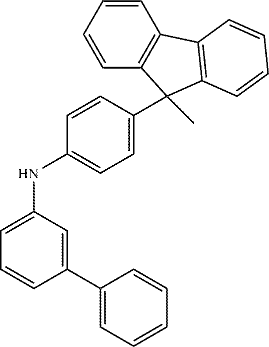 [SMILES: CC1(c2ccc(Nc3cccc(-c4ccccc4)c3)cc2)c2ccccc2-c2ccccc21]

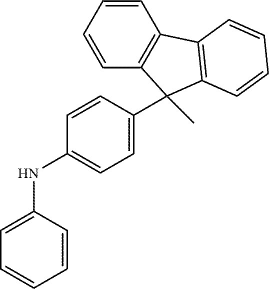 [SMILES: CC1(c2ccc(Nc3ccccc3)cc2)c2ccccc2-c2ccccc21]

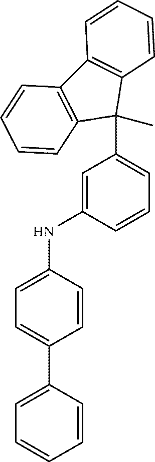 [SMILES: CC1(c2cccc(Nc3ccc(-c4ccccc4)cc3)c2)c2ccccc2-c2ccccc21]

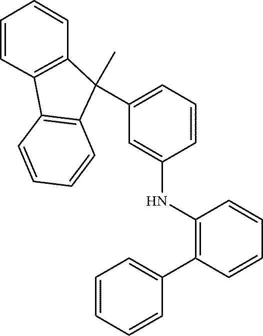 [SMILES: CC1(c2cccc(Nc3ccccc3-c3ccccc3)c2)c2ccccc2-c2ccccc21]

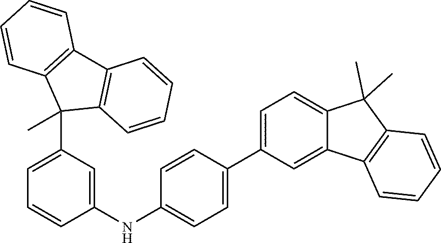 [SMILES: CC1(C)c2ccccc2-c2cc(-c3ccc(Nc4cccc(C5(C)c6ccccc6-c6ccccc65)c4)cc3)ccc21]

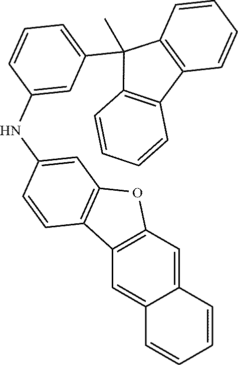 [SMILES: CC1(c2cccc(Nc3ccc4c(c3)oc3cc5ccccc5cc34)c2)c2ccccc2-c2ccccc21]

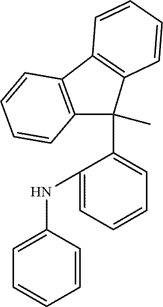 [SMILES: CC1(c2ccccc2Nc2ccccc2)c2ccccc2-c2ccccc21]

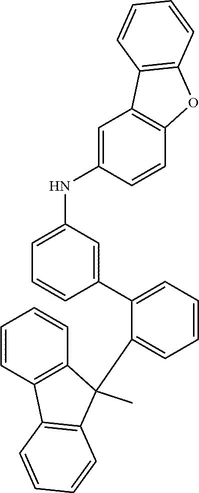 [SMILES: CC1(c2ccccc2-c2cccc(Nc3ccc4oc5ccccc5c4c3)c2)c2ccccc2-c2ccccc21]

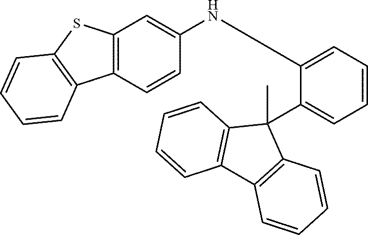 [SMILES: CC1(c2ccccc2Nc2ccc3c(c2)sc2ccccc23)c2ccccc2-c2ccccc21]

TABLE 2

| | | | |
|---|---|---|---|
| comp | FD-MS | comp | FD-MS |
| Sub2-1 | m/z=409.18(C₃₁H₂₃N=409.53) | Sub2 -2 | m/z=485.21 (C₃₇H₂₇N=485.63) |
| Sub2-3 | m/z=485.21 (C₃₇H₂₇N=485.63) | Sub2 -4 | m/z=414.21 (C₃₁H₁₈D₅N=414.56 ) |
| Sub2-5 | m/z=427.17(C₃₁H₂₂FN=427.52) | Sub2 -6 | m/z=434.18(C₃₂H₂₂N₂=434.54) |
| Sub2-7 | m/z=481.24(C₃₅H₃₁NO=481.64) | Sub2 -8 | m/z=427.17(C₃₁ H₂₂FN=427.52) |
| Sub2-9 | m/z=486.21 (C₃₆H₂₆N₂=486.62) | Sub2 -10 | m/z=459.20(C₃₅H₂₅N=459.59) |
| Sub2-11 | m/z=485.21 (C₃₇H₂₇N=485.63) | Sub2 -12 | m/z=503.20(C₃₇H₂₆FN=503.62) |
| Sub2-13 | m/z=485.21 (C₃₇H₂₇N=485.63) | Sub2 -14 | m/z=409.18(C₃₁H₂₃N=409.53) |
| Sub2-15 | m/z=485.21 (C₃₇H₂₇N=485.63) | Sub2 -16 | m/z=485.21 (C₃₇H₂₇N=485.63) |
| Sub2-17 | m/z=485.21 (C₃₇H₂₇N=485.63) | Sub2 -18 | m/z=459.20(C₃₅H₂₅N=459.59) |
| Sub2-19 | m/z=459.20(C₃₅H₂₅N=459.59) | Sub2 -20 | m/z=485.21 (C₃₇H₂₇N=485.63) |
| Sub2-21 | m/z=414.21 (C₃₁H₁₈D₅N=414.56) | Sub2 -22 | m/z=427.17(C₃₁H₂₂FN=427.52) |
| Sub2-23 | m/z=434.18(C₃₂H₂₂N₂=434.54) | Sub2 -24 | m/z=501.21 (C₃₇H₂₇NO=501.63) |
| Sub2-25 | m/z=459.20(C₃₅H₂₅N=459.59) | Sub2 -26 | m/z=459.20(C₃₅H₂₅N=459.59) |
| Sub2- | m/z=477.19(C₃₅H₂₄FN=477.58) | Sub2 | m/z=535.23(C₄₁H₂₉N=535.69) |
| 27 | | -28 | |
| Sub2-29 | m/z=485.21 (C₃₇H₂₇N=485.63) | Sub2 -30 | m/z=485.21 (C₃₇H₂₇N=485.63) |
| Sub2-31 | m/z=485.21 (C₃₇H₂₇N=485.63) | Sub2 -32 | m/z=409.18(C₃₁H₂₃N=409.53) |
| Sub2-33 | m/z=485.21 (C₃₇H₂₇N=485.63) | Sub2 -34 | m/z=485.21 (C₃₇H₂₇N=485.63) |
| Sub2-35 | m/z=459.20(C₃₅H₂₅N=459.59) | Sub2 -36 | m/z=459.20(C₃₅H₂₅N=459.59) |
| Sub2-37 | m/z=414.21 (C₃₁H₁₈D₅N=414.56) | Sub2 -38 | m/z=427.17(C₃₁H₂₂FN=427.52) |
| Sub2-39 | m/z=486.21 (C₃₆H₂₆N₂=486.62) | Sub2 -40 | m/z=459.20(C₃₅H₂₅N=459.59) |
| Sub2-41 | m/z=459.20(C₃₅H₂₅N=459.59) | Sub2 -42 | m/z=535.23(C₄₁H₂₉N=535.69) |
| Sub2-43 | m/z=485.21 (C₃₇H₂₇N=485.63) | Sub2 -44 | m/z=535.23(C₄₁H₂₉N=535.69) |
| Sub2-45 | m/z=535.23(C₄₁H₂₉N=535.69) | Sub2 -46 | m/z=509.21 (C₃₉H₂₇N=509.65) |
| Sub2-47 | m/z=515.17(C₃₇H₂₅NS=515.67) | Sub2 -48 | m/z=515.17(C₃₇H₂₅NS=515.67) |
| Sub2-49 | m/z=592.2(C₄₂H_{2S}N₂S=592.76) | Sub2 -50 | m/z=583.18(C₄₁H₂₆FNS=583.72) |
| Sub2-51 | m/z=499.19(C₃₇H₂₅NO=499.61) | Sub2 -52 | m/z=499.19(C₃₇H₂₅NO=499.61) |
| Sub2-53 | m/z=503.22(C₃₇H₂₁D₄NO=503.64 ) | Sub2 -54 | m/z=575.22(C₄₃H₂₉NO=575.71) |
| Sub2- | m/z=549.21(C₄₁H₂₇NO=549.67) | Sub2 | m/z=525.25(C₄₀H₃₁N=525.70) |
| 55 | | -56 | |
| Sub2-57 | m/z=647.26(C₅₀H₃₃N=647.82) | Sub2 -58 | m/z=574.24(C₄₃H₃₀N₂=574.73) |
| Sub2-59 | m/z=624.26(C₄₇H₃₂N₂=624.79) | Sub2 -60 | m/z=515.17(C₃₇H₂₅NS=515.67) |
| Sub2-61 | m/z=515.17(C₃₇H₂₅NS=515.67) | Sub2 -62 | m/z=515.17(C₃₇H₂₅NS=515.67) |
| Sub2-63 | m/z=515.17(C₃₇H₂₅NS=515.67) | Sub2 -64 | m/z=591.20(C₄₃H₂₉NS=591.77) |
| Sub2-65 | m/z=565.19(C₄₁H₂₇NS=565.73) | Sub2 -66 | m/z=499.19(C₃₇H₂₅NO=499.61) |
| Sub2-67 | m/z=499.19(C₃₇H₂₅NO=499.61) | Sub2 -68 | m/z=499.19(C₃₇H₂₅NO=499.61) |
| Sub2-69 | m/z=499.19(C₃₇H₂₅NO=499.61) | Sub2 -70 | m/z=575.22(C₄₃H₂₉NO=575.71) |
| Sub2-71 | m/z=549.21 (C₄₁H₂₇NO=549.67) | Sub2 -72 | m/z=574.24(C₄₃H₃₀N₂=574.73) |
| Sub2-73 | m/z=574.24(C₄₃H₃₀N₂=574.73) | Sub2 -74 | m/z=574.24(C₄₃H₃₀N₂=574.73) |
| Sub2-75 | m/z=624.26(C₄₇H₃₂N₂=624.79) | Sub2 -76 | m/z=525.25(C₄₀H₃₁N=525.70) |
| Sub2-77 | m/z=525.25(C₄₀H₃₁N=525.70) | Sub2 -78 | m/z=525.25(C₄₀H₃₁N=525.70) |
| Sub2-79 | m/z=649.28(C₅₀H_{3S}N=649.84) | Sub2 -80 | m/z=647.26(C₅₀H₃₃N=647.82) |
| Sub2-81 | m/z=601.28(C₄₆H₃₅N=601.79) | Sub2 -82 | m/z=575.26(C₄₄H₃₃N=575.76) |
| Sub2- | m/z=499.19(C₃₇H₂₅NO=499.61) | Sub2 | m/z=575.22(C₄₃H₂₉NO=575.71) |
| 83 | | -84 | |
| Sub2-85 | m/z=575.22(C₄₃H₂₉NO=575.71) | Sub2 -86 | m/z=549.21 (C₄₁H₂₇NO=549.67) |
| Sub2-87 | m/z=515.17(C₃₇H₂₅NS=515.67) | Sub2 -88 | m/z=515.17(C₃₇H₂₅NS=515.67) |
| Sub2-89 | m/z=515.17(C₃₇H₂₅NS=515.67) | Sub2 -90 | m/z=515.17(C₃₇H₂₅NS=515.67) |
| Sub2-91 | m/z=525.25(C₄₀H₃₁ N=525.70) | Sub2 -92 | m/z=649.28(C₅₀H₃₅N=649.84) |
| Sub2-93 | m/z=647.26(C₅₀H₃₃N=647.82) | Sub2 -94 | m/z=624.26(C₄₇H₃₂N₂=624.79) |
| Sub2-95 | m/z=624.26(C₄₇H₃₂N₂=624.79) | Sub2 -96 | m/z=575.22(C₄₃H₂₉NO=575.71) |
| Sub2-97 | m/z=650.27(C₄₉H₃₄N₂=650.83) | Sub2 -98 | m/z=601.28(C₄₆H₃₅N=601.79) |
| Sub2-99 | m/z=593.22(C₄₃H₂₈FNO=593.70) | Sub2 -100 | m/z=601.28(C₄₆H₃₅N=601.79) |
| Sub2-101 | m/z=667.23(C₄₉H₃₃NS=667.87) | Sub2 -102 | m/z=601.28(C₄₆H₃₅N=601.79) |
| Sub2-103 | m/z=423.20(C₃₂H₂₅N=423.56) | Sub2 -104 | m/z=347.17(C₂₆H₂₁ N=347.46) |
| Sub2-105 | m/z=423.20(C₃₂H₂₅N=423.56) | Sub2 -106 | m/z=423.20(C₃₂H₂₅N=423.56) |
| Sub2-107 | m/z=539.26(C₄₁H₃₃N=539.72) | Sub2 -108 | m/z=487.19(C₃₆H₂₅NO=487.60) |
| Sub2-109 | m/z=347.17(C₂₆H₂₁N=347.46) | Sub2 -110 | m/z=513.21 (C₃₈H₂₇NO=513.64) |
| Sub2-111 | m/z=453.16(C₃₂H₂₃NS=453.60) | | |

### III. Synthesis of Final Product

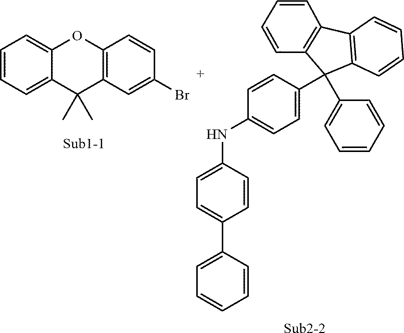

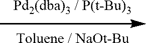

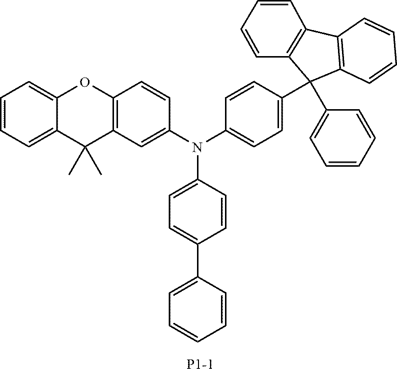

After dissolving Sub1-1 (2.0 g, 6.9 mmol) in toluene (35 mL) in a round-bottom flask, Sub2-2 (3.4 g, 6.9 mmol), Pd₂(dba)₃ (0.19 g, 0.21 mmol), P(t-Bu)₃ (0.08 g, 0.41 mmol), NaOt—Bu (1.3 g, 13.8 mmol) were added and refluxed. When the reaction was completed, the mixture was extracted with CH₂CI₂ and water, the organic layer was dried over MgSO₄, concentrated, and the resulting compound was recrystallized using silicagel column to obtain 3.5 g (yield 72%) of the product.

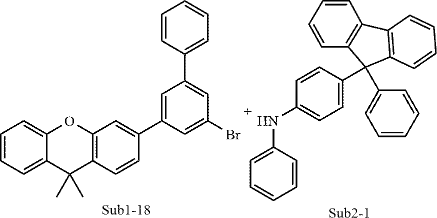

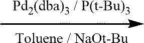

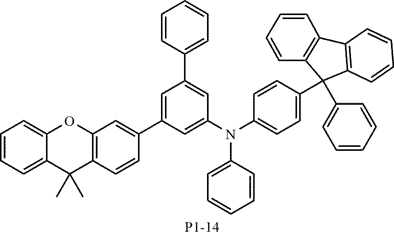

Sub1-18 (2.0 g, 4.5 mmol), Sub2-1 (1.9 g, 4.5 mmol), Pd₂(dba)₃ (0.12 g, 0.14 mmol), P(t-Bu)₃ (0.06 g, 0.27 mmol), NaOt—Bu (0.9 g, 9.1 mmol) were used to obtain 2.6 g (yield 74%) of the product using the synthesis method of P1-1

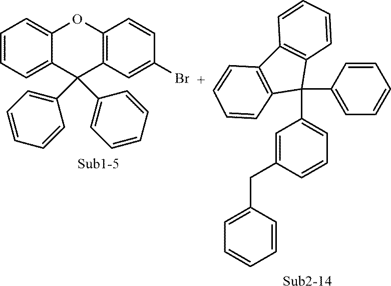

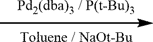

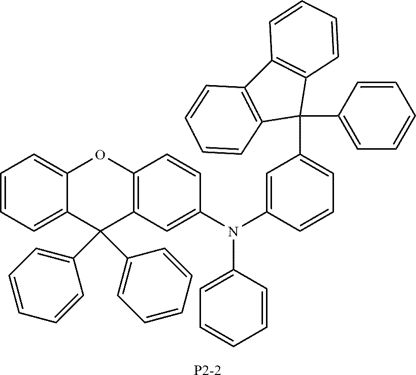

Sub1-5 (2.0 g, 4.8 mmol), Sub2-14 (2.0 g, 4.8 mmol), Pd₂(dba)₃ (0.13 g, 0.15 mmol), P(t-Bu)₃ (0.06 g, 0.29 mmol), NaOt—Bu (0.9 g, 9.7 mmol) were used to obtain 2.6 g (yield 72%) of the product using the synthesis method of P1-1

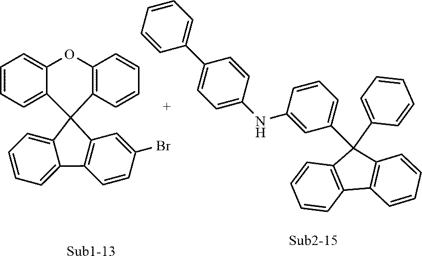

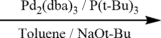

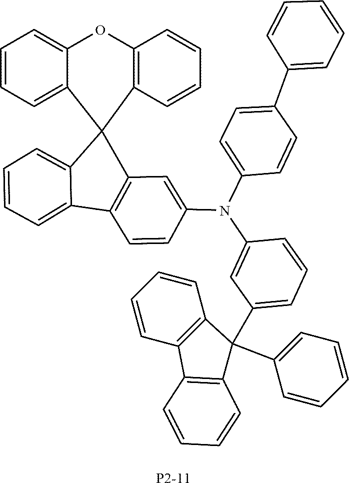

Sub1-13 (2.0 g, 4.9 mmol), Sub2-15 (2.4 g, 4.9 mmol), Pd₂(dba)₃ (0.13 g, 0.15 mmol), P(t-Bu)₃ (0.06 g, 0.29 mmol), NaOt—Bu (0.9 g, 9.7 mmol) were used to obtain 2.8 g (yield 70%) of the product using the synthesis method of P1-1

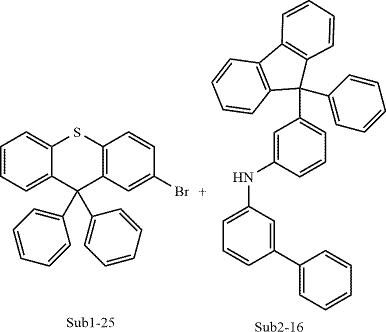

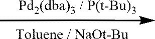

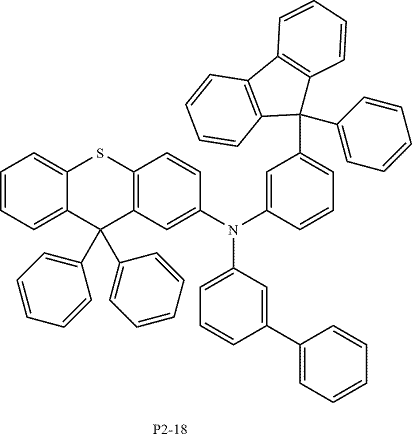

Sub1-25 (2.0 g, 4.7 mmol), Sub2-16 (2.3 g, 4.7 mmol), Pd₂(dba)₃ (0.13 g, 0.14 mmol), P(t-Bu)₃ (0.06 g, 0.28 mmol), NaOt—Bu (0.9 g, 9.3 mmol) were used to obtain 2.8 g (yield 72%) of the product using the synthesis method of P1-1

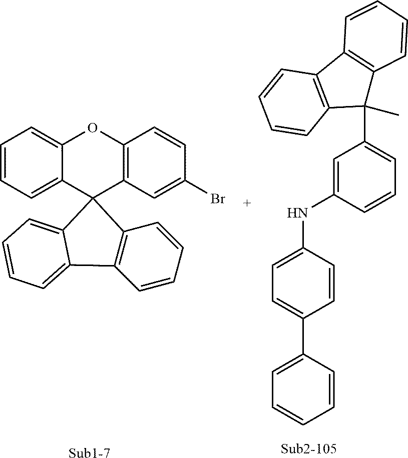

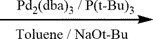

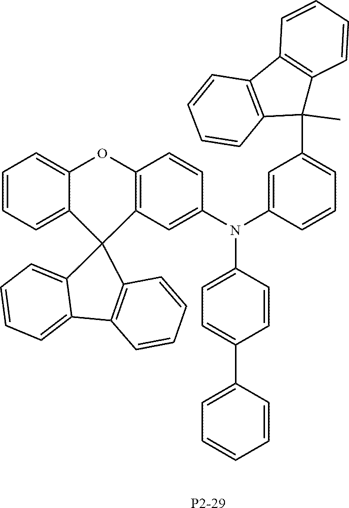

Sub1-7 (1.8 g, 4.4 mmol), Sub2-105 (1.9 g, 4.4 mmol), Pd₂(dba)₃ (0.12 g, 0.13 mmol), P(t-Bu)₃ (0.05 g, 0.26 mmol), NaOt—Bu (0.8 g, 8.8 mmol) were used to obtain 2.4 g (yield 73%) of the product using the synthesis method of P1-1

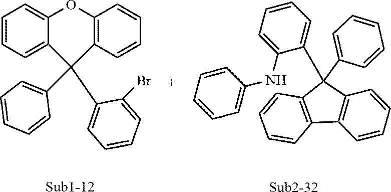

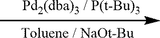

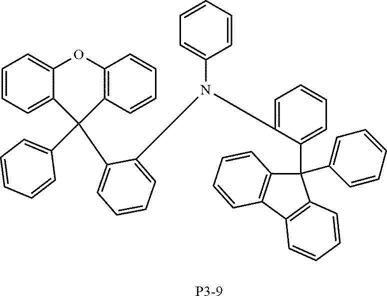

Sub1-12 (2.0 g, 4.8 mmol), Sub2-32 (2.0 g, 4.8 mmol), Pd₂(dba)₃ (0.13 g, 0.15 mmol), P(t-Bu)₃ (0.06 g, 0.29 mmol), NaOt—Bu (0.9 g, 9.7 mmol) were used to obtain 2.1 g (yield 58%) of the product using the synthesis method of P1-1

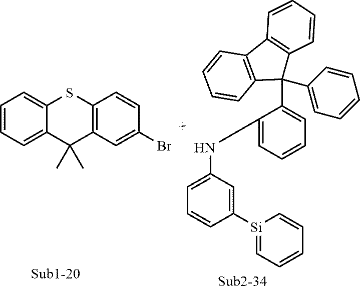

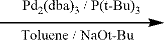

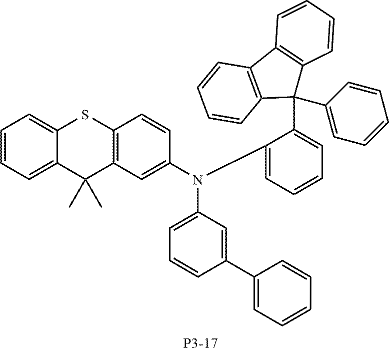

Sub1-20 (2.0 g, 6.6 mmol), Sub2-34 (3.2 g, 6.6 mmol), Pd₂(dba)₃ (0.18 g, 0.20 mmol), P(t-Bu)₃ (0.08 g, 0.39 mmol), NaOt—Bu (1.3 g, 13.1 mmol) were used to obtain 2.9 g (yield 62%) of the product using the synthesis method of P1-1

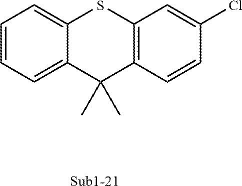

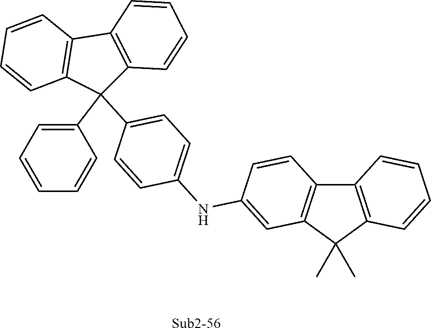

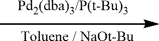

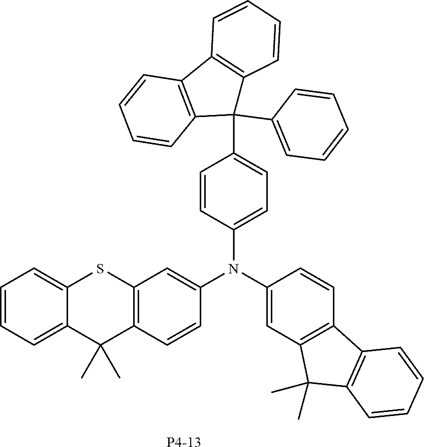

Sub1-21 (2.0 g, 7.7 mmol), Sub2-56 (4.0 g, 7.7 mmol), Pd₂(dba)₃ (0.21 g, 0.23 mmol), P(i-Bu)₃ (0.09 g, 0.46 mmol), NaOt—Bu (1.5 g, 15.3 mmol) were used to obtain 4.3 g (yield 75%) of the product using the synthesis method of P1-1

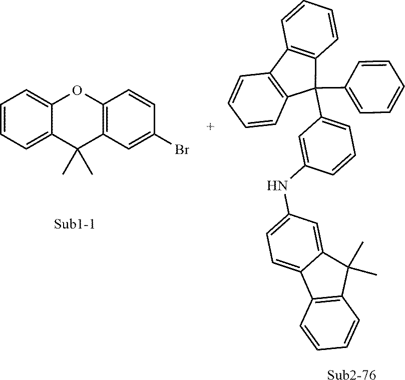

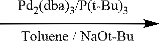

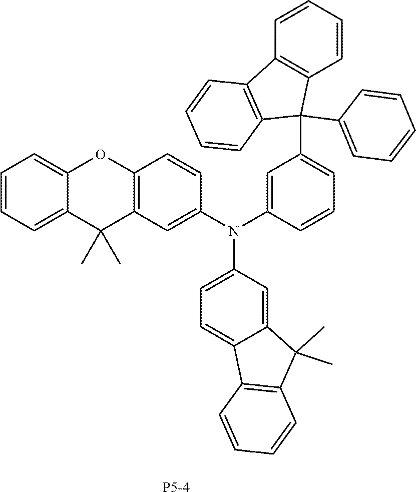 [SMILES: CC1(C)c2ccccc2Oc2ccc(N(c3cccc(C4(c5ccccc5)c5ccccc5-c5ccccc54)c3)c3ccc4c(c3)C(C)(C)c3ccccc3-4)cc21 ; "P5-4"]

Sub1-1 (2.0 g, 6.9 mmol), Sub2-76 (3.6 g, 6.9 mmol), Pd₂(dba)₃ (0.19 g, 0.21 mmol), P(t-Bu)₃ (0.08 g, 0.41 mmol), NaOt—Bu (1.3 g, 13.8 mmol) were used to obtain 3.6 g (yield 71%) of the product using the synthesis method of P1-1

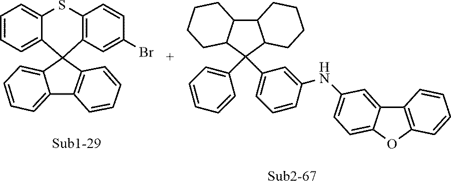

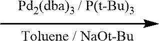

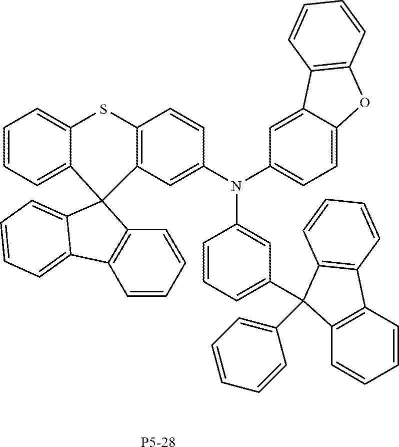

Sub1-29 (2.0 g, 4.7 mmol), Sub2-67 (2.3 g, 4.7 mmol), Pd₂(dba)₃ (0.13 g, 0.14 mmol), P(t-Bu)₃ (0.06 g, 0.28 mmol), NaOt—Bu (0.9 g, 9.4 mmol) were used to obtain 3.0 g (yield 75%) of the product using the synthesis method of P1-1

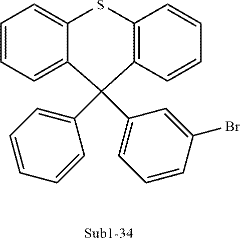

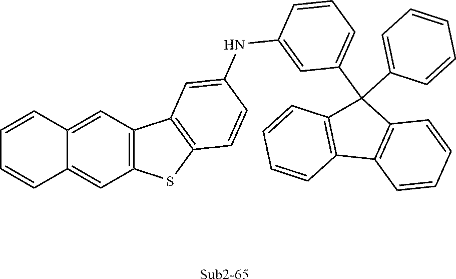

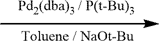

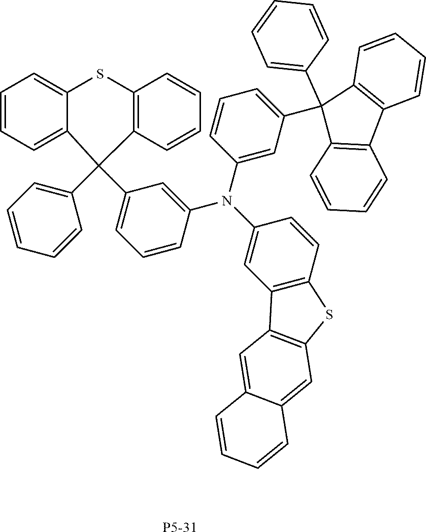

Sub1-34 (2.0 g, 4.7 mmol), Sub2-65 (2.6 g, 4.7 mmol), Pd₂(dba)₃ (0.13 g, 0.14 mmol), P(t-Bu)₃ (0.06 g, 0.28 mmol), NaOt—Bu (0.9 g, 9.3 mmol) were used to obtain 3.1 g (yield 72%) of the product using the synthesis method of P1-1

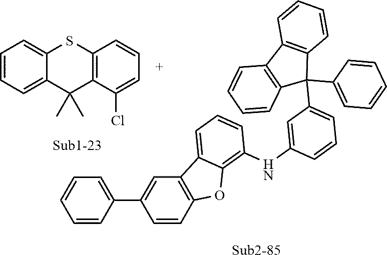

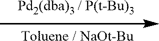

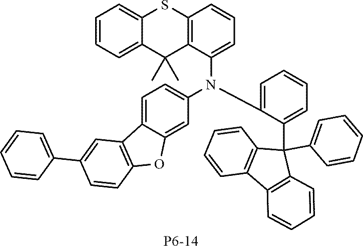

Sub1-23 (1.5 g, 5.8 mmol), Sub2-85 (3.3 g, 5.8 mmol), Pd₂(dba)₃ (0.16 g, 0.17 mmol), P(t-Bu)₃ (0.07 g, 0.35 mmol), NaOt—Bu (1.1 g, 11.5 mmol) were used to obtain 2.4 g (yield 52%) of the product using the synthesis method of P1-1

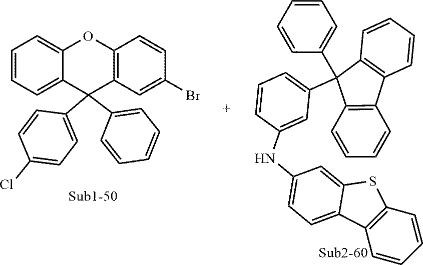

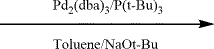

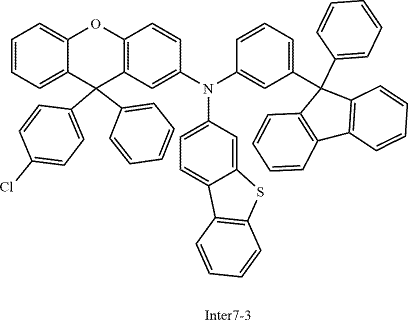

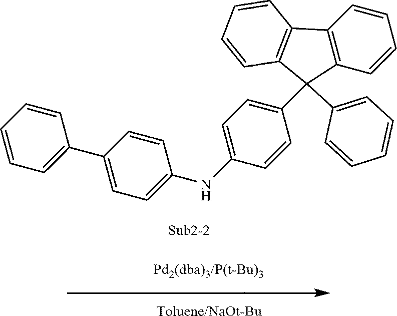

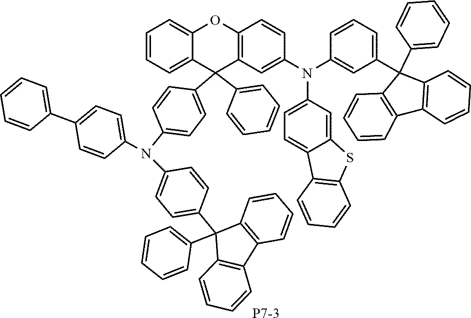

### Synthesis Example of Inter7-3

After dissolving Sub1-50 (1.5 g, 3.4 mmol) with toluene (17 mL) in a round-bottom flask, Sub2-60 (1.7 g, 3.4 mmol), Pd₂(dba)₃ (0.09 g, 0.10 mmol), P(t-Bu)₃ (0.04 g, 0.20 mmol), NaOt—Bu (0.6 g, 6.7 mmol) was added and stirred at 60° C. When the reaction was completed, the mixture was extracted with CH₂CI₂ and water, the organic layer was dried over MgSO₄, concentrated, and the resulting compound was recrystallized using silicagel column to obtain 2.1 g (yield 72%) of the product.

### Synthesis Example of P7-3

After dissolving Inter7-3 (2.1 g, 2.4 mmol) in toluene (12 mL) in a round-bottom flask, Sub2-2 (1.2 g, 2.4 mmol), Pd₂(dba)₃ (0.07 g, 0.07 mmol), P(t-Bu)₃ (0.03 g, 0.14 mmol), NaOt—Bu (0.5 g, 4.8 mmol) was added and refluxed. When the reaction was completed, the mixture was extracted with CH₂CI₂ and water, the organic layer was dried over MgSO₄, concentrated, and the resulting compound was recrystallized using silicagel column to obtain 2.5 g (yield 78%) of the product.

Meanwhile, FD-MS values of compounds P1-1 to P7-4 of the present invention prepared according to the above synthesis examples are shown in Table 3 below.

TABLE 3

| | | | |
|---|---|---|---|
| compound | FD-MS | compound | FD-MS |
| P1-1 | m/z=693.30(C₅₂H₃₉NO=693.89) | P1-2 | m/z=741.30(C₅₆H₃₉NO=741.93) |
| P1-3 | m/z=739.29(C₅₆H₃₇NO=739.92) | P1-4 | m/z=693.30(C₅₂H₃₉NO=693.89) |
| P1-5 | m/z=693.30(C₅₂H₃₉NO=693.89) | P1-6 | m/z=693.30(C₅₂H₃₉NO=693.89) |
| P1-7 | m/z=759.29(C₅₆H₃₈FNO=759.92) | P1-8 | m/z=817.33(C₆₂H₄₃NO=818.03) |
| P1-9 | m/z=766.30(C₅₇H₃₈N₂O=766.94) | P1-10 | m/z=744.32(C₅₆H₃₂D₅NO= 744.95) |
| P1-11 | m/z=816.31 (C₆₁H₄₀N₂O=817.00) | P1-12 | m/z=816.31 (C₆₁H₄₀N₂O=817.00) |
| P1-13 | m/z=693.30(C₅₂H₃₉NO=693.89) | P1-14 | m/z=769.33(C₅₈H₄₃NO=769.99) |
| P1-15 | m/z=711.29(C₅₂H₃₈FNO=711.88) | P1-16 | m/z=841.39(C₆₂H₅₁NO₂=842.10) |
| P1-17 | m/z=709.28(C₅₂H₃₉NS=709.95) | P1-18 | m/z=757.28(C₅₆H₃₉NS=758.00) |
| P1-19 | m/z=831.30(C₆₂H₄₁ NS=832.08) | P1-20 | m/z=709.28(C₅₂H₃₉NS=709.95) |
| P1-21 | m/z=638.28(C₄₆H₃₀D₅NS=638.88) | P1-22 | m/z=775.27(C₅₆H₃₈FNS=775.99) |
| P1-23 | m/z=755.26(C₅₆H₃₇NS=755.98) | P1-24 | m/z=805.28(C₆₀H₃₉NS=806.04) |
| P1-25 | m/z=709.28(C₅₂H₃₉NS=709.95) | P1-26 | m/z=727.27(C₅₂H₃₈FNS=727.94) |
| P1-27 | m/z=709.28(C₅₂H₃₉NS=709.95) | P1-28 | m/z=785.31(C₅₈H₄₃NS=786.05) |
| P1-29 | m/z=693.30(C₅₂H₃₉NO=693.89) | P1-30 | m/z=829.37(C₆₁H₅₁NS=830.15) |
| P1-31 | m/z=717.30(C₅₄H₃₉NO=717.91) | P1-32 | m/z=805.28(C₆₀H₃₉NS=806.04) |
| P1-33 | m/z=631.29(C₄₇H₃₇NO=631.82) | P1-34 | m/z=695.26(C₅₁H₃₇NS=695.92) |
| P2-1 | m/z=693.30(C₅₂H₃₉NO=693.89) | P2-2 | m/z=741.30(C₅₆H₃₉NO=741.93) |
| P2-3 | m/z=815.32(C₆₂H₄₁ NO=816.2) | P2-4 | m/z=693.30(C₅₂H₃₉NO=693.89) |
| P2-5 | m/z=635.26(C₄₆H₃₄FNO=635.78) | P2-6 | m/z=667.29(C₅₀H₃₇NO=667.85) |
| P2-7 | m/z=817.33(C₆₂H₄₃NO=818.03) | P2-8 | m/z=741.30(C₅₆H₃₉NO=741.93) |
| P2-9 | m/z=766.30(C₅₇H₃₈N₂O=766.94) | P2-10 | m/z=789.30(C₆₀H₃₉NO=789.98) |
| P2-11 | m/z=815.32(C₆₂H₄₁ NO=816.02) | P2-12 | m/z=831.31(C₆₂H₄₁NO₂=832.02) |
| P2-13 | m/z=698.33(C₅₂H₃₄D₅NO=698.92) | P2-14 | m/z=769.33(C₅₈H₄₃NO=769.99) |
| P2-15 | m/z=743.32(C₅₆H₄₁ NO=743.95) | P2-16 | m/z=769.33(C₅₈H₄₃NO=769.99) |
| P2-17 | m/z=633.25(C₄₆H₃₅NS=633.85) | P2-18 | m/z=833.31 (C₆₂H₄₃NS=834.09) |
| P2-19 | m/z=805.28(C₆₀H₃₉NS=806.04) | P2-20 | m/z=709.28(C₅₂H₃₉NS=709.95) |
| P2-21 | m/z=683.26(C₅₀H₃₇NS=683.91) | P2-22 | m/z=833.31 (C₆₂H₄₃NS=834.09) |
| P2-23 | m/z=823.27 (C₆₀H₃₈FNS=824.03) | P2-24 | m/z=780.26(C₅₇H₃₆N₂S=780.99) |
| P2-25 | m/z=709.28(C₅₂H₃₉NS=709.95) | P2-26 | m/z=709.28(C₅₂H₃₉NS=709.95) |
| P2-27 | m/z=709.28(C₅₂H₃₉NS=709.95) | P2-28 | m/z=785.31 (C₅₈H₄₃NS=786.05) |
| P2-29 | m/z=753.30(C₅₇H₃₉NO=753.95) | P2-30 | m/z=647.88(C₄₇H₃₇NS=647.88) |
| P3-1 | m/z=693.30(C₅₂H₃₉NO=693.89) | P3-2 | m/z=791.32(C₆₀H₄₁ NO=791.99) |
| P3-3 | m/z=815.32(C₆₂H₄₁ NO=816.02) | P3-4 | m/z=617.27(C₄₆H₃₅NO=617.79) |
| P3-5 | m/z=667.29(C₅₀H₃₇NO=667.85) | P3-6 | m/z=667.29(C₅₀H₃₇NO=667.85) |
| P3-7 | m/z=759.29(C₅₆H₃₈FNO=759.92) | P3-8 | m/z=817.33(C₆₂H₄₃NO=818.03) |
| P3-9 | m/z=741.30(C₅₆H₃₉NO=741.93) | P3-10 | m/z=744.32(C₅₆H₃₂D₅NO= 744.95) |
| P3-11 | m/z=789.30(C₆₀H₃₉NO=789.98) | P3-12 | m/z=739.29(C₅₆H₃₇NO=739.92) |
| P3-13 | m/z=693.30(C₅₂H₃₉NO=693.89) | P3-14 | m/z=793.33(C₆₀H₄₃NO=794.01) |
| P3-15 | m/z=743.32(C₅₆H₄₁ NO=743.95) | P3-16 | m/z=769.33(C₅₈H₄₃NO=769.99) |
| P3-17 | m/z=709.28(C₅₂H₃₉NS=709.95) | P3-18 | m/z=833.31 (C₆₂H₄₃NS=834.09) |
| P3-19 | m/z=831.30(C₆₂H₄₁ NS=832.08) | P3-20 | m/z=651.24(C₄₆H₃₄FNS=651.84) |
| P3-21 | m/z=633.25(C₄₆H₃₅NS=633.85) | P3-22 | m/z=757.28(C₅₆H₃₉NS=758.00) |
| P3-23 | m/z=832.29(C₆₁ H₄₀N₂S=833.07) | P3-24 | m/z=831.30(C₆₂H₄₁NS=832.08) |
| P3-25 | m/z=709.28(C₅₂H₃₉NS=709.95) | P3-26 | m/z=709.28(C₅₂H₃₉NS=709.95) |
| P3-27 | m/z=759.30(C₅₆H₄₁ NS=760.01) | P3-28 | m/z=835.33(C₆₂H₄₅NS=836.11) |
| P3-29 | m/z=647.26(C₄₇H₃₇NS=647.88) | P3-30 | m/z=631.29(C₄₇H₃₇NO=631.82) |
| P4-1 | m/z=723.26(C₅₂H₃₇NOS=723.93) | P4-2 | m/z=707.28(C₅₂H₃₇NO₂=707.87) |
| P4-3 | m/z=857.37(C₆₅H₄₇NO=858.10) | P4-4 | m/z=829.30(C₆₂H₃₉NO₂=830.00) |
| P4-5 | m/z=782.33(C₅₈H₄₂N₂O=782.99) | P4-6 | m/z=855.35(C₆₅H₄₅NO=856.08) |
| P4-7 | m/z=881.33(C₆₆H₄₃NO₂=882.08) | P4-8 | m/z=913.28(C₆₆H₄₀FNOS= 914.11) |
| P4-9 | m/z=739.24(C₅₂H₃₇NS₂=740.00) | P4-10 | m/z=798.31 (C₅₈H₄₂N₂S=799.05) |
| P4-11 | m/z=851.32(C₆₂H₃₇D₄NOS= 852.10) | P4-12 | m/z=861.25(C₆₂H₃₉NS₂=862.12) |
| P4-13 | m/z=749.31 (C₅₅H₄₃NS=750.02) | P4-14 | m/z=848.32(C₆₂H₄₄N₂S=849.11) |
| P4-15 | m/z=940.29(C₆₇H₄₄N₂S₂=941.22) | P4-16 | m/z=921.31 (C₆₈H₄₃NOS=922.16) |
| P4-17 | m/z=997.34(C₇₄H₄₇NOS=998.26) | P4-18 | m/z=923.32(C₆₈H₄₅NOS=924.17) |
| P4-19 | m/z=982.39(C₇₄H₅₀N₂O=983.23 | P4-20 | m/z=885.40(C₆₇H₅₁NO=886.15) |
| P5-1 | m/z=707.28(C₅₂H₃₇NO₂=707.87) | P5-2 | m/z=723.26(C₅₂H₃₇NOS= 723.93) |
| P5-3 | m/z=782.33(C₅₈H₄₂N₂O=782.99) | P5-4 | m/z=733.33(C₅₅H₄₃NO=733.96) |
| P5-5 | m/z=707.28(C₅₂H₃₇NO₂=707.87) | P5-6 | m/z=707.28(C₅₂H₃₇NO₂=707.87) |
| P5-7 | m/z=707.28(C₅₂H₃₇NO₂=707.87) | P5-8 | m/z=723.26(C₅₂H₃₇NOS= 723.93) |
| P5-9 | m/z=723.26(C₅₂H₃₇NOS=723.93) | P5-10 | m/z=782.33(C₅₈H₄₂N₂O=782.99) |
| P5-11 | m/z=782.33(C₅₈H₄₂N₂O=782.99) | P5-12 | m/z=733.33(C₅₅H₄₃NO=733.96) |
| P5-13 | m/z=733.33(C₅₅H₄₃NO=733.96) | P5-14 | m/z=857.37(C₆₅H₄₇NO=858.10) |
| P5-15 | m/z=855.35(C₆₅H₄₅NO=856.08) | P5-16 | m/z=831.31(C₆₂H₄₁NO₂=832.02) |
| P5-17 | m/z=829.30(C₆₂H₃₉NO₂=830.00) | P5-18 | m/z=723.26(C₅₂H₃₇NOS= 723.93) |
| P5-19 | m/z=733.33(C₅₅H₄₃NO=733.96) | P5-20 | m/z=782.33(C₅₈H₄₂N₂O=782.99) |
| P5-21 | m/z=831.31 (C₆₂H₄₁NO₂=832.02) | P5-22 | m/z=906.36(C₆₈H₄₆N₂O=907.13) |
| P5-23 | m/z=881.33(C₆₆H₄₃NO₂=882.08) | P5-24 | m/z=855.35(C₆₅H₄₅NO=856.08) |
| P5-25 | m/z=723.26(C₅₂H₃₇NOS=723.93) | P5-26 | m/z=848.32(C₆₂H₄₄N₂S=849.11) |
| P5-27 | m/z=939.30(C₆₈H₄₅NS₂=940.24) | P5-28 | m/z=845.28(C₆₂H₃₉NOS= 846.06) |
| P5-29 | m/z=799.29(C₅₈H₄₁ NOS=800.03) | P5-30 | m/z=825.34(C₆₁H₄₇NS=826.11) |
| P5-31 | m/z=913.28(C₆₆H₄₃NS₂=914.20) | P5-32 | m/z=921.34(C₆₉H₄₇NS=922.20) |
| P5-33 | m/z=923.32(C₆₈H₄₂FNO₂=924.09) | P5-34 | m/z=1025.41 (C₇₇H₅₅NS= 1026.35) |
| P5-35 | m/z=967.33(C₇₀H₄₉NS₂=968.29) | P5-36 | m/z=885.40(C₆₇H₅₁NO=886.15) |
| P5-37 | m/z=763.33(C₅₆H₄₅NS=764.04) | P5-38 | m/z=819.31 (C₆₁H₄₁NO₂=820.00) |
| P6-1 | m/z=723.26(C₅₂H₃₇NOS=723.93) | P6-2 | m/z=707.28(C₅₂H₃₇NO₂=707.87) |
| P6-3 | m/z=847.29(C₆₂H₄₁NOS=848.08) | P6-4 | m/z=879.31 (C₆₆H₄₁NO₂=880.06) |
| P6-5 | m/z=832.35(C₆₂H₄₄N₂O=833.05) | P6-6 | m/z=733.33(C₅₅H₄₃NO=733.96) |
| P6-7 | m/z=981.40(C₇₅H₅₁NO=982.24) | P6-8 | m/z=829.30(C₆₂H₃₉NO₂=830.00) |
| P6-9 | m/z=799.29(C₅₈H₄₁NOS=800.03) | P6-10 | m/z=848.32(C₆₂H₄₄N₂S=849.11) |
| P6-11 | m/z=881.26(C₆₂H₄₀FNS₂=882.13) | P6-12 | m/z=861.25(C₆₂H₃₉NS₂=862.12) |
| P6-13 | m/z=871.33(C₆₅H₄₅NS=872.14) | P6-14 | m/z=799.29(C₅₈H₄₁NOS=800.03) |
| P6-15 | m/z=863.27(C₆₂H₄₁NS₂=864.14) | P6-16 | m/z=871.33(C₆₅H₄₅NS=872.14) |
| P6-17 | m/z=1122.4(C₈₄H₅₄N₂S=1123.43) | P6-18 | m/z=875.32(C₆₄H₄₅NOS= 876.13) |
| P6-19 | m/z=1085.46(C₈₃H₅₉NO=1086.39) | P6-20 | m/z=875.32(C₆₄H₄₅NOS= 876.13) |
| P6-21 | m/z=721.30(C₅₃H₃₉NO₂=721.90) | P6-22 | m/z=753.25(C₅₃H₃₉NS₂=754.02) |
| P7-1 | m/z=1148.47(C₈₇H₆₀N₂O= 1149.45) | P7-2 | m/z=1162.43(C₈₇H₅₈N₂S= 1163.50) |
| P7-3 | m/z=1330.49(C₉₉H₆₆N₂OS= 1331.69) | P7-4 | m/z=1148.47(C₈₇H₆₀N₂O= 1149.45) |

### Evaluation of Manufacture of Organic Electronic Element

### [Example 1] Green Organic Light Emitting Diode (Hole Transport Layer)

An organic electroluminescent device was manufactured according to a conventional method using the compound of the present invention as a hole transport layer material.

First, on an ITO layer (anode) formed on a glass substrate, N1-(naphthalen-2-yl)-N4, N4-bis(4-(naphthalen-2-yl(phenyl)amino)phenyl)-N 1-phenylbenzene-1,4-diamine (Hereinafter, abbreviated as 2-TNATA) film as a hole injection layer was vacuum-deposited to form a thickness of 60 nm. On the hole injection layer, the compound P1-1 of the present invention was vacuum-deposited to a thickness of 60 nm to form a hole transport layer. After forming the hole transport layer, CBP[4,4′-N,N′-dicarbazole-biphenyl] was used as a host, and lr(ppy)₃ [tris(2-phenylpyridine)-iridium] was used as a dopant, doped at a weight ratio of 95:5, and vacuum deposited to a thickness of 30 nm to form an emitting layer on hole transport layer. Then (1,1′-bisphenyl)-4-oleato)bis(2-methyl-8-quinolineoleato)aluminum (hereinafter abbreviated as BAlq) as a hole blocking layer was vacuum-deposited to a thickness of 10 nm, as an electron transport layer, tris(8-quinolinol)aluminum (hereinafter, abbreviated as Alq3) was deposited to a thickness of 40 nm. Thereafter, LiF, which is an alkali metal halide, was deposited as an electron injection layer to a thickness of 0.2 nm, and then Al was deposited to a thickness of 150 nm and used as a cathode to prepare an organic electroluminescent device.

### [Example 2] to [Example 51] Green Organic Light Emitting Diode (Hole Transport Layer)

An organic electroluminescent device was manufactured in the same manner as in Example 1, except that the compound P1-2 to P7-3 of the present invention described in Table 4 was used instead of the compound P1-1 of the present invention as a hole transport layer material.

### [Comparative Example 1] or [Comparative Example 2]

An organic electroluminescent device was manufactured in the same manner as in Example 1, except for using Comparative Compound A or Comparative Compound B instead of Compound P1-1 of the present invention as the hole transport layer material

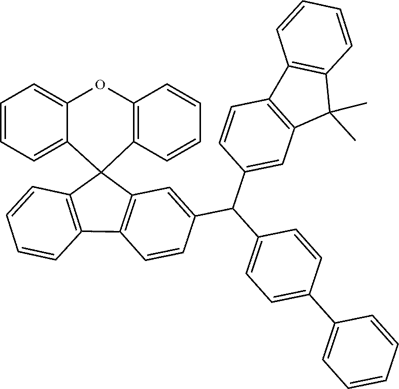 [SMILES: CC1(C)c2ccccc2-c2ccc(C(c3ccc(-c4ccccc4)cc3)c3ccc4c(c3)C3(c5ccccc5Oc5ccccc53)c3ccccc3-4)cc21]

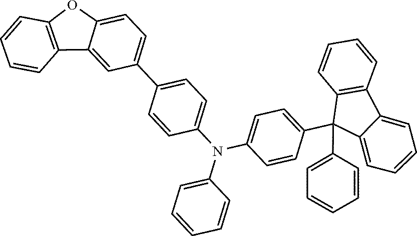 [SMILES: c1ccc(N(c2ccc(-c3ccc4oc5ccccc5c4c3)cc2)c2ccc(C3(c4ccccc4)c4ccccc4-c4ccccc43)cc2)cc1]

By applying a forward bias DC voltage to the organic electroluminescent devices prepared in Examples and Comparative Examples prepared in this way, Electroluminescence (EL) characteristics were measured with PR-650 from Photoresearch, and as a result of the measurement, the T95 lifetime was measured using a lifetime measuring device manufactured by McScience at 5000 cd/m² standard luminance. Table 4 below shows the device fabrication and evaluation results.

TABLE 4

| | compound | Voltage | Current Density (mA/cm ²) | Brightn ess (cd/m²) | Efficie ncy (cd/A) | T(95) | CIE | |
|---|---|---|---|---|---|---|---|---|
| | | | | | | | X | y |
| comparative example(1) | comparativ e compound | 5.3 | 17.6 | 5000 | 28.4 | 84.6 | 0.35 | 0.61 |
| | A | | | | | | | |
| comparative example(2) | comparativ e compound B | 5.5 | 18.2 | 5000 | 27.4 | 88.9 | 0.35 | 0.63 |
| example(1) | compound (P1-1) | 4.7 | 13.3 | 5000 | 37.6 | 107.0 | 0.34 | 0.61 |
| example(2) | compound (P1-4) | 4.7 | 13.7 | 5000 | 36.6 | 105.0 | 0.32 | 0.64 |
| example(3) | compound (P1-5) | 4.9 | 15.0 | 5000 | 33.2 | 99.2 | 0.33 | 0.60 |
| example(4) | compound (P1-13) | 4.7 | 13.6 | 5000 | 36.8 | 104.9 | 0.31 | 0.65 |
| example(5) | compound (P1-14) | 4.8 | 14.2 | 5000 | 35.2 | 107.2 | 0.31 | 0.61 |
| example(6) | compound (P1-17) | 4.8 | 14.1 | 5000 | 35.5 | 102.0 | 0.34 | 0.62 |
| example(7) | compound (P1-18) | 4.9 | 14.4 | 5000 | 34.8 | 104.0 | 0.31 | 0.64 |
| example(8) | compound (P1-24) | 5.2 | 16.6 | 5000 | 30.1 | 96.2 | 0.32 | 0.65 |
| example(9) | compound (P1-33) | 4.6 | 13.9 | 5000 | 36.1 | 111.3 | 0.32 | 0.63 |
| example(10) | compound (P2-1 ) | 4.6 | 12.8 | 5000 | 39.1 | 111.2 | 0.34 | 0.64 |
| example(11) | compound (P2-2) | 4.7 | 13.1 | 5000 | 38.3 | 113.8 | 0.34 | 0.61 |
| example(12) | compound (P2-3) | 4.6 | 13.3 | 5000 | 37.7 | 115.7 | 0.34 | 0.61 |
| example(13) | compound (P2-7) | 4.9 | 14.4 | 5000 | 34.7 | 105.2 | 0.31 | 0.63 |
| example(14) | compound (P2-8) | 4.9 | 14.3 | 5000 | 34.9 | 106.3 | 0.31 | 0.63 |
| example(15) | compound (P2-11 | 4.8 | 14.6 | 5000 | 34.2 | 108.3 | 0.31 | 0.62 |
| example(16) | compound (P2-17) | 4.7 | 13.2 | 5000 | 37.9 | 108.2 | 0.34 | 0.65 |
| example(17) | compound (P2-18) | 4.8 | 13.4 | 5000 | 37.2 | 110.4 | 0.32 | 0.64 |
| example(18) | compound (P2-20) | 4.7 | 13.5 | 5000 | 36.9 | 106.0 | 0.34 | 0.62 |
| example(19) | compound (P2-21) | 4.9 | 15.1 | 5000 | 33.1 | 98.2 | 0.31 | 0.62 |
| example(20) | compound (P2-22) | 5.0 | 14.7 | 5000 | 33.9 | 103.2 | 0.31 | 0.60 |
| example(21) | compound (P2-29) | 4.5 | 13.8 | 5000 | 36.3 | 120.3 | 0.31 | 0.64 |
| example(22) | compound (P3-1) | 4.7 | 12.6 | 5000 | 39.8 | 113.4 | 0.35 | 0.63 |
| example(23) | compound (P3-3) | 4.7 | 13.1 | 5000 | 38.2 | 117.9 | 0.34 | 0.62 |
| example(24) | compound (P3-4) | 4.7 | 12.9 | 5000 | 38.8 | 111.3 | 0.30 | 0.63 |
| example(25) | compound (P3-12) | 5.0 | 14.5 | 5000 | 34.5 | 107.2 | 0.34 | 0.61 |
| example(26) | compound (P3-17) | 4.7 | 13.0 | 5000 | 38.6 | 110.1 | 0.32 | 0.61 |
| example(27) | compound (P3-25) | 4.8 | 13.2 | 5000 | 38.0 | 108.0 | 0.32 | 0.61 |
| example(28) | compound (P3-26) | 4.9 | 14.0 | 5000 | 35.6 | 103.9 | 0.34 | 0.64 |
| example(29) | compound (P3-29) | 4.7 | 13.7 | 5000 | 36.4 | 112.2 | 0.32 | 0.61 |
| example(30) | compound (P4-2) | 4.6 | 13.2 | 5000 | 37.8 | 108.6 | 0.30 | 0.63 |
| example(31) | compound (P4-11) | 4.7 | 13.4 | 5000 | 37.4 | 111.0 | 0.32 | 0.60 |
| example(32) | compound (P4-13) | 4.7 | 13.8 | 5000 | 36.2 | 101.0 | 0.31 | 0.61 |
| example(33) | compound (P5-1) | 4.5 | 12.6 | 5000 | 39.6 | 113.0 | 0.34 | 0.62 |
| example(34) | compound (P5-2) | 4.6 | 12.9 | 5000 | 38.7 | 110.8 | 0.32 | 0.60 |
| example(35) | compound (P5-3) | 4.6 | 12.7 | 5000 | 39.3 | 111.5 | 0.30 | 0.60 |
| example(36) | compound (P5-4) | 4.5 | 12.5 | 5000 | 39.9 | 110.2 | 0.33 | 0.62 |
| example(37) | compound (P5-5) | 4.5 | 12.7 | 5000 | 39.5 | 112.9 | 0.32 | 0.64 |
| example(38) | compound (P5-6) | 4.5 | 12.5 | 5000 | 40.1 | 113.5 | 0.31 | 0.65 |
| example(39) | compound (P5-7) | 4.5 | 12.7 | 5000 | 39.4 | 112.3 | 0.35 | 0.61 |
| example(40) | compound (P5-8) | 4.6 | 13.0 | 5000 | 38.5 | 110.7 | 0.31 | 0.61 |
| example(41) | compound (P5-16) | 4.6 | 12.9 | 5000 | 38.9 | 115.2 | 0.31 | 0.62 |
| example(42) | compound (P5-17) | 4.6 | 13.1 | 5000 | 38.1 | 117.4 | 0.31 | 0.63 |
| example(43) | compound (P5-25) | 4.6 | 13.0 | 5000 | 38.4 | 109.6 | 0.35 | 0.64 |
| example(44) | compound (P5-28) | 4.7 | 13.6 | 5000 | 36.8 | 114.0 | 0.33 | 0.64 |
| example(45) | compound (P5-31) | 5.1 | 15.8 | 5000 | 31.7 | 100.6 | 0.32 | 0.60 |
| example(46) | compound (P5-38) | 4.8 | 15.5 | 5000 | 32.3 | 109.9 | 0.33 | 0.60 |
| example(47) | compound (P6-1) | 4.7 | 12.7 | 5000 | 39.2 | 112.8 | 0.33 | 0.62 |
| example(48) | compound (P6-12) | 4.8 | 13.5 | 5000 | 37.0 | 113.9 | 0.34 | 0.64 |
| example(49) | compound (P6-16) | 4.9 | 14.5 | 5000 | 34.6 | 106.2 | 0.30 | 0.64 |
| example(50) | compound (P6-21) | 4.6 | 13.5 | 5000 | 37.1 | 122.0 | 0.31 | 0.64 |
| example(51) | compound (P7-3) | 4.7 | 15.2 | 5000 | 32.8 | 97.7 | 0.30 | 0.62 |

As can be seen from the results in Table 4, when a green organic electric device is manufactured using the material for an organic electric device of the present invention as a hole transport layer material, the performance of the organic electroluminescent device can be improved compared to the comparative example using the comparative compound A or the comparative compound B.

In other words, if Comparative Example 1 using Comparative Compound A is compared with Comparative Example 2 using Comparative Compound B, the results of Comparative Example 1 showed excellent results in terms of driving voltage, and Comparative Example 2 showed excellent results in terms of efficiency and lifespan. Also, Examples 1 to 51 of the compound of the present invention showed remarkably excellent results in efficiency and lifetime.

### [Example 52] Green Organic Light Emitting Diode (Emitting Auxiliary Layer)

An organic electroluminescent device was manufactured according to a conventional method using the compound of the present invention as an emitting auxiliary layer material.

First, on an ITO layer (anode) formed on a glass substrate, N1-(naphthalen-2-yl)-N4, N4-bis(4-(naphthalen-2-yl(phenyl)amino)phenyl)-N 1-phenylbenzene-1,4-diamine (Hereinafter, abbreviated as 2-TNATA) film as a hole injection layer was vacuum-deposited to form a thickness of 60 nm. 4,4-bis[N-(1-naphthyl)-N-phenylamino]biphenyl (hereinafter abbreviated as NPB) was vacuum-deposited on the hole injection layer to a thickness of 60 nm to form a hole transport layer. Then, as a material for the emitting auxiliary layer, the compound P1-1 of the present invention was vacuum-deposited to a thickness of 20 nm to form an emitting auxiliary layer. After forming the emitting auxiliary layer, CBP[4,4′-N,N′-dicarbazole-biphenyl] was used as a host, and lr(ppy)₃ [tris(2-phenylpyridine)-iridium] was used as a dopant, doped at a weight ratio of 95:5, and vacuum deposited to a thickness of 30 nm to form an emitting layer on the emitting auxiliary layer. (1,1′-bisphenyl)-4-oleato)bis(2-methyl-8-quinolineoleato)aluminum (hereinafter abbreviated as BAlq) as a hole blocking layer was vacuum-deposited to a thickness of 10 nm, as an electron transport layer, tris(8-quinolinol)aluminum (hereinafter, abbreviated as Alq3) was deposited to a thickness of 40 nm. Thereafter, LiF, which is an alkali metal halide, was deposited as an electron injection layer to a thickness of 0.2 nm, and then Al was deposited to a thickness of 150 nm and used as a cathode to prepare an organic electroluminescent device.

### [Example 53] to [Example 102] Green Organic Light Emitting Diode (Emitting Auxiliary Layer)

An organic electroluminescent device was manufactured in the same manner as in Example 47, except that the compound P1-2 to P7-3 of the present invention described in Table 5 was used instead of the compound P1-1 of the present invention as an emitting auxiliary layer material.

### [Comparative Example 3]

An organic electroluminescent device was manufactured in the same manner as in Example 47, except that the emitting auxiliary layer was not used.

### [Comparative Example 4] or [Comparative Example 5]

An organic electroluminescent device was manufactured in the same manner as in Example 47, Except for using Comparative Compound A or Comparative Compound B instead of Compound P1-1 of the present invention as the emitting auxiliary layer material

By applying a forward bias DC voltage to the organic electroluminescent devices prepared in Examples and Comparative Examples prepared in this way, Electroluminescence (EL) characteristics were measured with PR-650 from Photoresearch, and as a result of the measurement, the T95 lifetime was measured using a lifetime measuring device manufactured by McScience at 5000 cd/m² standard luminance. Table 4 below shows the device fabrication and evaluation results.

TABLE 5

| | compound | Voltage | Current Density (mA/cm 2) | Bright ness (cd/m² ) | Effici ency (cd/A ) | T(95) | CIE | |
|---|---|---|---|---|---|---|---|---|
| | | | | | | | X | y |
| comparative example(1) | | 6.7 | 68.5 | 5000 | 7.3 | 62.9 | 0.31 | 0.63 |
| comparative example(2) | comparativ e compound A | 5.4 | 14.4 | 5000 | 34.6 | 97.9 | 0.30 | 0.63 |
| comparative example(3) | comparativ e compound B | 5.6 | 15.0 | 5000 | 33.4 | 102.9 | 0.30 | 0.61 |
| example(52) | compound (P1-1) | 4.9 | 10.9 | 5000 | 45.8 | 123.8 | 0.31 | 0.64 |
| example(53) | compound (P1-4) | 5.0 | 11.2 | 5000 | 44.6 | 121.5 | 0.31 | 0.63 |
| example(54) | compound (P1-5) | 5.2 | 12.3 | 5000 | 40.5 | 114.8 | 0.32 | 0.63 |
| example(55) | compound (P1-13) | 5.0 | 11.2 | 5000 | 44.8 | 121.4 | 0.31 | 0.64 |
| example(56) | compound (P1-14) | 5.1 | 11.7 | 5000 | 42.9 | 123.9 | 0.31 | 0.62 |
| example(57) | compound (P1-17) | 5.1 | 11.5 | 5000 | 43.3 | 118.0 | 0.33 | 0.65 |
| example(58) | compound (P1-18) | 5.2 | 11.8 | 5000 | 42.5 | 120.4 | 0.31 | 0.61 |
| example(59) | compound (P1-24) | 5.4 | 13.6 | 5000 | 36.7 | 111.4 | 0.32 | 0.64 |
| example(60) | compound (P1-33) | 4.8 | 11.4 | 5000 | 44.0 | 128.8 | 0.35 | 0.61 |
| example(61) | compound (P2-1) | 4.8 | 10.5 | 5000 | 47.6 | 128.7 | 0.31 | 0.61 |
| example(62) | compound (P2-2) | 5.0 | 10.7 | 5000 | 46.8 | 131.4 | 0.32 | 0.61 |
| example(63) | compound (P2-3) | 4.9 | 10.9 | 5000 | 45.9 | 133.9 | 0.35 | 0.63 |
| example(64) | compound (P2-7) | 5.2 | 11.8 | 5000 | 42.3 | 121.8 | 0.31 | 0.62 |
| example(65) | compound (P2-8) | 5.2 | 11.7 | 5000 | 42.6 | 123.0 | 0.30 | 0.63 |
| example(66) | compound (P2-11 | 5.1 | 12.0 | 5000 | 41.8 | 125.4 | 0.32 | 0.61 |
| example(67) | compound (P2-17) | 4.9 | 10.8 | 5000 | 46.2 | 125.0 | 0.32 | 0.63 |
| example(68) | compound (P2-18) | 5.1 | 11.0 | 5000 | 45.4 | 127.7 | 0.35 | 0.64 |
| example(69) | compound (P2-20) | 5.0 | 11.1 | 5000 | 45.0 | 122.7 | 0.34 | 0.63 |
| example(70) | compound (P2-21) | 5.2 | 12.5 | 5000 | 40.1 | 113.6 | 0.30 | 0.63 |
| example(71) | compound (P2-22) | 5.3 | 12.1 | 5000 | 41.3 | 119.4 | 0.34 | 0.61 |
| example(72) | compound (P2-29) | 4.8 | 11.4 | 5000 | 43.9 | 139.3 | 0.33 | 0.64 |
| example(73) | compound (P3-1 ) | 4.9 | 10.3 | 5000 | 48.6 | 131.2 | 0.31 | 0.61 |
| example(74) | compound (P3-3) | 5.0 | 10.7 | 5000 | 46.7 | 136.5 | 0.32 | 0.64 |
| example(75) | compound (P3-4) | 5.0 | 10.6 | 5000 | 47.3 | 128.8 | 0.31 | 0.61 |
| example(76) | compound (P3-12) | 5.3 | 11.9 | 5000 | 42.1 | 124.1 | 0.34 | 0.64 |
| example(77) | compound (P3-17) | 5.0 | 10.6 | 5000 | 47.1 | 127.4 | 0.32 | 0.62 |
| example(78) | compound (P3-25) | 5.1 | 10.8 | 5000 | 46.3 | 124.9 | 0.31 | 0.63 |
| example(79) | compound (P3-26) | 5.2 | 11.5 | 5000 | 43.4 | 120.3 | 0.34 | 0.62 |
| example(80) | compound (P3-29) | 5.0 | 11.3 | 5000 | 44.4 | 129.9 | 0.32 | 0.65 |
| example(81) | compound (P4-2) | 4.9 | 10.8 | 5000 | 46.1 | 125.6 | 0.32 | 0.61 |
| example(82) | compound (P4-11) | 5.0 | 11.0 | 5000 | 45.6 | 128.3 | 0.33 | 0.64 |
| example(83) | compound (P4-13) | 5.0 | 11.3 | 5000 | 44.2 | 116.8 | 0.34 | 0.61 |
| example(84) | compound (P5-1) | 4.7 | 10.3 | 5000 | 48.4 | 130.8 | 0.30 | 0.61 |
| example(85) | compound (P5-2) | 4.8 | 10.5 | 5000 | 47.4 | 128.2 | 0.34 | 0.63 |
| example(86) | compound (P5-3) | 4.8 | 10.5 | 5000 | 47.7 | 129.0 | 0.30 | 0.63 |
| example(87) | compound (P5-4) | 4.7 | 10.3 | 5000 | 48.7 | 127.5 | 0.35 | 0.62 |
| example(88) | compound (P5-5) | 4.8 | 10.4 | 5000 | 48.1 | 130.7 | 0.31 | 0.62 |
| example(89) | compound (P5-6) | 4.8 | 10.2 | 5000 | 48.9 | 131.3 | 0.33 | 0.61 |
| example(90) | compound (P5-7) | 4.8 | 10.4 | 5000 | 47.9 | 130.0 | 0.35 | 0.63 |
| example(91) | compound (P5-8) | 4.8 | 10.6 | 5000 | 47.2 | 128.1 | 0.35 | 0.64 |
| example(92) | compound (P5-16) | 4.9 | 10.5 | 5000 | 47.5 | 133.3 | 0.34 | 0.61 |
| example(93) | compound (P5-17) | 4.8 | 10.8 | 5000 | 46.5 | 135.9 | 0.31 | 0.62 |
| example(94) | compound (P5-25) | 4.8 | 10.7 | 5000 | 46.9 | 126.9 | 0.32 | 0.63 |
| example(95) | compound (P5-28) | 4.9 | 11.1 | 5000 | 44.9 | 131.9 | 0.33 | 0.62 |
| example(96) | compound (P5-31) | 5.3 | 12.9 | 5000 | 38.9 | 116.4 | 0.35 | 0.62 |
| example(97) | compound (P5-38) | 5.0 | 12.7 | 5000 | 39.4 | 127.2 | 0.35 | 0.64 |
| example(98) | compound (P6-1) | 4.9 | 10.4 | 5000 | 48.2 | 130.6 | 0.35 | 0.61 |
| example(99) | compound (P6-12) | 5.1 | 11.1 | 5000 | 45.1 | 132.1 | 0.31 | 0.64 |
| example(100) | compound (P6-16) | 5.2 | 11.8 | 5000 | 42.2 | 122.9 | 0.32 | 0.60 |
| example(101) | compound (P6-21) | 4.9 | 11.0 | 5000 | 45.3 | 141.2 | 0.32 | 0.62 |
| example(102) | compound (P7-3) | 5.0 | 12.4 | 5000 | 40.2 | 113.1 | 0.34 | 0.60 |

As can be seen from the results in Table 5, when a green organic electric device is manufactured using the material for an organic electric device of the present invention as an emitting auxiliary layer material, the efficiency and lifespan of the organic electroluminescent device can be improved compared to the comparative example in which the emitting auxiliary layer is not used or the comparative compound A or the comparative compound B is used.

In other words, the results of Comparative Example 4 or Comparative Example 5 using Comparative Compound A or Comparative Compound B were superior to Comparative Example 3 without using the emitting auxiliary layer, and Examples 52 to 102 of the compound of the present invention showed remarkably excellent results in terms of efficiency and lifespan.

First, if the comparative compound A and the compound of the present invention are compared, there is a difference between the comparative compound A and the compound of the present invention in that the bonding position of the amino group to the fluorene is different. When fluorene is bonded to an amino group, the energy level (especially HOMO and LUMO) is changed depending on the bonding position, and the physical properties of the compound are changed. Accordingly, as the physical properties of the compound are changed, the charge balance in the emitting layer is increased, and light emission is performed well inside the emitting layer rather than the interface of the emitting layer. As a result, degradation at the interface of the emitting layer is also reduced, and efficiency and lifespan are improved. Therefore, it is determined that this point acts as a major factor in improving device performance during device deposition.

Second, comparing the comparative compound B and the compound of the present invention, Comparative Compound B has a structure in which a dibenzofuran structure is bonded to an amino group, and the compound of the present invention has a structure in which xanthene and thioxanthene structures are bonded to an amino group. In other words, when comparing dibenzofuran and xanthene, the xanthene structure is judged to have improved electron donating ability and improved hole properties as sp3 carbon is added. As the xanthene structure was introduced, the hole characteristics were improved and the driving voltage, efficiency, and lifespan were improved.

As a result, when the diphenylfluorene and xanthene structures are simultaneously substituted with amino groups as in the compound of the present invention, it is judged that the device performance is remarkably excellent due to the synergy between diphenylfluorene and xanthene.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### Industrial Applicability

According to the present invention, it is possible to manufacture an organic device having excellent device characteristics of high luminance, high light emission and long lifespan, and thus there is industrial applicability.

1. A compound represented by Formula 1: 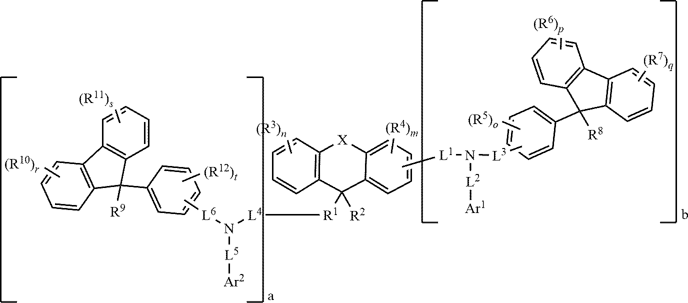
wherein:
1) X is O or S,
2) a and b are each independently 0 or 1, provided that a+b is 1 or more,
3) Ar¹ and Ar² are each independently selected from the group consisting of a C₆-C₆₀ aryl group; a fluorenyl group; a C₂-C₆₀ heterocyclic group including at least one heteroatom of O, N, S, Si or P; and a fused ring group of a C₃-C₆₀ aliphatic ring and a C₆-C₆₀ aromatic ring,
4) L¹ L², L³, L⁴, L⁵ and L⁶ are each independently selected from the group consisting of a single bond; an C₆-C₆₀ arylene group; a fluorenylene group; a C₂-C₆₀ heterocyclic group including at least one heteroatom of O, N, S, Si or P; and a fused ring group of a C₃-C₆₀ aliphatic ring and a C₆-C₆₀ aromatic ring,
5) i) where a is 0, R¹ is independently selected from the group consisting of hydrogen; deuterium; tritium; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heteroaryl group including at least one heteroatom of O, N, S, Si or P; a fused ring group of a C₃-C₆₀ aliphatic ring and a C₆-C₆₀ aromatic ring; or R¹ and R² are bonded to each other to form a ring,
ii) where a is 1, R¹ is Z; wherein Z is selected from the group consisting of a C₆-C₆₀ arylene group; a fluorenylene group; a C₂-C₆₀ heterocyclic group including at least one heteroatom of O, N, S, Si or P; a fused ring group of a C₃-C₆₀ aliphatic ring and a C₆-C₆₀ aromatic ring; or Z and R² may be bonded to each other to form a ring,
6) R² is independently selected from the group consisting of hydrogen; deuterium; tritium; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring,
7) R³, R⁴, R⁵, R⁶, R⁷, R⁸, R⁹, R¹⁰, R¹¹ and R¹² are independently selected from the group consisting of hydrogen; deuterium; tritium; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring, wherein in case m, n, o, p, q, r, s and t are 2 or more, R³, R⁴, R⁵, R⁶, R⁷, R¹⁰, R¹¹ and R¹² are each in plural being the same or different, and or a plurality of R³ or a plurality of R⁴ or a plurality of R⁵ or a plurality of R⁶ or a plurality of R⁷ or a plurality of R¹⁰ or a plurality of R¹¹ or a plurality of R¹² may be bonded to each other to form a ring,
8) R⁵, R⁶, R⁷, R⁸, R⁹, R¹⁰, R¹¹ and R¹² are may be bonded to each other to form a ring, with the proviso that R⁵ and R⁸, or R⁹ and R¹², are not bonded to each other to form a ring,
9) m, n, o, p, q, r, s and t are each independently an integer from 0 to 4,
10) wherein the aryl group, arylene group, heterocyclic group, fluorenyl group, fluorenylene group, fused ring group, alkyl group, alkenyl group, alkynyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group; and C₈-C₂₀ arylalkenyl group; also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

2. The compound of claim 1, wherein the compound represented by Formula 1 is represented by Formula 2 or Formula 3: 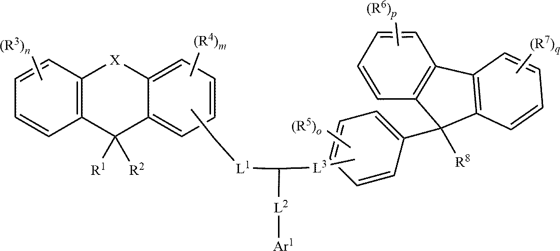 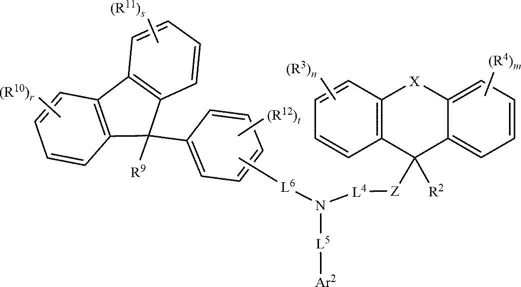
wherein X, R¹, R², R³, R⁴, R⁵, R⁶ R⁷ R⁸ R⁹ R¹⁰,R¹¹,R¹², L¹, L², L³, L⁴, L⁵, L⁶, Ar¹, Ar², Z, m, n, o, p, q, r, s and t are the same as defined in claim 1.

3. The compound of claim 1, wherein the compound represented by Formula 1 is represented by any one of Formulas 4 to 9: 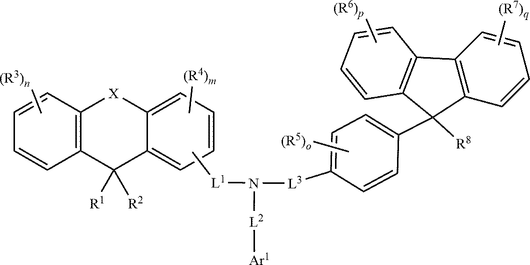 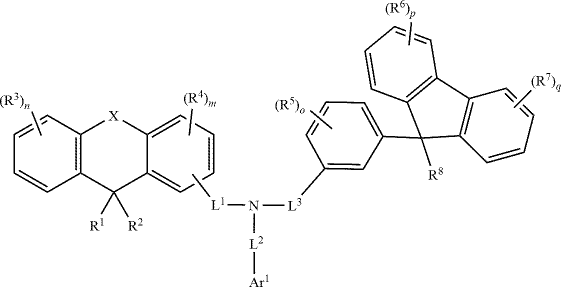 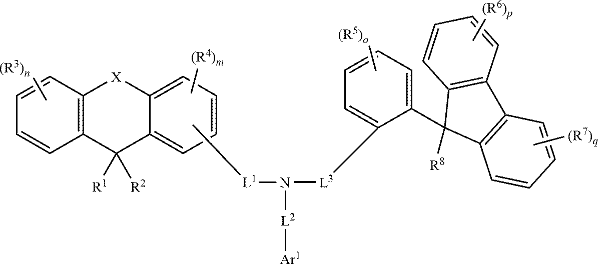 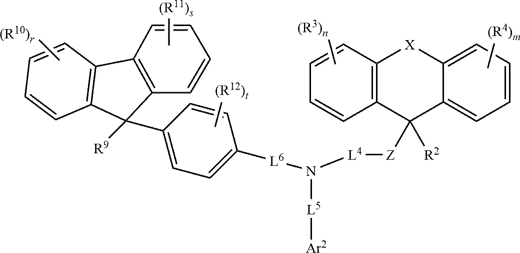 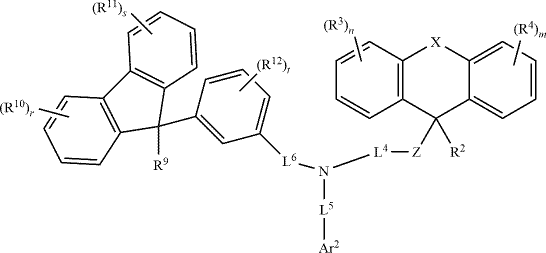 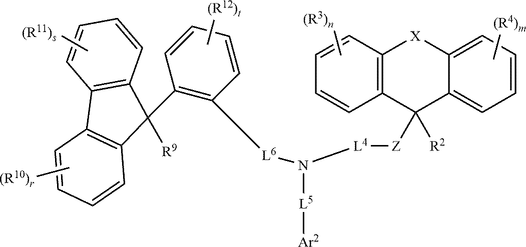
R¹, R², R³, R⁴, R⁵, R⁶, R⁷, R⁸, R⁹, R¹⁰,R¹¹, R¹², L¹, L², L³, L⁴, L⁵, L⁶, Ar¹, Ar², Z, m, n, o, p, q, r, s and t are the same as defined in claim 1.

4. The compound of claim 1, wherein the compound represented by Formula 1 is represented by any one of Formulas 10 to 13: 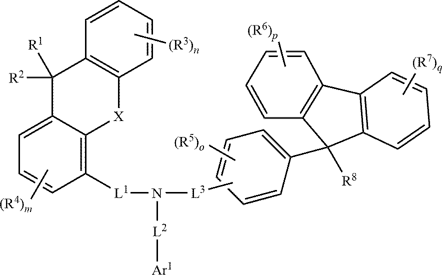 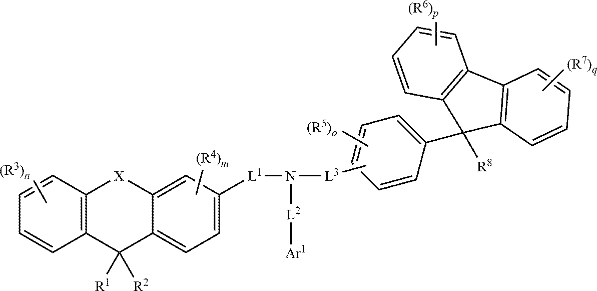 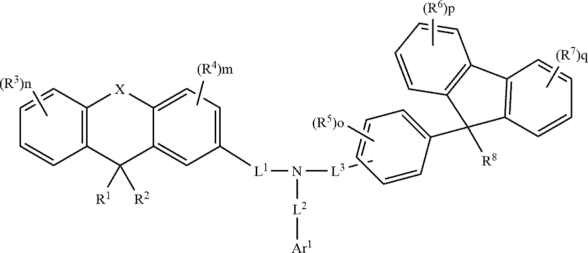 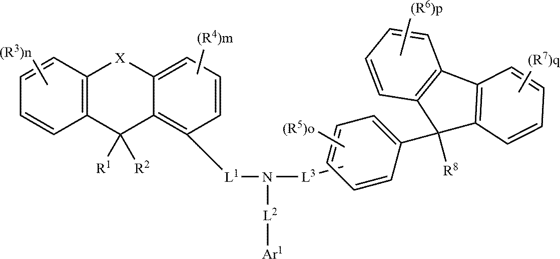
wherein X, R¹, R², R³, R⁴, R⁵, R⁶, R⁷, R⁸, L¹, L², L³, Ar¹, m, n, o, p, and q are the same as defined in claim 1.

5. The compound of claim 1, wherein Ar¹ or Ar² is represented by Formula 1-1: 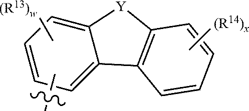
wherein:
1) Y is O, S, NR^{b} or CR’R″,
2) R¹³, R¹⁴, R^{b}, R′ and R″ are independently selected from the group consisting of hydrogen; deuterium; tritium; a C₁-C₆₀ alkyl group; a C₂-C₆₀ alkenyl group; a C₂-C₆₀ alkynyl group; a C₁-C60 alkoxyl group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring, or a plurality of adjacent R¹³, or a plurality of R¹⁴, or R′ and R″ may be bonded to each other to form a ring,
3) w is an integer of 0 to 3, x is an integer of 0 to 4,
4) 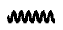 indicates the position to be bonded.

6. The compound of claim 1, wherein the compound represented by Formula 1 is represented by Formula 14 or Formula 15: 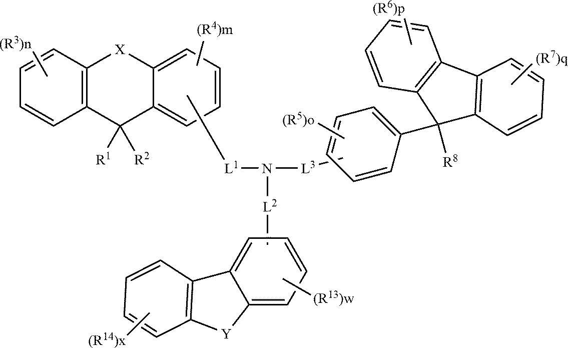 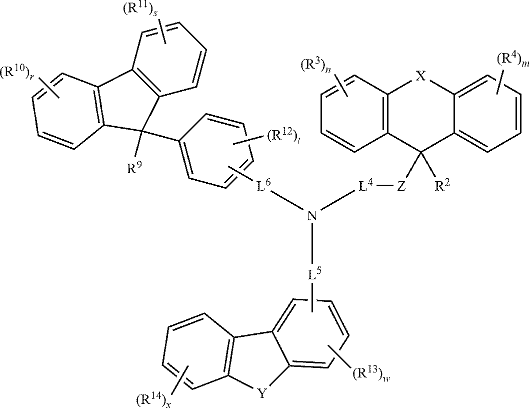
wherein:
1) X, R¹, R², R³, R⁴, R⁵, R⁶, R⁷, R⁸, R⁹, R¹⁰, R¹¹, R¹², L¹, L², L³, L⁴, L⁵, L⁶, Z, m, n, o, p, q, r, s, and t are the same as defined in claim 1,
2) Y is O, S, NR^{b} or CR’R″,
3) R¹³ and R¹⁴ are independently selected from the group consisting of hydrogen: deuterium; tritium; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring, or a plurality of adjacent R¹³, or a plurality of R¹⁴, or R′ and R″ may be bonded to each other to form a ring,
4) w is an integer of 0 to 3, and x is an integer of 0 to 4.

7. The compound of claim 1, wherein the compound represented by Formula 1 is represented by any one of Formulas 16 to 21: 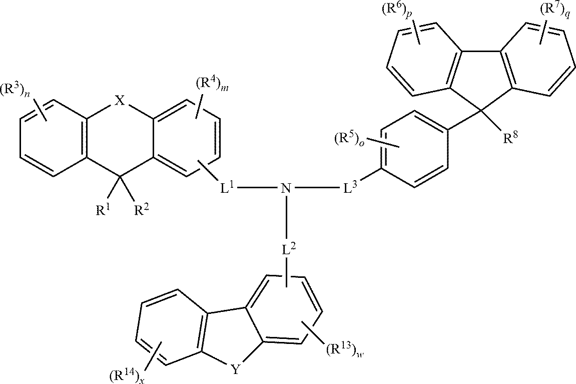 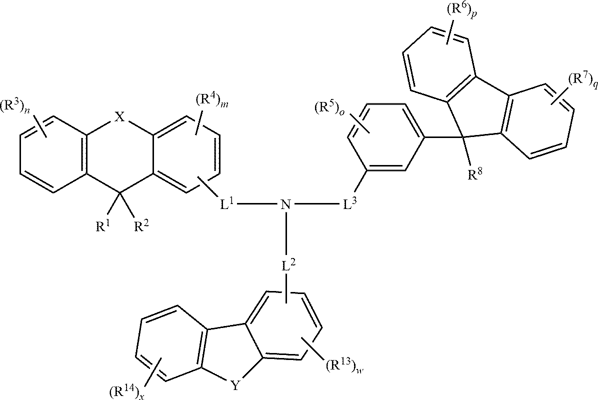 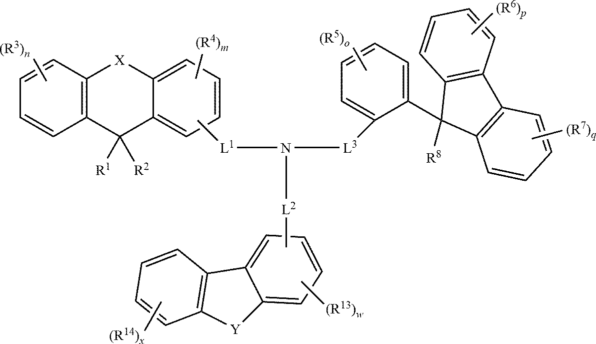 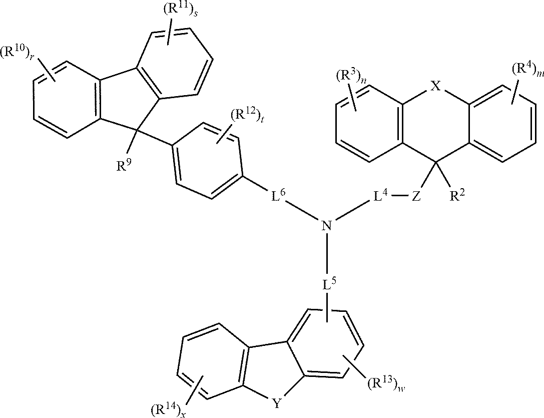 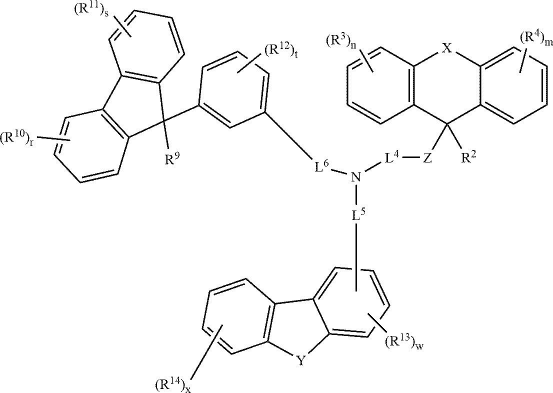 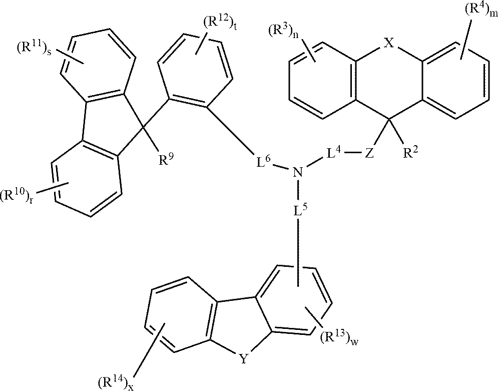
wherein:
1) X, R¹, R², R³, R⁴, R⁵, R⁶, R⁷, R⁸, R⁹, R¹⁰,R¹¹,R¹², L¹, L², L³, L⁴, L⁵, L⁶, Z, m, n, o, p, q, r, s and t are the same as defined in claim 1,
2) Y is O, S, NR^{b} or CR’R″,
3) R¹³ and R¹⁴ are independently selected from the group consisting of hydrogen: deuterium; tritium; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring, or a plurality of adjacent R¹³, or a plurality of R¹⁴, or R′ and R″ may be bonded to each other to form a ring,
4) w is an integer of 0 to 3, and x is an integer of 0 to 4.

8. The compound of claim 1, wherein the compound represented by Formula 1 is represented by any one of Formulas 22 to 25: 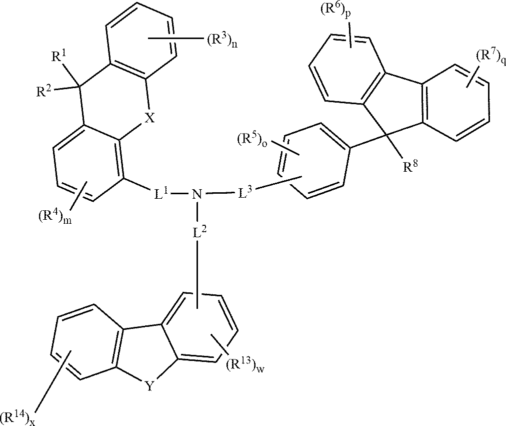 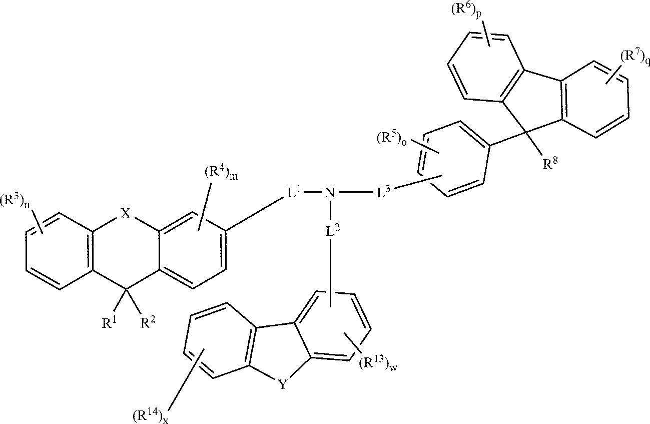 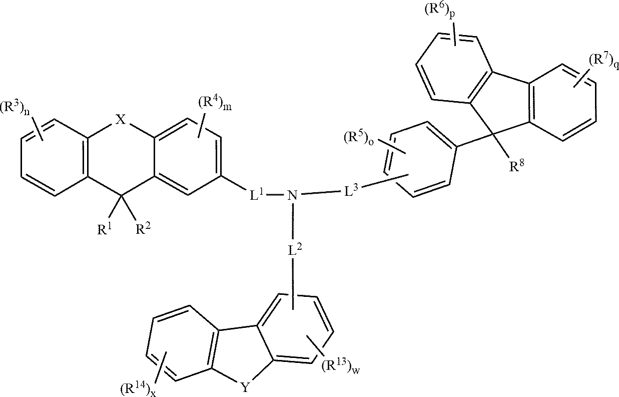 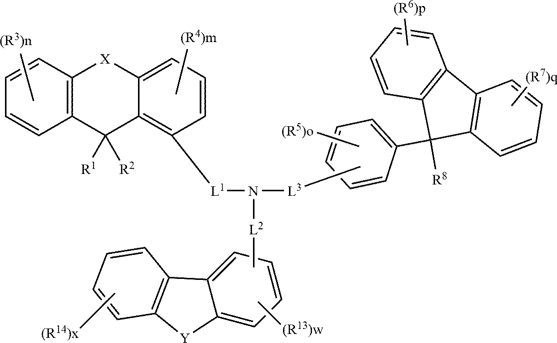
wherein:
1) X, R¹, R², R³, R⁴, R⁵, R⁶, R⁷, R⁸, L¹, L², L³, m, n, o, p and q are the same as defined in claim 1,
2) Y is O, S, NR^{b} or CR’R″,
3) R¹³ and R¹⁴ are independently selected from the group consisting of hydrogen: deuterium; tritium; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring, or a plurality of adjacent R¹³, or a plurality of R¹⁴, or R′ and R″ may be bonded to each other to form a ring,
4) w is an integer of 0 to 3, and x is an integer of 0 to 4.

9. The compound of claim 5, wherein the compound represented by Formula 1-1 is represented by any one of Formulas 1-2 to 1-5: 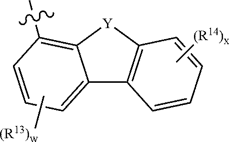 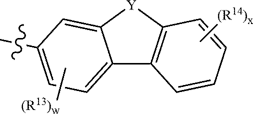 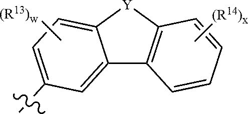 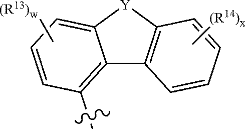
wherein:
Y, R¹³, R¹⁴, w and x are the same as defined in claim 5, 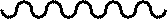 [SMILES: CC1(C)c2ccccc2Oc2ccc(N(c3ccc(-c4ccccc4)cc3)c3ccc(C4(c5ccccc5)c5ccccc5-c5ccccc54)cc3)cc21]
indicates the position to be bonded.

10. The compound of claim 1, wherein the compound represented by Formula 1 is any one of the following compounds P1-1 to P7-4: 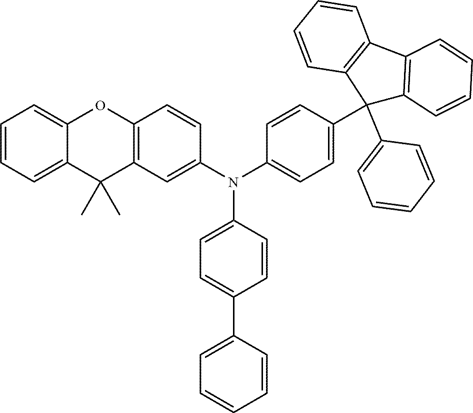 [SMILES: c1ccc(N(c2ccc(C3(c4ccccc4)c4ccccc4-c4ccccc43)cc2)c2ccc3c(c2)C(c2ccccc2)(c2ccccc2)c2ccccc2O3)cc1] 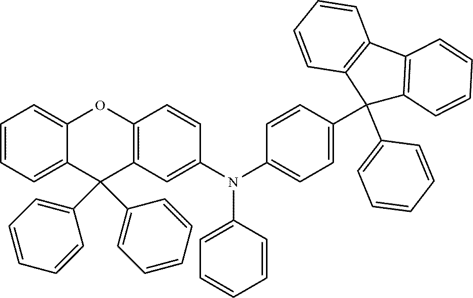 [SMILES: c1ccc(N(c2ccc(C3(c4ccccc4)c4ccccc4-c4ccccc43)cc2)c2ccc3c(c2)C2(c4ccccc4O3)c3ccccc3-c3ccccc32)cc1] 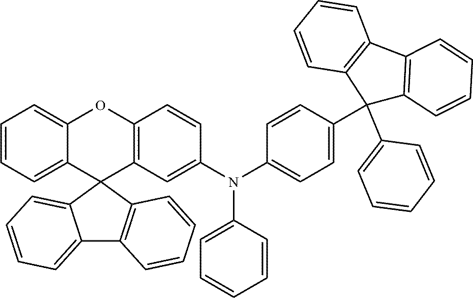 [SMILES: CC1(C)c2ccccc2Oc2cc(N(c3ccc(-c4ccccc4)cc3)c3ccc(C4(c5ccccc5)c5ccccc5-c5ccccc54)cc3)ccc21] 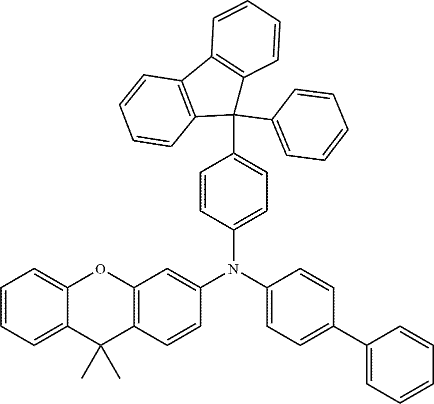 [SMILES: CC1(C)c2ccccc2Oc2c(N(c3ccc(-c4ccccc4)cc3)c3ccc(C4(c5ccccc5)c5ccccc5-c5ccccc54)cc3)cccc21] 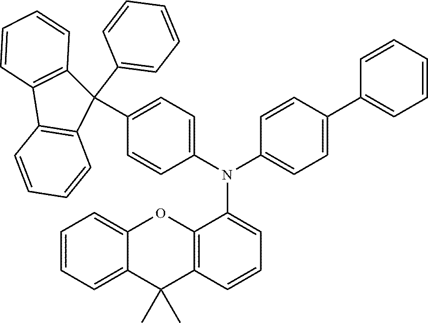 [SMILES: CC1(C)c2ccccc2Oc2cccc(N(c3ccc(-c4ccccc4)cc3)c3ccc(C4(c5ccccc5)c5ccccc5-c5ccccc54)cc3)c21] 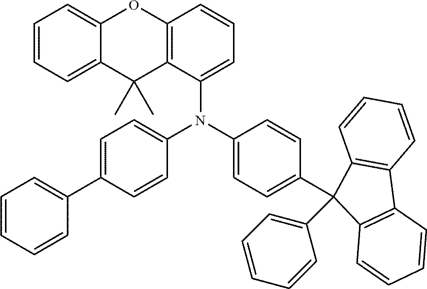 [SMILES: Fc1ccc(N(c2ccc(C3(c4ccccc4)c4ccccc4Oc4ccccc43)cc2)c2ccc(C3(c4ccccc4)c4ccccc4-c4ccccc43)cc2)cc1] 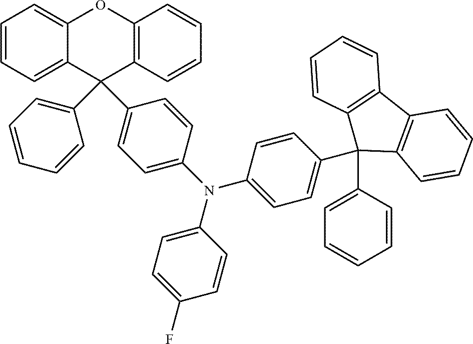 [SMILES: c1ccc(-c2cccc(N(c3ccc(C4(c5ccccc5)c5ccccc5-c5ccccc54)cc3)c3cccc(C4(c5ccccc5)c5ccccc5Oc5ccccc54)c3)c2)cc1] 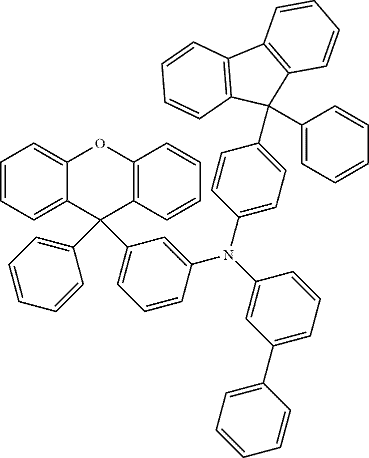 [SMILES: N#Cc1ccc(N(c2ccc(C3(c4ccccc4)c4ccccc4-c4ccccc43)cc2)c2ccccc2C2(c3ccccc3)c3ccccc3Oc3ccccc32)cc1] 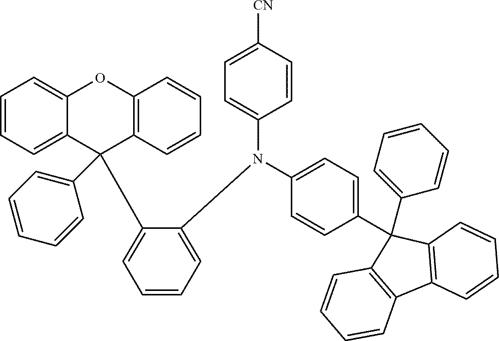 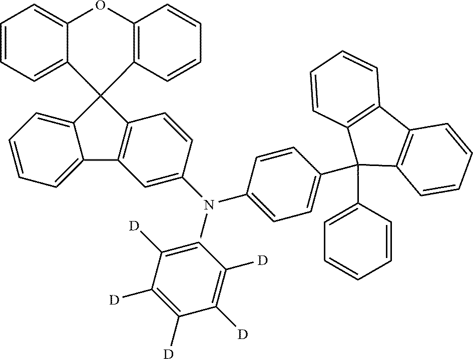 [SMILES: c1ccc(N(c2ccc(C3(c4ccc(-c5ccncc5)cc4)c4ccccc4-c4ccccc43)cc2)c2ccc3c(c2)C2(c4ccccc4Oc4ccccc42)c2ccccc2-3)cc1] 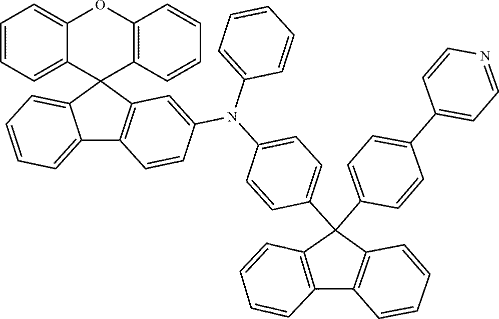 [SMILES: c1ccc(N(c2ccc(C3(c4ccccc4)c4ccccc4-c4ccc(-c5ccncc5)cc43)cc2)c2cccc3c2-c2ccccc2C32c3ccccc3Oc3ccccc32)cc1] 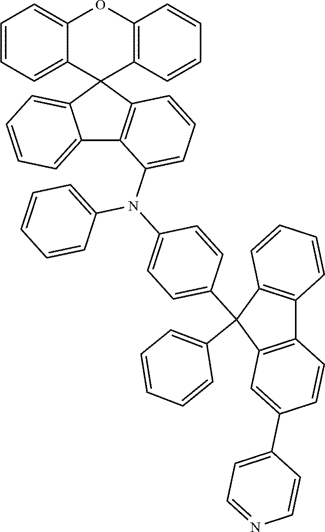 [SMILES: CC1(C)c2ccccc2Oc2ccc(-c3ccc(N(c4ccccc4)c4ccc(C5(c6ccccc6)c6ccccc6-c6ccccc65)cc4)cc3)cc21] 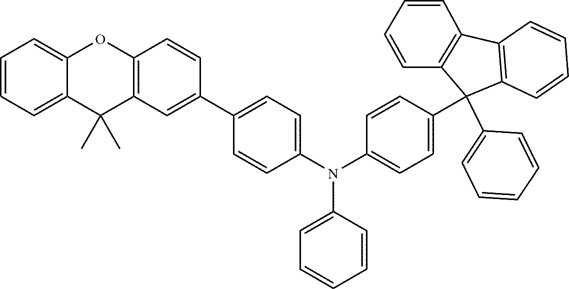 [SMILES: CC1(C)c2ccccc2Oc2cc(-c3cc(-c4ccccc4)cc(N(c4ccccc4)c4ccc(C5(c6ccccc6)c6ccccc6-c6ccccc65)cc4)c3)ccc21] 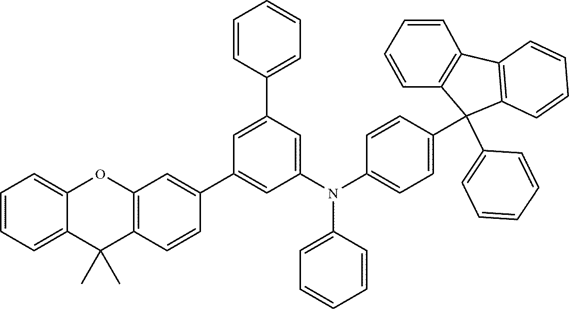 [SMILES: CC1(C)c2ccccc2Oc2cccc(N(c3ccc(F)cc3)c3ccc(-c4ccc(C5(c6ccccc6)c6ccccc6-c6ccccc65)cc4)cc3)c21] 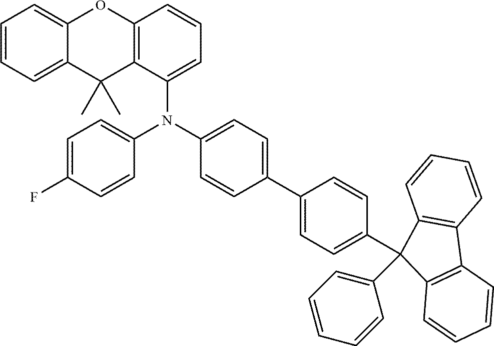 [SMILES: CC(C)(C)Oc1ccc(N(c2cccc(-c3ccc(C4(c5ccccc5)c5ccccc5-c5ccccc54)cc3)c2)c2cccc(-c3ccc4c(c3)Oc3ccccc3C4(C)C)c2)cc1] 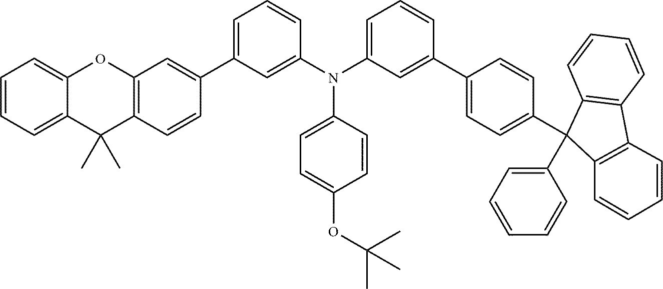 [SMILES: CC1(C)c2ccccc2Sc2ccc(N(c3ccc(-c4ccccc4)cc3)c3ccc(C4(c5ccccc5)c5ccccc5-c5ccccc54)cc3)cc21] 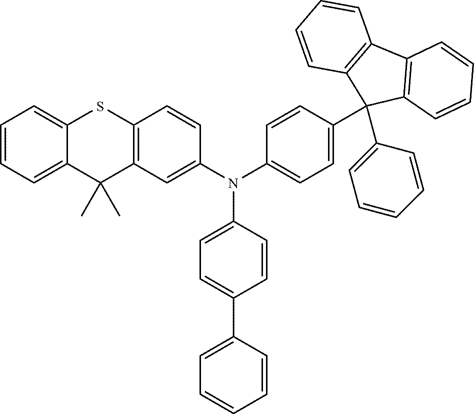 [SMILES: c1ccc(N(c2ccc(C3(c4ccccc4)c4ccccc4-c4ccccc43)cc2)c2ccc3c(c2)C(c2ccccc2)(c2ccccc2)c2ccccc2S3)cc1] 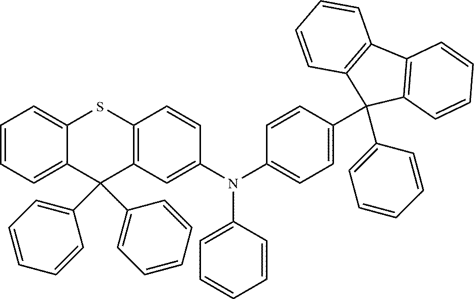 [SMILES: c1ccc(-c2ccc3c(c2)C2(c4cc(N(c5ccccc5)c5ccc(C6(c7ccccc7)c7ccccc7-c7ccccc76)cc5)ccc4S3)c3ccccc3-c3ccccc32)cc1] 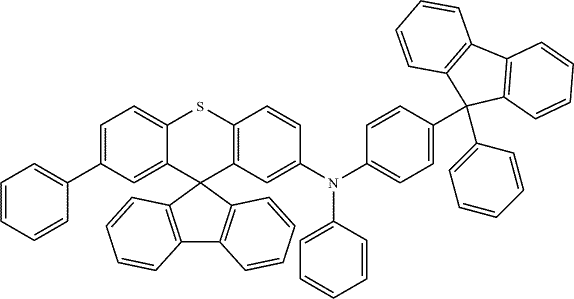 [SMILES: CC1(C)c2ccccc2Sc2cc(N(c3ccc(-c4ccccc4)cc3)c3ccc(C4(c5ccccc5)c5ccccc5-c5ccccc54)cc3)ccc21] 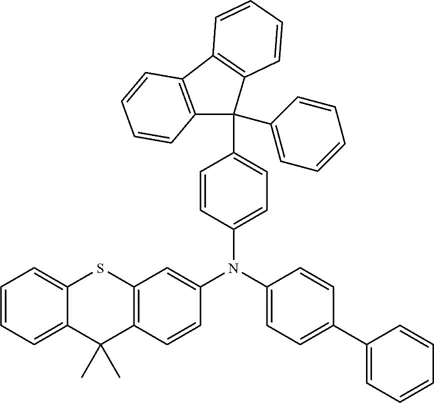 [SMILES: [2H]c1c([2H])c([2H])c(N(c2ccc(C3(c4ccccc4)c4ccccc4-c4ccccc43)cc2)c2cccc3c2Sc2ccccc2C3(C)C)c([2H])c1[2H]] 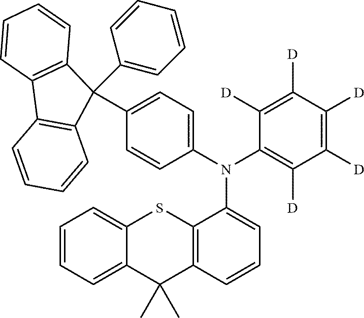 [SMILES: Fc1ccc2c(c1)C(c1ccccc1)(c1ccc(N(c3ccccc3)c3cccc(C4(c5ccccc5)c5ccccc5Sc5ccccc54)c3)cc1)c1ccccc1-2] 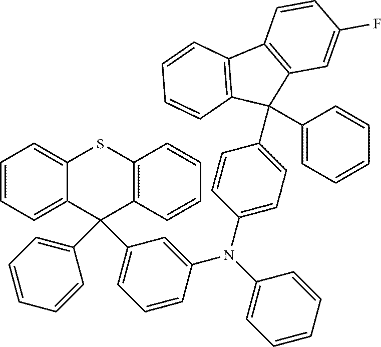 [SMILES: c1ccc(N(c2ccc(C3(c4ccccc4)c4ccccc4-c4ccccc43)cc2)c2ccc3c(c2)C2(c4ccccc4Sc4ccccc42)c2ccccc2-3)cc1] 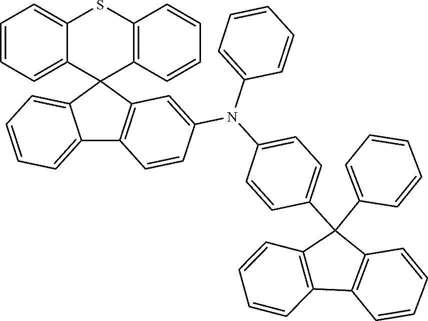 [SMILES: c1ccc(N(c2ccc(C3(c4ccccc4)c4ccccc4-c4c3ccc3ccccc43)cc2)c2cccc3c2-c2ccccc2C32c3ccccc3Sc3ccccc32)cc1] 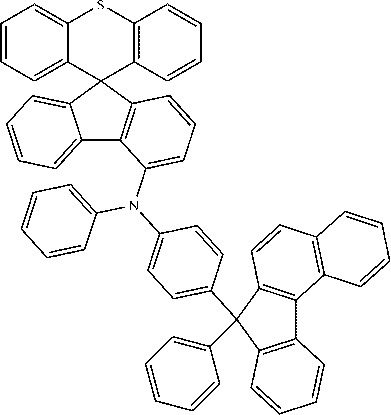 [SMILES: CC1(C)c2ccccc2Sc2ccc(-c3ccccc3N(c3ccccc3)c3ccc(C4(c5ccccc5)c5ccccc5-c5ccccc54)cc3)cc21] 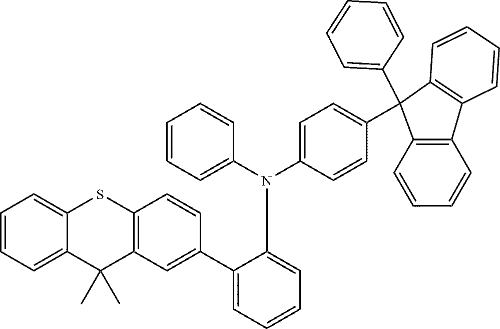 [SMILES: CC1(C)c2ccccc2Sc2cc(-c3cccc(N(c4ccc(F)cc4)c4ccc(C5(c6ccccc6)c6ccccc6-c6ccccc65)cc4)c3)ccc21] 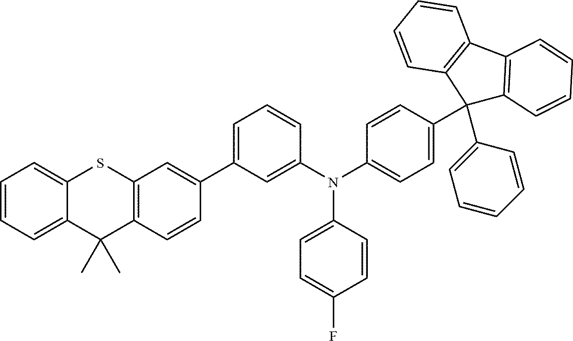 [SMILES: CC1(C)c2ccccc2Sc2cccc(N(c3ccccc3)c3ccc(-c4ccc(C5(c6ccccc6)c6ccccc6-c6ccccc65)cc4)cc3)c21] 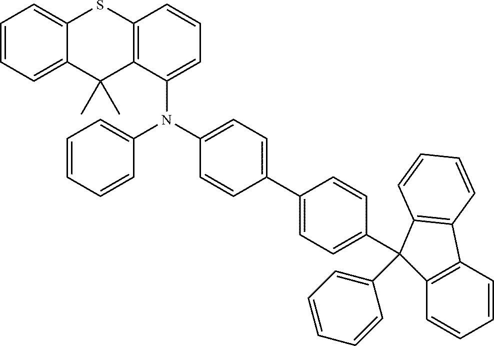 [SMILES: CC1(C)c2ccccc2Sc2cc(-c3cccc(N(c4ccccc4)c4cccc(-c5ccc(C6(c7ccccc7)c7ccccc7-c7ccccc76)cc5)c4)c3)ccc21] 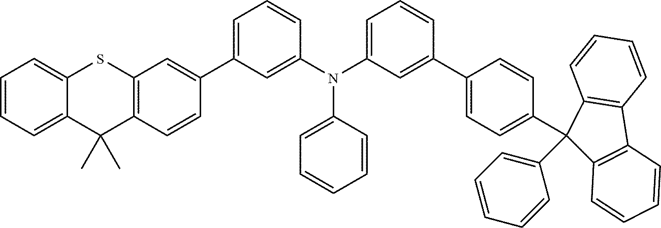 [SMILES: CC1(C)c2ccccc2Oc2ccc(N(c3ccc(C4(c5ccccc5)c5ccccc5-c5ccccc54)cc3)c3cccc(-c4ccccc4)c3)cc21] 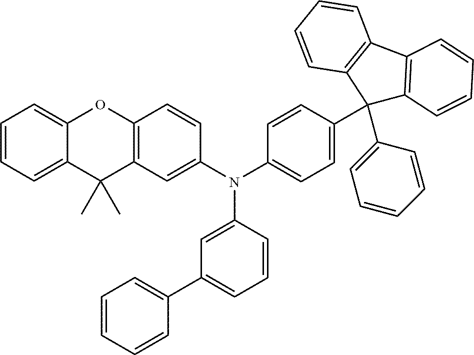 [SMILES: CC1(c2ccccc2)c2ccccc2Sc2ccc(N(c3ccc(C45CC6CC(CC(C6)C4)C5)cc3)c3ccc(C4(c5ccccc5)c5ccccc5-c5ccccc54)cc3)cc21] 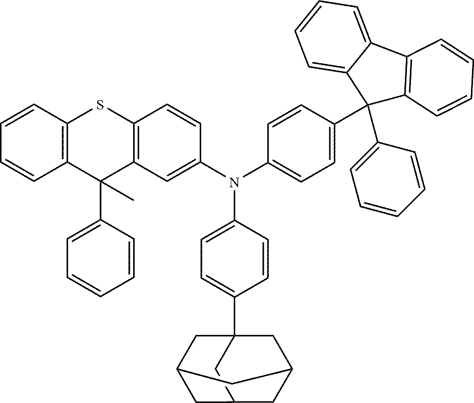 [SMILES: CC1(C)c2cc(N(c3ccc(C4(c5ccccc5)c5ccccc5-c5ccccc54)cc3)c3ccc4ccccc4c3)ccc2Oc2ccc3ccccc3c21] 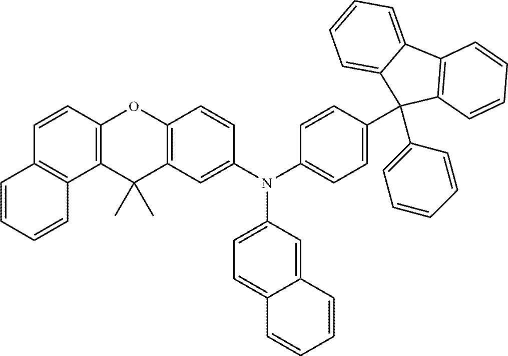 [SMILES: c1ccc(N(c2ccc(C3(c4ccccc4)c4ccccc4-c4ccccc43)cc2)c2ccc3c(c2)C2(c4ccccc4Sc4ccccc42)c2ccc4ccccc4c2-3)cc1] 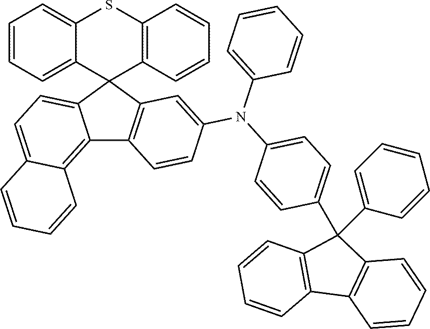 [SMILES: CC1(C)c2ccccc2Oc2ccc(N(c3ccc(C4(C)c5ccccc5-c5ccccc54)cc3)c3cccc(-c4ccccc4)c3)cc21] 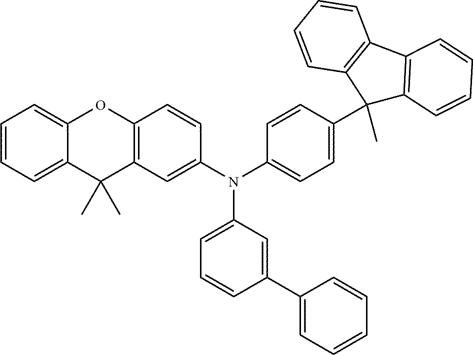 [SMILES: CC1(c2ccc(N(c3ccccc3)c3ccc4c(c3)C(c3ccccc3)(c3ccccc3)c3ccccc3S4)cc2)c2ccccc2-c2ccccc21] 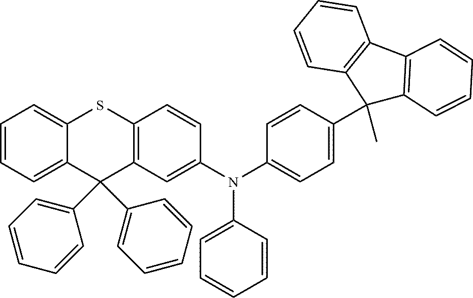 [SMILES: CC1(C)c2ccccc2Oc2ccc(N(c3cccc(-c4ccccc4)c3)c3cccc(C4(c5ccccc5)c5ccccc5-c5ccccc54)c3)cc21] 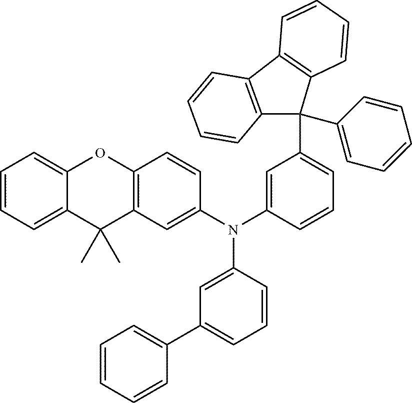 [SMILES: c1ccc(N(c2cccc(C3(c4ccccc4)c4ccccc4-c4ccccc43)c2)c2ccc3c(c2)C(c2ccccc2)(c2ccccc2)c2ccccc2O3)cc1] 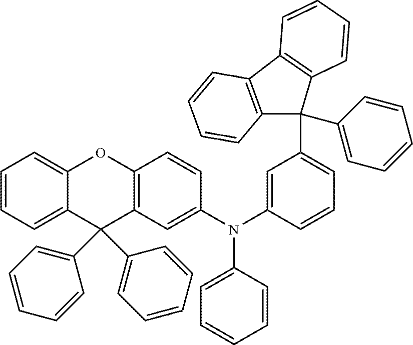 [SMILES: c1ccc(-c2ccc(N(c3cccc(C4(c5ccccc5)c5ccccc5-c5ccccc54)c3)c3ccc4c(c3)C3(c5ccccc5O4)c4ccccc4-c4ccccc43)cc2)cc1] 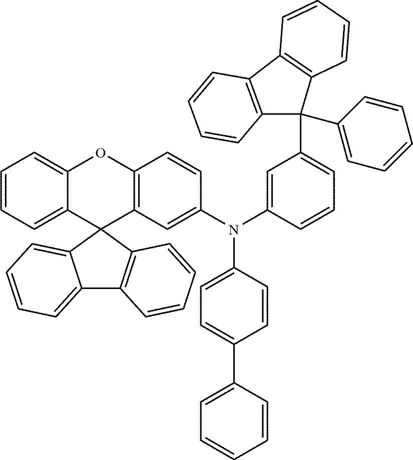 [SMILES: CC1(C)c2ccccc2Oc2cc(N(c3cccc(C4(c5ccccc5)c5ccccc5-c5ccccc54)c3)c3ccccc3-c3ccccc3)ccc21] 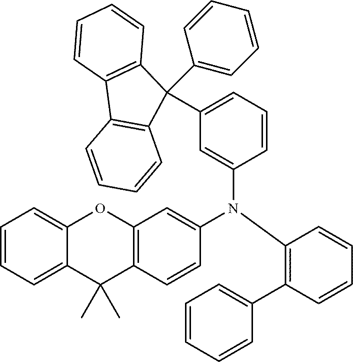 [SMILES: CC1(C)c2ccccc2Oc2c(N(c3ccc(F)cc3)c3cccc(C4(c5ccccc5)c5ccccc5-c5ccccc54)c3)cccc21] 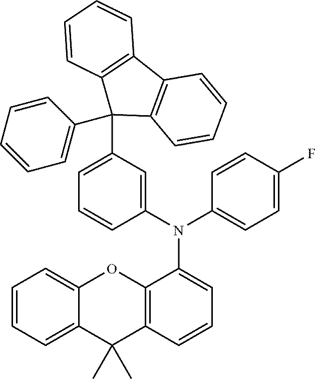 [SMILES: CC1(C)c2ccccc2Oc2cccc(N(c3cccc(C4(c5ccccc5)c5ccccc5-c5ccccc54)c3)c3cccc4ccccc34)c21] 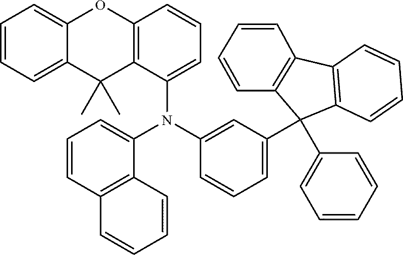 [SMILES: c1ccc(-c2ccc(C3(c4cccc(N(c5ccccc5)c5ccc(C6(c7ccccc7)c7ccccc7Oc7ccccc76)cc5)c4)c4ccccc4-c4ccccc43)cc2)cc1] 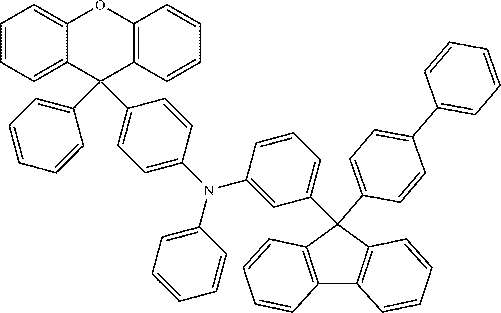 [SMILES: c1ccc(N(c2cccc(C3(c4ccccc4)c4ccccc4Oc4ccccc43)c2)c2cccc(C3(c4ccccc4)c4ccccc4-c4ccccc43)c2)cc1] 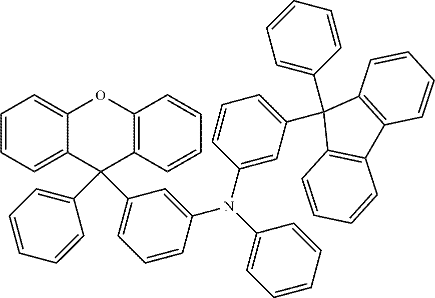 [SMILES: N#Cc1ccc(N(c2cccc(C3(c4ccccc4)c4ccccc4-c4ccccc43)c2)c2ccccc2C2(c3ccccc3)c3ccccc3Oc3ccccc32)cc1] 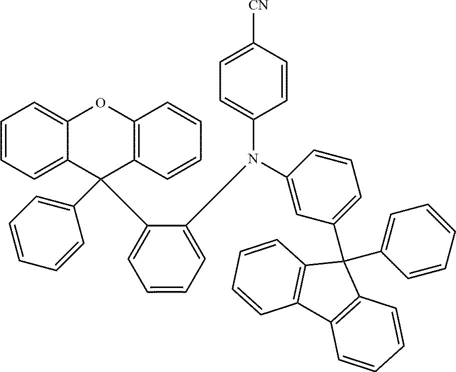 [SMILES: c1ccc(N(c2cccc(C3(c4ccccc4)c4ccccc4-c4ccc5ccccc5c43)c2)c2ccc3c(c2)-c2ccccc2C32c3ccccc3Oc3ccccc32)cc1] 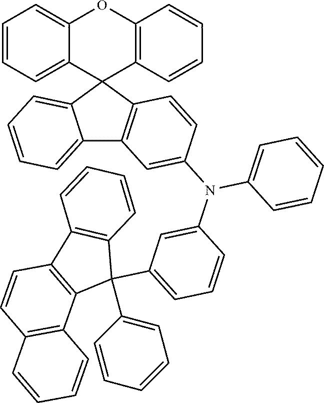 [SMILES: c1ccc(-c2ccc(N(c3cccc(C4(c5ccccc5)c5ccccc5-c5ccccc54)c3)c3ccc4c(c3)C3(c5ccccc5Oc5ccccc53)c3ccccc3-4)cc2)cc1] 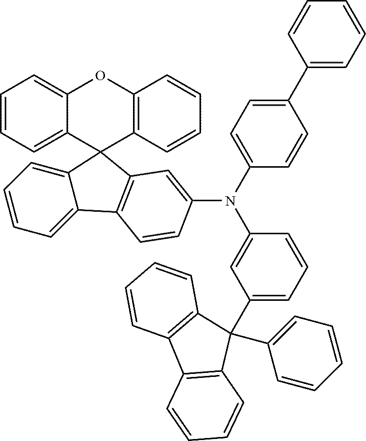 [SMILES: c1ccc(Oc2ccc(N(c3cccc(C4(c5ccccc5)c5ccccc5-c5ccccc54)c3)c3cccc4c3-c3ccccc3C43c4ccccc4Oc4ccccc43)cc2)cc1] 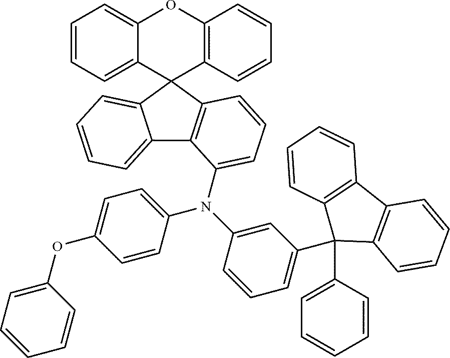 [SMILES: [2H]c1c([2H])c([2H])c(N(c2ccc(-c3ccc4c(c3)C(C)(C)c3ccccc3O4)cc2)c2cccc(C3(c4ccccc4)c4ccccc4-c4ccccc43)c2)c([2H])c1[2H]] 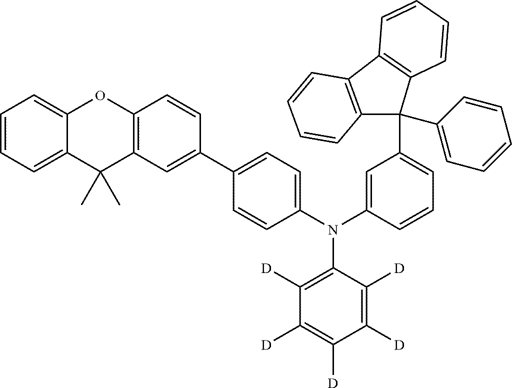 [SMILES: CC1(C)c2ccccc2Oc2ccc(-c3ccc(N(c4cccc(-c5ccccc5)c4)c4cccc(C5(c6ccccc6)c6ccccc6-c6ccccc65)c4)cc3)cc21] 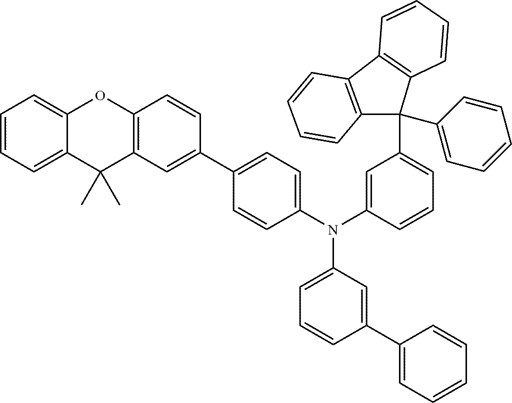 [SMILES: CC1(C)c2ccccc2Oc2cccc(N(c3ccc(-c4cccc(C5(c6ccccc6)c6ccccc6-c6ccccc65)c4)cc3)c3ccc4ccccc4c3)c21] 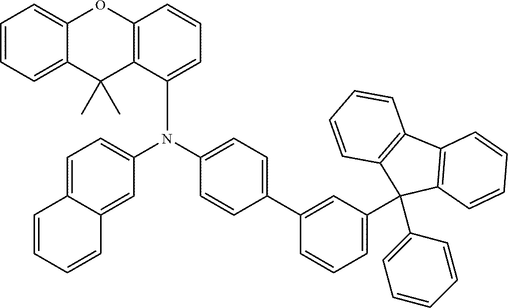 [SMILES: CC1(C)c2ccccc2Oc2cc(-c3cccc(N(c4ccccc4)c4ccc(C5(c6ccccc6)c6ccccc6-c6ccccc65)cc4)c3)ccc21] 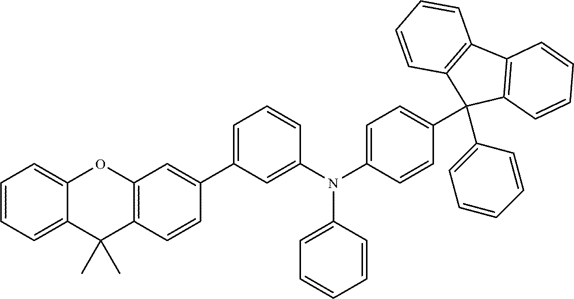 [SMILES: CC1(C)c2ccccc2Sc2ccc(N(c3ccccc3)c3cccc(C4(c5ccccc5)c5ccccc5-c5ccccc54)c3)cc21] 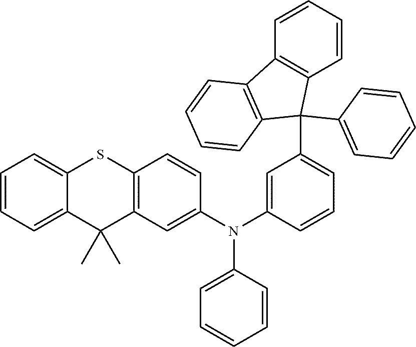 [SMILES: c1ccc(-c2cccc(N(c3cccc(C4(c5ccccc5)c5ccccc5-c5ccccc54)c3)c3ccc4c(c3)C(c3ccccc3)(c3ccccc3)c3ccccc3S4)c2)cc1] 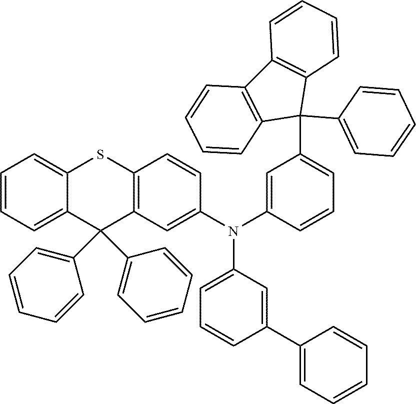 [SMILES: c1ccc(N(c2cccc(C3(c4ccc5ccccc5c4)c4ccccc4-c4ccccc43)c2)c2ccc3c(c2)C2(c4ccccc4S3)c3ccccc3-c3ccccc32)cc1] 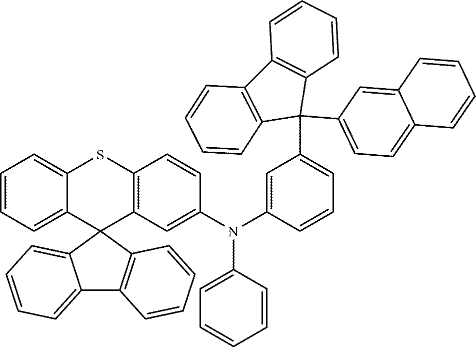 [SMILES: CC1(C)c2ccc(-c3ccccc3)cc2Sc2cc(N(c3ccccc3)c3cccc(C4(c5ccccc5)c5ccccc5-c5ccccc54)c3)ccc21] 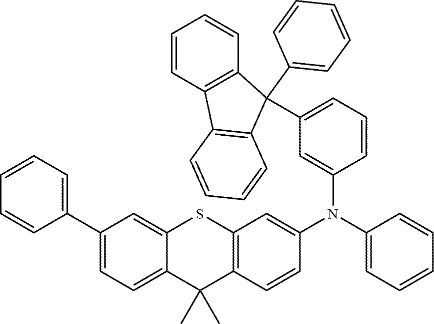 [SMILES: CC1(C)c2ccccc2Sc2c(N(c3cccc(C4(c5ccccc5)c5ccccc5-c5ccccc54)c3)c3ccc4ccccc4c3)cccc21] 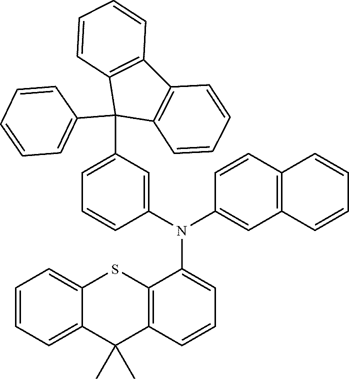 [SMILES: c1ccc(-c2ccccc2N(c2cccc(C3(c4ccccc4)c4ccccc4Sc4ccccc43)c2)c2cccc(C3(c4ccccc4)c4ccccc4-c4ccccc43)c2)cc1] 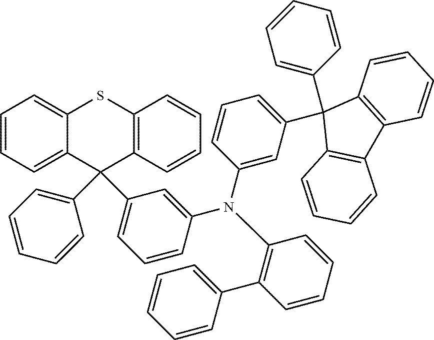 [SMILES: Fc1ccc(N(c2ccc3c(c2)C2(c4ccccc4Sc4ccccc42)c2ccccc2-3)c2cc(C3(c4ccccc4)c4ccccc4-c4ccccc43)cc3ccccc23)cc1] 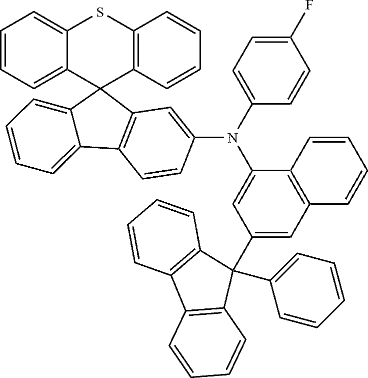 [SMILES: N#Cc1ccc(N(c2cccc(C3(c4ccccc4)c4ccccc4-c4ccccc43)c2)c2cccc3c2-c2ccccc2C32c3ccccc3Sc3ccccc32)cc1] 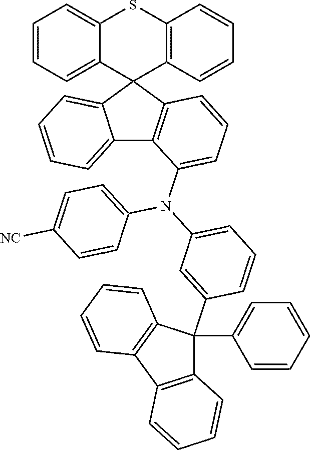 [SMILES: CC1(C)c2ccccc2Sc2ccc(-c3ccc(N(c4ccccc4)c4cccc(C5(c6ccccc6)c6ccccc6-c6ccccc65)c4)cc3)cc21] 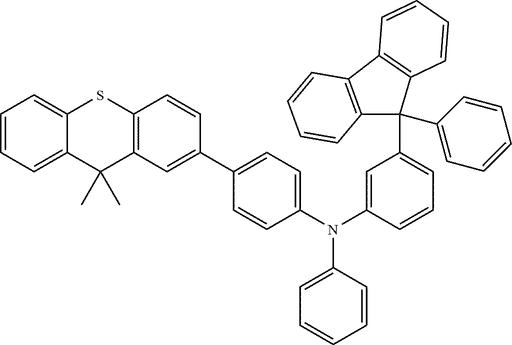 [SMILES: CC1(C)c2ccccc2Sc2cc(-c3cccc(N(c4ccccc4)c4cccc(C5(c6ccccc6)c6ccccc6-c6ccccc65)c4)c3)ccc21] 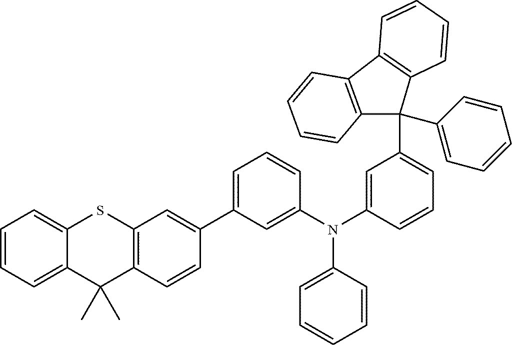 [SMILES: CC1(C)c2ccccc2Sc2cccc(N(c3ccccc3)c3ccc(-c4cccc(C5(c6ccccc6)c6ccccc6-c6ccccc65)c4)cc3)c21] 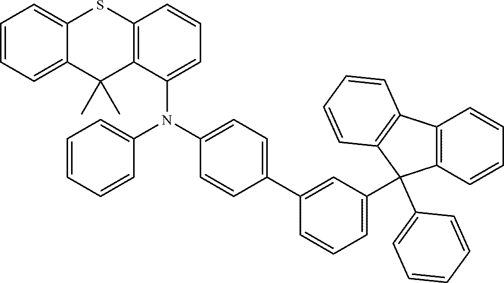 [SMILES: CC1(C)c2ccccc2Sc2cc(-c3cccc(N(c4ccccc4)c4ccccc4-c4cccc(C5(c6ccccc6)c6ccccc6-c6ccccc65)c4)c3)ccc21] 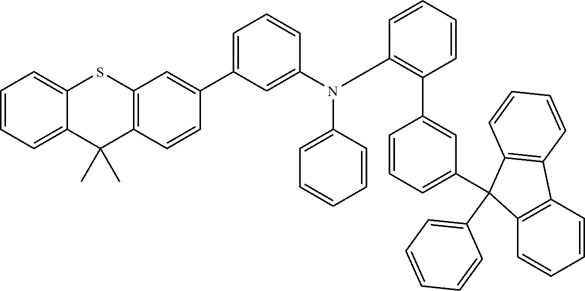 [SMILES: CC1(c2cccc(N(c3ccc(-c4ccccc4)cc3)c3ccc4c(c3)C3(c5ccccc5O4)c4ccccc4-c4ccccc43)c2)c2ccccc2-c2ccccc21] 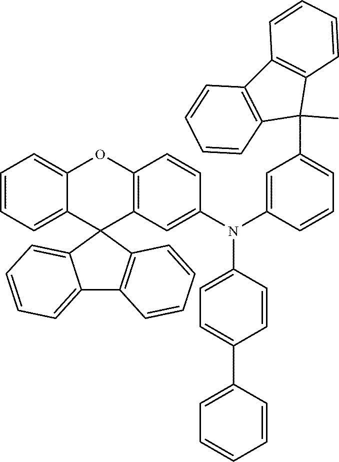 [SMILES: CC1(C)c2ccccc2Sc2cc(N(c3cccc(C4(C)c5ccccc5-c5ccccc54)c3)c3ccccc3-c3ccccc3)ccc21] 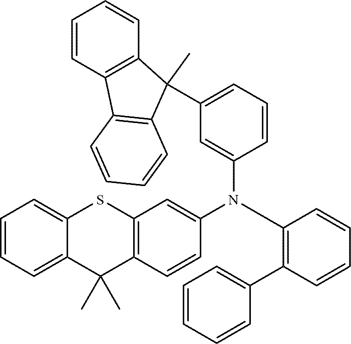 [SMILES: CC1(C)c2ccccc2Oc2ccc(N(c3cccc(-c4ccccc4)c3)c3ccccc3C3(c4ccccc4)c4ccccc4-c4ccccc43)cc21] 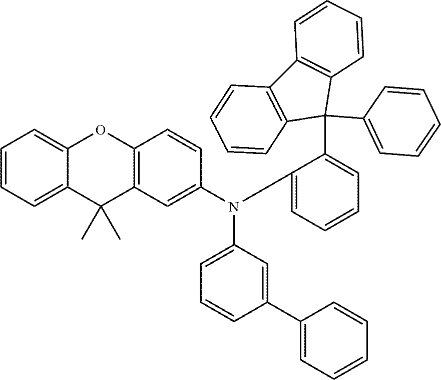 [SMILES: c1ccc(C2(c3ccccc3)c3ccccc3Oc3ccc(N(c4ccc5ccccc5c4)c4ccccc4C4(c5ccccc5)c5ccccc5-c5ccccc54)cc32)cc1] 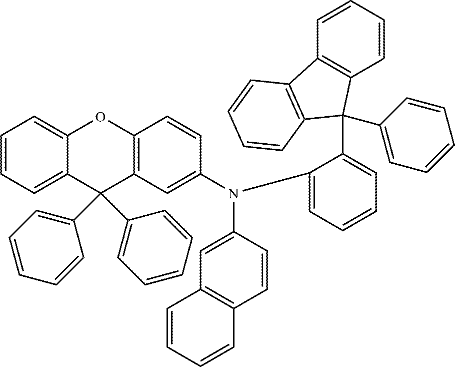 [SMILES: c1ccc(-c2cccc(N(c3ccc4c(c3)C3(c5ccccc5O4)c4ccccc4-c4ccccc43)c3ccccc3C3(c4ccccc4)c4ccccc4-c4ccccc43)c2)cc1] 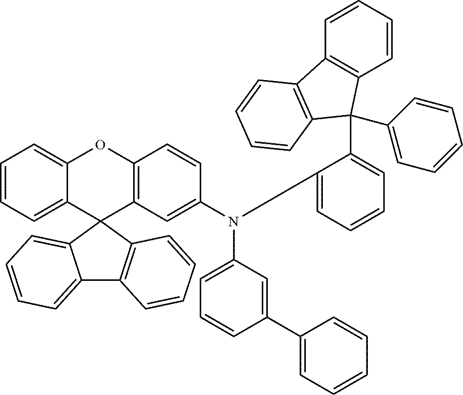 [SMILES: CC1(C)c2ccccc2Oc2cc(N(c3ccccc3)c3ccccc3C3(c4ccccc4)c4ccccc4-c4ccccc43)ccc21] 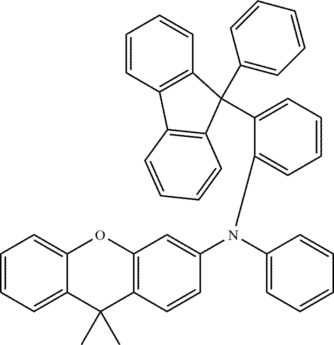 [SMILES: CC1(C)c2ccccc2Oc2c(N(c3ccccc3C3(c4ccccc4)c4ccccc4-c4ccccc43)c3cccc4ccccc34)cccc21] 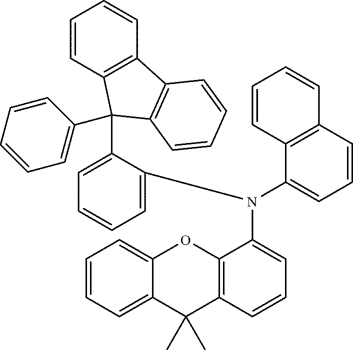 [SMILES: CC1(C)c2ccccc2Oc2cccc(N(c3ccccc3)c3cc(C4(c5ccccc5)c5ccccc5-c5ccccc54)cc4ccccc34)c21] 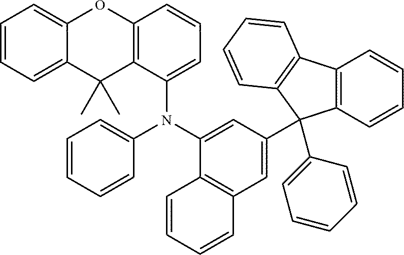 [SMILES: Fc1ccc(N(c2ccc(C3(c4ccccc4)c4ccccc4Oc4ccccc43)cc2)c2ccccc2C2(c3ccccc3)c3ccccc3-c3ccccc32)cc1] 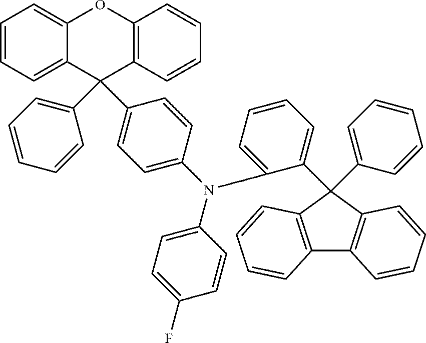 [SMILES: c1ccc(-c2ccc(N(c3cccc(C4(c5ccccc5)c5ccccc5Oc5ccccc54)c3)c3ccccc3C3(c4ccccc4)c4ccccc4-c4ccccc43)cc2)cc1] 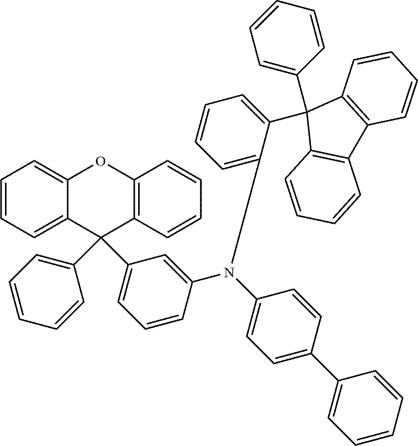 [SMILES: c1ccc(N(c2ccccc2C2(c3ccccc3)c3ccccc3Oc3ccccc32)c2ccccc2C2(c3ccccc3)c3ccccc3-c3ccccc32)cc1] 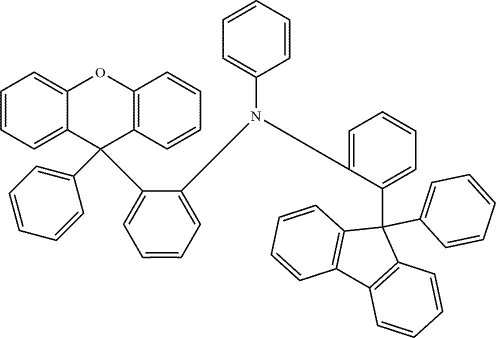 [SMILES: [2H]c1c([2H])c([2H])c(N(c2ccc3c(c2)-c2ccccc2C32c3ccccc3Oc3ccccc32)c2ccccc2C2(c3ccccc3)c3ccccc3-c3ccccc32)c([2H])c1[2H]] 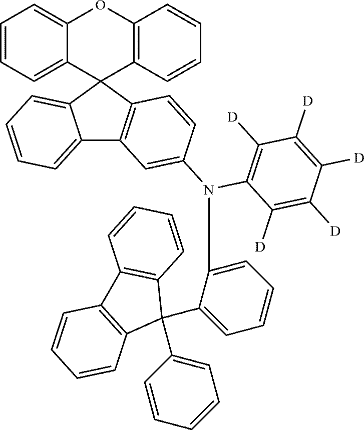 [SMILES: c1ccc(N(c2ccc3c(c2)C2(c4ccccc4Oc4ccccc42)c2ccccc2-3)c2ccccc2C2(c3ccccc3)c3ccccc3-c3cc4ccccc4cc32)cc1] 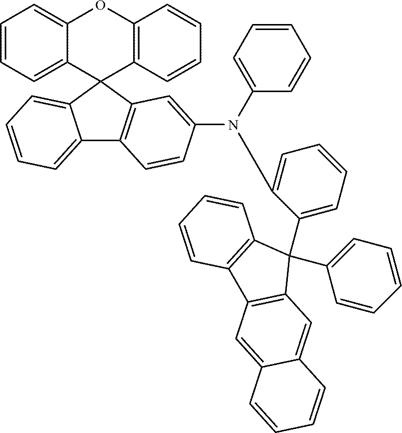 [SMILES: c1ccc(N(c2ccccc2C2(c3ccccc3)c3ccccc3-c3ccccc32)c2cccc3c2-c2ccccc2C32c3ccccc3Oc3ccccc32)cc1] 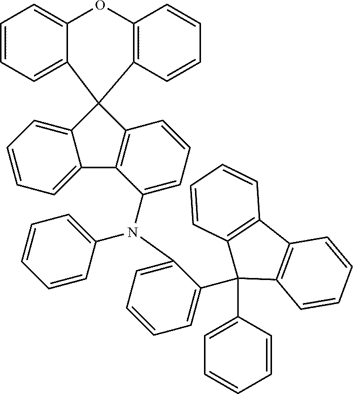 [SMILES: CC1(C)c2ccccc2Oc2ccc(-c3ccc(N(c4ccccc4)c4ccccc4C4(c5ccccc5)c5ccccc5-c5ccccc54)cc3)cc21] 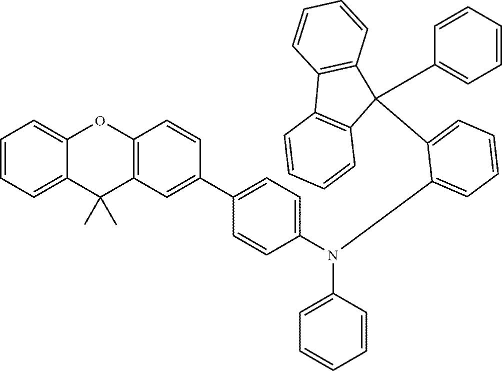 [SMILES: CC1(C)c2ccccc2Oc2cc(-c3cccc(N(c4ccccc4C4(c5ccccc5)c5ccccc5-c5cc6ccccc6cc54)c4cccc5ccccc45)c3)ccc21] 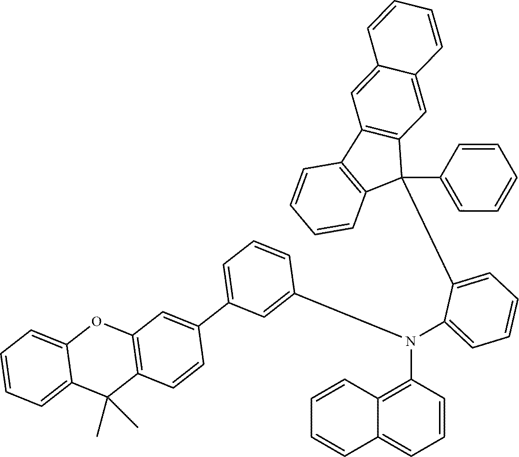 [SMILES: CC1(C)c2ccccc2Oc2cccc(N(c3ccc(-c4ccccc4C4(c5ccccc5)c5ccccc5-c5ccccc54)cc3)c3ccc4ccccc4c3)c21] 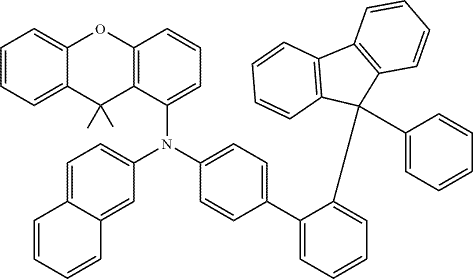 [SMILES: CC1(C)c2ccccc2Oc2c(-c3ccccc3N(c3ccccc3)c3cccc(-c4ccccc4C4(c5ccccc5)c5ccccc5-c5ccccc54)c3)cccc21] 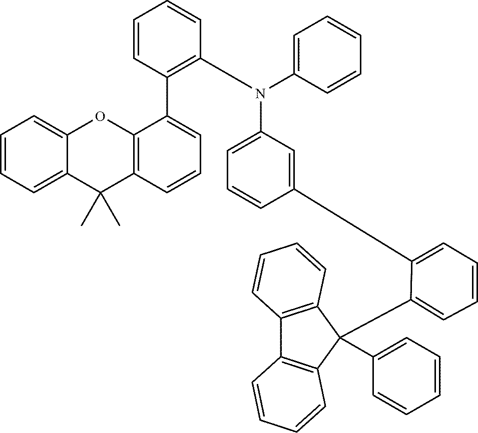 [SMILES: CC1(C)c2ccccc2Sc2ccc(N(c3cccc(-c4ccccc4)c3)c3ccccc3C3(c4ccccc4)c4ccccc4-c4ccccc43)cc21] 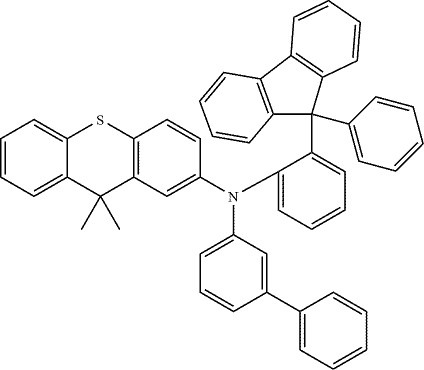 [SMILES: c1ccc(-c2ccc3c(c2)C(c2ccccc2)(c2ccccc2)c2cc(N(c4ccccc4)c4ccccc4C4(c5ccccc5)c5ccccc5-c5ccccc54)ccc2S3)cc1] 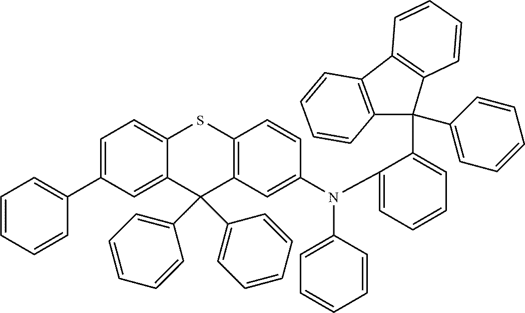 [SMILES: c1ccc(-c2ccc(N(c3ccc4c(c3)C3(c5ccccc5S4)c4ccccc4-c4ccccc43)c3ccccc3C3(c4ccccc4)c4ccccc4-c4ccccc43)cc2)cc1] 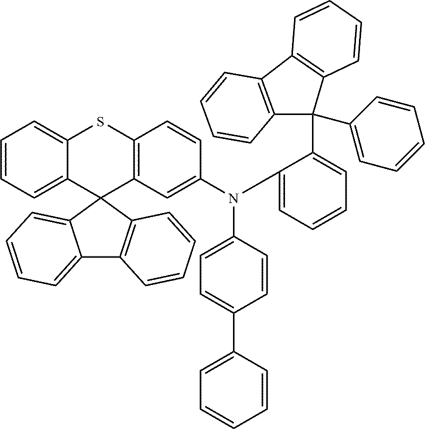 [SMILES: CC1(C)c2ccccc2Sc2cc(N(c3ccc(F)cc3)c3ccccc3C3(c4ccccc4)c4ccccc4-c4ccccc43)ccc21] 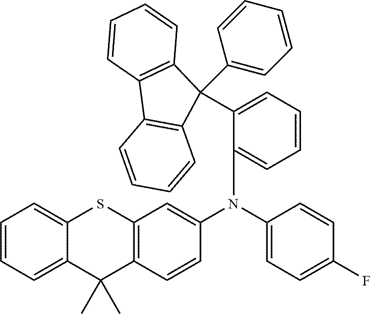 [SMILES: CC1(C)c2ccccc2Sc2c(N(c3ccccc3)c3ccccc3C3(c4ccccc4)c4ccccc4-c4ccccc43)cccc21] 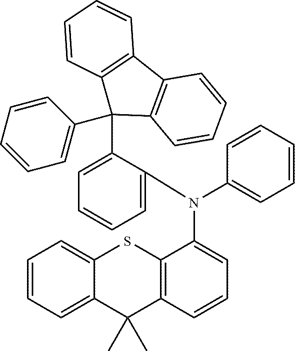 [SMILES: c1ccc(N(c2cccc(C3(c4ccccc4)c4ccccc4Sc4ccccc43)c2)c2ccccc2C2(c3ccccc3)c3ccccc3-c3ccccc32)cc1] 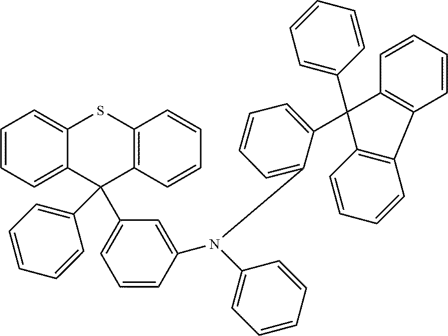 [SMILES: c1ccc(N(c2ccc3c(c2)C2(c4ccccc4Sc4ccccc42)c2ccccc2-3)c2ccccc2C2(c3ccccc3)c3ccccc3-c3ccc(-c4ccncc4)cc32)cc1] 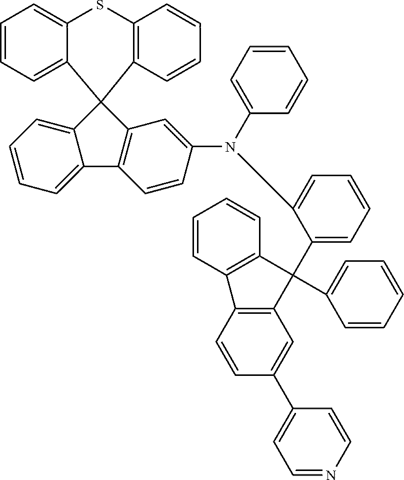 [SMILES: c1ccc(-c2ccc(N(c3ccccc3C3(c4ccccc4)c4ccccc4-c4ccccc43)c3cccc4c3-c3ccccc3C43c4ccccc4Sc4ccccc43)cc2)cc1] 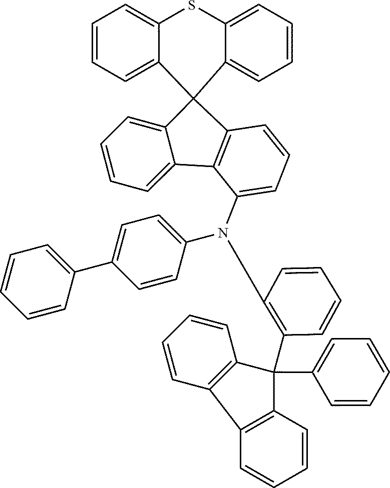 [SMILES: CC1(C)c2ccccc2Sc2ccc(-c3ccc(N(c4ccccc4)c4ccccc4C4(c5ccccc5)c5ccccc5-c5ccccc54)cc3)cc21] 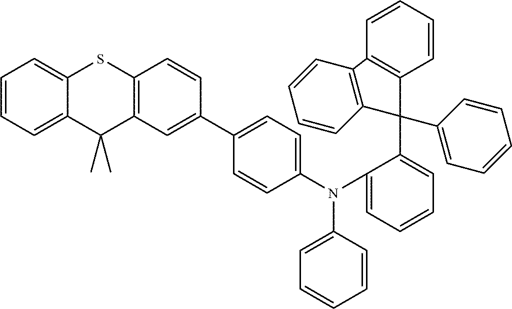 [SMILES: CC1(C)c2ccccc2Sc2cc(-c3ccccc3N(c3ccccc3)c3ccccc3C3(c4ccccc4)c4ccccc4-c4ccccc43)ccc21] 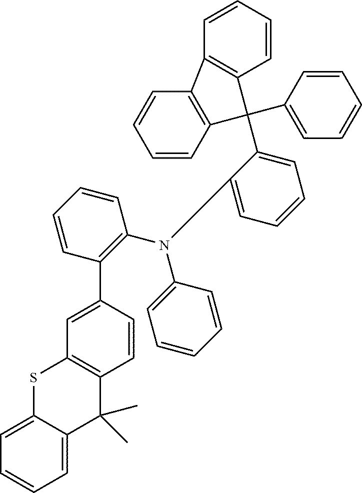 [SMILES: CC1(C)c2ccccc2Sc2cccc(N(c3ccccc3)c3ccc(-c4ccccc4C4(c5ccccc5)c5ccccc5-c5ccccc54)c4ccccc34)c21] 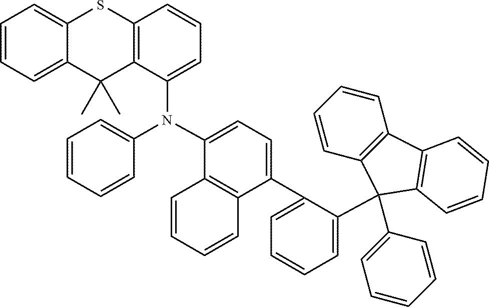 [SMILES: CC1(C)c2ccccc2Sc2cc(-c3cccc(N(c4ccc5ccccc5c4)c4ccccc4-c4ccccc4C4(c5ccccc5)c5ccccc5-c5ccccc54)c3)ccc21] 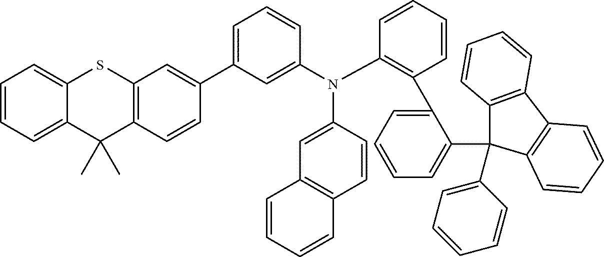 [SMILES: CC1(C)c2ccccc2Sc2ccc(-c3ccc(N(c4ccccc4)c4ccccc4C4(C)c5ccccc5-c5ccccc54)cc3)cc21] 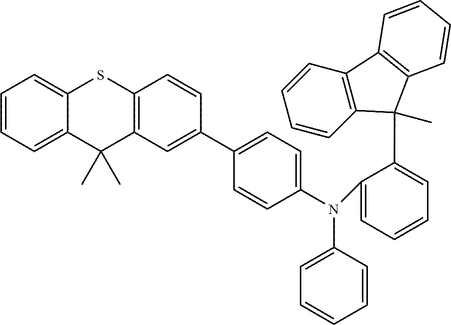 [SMILES: CC1(C)c2ccccc2Oc2cc(-c3ccccc3N(c3ccccc3)c3ccccc3C3(C)c4ccccc4-c4ccccc43)ccc21] 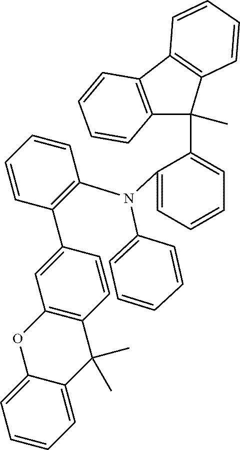 [SMILES: CC1(C)c2ccccc2Oc2ccc(N(c3ccc(C4(c5ccccc5)c5ccccc5-c5ccccc54)cc3)c3cccc4c3sc3ccccc34)cc21] 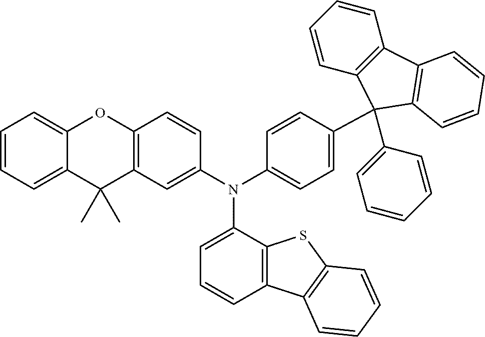 [SMILES: CC1(C)c2ccccc2Oc2ccc(N(c3ccc(C4(c5ccccc5)c5ccccc5-c5ccccc54)cc3)c3ccc4oc5ccccc5c4c3)cc21] 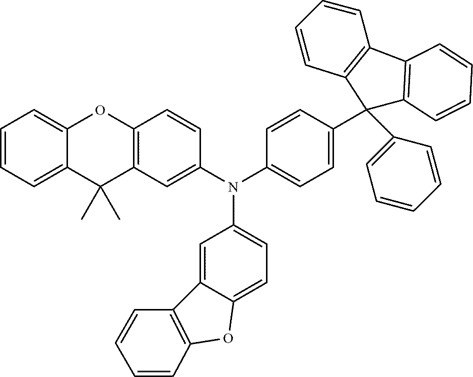 [SMILES: CC1(C)c2ccccc2-c2ccc(N(c3ccc(C4(c5ccccc5)c5ccccc5-c5ccccc54)cc3)c3ccc4c(c3)C(c3ccccc3)(c3ccccc3)c3ccccc3O4)cc21] 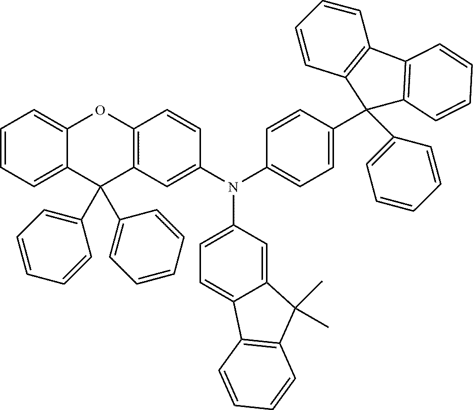 [SMILES: c1ccc(C2(c3ccc(N(c4ccc5c(c4)C4(c6ccccc6O5)c5ccccc5-c5ccccc54)c4cccc5oc6ccccc6c45)cc3)c3ccccc3-c3ccccc32)cc1] 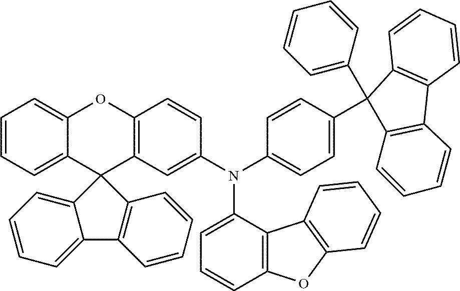 [SMILES: CC1(C)c2ccccc2Oc2cc(N(c3ccc(C4(c5ccccc5)c5ccccc5-c5ccccc54)cc3)c3ccc4c5ccccc5n(-c5ccccc5)c4c3)ccc21] 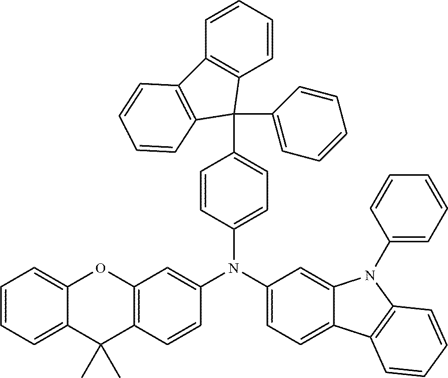 [SMILES: CC1(C)c2ccccc2Oc2cccc(N(c3ccc(C4(c5ccccc5)c5ccccc5-c5ccccc54)cc3)c3ccc4c(c3)-c3ccccc3C43c4ccccc4-c4ccccc43)c21] 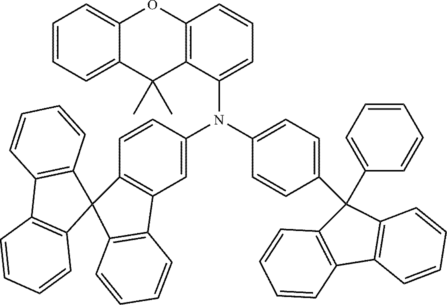 [SMILES: c1ccc(C2(c3cccc(N(c4ccc(C5(c6ccccc6)c6ccccc6-c6ccccc65)cc4)c4ccc5c(c4)oc4c6ccccc6ccc54)c3)c3ccccc3Oc3ccccc32)cc1] 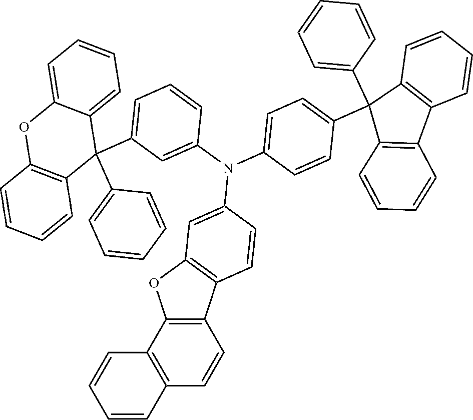 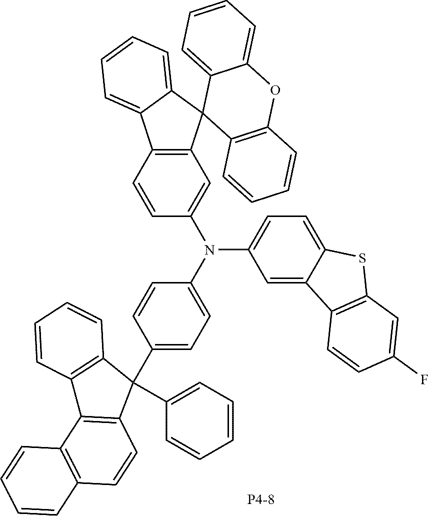 [SMILES: CC1(C)c2ccccc2Sc2ccc(N(c3ccc(C4(c5ccccc5)c5ccccc5-c5ccccc54)cc3)c3cccc4sc5ccccc5c34)cc21] 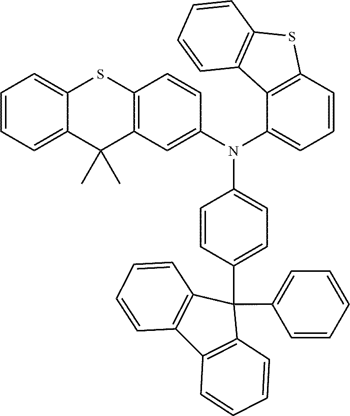 [SMILES: CC1(C)c2ccccc2Sc2ccc(N(c3ccc(C4(c5ccccc5)c5ccccc5-c5ccccc54)cc3)c3ccc4c5ccccc5n(-c5ccccc5)c4c3)cc21] 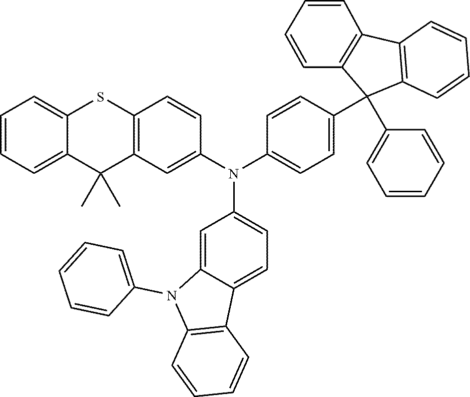 [SMILES: [2H]c1c([2H])c([2H])c2c(oc3cc(N(c4ccc(C5(c6ccccc6)c6ccccc6-c6ccccc65)cc4)c4ccc5c(c4)C(c4ccccc4)(c4ccccc4)c4ccccc4S5)ccc32)c1[2H]] 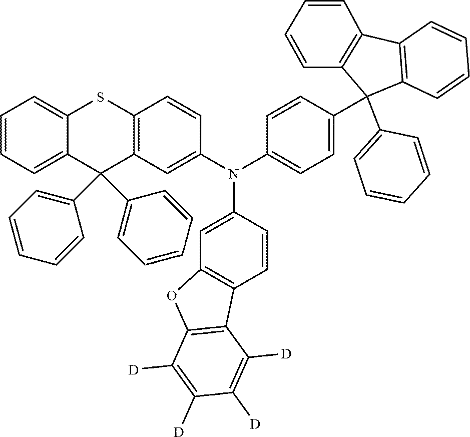 [SMILES: c1ccc(C2(c3ccc(N(c4ccc5c(c4)C4(c6ccccc6S5)c5ccccc5-c5ccccc54)c4cccc5c4sc4ccccc45)cc3)c3ccccc3-c3ccccc32)cc1] 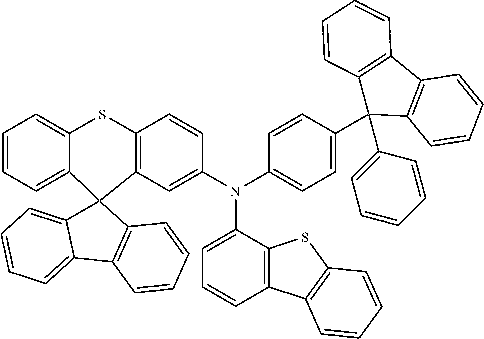 [SMILES: CC1(C)c2ccccc2Sc2cc(N(c3ccc(C4(c5ccccc5)c5ccccc5-c5ccccc54)cc3)c3ccc4c(c3)C(C)(C)c3ccccc3-4)ccc21] 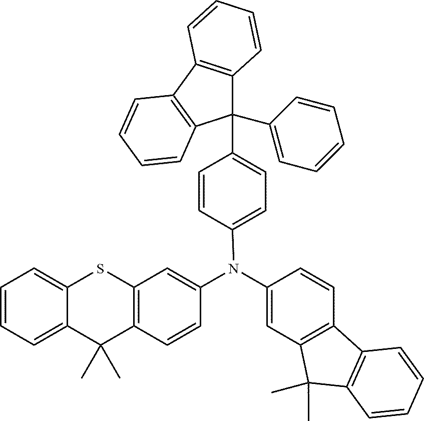 [SMILES: CC1(C)c2ccccc2Sc2cccc(N(c3ccc(C4(c5ccccc5)c5ccccc5-c5ccccc54)cc3)c3ccc4c(c3)c3ccccc3n4-c3cccc4ccccc34)c21] 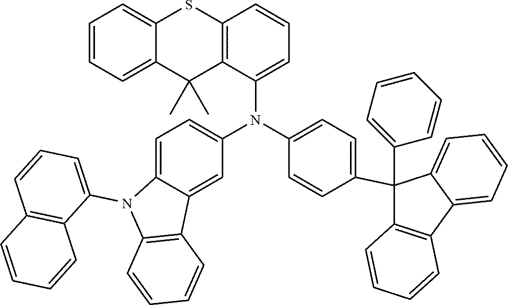 [SMILES: c1ccc(C2(c3cccc(N(c4ccc(C5(c6ccccc6)c6ccccc6-c6ccccc65)cc4)c4ccc5sc6cc(-c7ccncc7)ccc6c5c4)c3)c3ccccc3Sc3ccccc32)cc1] 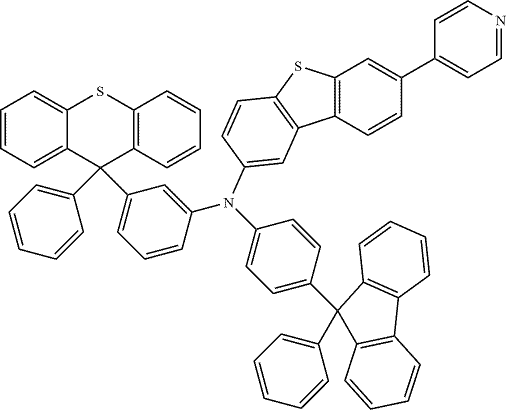 [SMILES: c1ccc(-c2ccc3oc4cc(N(c5ccc(C6(c7ccccc7)c7ccccc7-c7ccccc76)cc5)c5ccc6c(c5)C5(c7ccccc7Sc7ccccc75)c5ccccc5-6)ccc4c3c2)cc1] 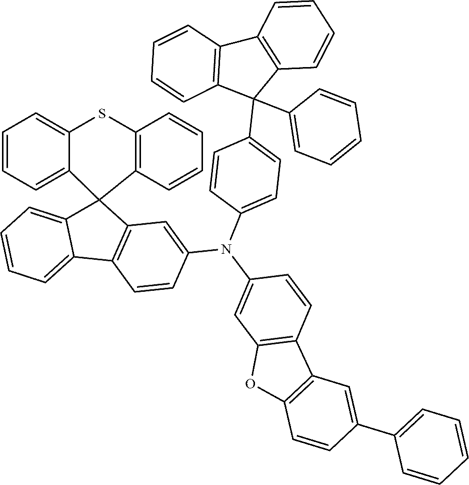 [SMILES: c1ccc(C2(c3ccc(N(c4ccc(-c5ccc6oc7ccccc7c6c5)cc4)c4cccc(-c5ccc6c(c5)C5(c7ccccc7Sc7ccccc75)c5ccccc5-6)c4)cc3)c3ccccc3-c3ccccc32)cc1] 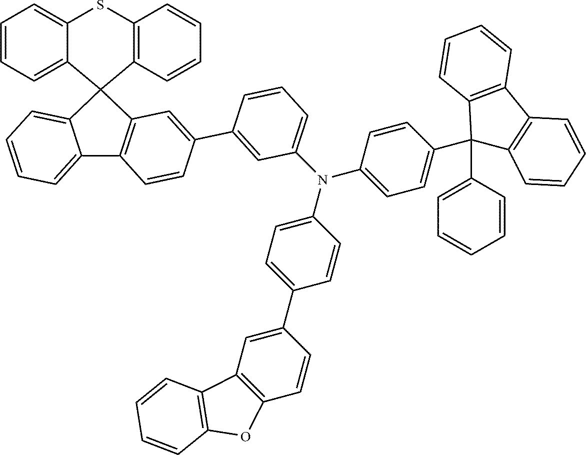 [SMILES: c1ccc(C2(c3ccccc3)c3ccccc3Oc3ccc(-c4ccc(N(c5ccc(C6(c7ccccc7)c7ccccc7-c7ccccc76)cc5)c5ccc6c(c5)sc5ccccc56)cc4)cc32)cc1] 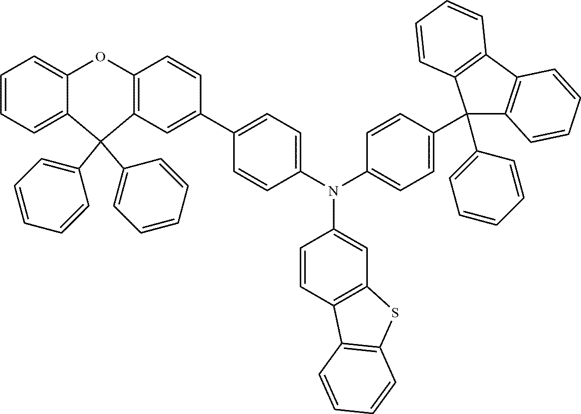 [SMILES: c1ccc(-n2c3ccccc3c3ccc(N(c4cccc(-c5ccc(C6(c7ccccc7)c7ccccc7-c7ccccc76)cc5)c4)c4ccc5c(c4)C(c4ccccc4)(c4ccccc4)c4ccccc4O5)cc32)cc1] 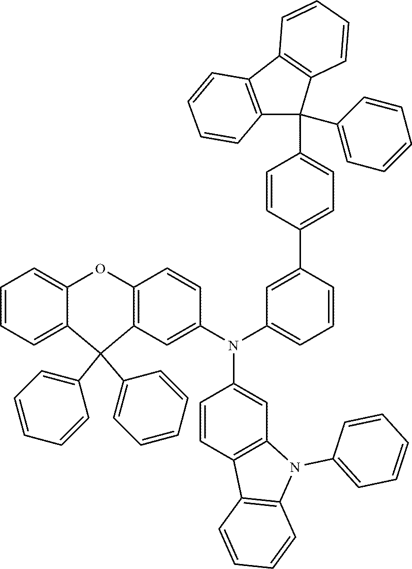 [SMILES: CC1(C)c2ccccc2Oc2cc(-c3ccc(N(c4ccc(-c5ccc(C6(c7ccccc7)c7ccccc7-c7ccccc76)cc5)cc4)c4ccc5c(c4)C(C)(C)c4ccccc4-5)cc3)ccc21] 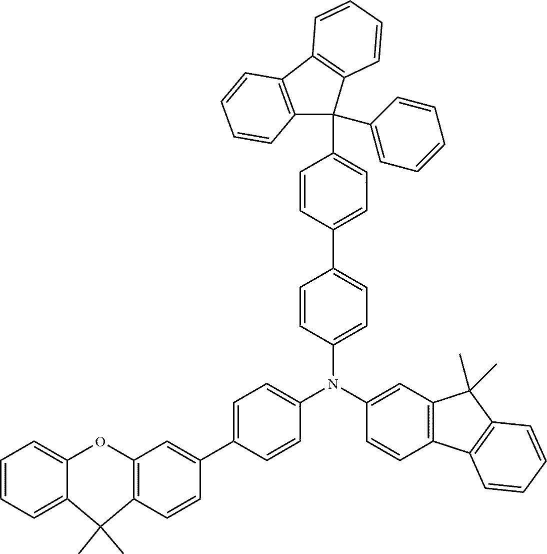 [SMILES: CC1(C)c2ccccc2Oc2ccc(N(c3cccc(C4(c5ccccc5)c5ccccc5-c5ccccc54)c3)c3ccc4c(c3)oc3ccccc34)cc21] 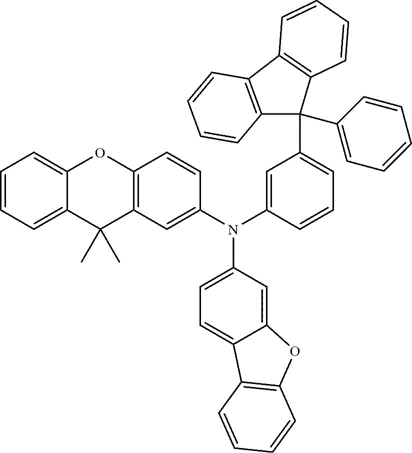 [SMILES: CC1(C)c2ccccc2Oc2ccc(N(c3cccc(C4(c5ccccc5)c5ccccc5-c5ccccc54)c3)c3ccc4c(c3)sc3ccccc34)cc21] 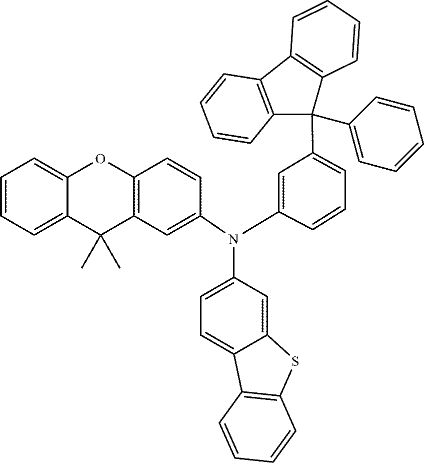 [SMILES: CC1(C)c2ccccc2Oc2ccc(N(c3cccc(C4(c5ccccc5)c5ccccc5-c5ccccc54)c3)c3ccc4c5ccccc5n(-c5ccccc5)c4c3)cc21] 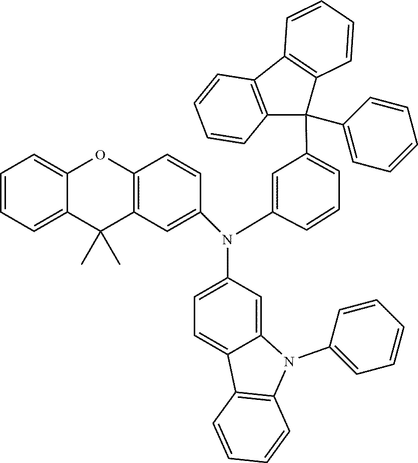 [SMILES: CC1(C)c2ccccc2Oc2ccc(N(c3cccc(C4(c5ccccc5)c5ccccc5-c5ccccc54)c3)c3ccc4c(c3)C(C)(C)c3ccccc3-4)cc21] 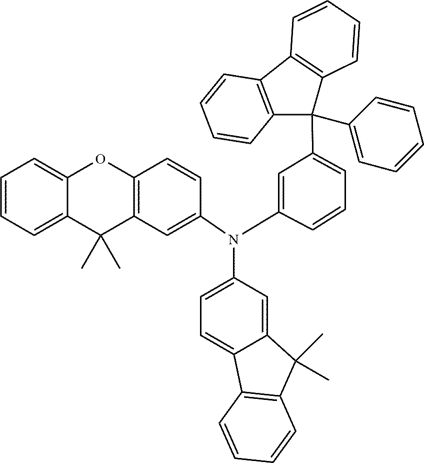 [SMILES: CC1(C)c2ccccc2Oc2ccc(N(c3cccc(C4(c5ccccc5)c5ccccc5-c5ccccc54)c3)c3ccc4oc5ccccc5c4c3)cc21] 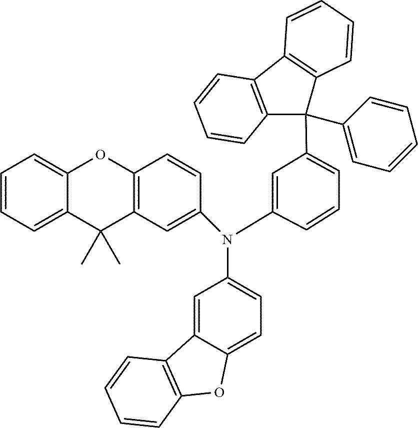 [SMILES: CC1(C)c2ccccc2Oc2ccc(N(c3cccc(C4(c5ccccc5)c5ccccc5-c5ccccc54)c3)c3cccc4oc5ccccc5c34)cc21] 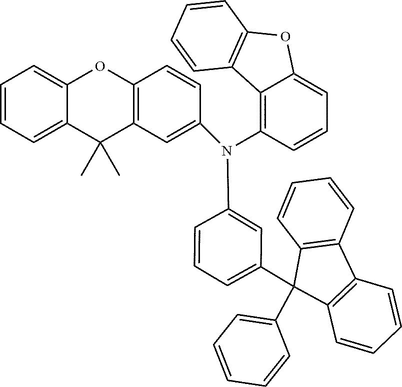 [SMILES: CC1(C)c2ccccc2Oc2ccc(N(c3cccc(C4(c5ccccc5)c5ccccc5-c5ccccc54)c3)c3cccc4c3oc3ccccc34)cc21] 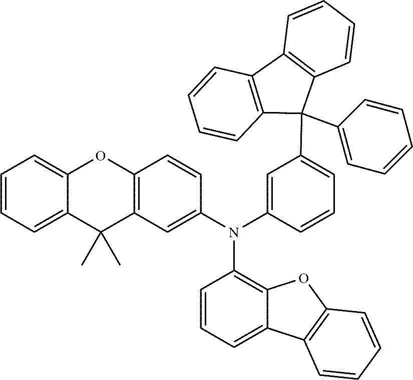 [SMILES: CC1(C)c2ccccc2Oc2ccc(N(c3cccc(C4(c5ccccc5)c5ccccc5-c5ccccc54)c3)c3ccc4sc5ccccc5c4c3)cc21] 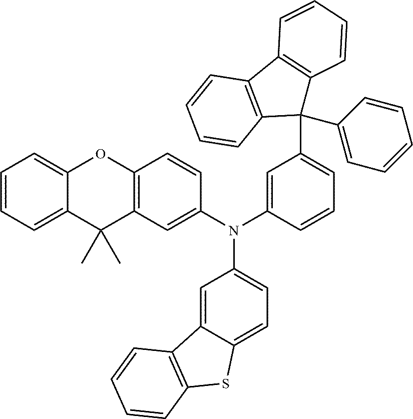 [SMILES: CC1(C)c2ccccc2Oc2ccc(N(c3cccc(C4(c5ccccc5)c5ccccc5-c5ccccc54)c3)c3cccc4sc5ccccc5c34)cc21] 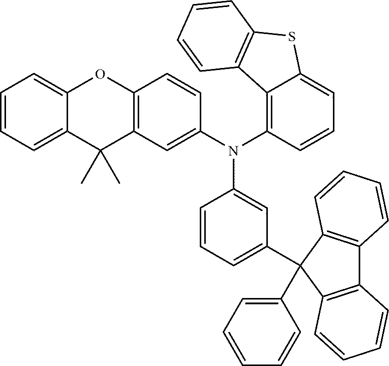 [SMILES: CC1(C)c2ccccc2Oc2ccc(N(c3cccc(C4(c5ccccc5)c5ccccc5-c5ccccc54)c3)c3ccc4c(c3)c3ccccc3n4-c3ccccc3)cc21] 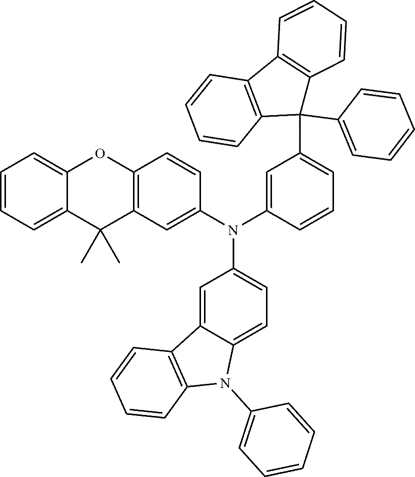 [SMILES: CC1(C)c2ccccc2Oc2ccc(N(c3cccc(C4(c5ccccc5)c5ccccc5-c5ccccc54)c3)c3cccc4c3c3ccccc3n4-c3ccccc3)cc21] 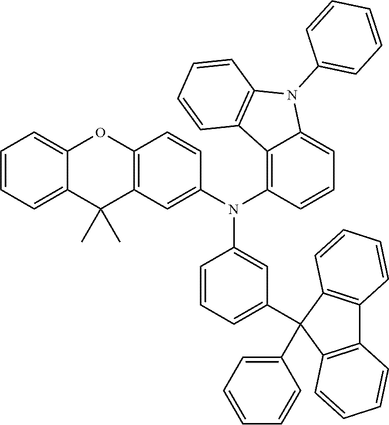 [SMILES: CC1(C)c2ccccc2Oc2ccc(N(c3cccc(C4(c5ccccc5)c5ccccc5-c5ccccc54)c3)c3ccc4c(c3)-c3ccccc3C4(C)C)cc21] 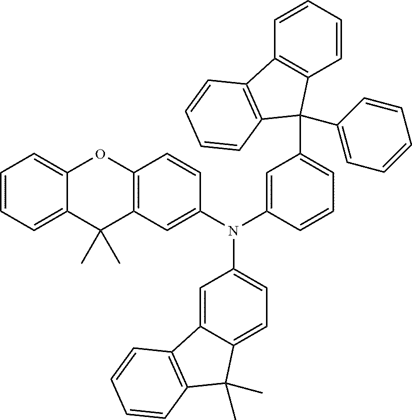 [SMILES: CC1(C)c2ccccc2Oc2ccc(N(c3cccc(C4(c5ccccc5)c5ccccc5-c5ccccc54)c3)c3cccc4c3-c3ccccc3C4(C)C)cc21] 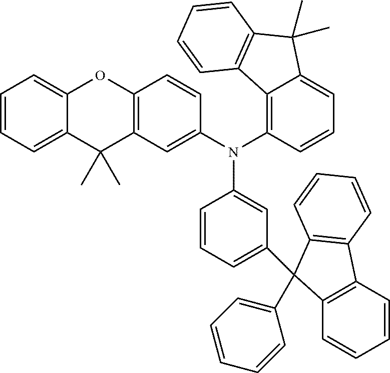 [SMILES: CC1(C)c2ccccc2Oc2ccc(N(c3cccc(C4(c5ccccc5)c5ccccc5-c5ccccc54)c3)c3cccc4c3-c3ccccc3C4(c3ccccc3)c3ccccc3)cc21] 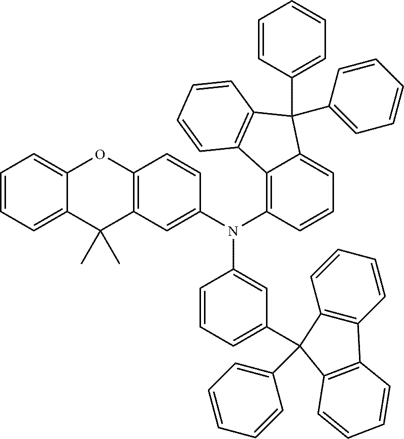 [SMILES: CC1(C)c2ccccc2Oc2ccc(N(c3cccc(C4(c5ccccc5)c5ccccc5-c5ccccc54)c3)c3cccc4c3-c3ccccc3C43c4ccccc4-c4ccccc43)cc21] 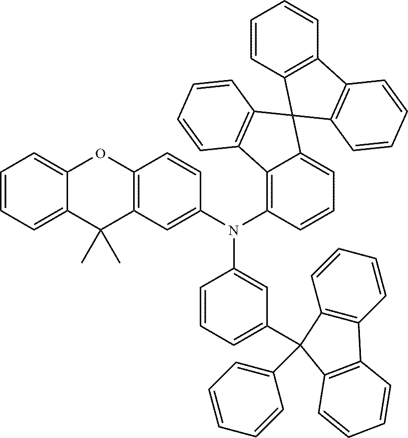 [SMILES: c1ccc(C2(c3ccccc3)c3ccccc3Oc3ccc(N(c4cccc(C5(c6ccccc6)c6ccccc6-c6ccccc65)c4)c4ccc5c(c4)oc4ccccc45)cc32)cc1] 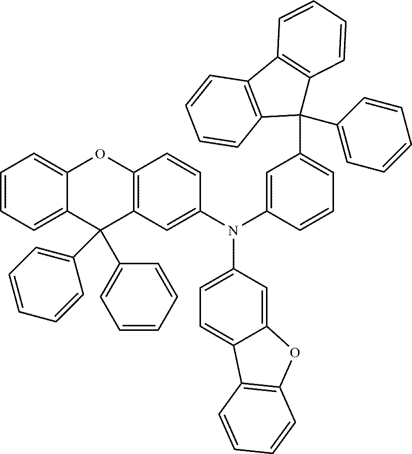 [SMILES: c1ccc(C2(c3cccc(N(c4ccc5c(c4)C4(c6ccccc6O5)c5ccccc5-c5ccccc54)c4ccc5c(c4)oc4ccccc45)c3)c3ccccc3-c3ccccc32)cc1] 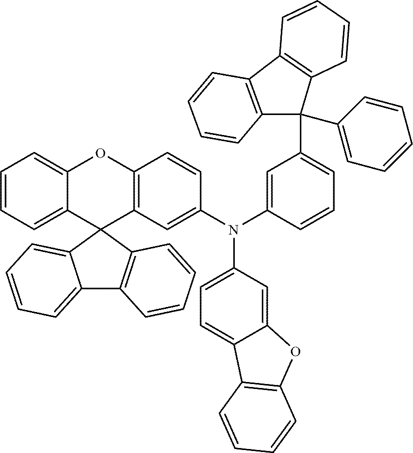 [SMILES: CC1(C)c2ccccc2Oc2cc(N(c3cccc(C4(c5ccccc5)c5ccccc5-c5ccccc54)c3)c3ccc4sc5ccccc5c4c3)ccc21] 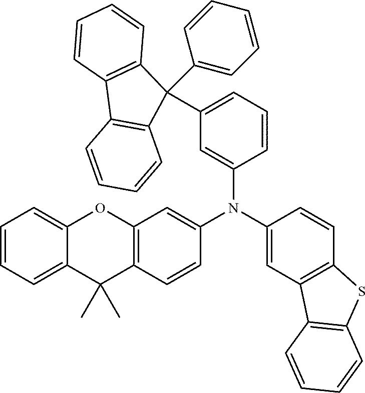 [SMILES: CC1(C)c2ccccc2Oc2c(N(c3cccc(C4(c5ccccc5)c5ccccc5-c5ccccc54)c3)c3cccc4c3-c3ccccc3C4(C)C)cccc21] 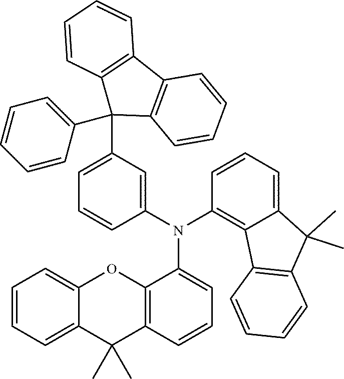 [SMILES: CC1(C)c2ccccc2Oc2cccc(N(c3cccc(C4(c5ccccc5)c5ccccc5-c5ccccc54)c3)c3ccc4c5ccccc5n(-c5ccccc5)c4c3)c21] 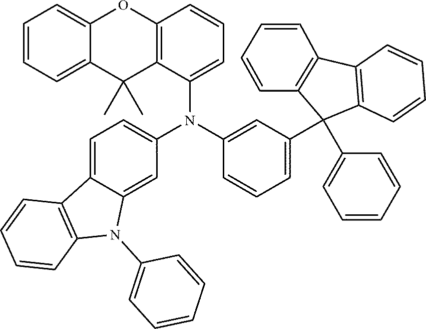 [SMILES: c1ccc(C2(c3ccc(N(c4cccc(C5(c6ccccc6)c6ccccc6-c6ccccc65)c4)c4cccc5oc6ccccc6c45)cc3)c3ccccc3Oc3ccccc32)cc1] 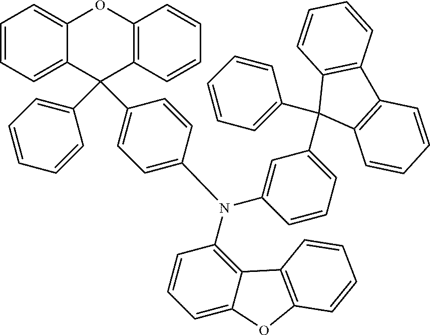 [SMILES: c1ccc(-n2c3ccccc3c3cc(N(c4cccc(C5(c6ccccc6)c6ccccc6Oc6ccccc65)c4)c4cccc(C5(c6ccccc6)c6ccccc6-c6ccccc65)c4)ccc32)cc1] 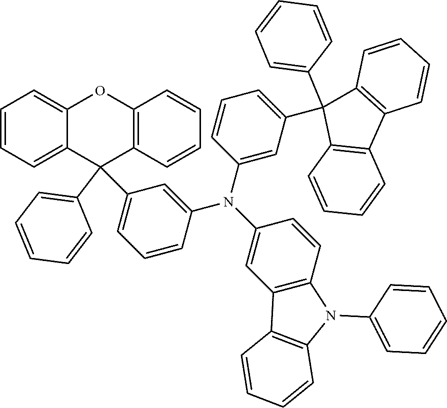 [SMILES: c1ccc(C2(c3cccc(N(c4ccc5c(c4)oc4cc6ccccc6cc45)c4ccccc4C4(c5ccccc5)c5ccccc5Oc5ccccc54)c3)c3ccccc3-c3ccccc32)cc1] 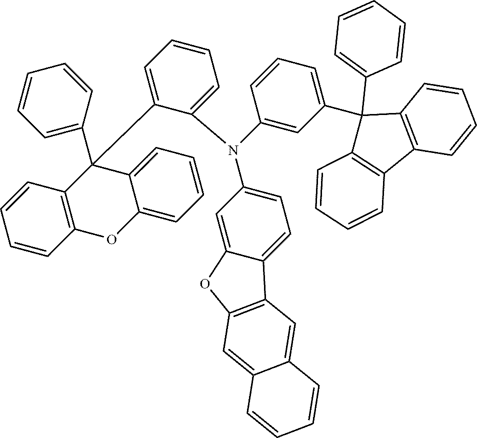 [SMILES: CC1(C)c2ccccc2-c2ccc(N(c3cccc(C4(c5ccccc5)c5ccccc5-c5ccccc54)c3)c3ccc4c(c3)C3(c5ccccc5Oc5ccccc53)c3ccccc3-4)cc21] 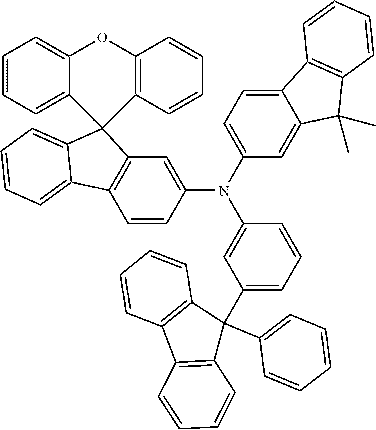 [SMILES: CC1(C)c2ccccc2Sc2ccc(N(c3cccc(C4(c5ccccc5)c5ccccc5-c5ccccc54)c3)c3ccc4c(c3)oc3ccccc34)cc21] 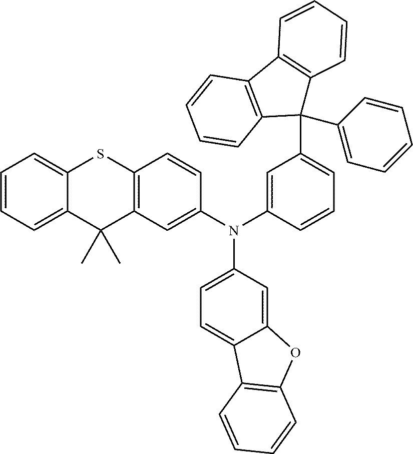 [SMILES: CC1(C)c2ccccc2Sc2ccc(N(c3cccc(C4(c5ccccc5)c5ccccc5-c5ccccc54)c3)c3ccc4c(c3)c3ccccc3n4-c3ccc4ccccc4c3)cc21] 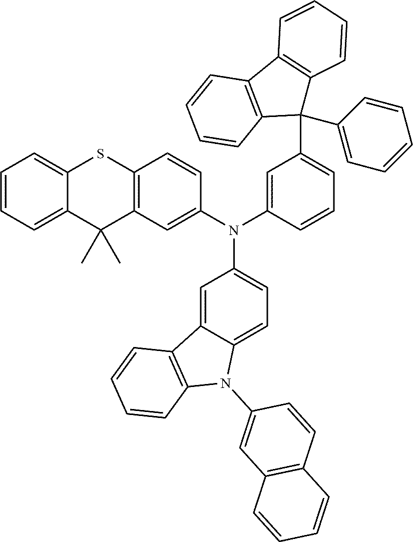 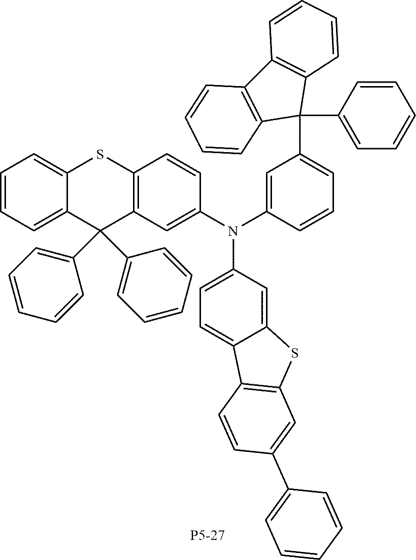 [SMILES: c1ccc(C2(c3cccc(N(c4ccc5c(c4)C4(c6ccccc6S5)c5ccccc5-c5ccccc54)c4ccc5oc6ccccc6c5c4)c3)c3ccccc3-c3ccccc32)cc1] 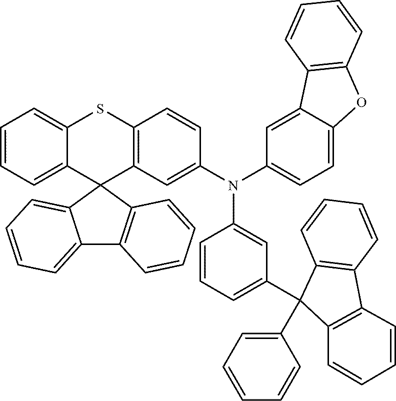 [SMILES: CC1(C)c2ccccc2Sc2cc(N(c3cccc(C4(c5ccccc5)c5ccccc5-c5ccccc54)c3)c3cc(-c4ccccc4)c4oc5ccccc5c4c3)ccc21] 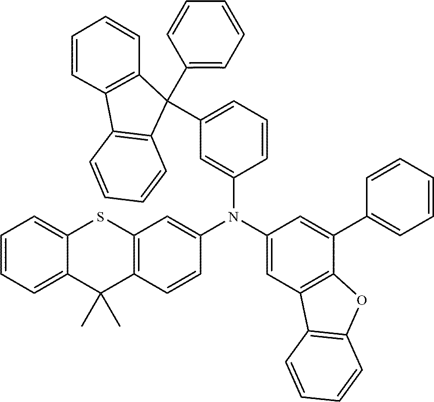 [SMILES: CC1(C)c2ccccc2Sc2c(N(c3cccc(-c4cccc5c4-c4ccccc4C5(C)C)c3)c3cccc(C4(c5ccccc5)c5ccccc5-c5ccccc54)c3)cccc21] 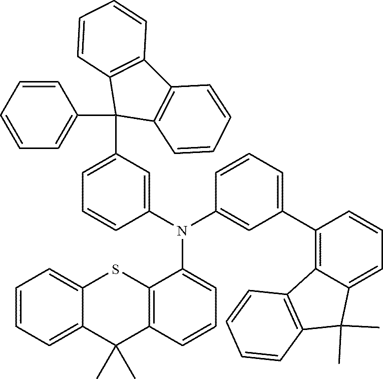 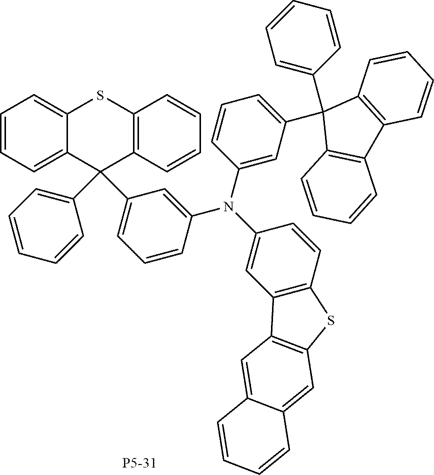 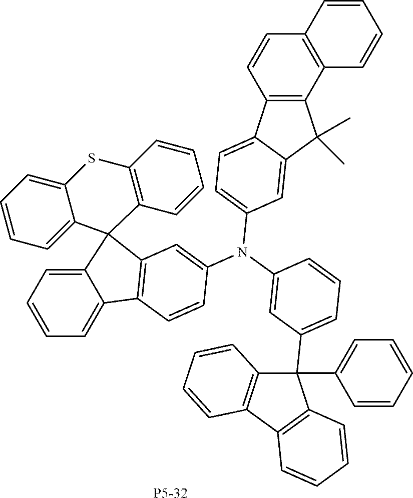 [SMILES: Fc1ccc2c(c1)oc1ccc(N(c3cccc(-c4cccc(C5(c6ccccc6)c6ccccc6-c6ccccc65)c4)c3)c3ccc4c(c3)C3(c5ccccc5O4)c4ccccc4-c4ccccc43)cc12] 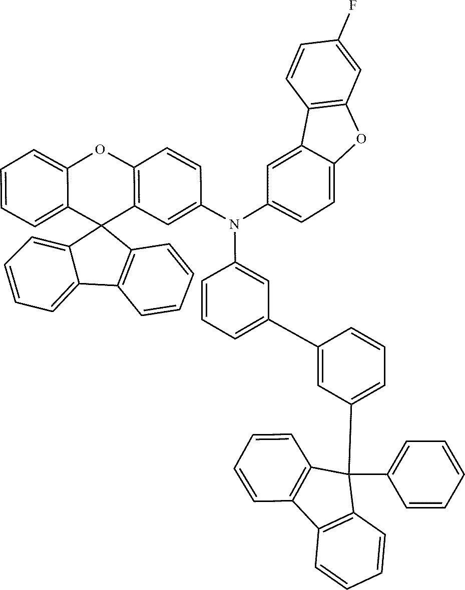 [SMILES: CC1(C)c2ccccc2-c2c(-c3ccc(N(c4cccc(-c5cccc(C6(c7ccccc7)c7ccccc7Sc7ccccc76)c5)c4)c4cccc(C5(c6ccccc6)c6ccccc6-c6ccccc65)c4)cc3)cccc21] 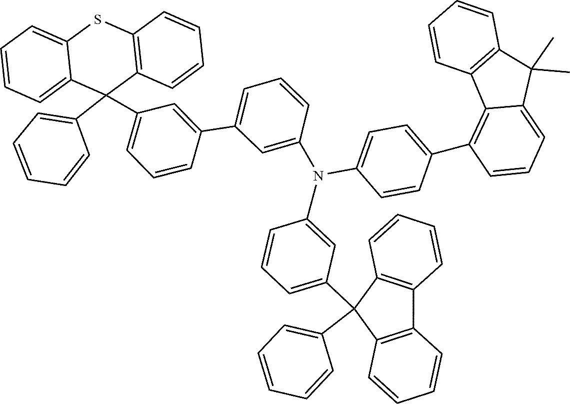 [SMILES: CC1(C)c2ccccc2Sc2ccc(-c3cccc(N(c4cccc(-c5cccc(C6(c7ccccc7)c7ccccc7-c7ccccc76)c5)c4)c4cccc(-c5cccc6c5sc5ccccc56)c4)c3)cc21] 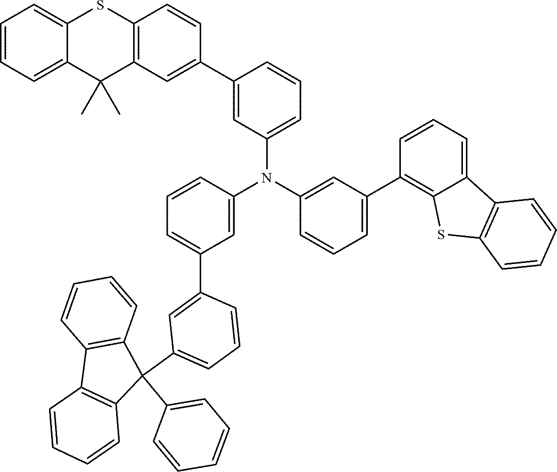 [SMILES: CC1(C)c2ccccc2Oc2cc(-c3ccc(N(c4cccc(-c5cccc(C6(c7ccccc7)c7ccccc7-c7ccccc76)c5)c4)c4ccc5c(c4)C(C)(C)c4ccccc4-5)cc3)ccc21] 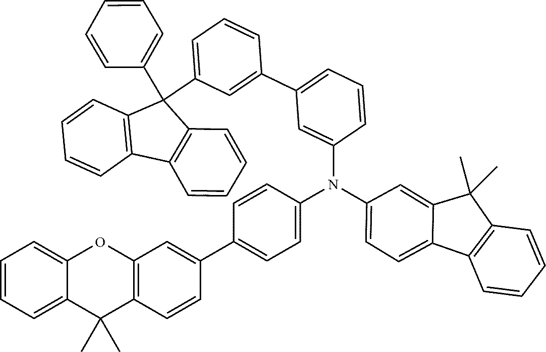 [SMILES: CC1(C)c2ccccc2-c2cc(-c3ccc(N(c4cccc(C5(C)c6ccccc6-c6ccccc65)c4)c4cccc5c4Sc4ccccc4C5(C)C)cc3)ccc21] 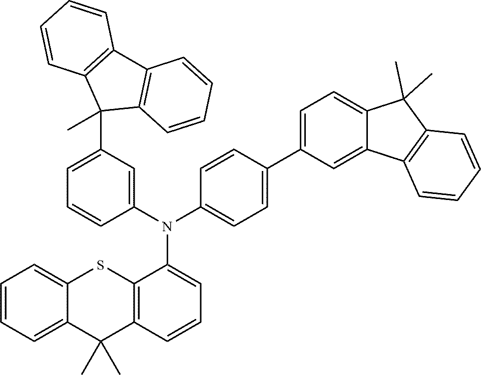 [SMILES: CC1(c2cccc(N(c3cccc(C4(c5ccccc5)c5ccccc5Oc5ccccc54)c3)c3ccc4c(c3)oc3cc5ccccc5cc34)c2)c2ccccc2-c2ccccc21] 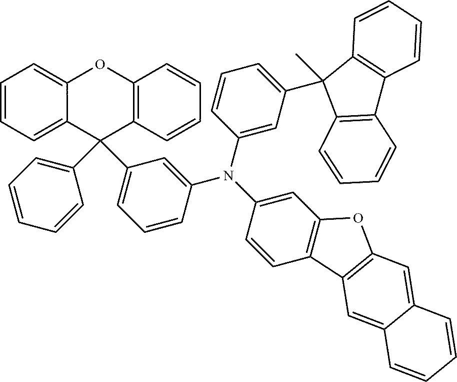 [SMILES: CC1(C)c2ccccc2Oc2ccc(N(c3ccc4sc5ccccc5c4c3)c3ccccc3C3(c4ccccc4)c4ccccc4-c4ccccc43)cc21] 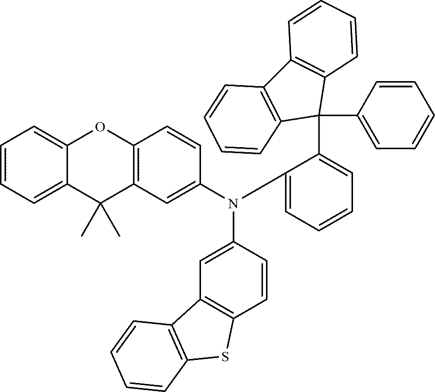 [SMILES: CC1(C)c2ccccc2Oc2ccc(N(c3ccc4c(c3)oc3ccccc34)c3ccccc3C3(c4ccccc4)c4ccccc4-c4ccccc43)cc21] 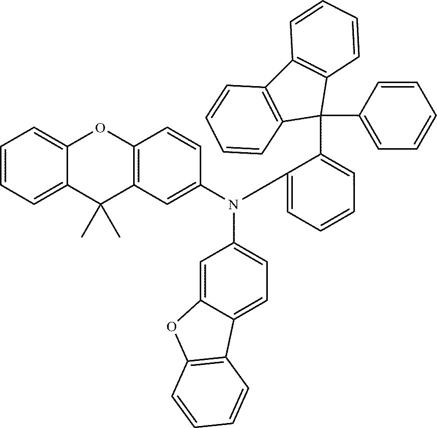 [SMILES: c1ccc(C2(c3ccccc3)c3ccccc3Oc3ccc(N(c4ccccc4C4(c5ccccc5)c5ccccc5-c5ccccc54)c4cccc5c4sc4ccccc45)cc32)cc1] 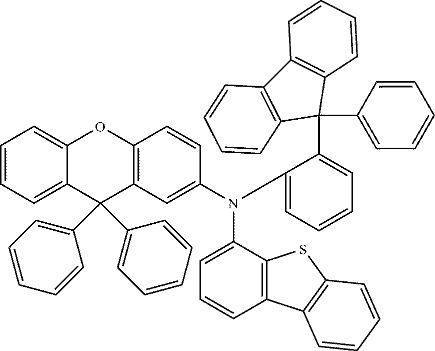 [SMILES: c1ccc(C2(c3ccccc3N(c3ccc4c(c3)C3(c5ccccc5O4)c4ccccc4-c4ccccc43)c3ccc4oc5ccc6ccccc6c5c4c3)c3ccccc3-c3ccccc32)cc1] 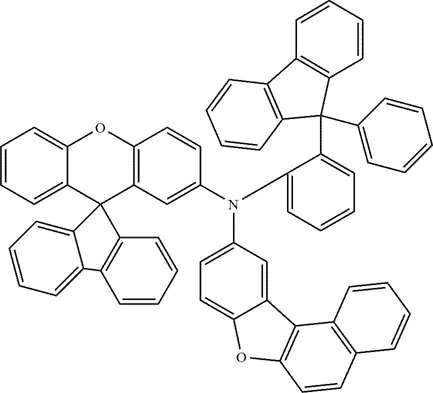 [SMILES: CC1(C)c2ccccc2Oc2cc(N(c3ccc4c5cc6ccccc6cc5n(-c5ccccc5)c4c3)c3ccccc3C3(c4ccccc4)c4ccccc4-c4ccccc43)ccc21] 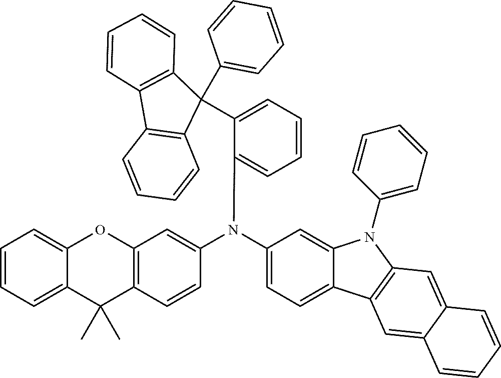 [SMILES: CC1(C)c2ccccc2-c2ccc(N(c3ccccc3C3(c4ccccc4)c4ccccc4-c4ccccc43)c3cccc4c3C(C)(C)c3ccccc3O4)cc21] 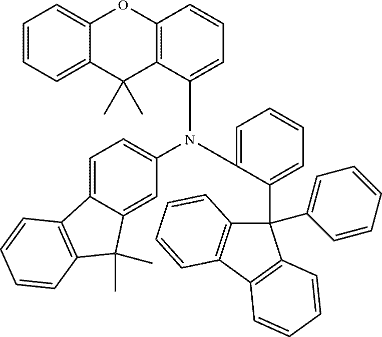 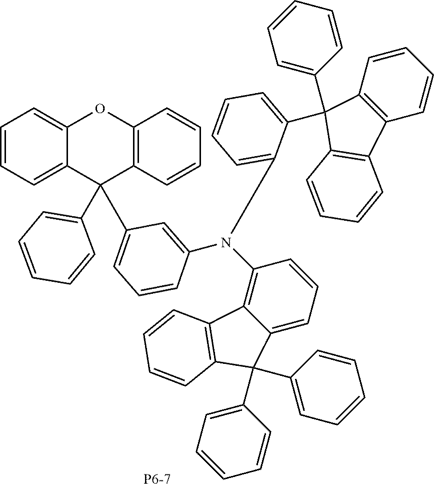 [SMILES: c1ccc(C2(c3ccccc3N(c3ccc4c(c3)C3(c5ccccc5Oc5ccccc53)c3ccccc3-4)c3ccc4c(c3)oc3ccccc34)c3ccccc3-c3ccccc32)cc1] 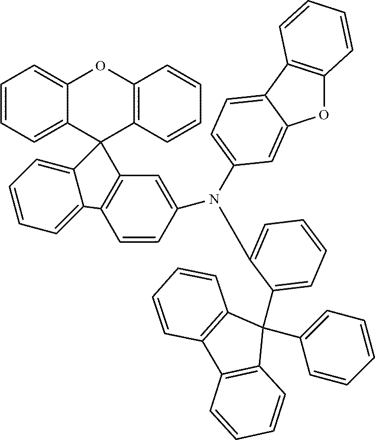 [SMILES: CC1(C)c2ccccc2Sc2ccc(N(c3ccc4c(c3)oc3cccc(-c5ccccc5)c34)c3ccccc3C3(c4ccccc4)c4ccccc4-c4ccccc43)cc21] 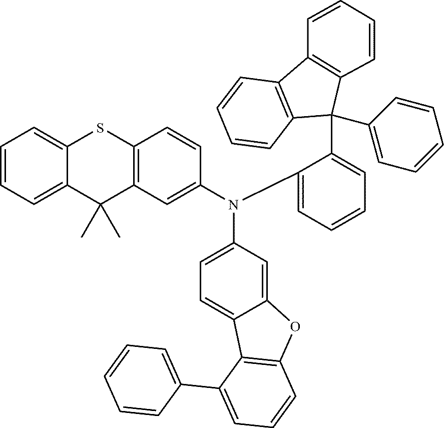 [SMILES: CC1(C)c2ccccc2Sc2ccc(N(c3ccccc3C3(c4ccccc4)c4ccccc4-c4ccccc43)c3cc4c5ccccc5n(-c5ccccc5)c4c4ccccc34)cc21] 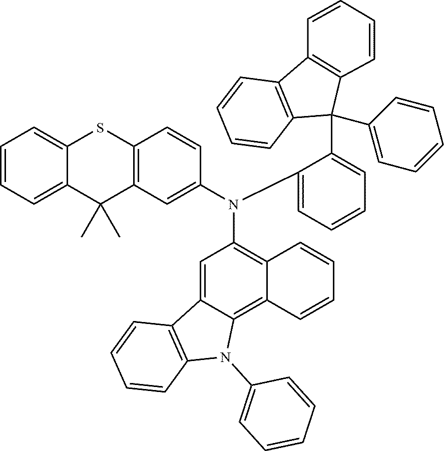 [SMILES: Fc1ccc2c(c1)Sc1ccc(N(c3ccc4sc5ccccc5c4c3)c3ccccc3C3(c4ccccc4)c4ccccc4-c4ccccc43)cc1C2(c1ccccc1)c1ccccc1] 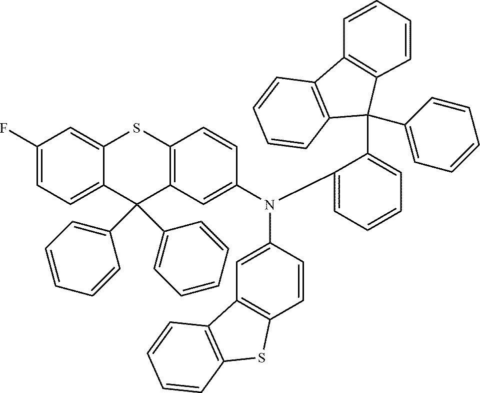 [SMILES: c1ccc(C2(c3ccccc3N(c3ccc4c(c3)C3(c5ccccc5S4)c4ccccc4-c4ccccc43)c3ccc4c(c3)sc3ccccc34)c3ccccc3-c3ccccc32)cc1] 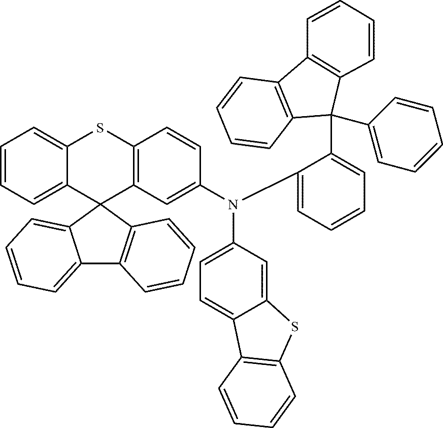 [SMILES: CC1(C)c2ccccc2Sc2cc(N(c3ccccc3C3(c4ccccc4)c4ccccc4-c4ccccc43)c3cccc4c3-c3ccccc3C43c4ccccc4-c4ccccc43)ccc21] 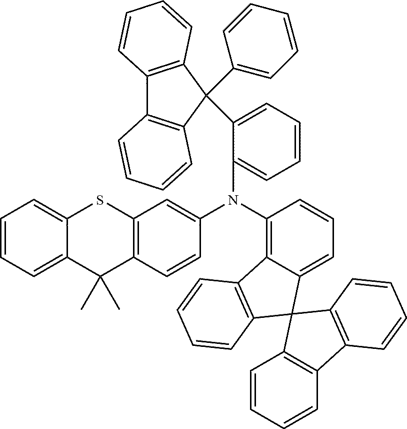 [SMILES: CC1(C)c2ccccc2Sc2cccc(N(c3ccc4c(c3)oc3ccc(-c5ccccc5)cc34)c3ccccc3C3(c4ccccc4)c4ccccc4-c4ccccc43)c21] 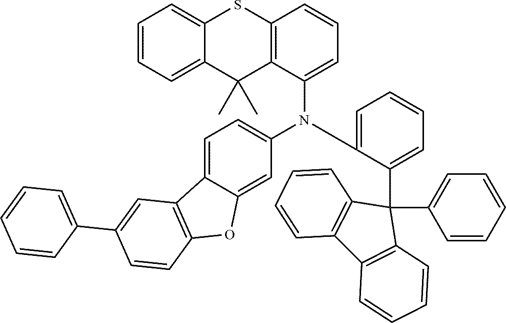 [SMILES: c1ccc(C2(c3cccc(N(c4ccccc4C4(c5ccccc5)c5ccccc5-c5ccccc54)c4cccc5sc6ccccc6c45)c3)c3ccccc3Sc3ccccc32)cc1] 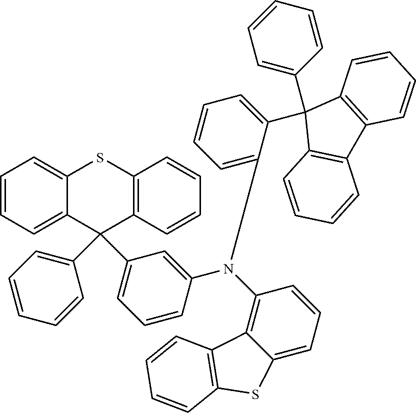 [SMILES: CC1(C)c2ccccc2-c2ccc(N(c3ccc4c(c3)C3(c5ccccc5Sc5ccccc53)c3ccccc3-4)c3ccccc3C3(c4ccccc4)c4ccccc4-c4ccccc43)cc21] 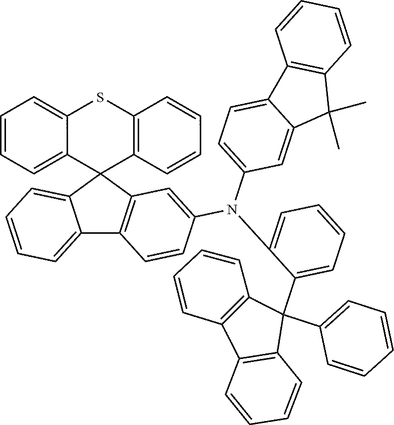 [SMILES: c1ccc(-n2c3cc4ccccc4cc3c3c(N(c4ccc(-c5ccc6c(c5)C5(c7ccccc7Sc7ccccc75)c5ccccc5-6)cc4)c4cccc(-c5ccccc5C5(c6ccccc6)c6ccccc6-c6ccccc65)c4)cccc32)cc1] 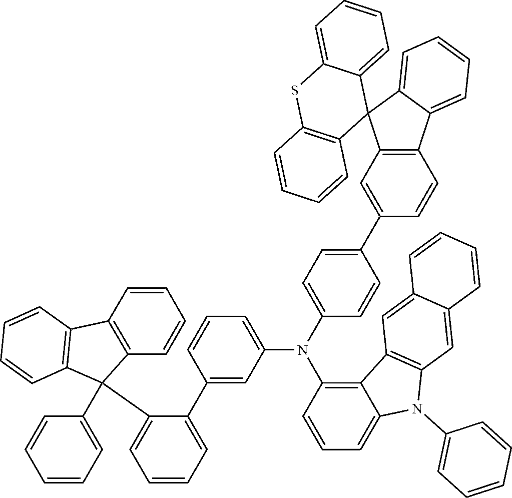 [SMILES: CC1(C)c2ccccc2Sc2ccc(N(c3ccc(-c4ccc5c(c4)oc4ccccc45)cc3)c3cccc(-c4ccccc4C4(c5ccccc5)c5ccccc5-c5ccccc54)c3)cc21] 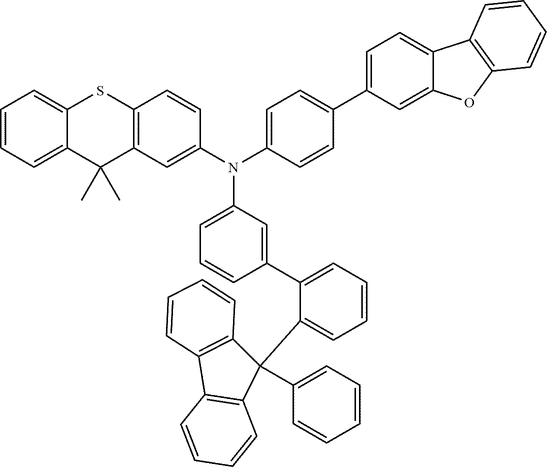 [SMILES: CC1(C)c2ccccc2-c2ccc(-c3ccc(N(c4cccc(-c5ccc6c(c5)C(c5ccccc5)(c5ccccc5)c5ccccc5O6)c4)c4ccccc4-c4ccccc4C4(c5ccccc5)c5ccccc5-c5ccccc54)cc3)cc21] 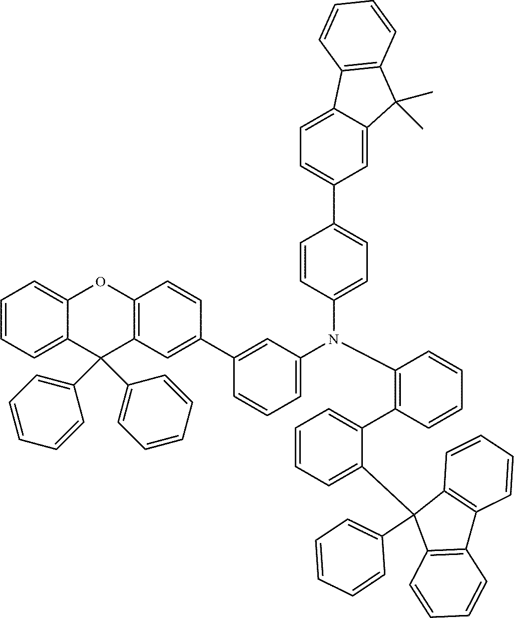 [SMILES: CC1(C)c2ccccc2Oc2c(-c3ccc(N(c4ccc(-c5cccc6c5sc5ccccc56)cc4)c4ccccc4C4(c5ccccc5)c5ccccc5-c5ccccc54)cc3)cccc21] 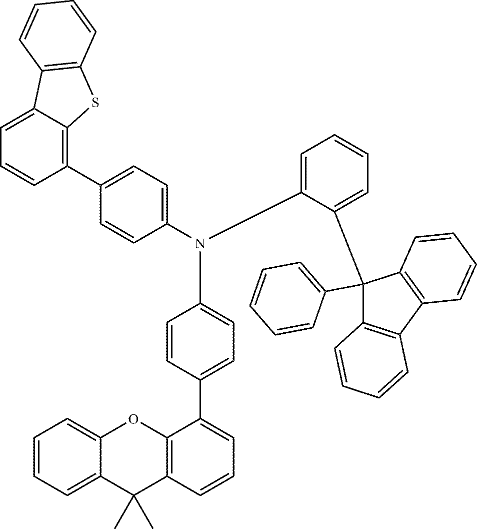 [SMILES: CC1(C)c2ccccc2Oc2ccc(N(c3cccc(-c4ccccc4C4(C)c5ccccc5-c5ccccc54)c3)c3ccc4oc5ccccc5c4c3)cc21] 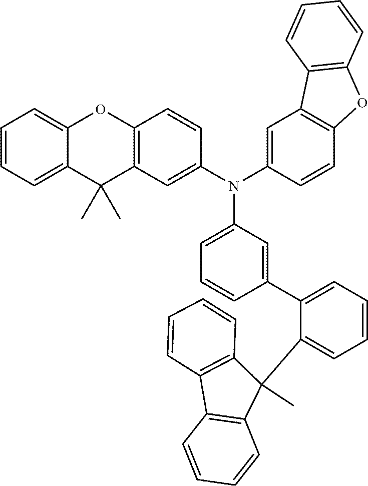 [SMILES: CC1(C)c2ccccc2Sc2c(-c3ccc(N(c4ccc5c(c4)sc4ccccc45)c4ccccc4C4(C)c5ccccc5-c5ccccc54)cc3)cccc21] 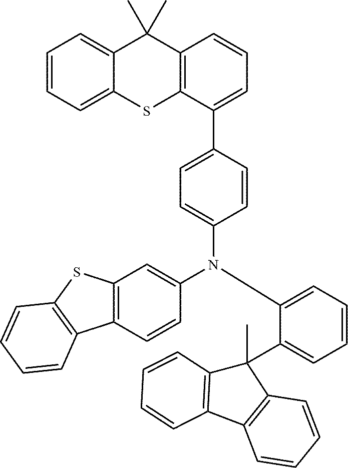 [SMILES: c1ccc(N(c2ccc(C3(c4ccccc4)c4ccccc4-c4ccccc43)cc2)c2cccc(C3(c4ccccc4)c4ccccc4Oc4ccc(N(c5ccccc5)c5ccc(C6(c7ccccc7)c7ccccc7-c7ccccc76)cc5)cc43)c2)cc1] 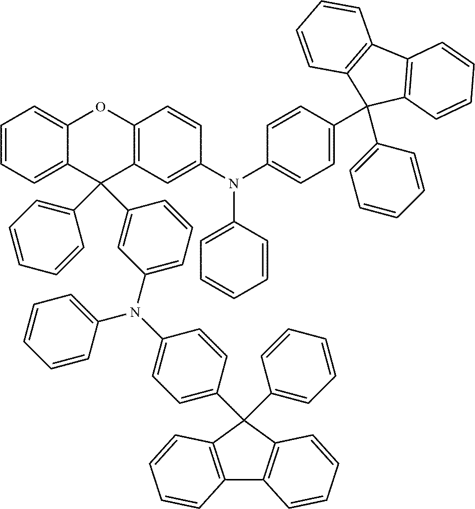 [SMILES: c1ccc(N(c2cccc(C3(c4ccccc4)c4ccccc4-c4ccccc43)c2)c2ccc3c(c2)C2(c4ccccc4S3)c3ccccc3-c3c(N(c4ccccc4)c4cccc(C5(c6ccccc6)c6ccccc6-c6ccccc65)c4)cccc32)cc1] 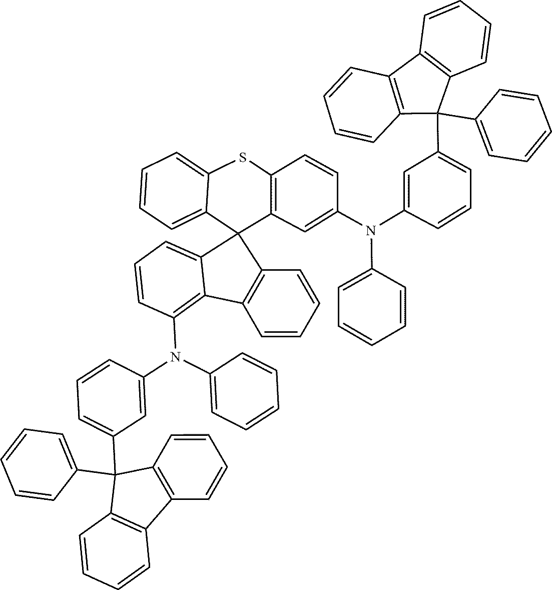 [SMILES: c1ccc(-c2ccc(N(c3ccc(C4(c5ccccc5)c5ccccc5Oc5ccc(N(c6cccc(C7(c8ccccc8)c8ccccc8-c8ccccc87)c6)c6ccc7c(c6)sc6ccccc67)cc54)cc3)c3ccc(C4(c5ccccc5)c5ccccc5-c5ccccc54)cc3)cc2)cc1] 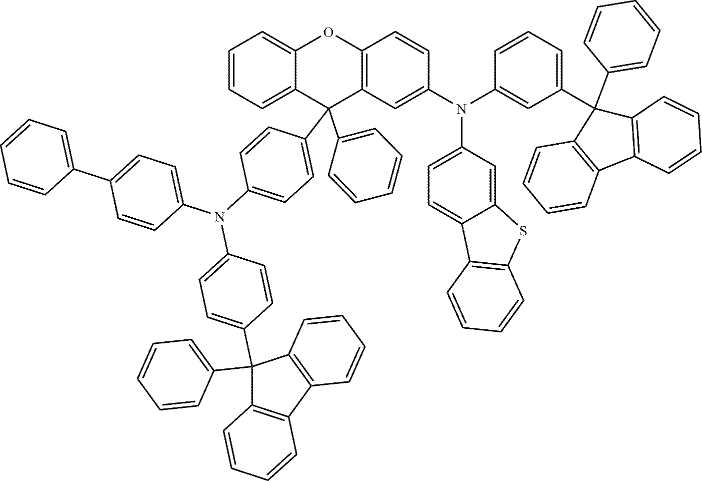 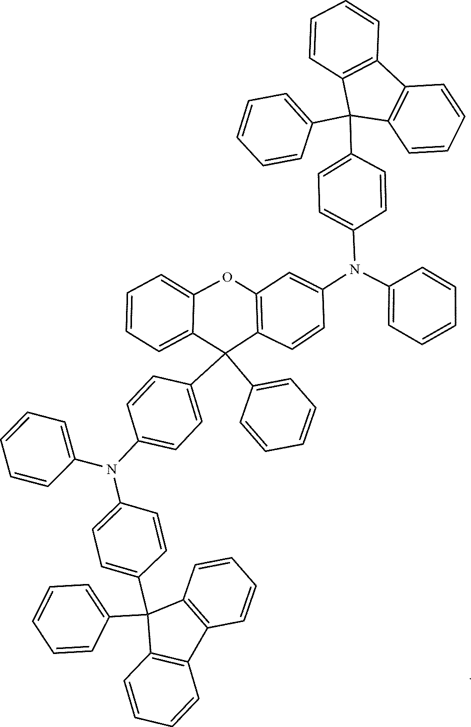
.

11. An organic electronic element comprising an anode, a cathode, and an organic material layer formed between the anode and the cathode, wherein the organic material layer comprises a single compound or 2 or more compounds represented by Formula 1 of claim 1.

12. The organic electronic element of claim 11, wherein the organic material layer comprises at least one of a hole injection layer, a hole transport layer, an emitting-auxiliary layer, an emitting layer, an electron transport auxiliary layer, an electron transport layer, and an electron injection layer.

13. The organic electronic element of claim 11, wherein the organic material layer comprises a hole transport layer comprising the compound.

14. The organic electronic element of claim 11, wherein the organic material layer comprises an emitting auxiliary layer comprising the compound.

15. The organic electronic element of claim 11, wherein the organic material layer comprises an emitting layer comprising the compound as a phosphorescent host material .

16. The organic electronic element of claim 11, wherein the organic electronic element further comprises a light efficiency enhancing layer formed on at least one surface of the anode and the cathode, the surface being opposite to the organic material layer.

17. The organic electronic element of claim 11, wherein the organic material layer comprises 2 or more stacks, each stack comprising a hole transport layer, an emitting layer, and an electron transport layer sequentially formed on the anode.

18. The organic electronic element of claim 17, wherein the organic material layer further comprises a charge generation layer formed between the 2 or more stacks.

19. An electronic device comprising: a display device including the organic electronic element of claim 11; and a control unit for driving the display device.

20. The organic electronic device of claim 19, wherein the organic electronic element is any one of an organic electroluminescent device (OLED), an organic solar cell, an organic photo conductor (OPC), an organic transistor (organic TFT), and an element for monochromic or white illumination.
